(19) United States
(12) Patent Application Publication
(10) Pub. No.: US 2010/0167270 A1
(43) Pub. Date: Jul. 1, 2010

(54) METAPNEUMOVIRUS STRAINS AND THEIR USE IN VACCINE FORMULATIONS AND AS VECTORS FOR EXPRESSION OF ANTIGENIC SEQUENCES AND METHODS FOR PROPAGATING VIRUS

(75) Inventor: Jeanne H. Schickli, Sunnyvale, CA (US)

Correspondence Address: MEDIMMUNE, LLC;Patrick Scott Alban, ONE MEDIMMUNE WAY, GAITHERSBURG, MD, 20878, US

(73) Assignee: Medlmmune,LLC, Gaithersburg, MD (US)

(21) Appl. No.: 12/640,808

(22) Filed: Dec. 17, 2009

Related U.S. Application Data
(63) Continuation of application No. 11/373,686, filed on Mar. 9, 2006, now abandoned.
(60) Provisional application No. 60/660,735, filed on Mar. 10, 2005.

(51) Int. Cl. C12Q 1/70; C12N 7/01; C07H 21/04

(52) U.S. Cl. 435/5; 435/235.1; 536/23.72

(57) ABSTRACT

The invention relates to improved strains of mammalian negative strand RNA virus, metapneumovirus (MPV), within the sub-family Pneumoviridae, of the family Paramyxoviridae. The invention further relates to methods for propagating mammalian MPV in the absence of trypsin. The methods and compositions of the invention can be used for the preparation of vaccines against, e.g., MPV infections.

### RELATED APPLICATIONS

This application claims the benefit of priority of U.S. provisional application No. 60/660,735 filed Mar. 10, 2005, the entire disclosure of which is incorporated by reference herein in its entirety.

### 1. INTRODUCTION

The invention relates to improved strains of mammalian negative strand RNA virus, metapneumovirus (MPV), within the sub-family Pneumoviridae, of the family Paramyxoviridae. The invention further relates to methods for propagating mammalian MPV in the absence of trypsin. The methods and compositions of the invention can be used for the preparation of vaccines against, e.g., MPV infections.

### 2. BACKGROUND OF THE INVENTION

Human metapneumovirus (hMPV) is a recently identified respiratory virus that was initially isolated from children in the Netherlands experiencing symptoms of acute respiratory disease with undetermined etiology. hMPV causes respiratory illness ranging from mild upper respiratory symptoms to severe lower respiratory disease such as bronchiolitis and pneumonia (Boivin et al, 2002; van den Hoogen et al, 2001, 2003;). Depending on the patient population sampled, between 5 and 15% of respiratory infections in young children may be attributable to hMPV infection (Boivin, 2003; Williams et al, 2004; van den Hoogen, 2004b). hMPV is also associated with 12 to 50% of otitis media in children. (Boivin 2003; Peiris 2003; van den Hoogen, 2004b). In the Netherlands, 55% of tested individuals were seropositive for hMPV by age 2 and nearly all individuals 5 years and older were seropositive (van den Hoogen, 2001). The distribution of hMPV is worldwide, with reports from Europe, North America, Australia, Africa, Israel, Japan and Hong Kong (Bastien et al, 2003b; Howe, 2002; Hamelin et al 2004; Upma et al 2004; Maggi et al, 2003; Nissen et al, 2002; Peiris 2003; Peret et al, 2002; Stockton et al, 2002; Wolf et al 2003). Testing of archived serum samples indicated that hMPV has been circulating in the population for at least 50 years (van de Hoogen et al, 2001). One reason why it has only been recently identified is that it grows poorly in cell culture with minimal cytopathetic effects (Hamelin et al, 2004;van den Hoogen et al 2001).

hMPV is an enveloped single-stranded negative-sense RNA virus of the Pneumovirinae subfamily in the Paramyxoviridae family that also includes respiratory syncytial virus (RSV), avian pneumovirus (APV) and pneumovirus of mice (Van den Hoogen et al, 2001). Based on homology with other pneumoviruses, 8 transcription units have been identified in the following order: 3′ N-P-M-F-M2-SH-G-L 5′ (Toquin et al, 2003; van den Hoogen 2002). Phylogenetic analysis divides the hMPV strains into two genetic clusters, designated subgroups A and B that are distinct from APV viruses (Bastien et al 2003a and b; Biacchesi et al, 2003; Peret et al 2002 and 2004; van den Hoogen, 2002). Within these subgroups, hMPV can be further subdivided into A1, A2, B1, and B2 subtypes (van den Hoogen, 2003).

The fusion glycoprotein (F), which is highly conserved between subgroups A and B, presumably mediates virus penetration and syncytia formation. F proteins of pneumoviruses such as RSV and APV are synthesized as full-length precursors (F₀) that are subsequently cleaved at a polybasic furin-like cleavage site to form F₁ and F₂. Cleavage of F₀ exposes a fusion peptide at the N terminus of F₁ (Collins 2001; Lamb 1993; Morrison 2003, Russell et al 2001; White 1990). Unlike RSV and APV, hMPV contains a putative cleavage site RQS/PR that does not conform to the furin-like motif (Barr, 1991).

Isolation of hMPV from clinical samples in cell culture has been reported to be trypsin dependent (Bastien et al 2003a, Biacchesi et al, 2003; Boivin et al, 2002; Skiadopoulos et al, 2004; van den Hoogen et al 2001 and 2004a). Therefore, it was unexpected that two isolates of hMPV, strains hMPV/NL/1/00 and hMPV/NL/1/99, grew in Vero cells without addition of trypsin. Equally high titers were achieved in the absence or presence of trypsin.

RT-PCR products of wild type (wt) hMPV/NL/1/00 and wt hMPV/NL/1/99 were sequenced and it was found that a mutation that encodes the amino acid substitution S101P in the RQSR motif at the putative cleavage site of F protein, when compared to published sequences GI:20150834 and GI:50059145. In the results reported here, it is demonstrated that for both strains hMPV/NL/1/00 and hMPV/NL/1/99, representing A1 and B1 subtypes of hMPV, respectively, viruses harboring 101P in the RQSR motif at the putative cleavage site of the F glycoprotein was able to replicate in Vero cells without exogenously added trypsin. In contrast, hMPV harboring 101S in the F protein required addition of a protease such as trypsin for viral growth. In this report, in vitro growth properties, cleavage properties of hMPV F glycoprotein variants and syncytia formation of recombinant viruses with amino acid substitutions near the putative cleavage site in the absence and presence of trypsin were evaluated. S101P in hMPV F was found to be the major genetic determinant that enhanced the cleavage efficiency of F and increased its fusion activity, both of which likely contributed to efficient Vero cell growth of wt hMPV/NL/1/00 and wt hMPV/NL/1/99 in the absence of trypsin. The bibliography of the cited references is set forth at the end of Section 6.

### 3. SUMMARY OF THE INVENTION

The present invention provides a method for propagating mammalian metapneumovirus, wherein the method comprises culturing the mammalian metapneumovirus in medium with a specific trypsin activity of less than 20 milliunits per milliliter of medium. In certain aspects, the mammalian metapneumovirus is human metapneumovirus. In certain aspects, no trypsin is added exogenously to the medium. In certain aspects, no serum is added to the medium. In certain aspects, an RQSR cleavage motif in the cleavage site of the F protein of mammalian metapneumovirus comprises at least one amino acid substitution. In certain aspects, the F protein of mammalian metapneumovirus comprises at least one additional amino acid substitution relative to SEQ ID NO:314. In certain aspects, the amino acid substitution in the RQSR cleavage motif is a serine to proline substitution resulting in a RQPR sequence. In certain aspects, the additional amino acid substitution in the F protein is at least one of the following E93K, Q100K, E92K, E93V, I95S, E96K, Q94K, Q94H, I95S, N97K or N97H. In certain aspects, the additional amino acid substitution in the F protein is E93K. In certain aspects, the additional amino acid substitution stabilizes the amino acid substitution in the RQSR motif.

In certain embodiments, the invention provides an isolated mammalian metapneumovirus, wherein the mammalian metapneumovirus is capable of growth in the absence of trypsin. In certain aspects, the mammalian metapneumovirus is human metapneumovirus. In certain aspects, an RQSR cleavage motif in the cleavage site of the F protein of mammalian metapneumovirus comprises at least one amino acid substitution. In certain aspects, the F protein of mammalian metapneumovirus comprises at least one additional amino acid substitution relative to SEQ ID NO:314. In certain aspects, the amino acid substitution in the RQSR cleavage motif is a serine to proline substitution resulting in a RQPR sequence. In certain aspects, the additional amino acid substitution in the F protein is at least one of the following E93K, Q100K, E92K, E93V, I95S, E96K, Q94K, Q94H, I95S, N97K or N97H. In certain aspects, the additional amino acid substitution stabilizes the amino acid substitution in the RQSR motif. In certain aspects, the additional amino acid substitution in the F protein is E93K. In certain aspects, the isolated nucleic acid encodes an F protein of a mammalian metapneumovirus, wherein the F protein comprises the S101P amino acid substitution and at least one of the following amino acid substitutions E93K, Q100K, E92K, E93V, I95S, E96K, Q94K, Q94H, I95S, N97K or N97H. In certain aspects, the mammalian metapneumovirus is human metapneumovirus.

In certain embodiments, the invention provides a method for identifying an F protein of a mammalian metapneumovirus that supports stable growth of the mammalian metapneumovirus in the absence of trypsin, the method comprising: (a) growing the mammalian metapneumovirus in the absence of trypsin for at least two passages, wherein the mammalian metapneumovirus comprises a RQPR motif in the cleavage site of the F protein; and (b) measuring syncytia formation; wherein increased syncytia formation relative to syncytia formation by a mammalian metapneumovirus prior to step (a) indicates that the F protein of the mammalian metapneumovirus has acquired an additional amino acid substitution that supports stable growth of the mammalian metapneumovirus in the absence of trypsin. In certain aspects, the mammalian metapneumovirus is human metapneumovirus. In certain aspects, the mammalian metapneumovirus carries the S101P mutation.

In certain embodiments, the invention provides a method for identifying an F protein of a mammalian metapneumovirus that supports stable growth of the mammalian metapneumovirus in the absence of trypsin, the method comprising: (a) growing the mammalian metapneumovirus in the absence of trypsin for at least two passages, wherein the mammalian metapneumovirus comprises a RQPR motif in the cleavage site of the F protein; and (b)

measuring F protein cleavage; wherein increased F protein cleavage relative to F protein cleavage by mammalian metapneumovirus prior to step (a) indicates that the F protein of the mammalian metapneumovirus has acquired an additional amino acid substitution that supports stable growth of the mammalian metapneumovirus in the absence of trypsin. In certain aspects, the mammalian metapneumovirus is human metapneumovirus. In certain aspects, the mammalian metapneumovirus carries the S101P mutation.

In certain embodiments, the invention provides a method for identifying an F protein mutant of a mammalian metapneumovirus that enhances trypsin-independent cleavage of the F protein, wherein the F protein comprises a RQPR motif in the cleavage site, said method comprising: (a) growing the mammalian metapneumovirus in the absence of trypsin for at least two passages; and (b) determining the cleave efficiency of the F protein, wherein increased cleavage efficiency of the F protein indicates that the F protein has acquired a mutation that enhances trypsin-independent cleavage of the F protein. In certain aspects, the mammalian metapneumovirus is human metapneumovirus. In certain aspects, the mammalian metapneumovirus carries the S101P mutation.

In certain embodiments, the invention provides a method for identifying a protease that catalyzes the cleavage of an F protein of mammalian metapneumovirus, wherein the F protein comprises a RQPR motif in the cleavage site, said method comprising: (a) contacting the F protein with a test protease; and (b) determining whether cleavage of the F protein has occurred; wherein the occurrence of cleavage of the F protein indicates that the protease catalyzes the cleavage of the F protein. In certain aspects, the mammalian metapneumovirus is human metapneumovirus. In certain aspects, the mammalian metapneumovirus carries the S101P mutation.

### 3.1 CONVENTIONS AND ABBREVIATIONS

cDNA complementary DNA
L large protein
M matrix protein (lines inside of envelope)
F fusion glycoprotein
HN hemagglutinin-neuraminidase glycoprotein
N, NP or NC nucleoprotein (associated with RNA and required for polymerase activity)
P phosphoprotein
MOI multiplicity of infection
NA neuraminidase (envelope glycoprotein)
PIV parainfluenza virus
hPIV human parainfluenza virus
hPIV3 human parainfluenza virus type 3
APV/hMPV recombinant APV with hMPV sequences
hMPV/APV recombinant hMPV with APV sequences
Mammalian MPV mammalian metapneumovirus
nt nucleotide
RNP ribonucleoprotein
rRNP recombinant RNP
vRNA genomic virus RNA
cRNA antigenomic virus RNA
hMPV human metapneumovirus
APV avian pneumovirus
MVA modified vaccinia virus Ankara
FACS Fluorescence Activated Cell Sorter
CPE cytopathic effects
Position 1 Position of the first gene of the viral genome to be transcribed
Position 2 Position between the first and the second open reading frame of the native viral genome, or alternatively, the position of the second gene of the viral genome to be transcribed
Position 3 Position between the second and the third open reading frame of the native viral genome, or alternatively, the position of the third gene of the viral genome to be transcribed.
Position 4 Position between the third and the fourth open reading frame of the native viral genome, or alternatively, the position of the fourth gene of the viral genome to be transcribed.
Position 5 Position between the fourth and the fifth open reading frame of the native viral genome, or alternatively, the position of the fifth gene of the viral genome to be transcribed.
Position 6 Position between the fifth and the sixth open reading frame of the native viral genome, or alternatively, the position of the sixth gene of the viral genome to be transcribed.
Ab antibody
dpi days post-infection
F fusion
HAI hemagglutination-inhibition
HN hemagglutinin-neuraminidase
hpi hours post-infection
MOI multiplicity of infection
POI point of infection
bPIV-3 bovine parainfluenza virus type 3)
hPIV-3 human parainfluenza virus type 3
RSV respiratory syncytial virus
SFM serum-free medium
TCID₅₀ 50% tissue culture infective dose

### 4. DESCRIPTION OF THE FIGURES

FIG. 1: Titers and Plaques of 4 subtypes of hMPV. Subconfluent monolayers of Vero cells were inoculated with each of the indicated biologically derived viruses at a MOI of 0.1 PFU/cell and +/−0.2 ug/ml TPCK trypsin. The cells and supernatant were collected 6 days post inoculation, frozen at −70 C and titered in Vero cells by plaque assay. Infected cell monolayers were grown under 1% methylcellulose, fixed in methanol 6 days post inoculation and immunostained with ferret anti-hMPV polyclonal Ab, followed by horse-radish peroxidase-conjugated anti-ferret Ab. Plaques were visualized with 3-amino-9-ethylcarbazole (AEC) and photographed using a Nikon eclipse TE2000-U microscope. Titers are expressed as log₁₀ PFU/ml.

FIG. 2: Comparison of growth properties of rhMPV/NL/1/00/101P and rhMPV/NL/1/00/101S, representative of subtype A1. (A) Plaques produced by rhMPV/NL/1/00/101P and rhMPV/NL/1/00/101S grown in Vero cells +/−0.2 ug/ml trypsin and immunostained 6 days post inoculation with ferret anti-hMPV polyclonal Ab followed by horse radish peroxidase-conjugated anti-ferret Ab and color was developed by addition of 3-amino-9-ethylcarbazole (AEC) chromogen (Dako). (B) 6-day growth curves of Vero cells infected with either rhMPV/NL/1/00/101P (open squares) or rhMPV/NL/1/00/101S (closed triangles). In the graph on the left, 0.2 ug/ml trypsin was added during virus propagation and during plaque assay in Vero cells. In the middle graph, no trypsin was used. In the graph on the right, no trypsin was used during virus propagation, but 0.2 ug/ml trypsin was added during the plaque assay procedure. Titers were determined by plaque assay as described in materials and methods. (C) Vero cell monolayers were inoculated with either rhMPV/NL/1/00/101P or rhMPV/NL/1/00/101S +/−0.2 ug/ml trypsin. Infected cell monolayers were fixed in 3% paraformaldehyde and immunostained with hamster Mab 121-1017-133 directed to hMPV F followed by FITC-conjugated anti-hamster Ab to visualize surface expression of hMPV F with a Nikon TE2000-U microscope. (D) Western blot of Vero cell monolayers infected with either rhMPV/NL/1/00/101P or rhMPV/NL/1/00/101S with +/−0.2 ug/ml trypsin as described in materials and methods. Virus samples were separated on a 12% SDS-PAGE gel, transferred to a PVDF membrane, immunoblotted with hamster Mab 121-1017-133 directed to hMPV F followed by HRP-conjugated anti-hamster Ab, treated with electrochemoluminescence solution and exposed to film. The numbers at left are molecular mass of markers in kilodaltons. The arrows at right indicate positions of two bands corresponding to the predicted sizes of full-length hMPV F (F₀) and cleavage fragment hMPV F₁.

FIG. 3: Expression of hMPV F vectored in b/h PIV3 as detected by Western blot. Subconfluent monolayers of Vero cells were inoculated with wt hMPV/NL/1/00, b/h PIV3/hMPV F/101P, or b/h PIV3/hMPV F/101S with +/−trypsin as described in the text. Western blot analysis using Mab 121-1017-133 directed to hMPV F was done as described in materials and methods. Numbers at left are the molecular mass of the markers in kilodaltons. The arrows at right indicate positions of two bands corresponding to the predicted sizes of full-length hMPV F (F₀) and cleavage fragment hMPV F₁.

FIG. 4: Chromatograms of nucleotide sequences derived from recombinant, variant and wild type hMPV viruses. RT-PCR was done as described in material and methods. The chromatograms shown extend from nucleotides 3348 to 3373. The codons corresponding to the predicted amino acids 93 (rectangles), 100 (ovals) and 101 (underlined) of F glycoprotein are indicated. An asterisk indicates either a mutation or polymorphism.

FIG. 5: Relative cleavage efficiencies of hMPV F protein as detected by Western blot. Vero cells were inoculated with the indicated hMPV virus either +/−0.2 ug/ml trypsin, at a MOI of 0.1 PFU/cell. The viruses were: rhMPV/NL/1/00/101S (lanes 1, 6, 13 and 18), rhMPV/NL/1/00/101P (lanes 2 and 7, 11 and 16), vhMPV/93K/101P (lanes 3 and 8), vhMPV/100K/101P (lanes 4 and 9), wt hMPV/NL/1/00 (lanes 5, 10,15 and 20), rhMPV/93K/101P (lanes 12 and 17), or rhMPV/93K/101S (lanes 14 and 19). Note that wt hMPV/NL/1/00 is a mixture of hMPV with E93K and hMPV with Q100K as described in the text. 6 days post inoculation, cells and supernatants were collected, frozen at −70° C., thawed and separated on a 12% SDS-PAGE gel. Proteins were transferred to a PVDF membrane and probed with Mab 122-1017-133 directed to hMPV F. Numbers at left are molecular mass of markers in kilodaltons. Arrows at right indicate two bands corresponding to the predicted sizes of full-length hMPV F (F₀) and cleavage fragment hMPV F₁. The hMPV F amino acids in positions 93, 100 and 101 of each virus are indicated for each lane above the blot. The presence or absence of trypsin is indicated below the blot.

FIG. 6: Multicycle growth curves of recombinant, variant and wild type hMPV viruses containing 101P in the F protein. Subconfluent monolayers of Vero cells were inoculated at a MOI of 0.1 PFU/cell without trypsin. Cells and supernatants were collected over 6 days at 24 h intervals. The titers of the collected viruses were determined by plaque assay.

FIG. 7: Relative fusion efficiencies of Vero cell monolayers infected with hMPV viruses. Confluent monolayers of Vero cells were inoculated with the indicated hMPV viruses at MOI of 3 PFU/cell +/−0.2 ug/ml TPCK trypsin and grown under medium containing 1% methyl cellulose. The monolayers were fixed in methanol at 48 h. The nuclei were visualized by incubation with Heochst stain and imaged by a DAPI lens on a Nikon eclipse TE2000-U fluorescence microscope. The photos shown are representative of one field of view from one of three independent experiments. Aggregated nuclei of fused cells and single nuclei of unfused cells were counted in 10 fields of view and the percentage of fused cells was graphed. The data shown is from one of three experiments.

FIG. 8: Comparison of growth properties of wt hMPV/1/99/101P and rhMPV/1/99/101S, representative of subtype B1. (A) Plaques produced by wt hMPV/NL/1/99/101P or rhMPV/NL/99/101S, each +/−0.2 ug/ml trypsin in Vero cells immunostained 6 days post inoculation. (B) 6-day growth curves of Vero cells infected with either wt hMPV/NL/1/99/101P (open squares) or rhMPV/NL/1/99/101S (closed triangles). In the graph on the left, 0.2 ug/ml trypsin was added during virus propagation and plaquing. In the middle graph, no trypsin was used. In the graph on the right, no trypsin was used during virus propagation, but 0.2 ug/ml trypsin was added during the plaquing procedure. Titers were determined by plaque assay as described in materials and methods. (C) Vero cell monolayers were inoculated with either wt hMPV/NL/1/99/101P or rhMPV/NL/1/99/101S +/−0.2 ug/ml trypsin. Infected cell monolayers were fixed in 3% paraformaldehyde and immunostained with hamster Mab 121-1017-133 directed to hMPV F followed by FITC-conjugated anti-hamster Ab to visualize surface expression of hMPV F with a Nikon TE2000-U microscope. (D) Western blot of Vero cell monolayers infected with either wt hMPV/NL/1/99/101P or rhMPV/NL/1/99/101S +/−0.2 ug/ml trypsin as described in material and methods. Virus samples were separated on a 12% SDS-PAGE gel, transferred to a PVDF membrane, immunoblotted with hamster Mab 121-1017-133 directed to hMPV F, followed by HRP-conjugated anti-hamster Ab, treated with electrochemoluminescence solution and exposed to film. Numbers at left are molecular mass of markers in kilodaltons. The arrows at right indicate positions of two bands corresponding to the predicted sizes of full-length hMPV F (F₀) and cleavage fragment hMPV F₁.

FIG. 9: hMPV genome analysis: PCR fragments of hMPV genomic sequence relative to the hMPV genomic organization are shown. The position of mutations are shown underneath the vertical bars indicating the PCR fragments.

FIG. 10: Restriction maps of hMPV isolate 00-1 (A1) and hMPV isolate 99-1 (B1). Restriction sites in the respective isolates are indicated underneath the diagram showing the genomic organization of hMPV. The scale on top of the diagram indicates the position in the hMPV genome in kb.

### 5. DETAILED DESCRIPTION OF THE INVENTION

Metanpneumovirus Strains

The present invention provides isolated mammalian metapneumovirus strains that can be propagated in the absence of trypsin. In certain embodiments, the invention provides a recombinant mammalian, e.g., human, metapneumovirus that has been engineered to be able to propagate in the absence of trypsin. Without being bound by theory, the mammalian metapneumovirus strains of the invention can be propagated in the absence of trypsin because the F protein is cleaved trypsin-independently. In certain specific embodiments, the mammalian metapneumovirus is a human metapneumovirus. In certain aspects, the mammalian metapneumovirus is a recombinant metapneumovirus. In certain specific embodiments, the mammalian metapneumovirus is a recombinant human metapneumovirus (rhMPV).

In certain embodiments, the invention provides mammalian metapneumovirus strains that can be propagated without exogenously added trypsin. In certain embodiments, the invention provides mammalian metapneumovirus strains that can be propagated at trypsin concentrations which would result in a specific trypsin activity of less than 40 milliunits per milliliter of medium, less than 35 milliunits per milliliter of medium, less than 30 milliunits per milliliter of medium, less than 25 milliunits per milliliter of medium, less than 20 milliunits per milliliter of medium, less than 15 milliunits per milliliter of medium, less than 10 milliunits per milliliter of medium, less than 5 milliunits per milliliter of medium, less than 2 milliunits per milliliter of medium, less than 1 milliunit per milliliter of medium, or less than 0.5 milliunits per milliliter of medium. In certain embodiments, the invention provides mammalian metapneumovirus strains that can be propagated at trypsin concentrations in the medium at less than 0.1 microgram of trypsin per milliliter of medium, at less than 0.05 microgram of trypsin per milliliter of medium; at less than 0.01 microgram of trypsin per milliliter of medium; at less than 0.005 microgram of trypsin per milliliter of medium; at less than 0.001 microgram of trypsin per milliliter of medium; or at less than 0.0005 microgram of trypsin per milliliter of medium.

In certain embodiments of the invention one or more amino acid(s) in the RQSR motif in the cleavage site of the F protein is substituted or deleted. In certain embodiments, the serine of the RQSR motif in the cleavage site of the F protein in a mammalian metapneumovirus of the invention is substituted with a different amino acid. In more specific embodiments, the serine in the RQSR motif in the cleavage site of the F protein is substituted with a proline resulting in an RQPR motif. In order to reduce the likelihood of reversion to the wild-type genotype, an amino acid substitution can be engineered by introducing at least 2 nucleotide exchanges in the codon that encodes the amino acid.

In an illustrative example, the F protein has the amino acid sequence of SEQ ID NO: 314 (amino acid sequence of the F protein of human metapneumovirus strain NL/1/00) and the serine at amino acid position 101 is replaced by a proline to obtain a mammalian metapneumovirus that can be propagated trypsin-independently. The skilled artisan knows how to identify the homologous amino acid positions in the F protein of a different strain of mammalian metapneumovirus by aligning the amino sequences of the F protein of the different strain with, e.g., the amino acid sequence of SEQ ID NO:314. For example, SEQ ID NO:314 is aligned with the amino acid sequence of the F protein of another human metapneumovirus strain, the RQSR sequence of SEQ ID NO:314 (amino acid positions 99 to 102) is located and the corresponding amino acids in the F protein of a different strain of mammalian metapneumovirus are identified.

In certain embodiments of the invention, the F protein comprises one or more additional mutations (“second site mutations”), such as amino acid substitutions, additions, or deletions, relative to SEQ ID NO:314 in addition to the substitution of the serine in the RQSR motif of the cleavage site in the F protein. Without being bound by theory, such a second site mutation stabilizes the substitution of the serine in the RQSR motif of the cleavage site in the F protein such that any further mutations in the F protein of the mammalian metapneumovirus strain occur less frequently than in the mammalian metapneumovirus strain without the second site mutation when grown in the absence of trypsin. Further, without being bound by theory, such second site mutations enhance the trypsin independent cleavage of the F protein. In certain embodiments, a mammalian metapneumovirus strain of the invention that carries a second site mutation can go through at least 2, 3, 4, 5, 6, 7, 8, 9, 10, 15, 20, or at least 25 passages in the absence of trypsin without acquiring any spontaneous mutations in the F protein in addition to the substitution of the serine in the RQSR motif of the cleavage site in the F protein.

In certain embodiments of the invention, a second site mutation is in a gene different from the F gene. Without being bound by theory, cleavage of the F protein is dependent from the molecular context of the F protein such that alterations in proteins that affect, e.g., the folding of the F protein or the orientation of the F protein in the viral particle can also affect the cleavage of the F protein.

In certain embodiments, the second site mutation is in the vicinity of the RQPR motif in the cleavage site of the F protein. In certain embodiments, the second site mutation is within 20 amino acids, within 15 amino acids, within 10 amino acids, or within 5 amino acid amino-terminal from the RQPR motif. In certain embodiments, the second site mutation is within 20 amino acids, within 15 amino acids, within 10 amino acids, or within 5 amino acid carboxy-terminal from the RQPR motif.

In certain more specific embodiments, the second site mutation is at an amino acid position of the F protein that corresponds to amino acid position 92, 93, 94, 95, 96, 97, or 100 of SEQ ID NO:314. In certain, even more specific embodiments, the additional mutation can be E93K, Q100K, E92K, E93V, I95S, E96K, Q94K, Q94H, I95S, N97K or N97H, wherein the first letter refers to the amino acid in SEQ ID NO:314, the number refers to the amino acid position, and the second letter refers to the amino acid that replaces the amino acid of SEQ ID NO:314 at the respective position.

In certain embodiments, a metapneumovirus of the invention has the RQPR motif, e.g., by carrying the S101P mutation, and a second site mutation. In a specific, illustrative embodiment, the invention provides a recombinant human metapneumovirus that comprises an F protein, wherein the F protein comprises the E93K and S101P amino acid substitutions.

In certain embodiments, the mutations in the F protein of the viruses of the invention do not result in a change in host specificity of the mammalian metapneumovirus. In certain embodiments, the mutations in the F protein of the viruses of the invention do not result in a change in host cell specificity of the mammalian metapneumovirus.

The mammalian metapneumovirus strains of the invention are useful, e.g., for the development of live attenuated virus vaccines.

In certain embodiments, two or three mutations are introduced into one codon to effect the amino acid substitution. Without being bound by theory, having more than one mutation in one codon will reduce the reversion rate to the wild type genotype.

The metapneumovirus strains of the invention can be geneticall modified to encode a heterologous sequence. In certain embodiments, the metapneumovirus strains of the invention can be modified to encode an antigenic peptide, polypeptide or protein. Such modified metapneumoviruses can be used in vaccines as further described hereinbelow. The metapneumovirus strains of the invention can further be geneticall modified to be attenuated in a specific host (see hereinbelow; see section 5.7).

Methods of Propagating

The present invention provides methods for propagating mammalian metapneumovirus in the absence of trypsin. In certain embodiments, the mammalian metapneumovirus is a recombinant mammalian, e.g., human, metapneumovirus that has been engineered to be able to propagate in the absence of trypsin. Without being bound by theory, mammalian metapneumovirus strains can be propagated in the absence of trypsin if their F protein is cleaved trypsin independently. In certain more specific embodiments, the mammalian metapneumovirus is a human metapneumovirus. In certain aspects, the mammalian metapneumovirus is a recombinant metapneumovirus. In certain specific embodiments, the mammalian metapneumovirus is a recombinant human metapneumovirus (rhMPV).

In certain embodiments, the invention provides methods for propagating mammalian metapneumovirus without exogenously adding trypsin to the medium. In certain embodiments, the invention provides methods for propagating mammalian metapneumovirus strains that can be propagated at trypsin concentrations which would result in a specific trypsin activity of less than 40 milliunits per milliliter of medium, less than 35 milliunits per milliliter of medium, less than 30 milliunits per milliliter of medium, less than 25 milliunits per milliliter of medium, less than 20 milliunits per milliliter of medium, less than 15 milliunits per milliliter of medium, less than 10 milliunits per milliliter of medium, less than 5 milliunits per milliliter of medium, less than 2 milliunits per milliliter of medium, less than 1 milliunit per milliliter of medium, or less than 0.5 milliunits per milliliter of medium. In certain embodiments, the invention provides methods for propagating mammalian metapneumovirus at trypsin concentrations in the medium at less than 0.1 microgram of trypsin per milliliter of medium, at less than 0.05 microgram of trypsin per milliliter of medium; at less than 0.01 microgram of trypsin per milliliter of medium; at less than 0.005 microgram of trypsin per milliliter of medium; at less than 0.001 microgram of trypsin per milliliter of medium; or at less than 0.0005 microgram of trypsin per milliliter of medium. In certain other embodiments, trypsin is inactivated with an inhibitor of trypsin activity.

In certain embodiments of the invention one or more amino acid(s) in the RQSR motif in the cleavage site of the F protein is substituted or deleted. In certain embodiments, the serine of the RQSR motif in the cleavage site of the F protein of the mammalian metapneumovirus that is propagated using the methods of the invention is substituted with a different amino acid to confer trypsin-independent growth on the metapneumovirus. In more specific embodiments, the serine in the RQSR motif in the cleavage site of the F protein of the mammalian metapneumovirus that is propagated using the methods of the invention is substituted with a proline.

In an illustrative example, the F protein of the mammalian metapneumovirus that is propagated using the methods of the invention has the amino acid sequence of SEQ ID NO: 314 and the serine at amino acid position 101 is replaced by a proline. The skilled artisan knows how to identify the homologous amino acid positions in the F protein of a different strain of mammalian metapneumovirus by aligning the amino sequences of the F protein of the different strain with, e.g., the amino acid sequence of SEQ ID NO:314.

In certain embodiments of the invention, the F protein of the mammalian metapneumovirus that is propagated using the methods of the invention comprises one or more mutations (“second site mutations”), such as amino acid substitutions, additions, or deletions, relative to SEQ ID NO:314 in addition to the substitution of the serine in the RQSR motif of the cleavage site in the F protein, e.g., the RQPR motif. Without being bound by theory, such a second site mutation stabilizes the substitution of the serine in the RQSR motif of the cleavage site in the F protein such that any further mutations in the F protein of the mammalian metapneumovirus strain occur less frequently than in the mammalian metapneumovirus strain without the second site mutation when the virus is grown in the absence of trypsin. In certain embodiments, a mammalian metapneumovirus strain with such a second site mutation and the substitution of the serine in the RQSR motif of the cleavage site in the F protein can go through at least 2, 3, 4, 5, 6, 7, 8, 9, 10, 15, 20, or at least 25 passages in the absence of trypsin without acquiring any spontaneous mutations in the F protein.

In certain embodiments, the second site mutation is in the vicinity of the RQPR motif in the cleavage site of the F protein. In certain embodiments, the second site mutation is within 20 amino acids, within 15 amino acids, within 10 amino acids, or within 5 amino acid amino-terminal from the RQPR motif. In certain embodiments, the second site mutation is within 20 amino acids, within 15 amino acids, within 10 amino acids, or within 5 amino acid carboxy-terminal from the RQPR motif.

In certain more specific embodiments, the second site mutation is at an amino acid position of the F protein that corresponds to amino acid position 92, 93, 94, 95, 96, 97, or 100 of SEQ ID NO:314. In certain, even more specific embodiments, the second site mutation can be E93K, Q100K, E92K, E93V, I95S, E96K, Q94K, Q94H, I95S, N97K or N97H, wherein the first letter refers to the amino acid in SEQ ID NO:314, the number refers to the amino acid position, and the second letter refers to the amino acid that replaces the amino acid of SEQ ID NO:314 at the respective position.

In certain embodiments of the invention, a second site mutation is in a gene different from the F gene. Without being bound by theory, cleavage of the F protein is dependent from the molecular context of the F protein such that alterations in proteins that affect, e.g., the folding of the F protein or the orientation of the F protein in the viral particle can also affect the cleavage of the F protein.

In a specific, illustrative embodiment, the invention provides a method for propagating a recombinant human metapneumovirus that comprises an F protein, wherein the F protein comprises the E93K and S101P amino acid substitutions in the absence of trypsin.

In certain embodiments, the invention provides methods for propagating a mammalian metapneumovirus without the addition of serum to the medium. For a more detailed description of growing infected cells in the absence of serum, see the section 5.6.

Illustrative cell lines that can be used with the methods of the invention include, but are not limited to, Vero cells and LLC-MK2 Rhesus Monkey Kidney. BHK cells can be used for the rescue of the mammalian metapneumovirus if recombinant virus is used with the methods of the invention.

In certain embodiments, the mutations in the F protein of the viruses that can be used with the methods of the invention do not result in a change in host specificity of the mammalian metapneumovirus. In certain embodiments, the mutations in the F protein of the viruses that can be used with the methods of the invention do not result in a change in host cell specificity of the mammalian metapneumovirus.

Screening Assays

The invention also provides methods for identifying second site mutations of trypsin-independent cleavage of the mammalian metapneumoviral F protein with the RQPR motif in the cleavage site. In certain embodiments, the invention provides screening methods for the identification of enhancers of the trypsin-independent cleavage of the mammalian metapneumoviral F protein with the RQPR motif in the cleavage site. Without being bound by any particular mechanism or theory, such second site mutations of trypsin-independent cleavage of the mammalian metapneumoviral F protein with the RQPR motif, e.g., enhancers, stabilize the viral genome such that growth in the absence of trypsin does not result in the accumulation of spontaneous additional mutations in the F gene. In certain embodiments, such second site modifiers are in the F gene. In certain other embodiments, such second site modifiers are in other genes of the mammalian metapneumovirus.

Mutations can be introduced into the F gene of the mammalian metapneumovirus by any method known to the skilled artisan. Mutations can be introduced by, e.g., random mutagenesis of the DNA and use of reverse genetics to rescue viral particles with the mutations; site-directed mutagenesis of the DNA and use of reverse genetics to rescue viral particles with the mutations; or growth of the virus under selective pressure, i.e., in the absence of trypsin.

Suitable second site mutations can be selected at different levels. In certain embodiments, DNA encoding the F protein is mutagenized, the F protein is expressed and tested for its ability to be cleaved trypsin independently (illustrative assays are described hereinbelow). Increase in trypsin independent cleavage indicates that the second site mutation is an enhancer of trypsin independent cleavage of the F protein. In other embodiments, DNA encoding the F gene is mutagenized, virus is rescued using reverse genetics, and the virus is tested for enhanced trypsin-independent F protein cleavage or increased syncytia formation. In even other embodiments, the virus is grown in the absence of trypsin, i.e., under selective pressure, and subsequently tested for the effect of any second site mutations, such as enhanced trypsin-independent F protein cleavage or increased syncytia formation.

Once mutants carrying second site modifiers in the F gene are selected, the F gene can be sequenced. Subsequently, the mutation can be introduced into a well-characterized strain, such as, but not limited to, rhMPV/NL/1/00/101P, to validate the effect of the second site mutation and to generate a viral strain that is suitable for vaccine production.

To identify a protease that cleaves the metapneumoviral F protein with the RQPR motif any method known to the skilled artisan can be employed to detect and quantify protease activity. In certain embodiments, detectably labeled F protein with the RQPR motif in the cleavage site is immobilized on a solid support such that cleavage of the F protein would result in loss of the label (i.e., the label is distal from the immobilization site relative to the cleavage site). Accordingly, protease activity can be detected and quantified by virtue of a decrease in detectable label. In other embodiments, the release of the detectably labeled amino acids or peptides of the polypeptide into the reaction buffer is measured. In certain other embodiments, FRET or fluorescence polarization is used to detect and quantify a protease reaction. In an illustrative example, the F protein is fluorescently labeled at the end not attached to the solid support. Upon incubation with the test protease, the fluorescent label is lost upon proteolysis, such that a decrease in fluorescence indicates the presence of protease activity capable of cleaving the F protein with the RQPR motif. In certain embodiments, the solid support is a bead.

The F protein can be detectably labeled by any method known to the skilled artisan. In certain embodiments, the protein or polypeptide is radioactively labeled. In certain embodiments, the protein or polypeptide is attached to the surface of the solid support on one end and is detectably labeled on the other end. The decrease of detectable label on the surface of the solid support is a measure for the activity of the protease activity.

Classes of proteases that can be used as test proteases include, but are not limited to, Bromelain, Cathepsins, Chymotrypsin, Collagenase, Elastase, Kallikrein, Papain, Pepsin, Plasmin, Renin, Streptokinase, Subtilisin, Thermolysin, Thrombin, Trypsin, and Urokinase. In a specific embodiments, the protease is Tryptase Clara or a homolog thereof.

### 5.1 Mammalian Methapneumovirus Structural Characteristics of a Mammalian Metapneumovirus

The invention provides a mammalian MPV. The mammalian MPV is a negative-sense single stranded RNA virus belonging to the sub-family Pneumovirinae of the family Paramyxoviridae. Moreover, the mammalian MPV is identifiable as phylogenetically corresponding to the genus Metapneumovirus, wherein the mammalian MPV is phylogenetically more closely related to a virus isolate deposited as 1-2614 with CNCM, Paris (SEQ ID NO:19) than to turkey rhinotracheitis virus, the etiological agent of avian rhinotracheitis. A virus is identifiable as phylogenetically corresponding to the genus Metapneumovirus by, e.g., obtaining nucleic acid sequence information of the virus and testing it in phylogenetic analyses. Any technique known to the skilled artisan can be used to determine phylogenetic relationships between strains of viruses. For exemplary methods see section 5.9. Other techniques are disclosed in International Patent Application PCT/NL02/00040, published as WO 02/057302, which is incorporated by reference in its entirety herein. In particular, PCT/NL02/00040 discloses nucleic acid sequences that are suitable for phylogenetic analysis at page 12, line 27 to page 19, line 29, which are incorporated by reference herein. A virus can further be identified as a mammalian MPV on the basis of sequence similarity as described in more detail below.

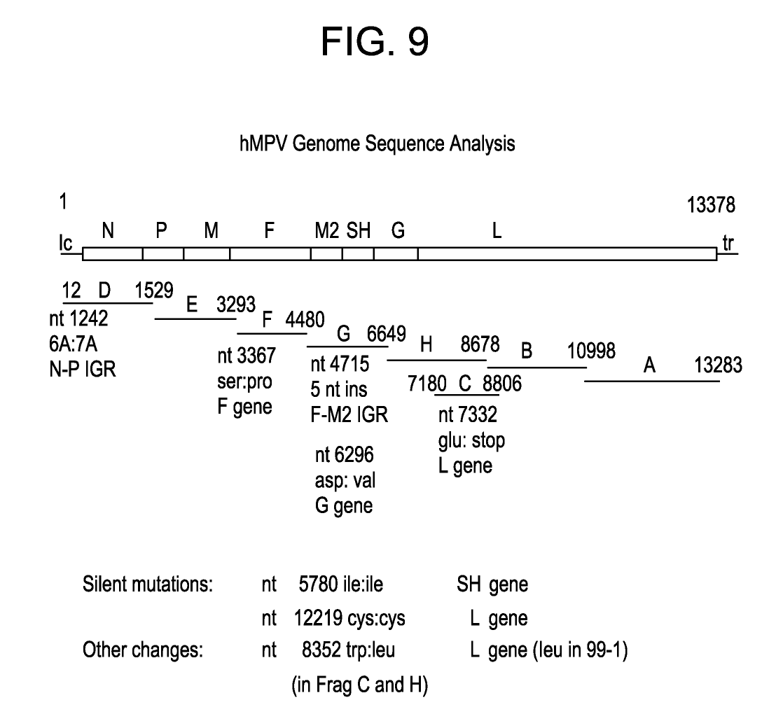

In addition to phylogenetic relatedness and sequence similarity of a virus to a mammalian MPV as disclosed herein, the similarity of the genomic organization of a virus to the genomic organization of a mammalian MPV disclosed herein can also be used to identify the virus as a mammalian MPV. For a representative genomic organization of a mammalian MPV see FIG. 9. In certain embodiments, the genomic organization of a mammalian MPV is different from the genomic organization of pneumoviruses within the sub-family Pneumovirinae of the family Paramyxoviridae. The classification of the two genera, metapneumovirus and pneumovirus, is based primarily on their gene constellation; metapneumoviruses generally lack non-structural proteins such as NS 1 or NS2 (see also Randhawa et al., 1997, J. Virol. 71:9849-9854) and the gene order is different from that of pneumoviruses (RSV: ‘3-NS1-NS2-N-P-M-SH-G-F-M2-L-5’, APV: ‘3-N-P-M-F-M2-SH-G-L-5’) (Lung, et al., 1992 J. Gen. Virol. 73:1709-17 15; Yu, et al., 1992, Virology 186:426-434; Randhawa, et al., 1997, J. Virol. 71:9849-9854).

Further, a mammalian MPV of the invention can be identified by its immunological properties. In certain embodiments, specific anti-sera can be raised against mammalian MPV that can neutralize mammalian MPV. Monoclonal and polyclonal antibodies can be raised against MPV that can also neutralize mammalian MPV. (See, PCT WO 02/057302 at pages ______ to ______, which is incorporated by reference herein.

The mammalian MPV of the invention is further characterized by its ability to infect a mammalian host, i.e., a mammalian cultured cell or a mammal. Unlike APV, mammalian MPV does not replicate or replicates only at low levels in chickens and turkeys. Mammalian MPV replicates, however, in mammalian hosts, such as cynomolgous macaques. In certain, more specific, embodiments, a mammalian MPV is further characterized by its ability to replicate in a mammalian host. In certain, more specific embodiments, a mammalian MPV is further characterized by its ability to cause the mammalian host to express proteins encoded by the genome of the mammalian MPV. In even more specific embodiments, the viral proteins expressed by the mammalian MPV are inserted into the cytoplasmic membranes of the mammalian host. In certain embodiments, the mammalian MPV of the invention can infect a mammalian host and cause the mammalian host to produce new infectious viral particles of the mammalian MPV. For a more detailed description of the functional characteristics of the mammalian MPV of the invention, see section 5.1.2.

In certain embodiments, the appearance of a virus in an electron microscope or its sensitivity to chloroform can be used to identify the virus as a mammalian MPV. The mammalian MPV of the invention appears in an electron microscope as paramyxovirus-like particle. Consistently, a mammalian MPV is sensitive to treatment with chloroform; a mammalian MPV is cultured optimally on tMK cells or cells functionally equivalent thereto and it is essentially trypsine dependent in most cell cultures. Furthermore, a mammalian MPV has a typical cytopathic effects (CPE) and lacks haemagglutinating activity against species of red blood cells. The CPE induced by MPV isolates are similar to the CPE induced by hRSV, with characteristic syncytia formation followed by rapid internal disruption of the cells and subsequent detachment from the culture plates. Although most paramyxoviruses have haemagglutinating activity, most of the pneumoviruses do not (Pringle, C. R. In: The Paramyxoviruses; (ed. D. W. Kingsbury) 1-39 (Plenum Press, New York, 1991)). A mammalian MPV contains a second overlapping ORF (M2-2) in the nucleic acid fragment encoding the M2 protein. The occurrence of this second overlapping ORF occurs in other pneumoviruses as shown in Alunadian et al., 1999, J. Gen. Vir. 80:2011-2016.

In certain embodiments, the invention provides methods to identify a viral isolate as a mammalian MPV. A test sample can, e.g., be obtained from an animal or human. The sample is then tested for the presence of a virus of the sub-family Pneumovirinae. If a virus of the sub-family Pneumovirinae is present, the virus can be tested for any of the characteristics of a mammalian MPV as discussed herein, such as, but not limited to, phylogenetic relatedness to a mammalian MPV, nucleotide sequence identity to a nucleotide sequence of a mammalian MPV, amino acid sequence identity/homology to a amino acid sequence of a mammalian MPV, and genomic organization. Furthermore, the virus can be identified as a mammalian MPV by cross-hybridization experiments using nucleic acid sequences from a MPV isolate, RT-PCR using primers specific to mammalian MPV, or in classical cross-serology experiments using antibodies directed against a mammalian MPV isolate. In certain other embodiments, a mammalian MPV can be identified on the basis of its immunological distinctiveness, as determined by quantitative neutralization with animal antisera. The antisera can be obtained from, e.g., ferrets, pigs or macaques that are infected with a mammalian MPV (see, e.g., Example 8).

In certain embodiments, the serotype does not cross-react with viruses other than mammalian MPV. In other embodiments, the serotype shows a homologous-to-heterologous titer ratio>16 in both directions If neutralization shows a certain degree of cross-reaction between two viruses in either or both directions (homologous-to-heterologous titer ration of eight or sixteen), distinctiveness of serotype is assumed if substantial biophysical/biochemical differences of DNA sequences exist. If neutralization shows a distinct degree of cross-reaction between two viruses in either or both directions (homologous-to-heterologous titer ratio of smaller than eight), identity of serotype of the isolates under study is assumed. Isolate I-2614, herein also known as MPV isolate 00-1, can be used as prototype.

In certain embodiments, a virus can be identified as a mammalian MPV by means of sequence homology/identity of the viral proteins or nucleic acids in comparison with the amino acid sequence and nucleotide sequences of the viral isolates disclosed herein by sequence or deposit. In particular, a virus is identified as a mammalian MPV when the genome of the virus contains a nucleic acid sequence that has a percentage nucleic acid identity to a virus isolate deposited as I-2614 with CNCM, Paris which is higher than the percentages identified herein for the nucleic acids encoding the L protein, the M protein, the N protein, the P protein, or the F protein as identified herein below in comparison with APV-C (see Table 1). (See, PCT WO 02/05302, at pp. 12 to 19, which is incorporated by reference herein. Without being bound by theory, it is generally known that viral species, especially RNA virus species, often constitute a quasi species wherein the members of a cluster of the viruses display sequence heterogeneity. Thus, it is expected that each individual isolate may have a somewhat different percentage of sequence identity when compared to APV-C.

The highest amino sequence identity between the proteins of MPV and any of the known other viruses of the same family to date is the identity between APV-C and human MPV. Between human MPV and APV-C, the amino acid sequence identity for the matrix protein is 87%, 88% for the nucleoprotein, 68% for the phosphoprotein, 81% for the fusion protein and 56-64% for parts of the polymerase protein, as can be deduced when comparing the sequences given in the Sequence Listing, see also Table 1. Viral isolates that contain ORFs that encode proteins with higher homology compared to these maximum values are considered mammalian MPVs. It should be noted that, similar to other viruses, a certain degree of variation is found between different isolated of mammalian MPVs.

TABLE 1
Amino acid sequence identity between the ORFs of MPV and
those of other paramyxoviruses.

| | N | P | M | F | M2-1 | M2-2 | L |
|---|---|---|---|---|---|---|---|
| APV A | 69 | 55 | 78 | 67 | 72 | 26 | 64 |
| APV B | 69 | 51 | 76 | 67 | 71 | 27 | —² |
| APV C | 88 | 68 | 87 | 81 | 84 | 56 | —² |
| hRSVA | 42 | 24 | 38 | 34 | 36 | 18 | 42 |
| hRSV B | 41 | 23 | 37 | 33 | 35 | 19 | 44 |
| bRSV | 42 | 22 | 38 | 34 | 35 | 13 | 44 |
| PVM | 45 | 26 | 37 | 39 | 33 | 12 | —² |
| others³ | 7-11 | 4-9 | 7-10 | 10-18 | —⁴ | —⁴ | 13-14 |

Footnotes:
1. No sequence homologies were found with known G and SH proteins and were thus excluded
²Sequences not available.
³others: human parainfluenza virus type 2 and 3, Sendai virus, measles virus, nipah virus, phocine distemper virus, and New Castle Disease virus.
⁴ORF absent in viral genome.

In certain embodiments, the invention provides a mammalian MPV, wherein the amino acid sequence of the SH protein of the mammalian MPV is at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or at least 99.5% identical to the amino acid sequence of SEQ ID NO:382 (SH protein of isolate NL/1/00; see Table 14). The isolated negative-sense single stranded RNA metapneumovirus that comprises the SH protein that is at least 30% identical to SEQ ID NO:382 (SH protein of isolate NL/1/00; see Table 14) is capable of infecting a mammalian host. In certain embodiments, the isolated negative-sense single stranded RNA metapneumovirus that comprises the SH protein that is at least 30% identical to SEQ ID NO:382 (SH protein of isolate NL/1/00; see Table 14) is capable of replicating in a mammalian host. In certain embodiments, a mammalian MPV contains a nucleotide sequence that encodes a SH protein that is at least 30% identical to SEQ ID NO:382 (SH protein of isolate NL/1/00; see Table 14).

In certain embodiments, the invention provides a mammalian MPV, wherein the amino acid sequence of the G protein of the mammalian MPV is at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or at least 99.5% identical to the amino acid sequence of SEQ ID NO:322 (G protein of isolate NL/1/00; see Table 14). The isolated negative-sense single stranded RNA metapneumovirus that comprises the G protein that is at least 20% identical to SEQ ID NO:322 (G protein of isolate NL/1/00; see Table 14) is capable of infecting a mammalian host. In certain embodiments, the isolated negative-sense single stranded RNA metapneumovirus that comprises the G protein that is at least 20% identical to SEQ ID NO:322 (G protein of isolate NL/1/00; see Table 14) is capable of replicating in a mammalian host. In certain embodiments, a mammalian MPV contains a nucleotide sequence that encodes a G protein that is at least 20% identical to SEQ ID NO:322 (G protein of isolate NL/1/00; see Table 14).

In certain embodiments, the invention provides a mammalian MPV, wherein the amino acid sequence of the L protein of the mammalian MPV is at least 85%, at least 90%, at least 95%, at least 98%, at least 99%, or at least 99.5% identical to the amino acid sequence of SEQ ID NO:330 (L protein of isolate NL/1/00; see Table 14). The isolated negative-sense single stranded RNA metapneumovirus that comprises the L protein that is at least 85% identical to SEQ ID NO:330 (L protein of isolate NL/1/00; see Table 14) is capable of infecting a mammalian host. In certain embodiments, the isolated negative-sense single stranded RNA metapneumovirus that comprises the L protein that is at least 85% identical to SEQ ID NO:330 (L protein of isolate NL/1/00; see Table 14) is capable of replicating in a mammalian host. In certain embodiments, a mammalian MPV contains a nucleotide sequence that encodes a L protein that is at least 20% identical to SEQ ID NO:330 (L protein of isolate NL/1/00; see Table 14).

In certain embodiments, the invention provides a mammalian MPV, wherein the amino acid sequence of the N protein of the mammalian MPV is at least 90%, at least 95%, or at least 98% identical to the amino acid sequence of SEQ ID NO:366. The isolated negative-sense single stranded RNA metapneumovirus that comprises the N protein that is at least 90% identical in amino acid sequence to SEQ ID NO:366 is capable of infecting mammalian host. In certain embodiments, the isolated negative-sense single stranded RNA metapneumovirus that comprises the N protein that is 90% identical in amino acid sequence to SEQ ID NO:366 is capable of replicating in a mammalian host. The amino acid identity is calculated over the entire length of the N protein. In certain embodiments, a mammalian MPV contains a nucleotide sequence that encodes a N protein that is at least 90%, at least 95%, or at least 98% identical to the amino acid sequence of SEQ ID NO:366.

The invention further provides mammalian MPV, wherein the amino acid sequence of the P protein of the mammalian MPV is at least 70%, at least 80%, at least 90%, at least 95% or at least 98% identical to the amino acid sequence of SEQ ID NO:374. The mammalian MPV that comprises the P protein that is at least 70% identical in amino acid sequence to SEQ ID NO:374 is capable of infecting a mammalian host. In certain embodiments, the mammalian MPV that comprises the P protein that is at least 70% identical in amino acid sequence to SEQ ID NO:374 is capable of replicating in a mammalian host. The amino acid identity is calculated over the entire length of the P protein. In certain embodiments, a mammalian MPV contains a nucleotide sequence that encodes a P protein that is at least 70%, at least 80%, at least 90%, at least 95% or at least 98% identical to the amino acid sequence of SEQ ID NO:374.

The invention further provides, mammalian MPV, wherein the amino acid sequence of the M protein of the mammalian MPV is at least 90%, at least 95% or at least 98% identical to the amino acid sequence of SEQ ID NO:358. The mammalian MPV that comprises the M protein that is at least 90% identical in amino acid sequence to SEQ ID NO:358 is capable of infecting mammalian host. In certain embodiments, the isolated negative-sense single stranded RNA metapneumovirus that comprises the M protein that is 90% identical in amino acid sequence to SEQ ID NO:358 is capable of replicating in a mammalian host. The amino acid identity is calculated over the entire length of the M protein. In certain embodiments, a mammalian MPV contains a nucleotide sequence that encodes a M protein that is at least 90%, at least 95% or at least 98% identical to the amino acid sequence of SEQ ID NO:358.

The invention further provides mammalian MPV, wherein the amino acid sequence of the F protein of the mammalian MPV is at least 85%, at least 90%, at least 95% or at least 98% identical to the amino acid sequence of SEQ ID NO:314. The mammalian MPV that comprises the F protein that is at least 85% identical in amino acid sequence to SEQ ID NO:314 is capable of infecting a mammalian host. In certain embodiments, the isolated negative-sense single stranded RNA metapneumovirus that comprises the F protein that is 85% identical in amino acid sequence to SEQ ID NO:314 is capable of replicating in mammalian host. The amino acid identity is calculated over the entire length of the F protein. In certain embodiments, a mammalian MPV contains a nucleotide sequence that encodes a F protein that is at least 85%, at least 90%, at least 95% or at least 98% identical to the amino acid sequence of SEQ ID NO:314.

The invention further provides mammalian MPV, wherein the amino acid sequence of the M2-1 protein of the mammalian MPV is at least 85%, at least 90%, at least 95% or at least 98% identical to the amino acid sequence of SEQ ID NO:338. The mammalian MPV that comprises the M2-1 protein that is at least 85% identical in amino acid sequence to SEQ ID NO:338 is capable of infecting a mammalian host. In certain embodiments, the isolated negative-sense single stranded RNA metapneumovirus that comprises the M2-1 protein that is 85% identical in amino acid sequence to SEQ ID NO:338 is capable of replicating in a mammalian host. The amino acid identity is calculated over the entire length of the M2-1 protein. In certain embodiments, a mammalian MPV contains a nucleotide sequence that encodes a M2-1 protein that is at least 85%, at least 90%, at least 95% or at least 98% identical to the amino acid sequence of SEQ ID NO:338.

The invention further provides mammalian MPV, wherein the amino acid sequence of the M2-2 protein of the mammalian MPV is at least 60%, at least 70%, at least 80%, at least 90%, at least 95% or at least 98% identical to the amino acid sequence of SEQ ID NO:346 The isolated mammalian MPV that comprises the M2-2 protein that is at least 60% identical in amino acid sequence to SEQ ID NO:346 is capable of infecting mammalian host. In certain embodiments, the isolated negative-sense single stranded RNA metapneumovirus that comprises the M2-2 protein that is 60% identical in amino acid sequence to SEQ ID NO:346 is capable of replicating in a mammalian host. The amino acid identity is calculated over the entire length of the M2-2 protein. In certain embodiments, a mammalian MPV contains a nucleotide sequence that encodes a M2-1 protein that is is at least 60%, at least 70%, at least 80%, at least 90%, at least 95% or at least 98% identical to the amino acid sequence of SEQ ID NO:346.

In certain embodiments, the invention provides mammalian MPV, wherein the negative-sense single stranded RNA metapneumovirus encodes at least two proteins, at least three proteins, at least four proteins, at least five proteins, or six proteins selected from the group consisting of (i) a N protein with at least 90% amino acid sequence identity to SEQ ID NO:366; (ii) a P protein with at least 70% amino acid sequence identity to SEQ ID NO:374 (iii) a M protein with at least 90% amino acid sequence identity to SEQ ID NO:358 (iv) a F protein with at least 85% amino acid sequence identity to SEQ ID NO:314 (v) a M2-1 protein with at least 85% amino acid sequence identity to SEQ ID NO:338; and (vi) a M2-2 protein with at least 60% amino acid sequence identity to SEQ ID NO:346.

The invention provides two subgroups of mammalian MPV, subgroup A and subgroup B. The invention also provides four variants A1, A2, B1 and B2. A mammalian MPV can be identified as a member of subgroup A if it is phylogenetically closer related to the isolate 00-1 (SEQ ID NO:19) than to the isolate 99-1 (SEQ ID NO:18). A mammalian MPV can be identified as a member of subgroup B if it is phylogenetically closer related to the isolate 99-1 (SEQ ID NO:18) than to the isolate 00-1 (SEQ ID NO:19). In other embodiments, nucleotide or amino acid sequence homologies of individual ORFs can be used to classify a mammalian MPV as belonging to subgroup A or B.

The different isolates of mammalian MPV can be divided into four different variants, variant A1, variant A2, variant B1 and variant B2. The isolate 00-1 (SEQ ID NO:19) is an example of the variant A1of mammalian MPV. The isolate 99-1 (SEQ ID NO:18) is an example of the variant B1 of mammalian MPV. A mammalian MPV can be grouped into one of the four variants using a phylogenetic analysis. Thus, a mammalian MPV belongs to a specific variant if it is phylogenetically closer related to a known member of that variant than it is phylogenetically related to a member of another variant of mammalian MPV. The sequence of any ORF and the encoded polypeptide may be used to type a MPV isolate as belonging to a particular subgroup or variant, including N, P, L, M, SH, G, M2 or F polypeptides. In a specific embodiment, the classification of a mammalian MPV into a variant is based on the sequence of the G protein. Without being bound by theory, the G protein sequence is well suited for phylogenetic analysis because of the high degree of variation among G proteins of the different variants of mammalian MPV.

In certain embodiments of the invention, sequence homology may be determined by the ability of two sequences to hybridize under certain conditions, as set forth below. A nucleic acid which is hybridizable to a nucleic acid of a mammalian MPV, or to its reverse complement, or to its complement can be used in the methods of the invention to determine their sequence homology and identities to each other. In certain embodiments, the nucleic acids are hybridized under conditions of high stringency.

It is well-known to the skilled artisan that hybridization conditions, such as, but not limited to, temperature, salt concentration, pH, formamide concentration (see, e.g., Sambrook et al., 1989, Chapters 9 to 11, Molecular Cloning, A Laboratory Manual, 2d Ed., Cold Spring Harbor Laboratory Press, Cold Spring Harbor, N.Y., incorporated herein by reference in its entirety). In certain embodiments, hybridization is performed in aqueous solution and the ionic strength of the solution is kept constant while the hybridization temperature is varied dependent on the degree of sequence homology between the sequences that are to be hybridized. For DNA sequences that 100% identical to each other and are longer than 200 basebairs, hybridization is carried out at approximately 15-25° C. below the melting temperature (Tm) of the perfect hybrid. The melting temperature (Tm) can be calculated using the following equation (Bolton and McCarthy, 1962, Proc. Natl. Acad. Sci. USA 84:1390):

Tm=81.5° C.−16.6(log 10[Na+])+(% G+C)−0.63(% formamide)−(600/1)

Wherein (Tm) is the melting temperature, [Na+] is the sodium concentration, G+C is the Guanine and Cytosine content, and 1 is the length of the hybrid in basepairs. The effect of mismatches between the sequences can be calculated using the formula by Bonner et al. (Bonner et al., 1973, J. Mol. Biol. 81:123-135): for every 1% of mismatching of bases in the hybrid, the melting temperature is reduced by 1-1.5° C.
Thus, by determining the temperature at which two sequences hybridize, one of skill in the art can estimate how similar a sequence is to a known sequence. This can be done, e.g., by comparison of the empirically determined hybridization temperature with the hybridization temperature calculated for the know sequence to hybridize with its perfect match. Through the use of the formula by Bonner et al., the relationship between hybridization temperature and per cent mismatch can be exploited to provide information about sequence similarity.

By way of example and not limitation, procedures using such conditions of high stringency are as follows. Prehybridization of filters containing DNA is carried out for 8 h to overnight at 65 C in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65 C in prehybridization mixture containing 100 μg/ml denatured salmon sperm DNA and 5-20×106 cpm of 32P-labeled probe. Washing of filters is done at 37 C for 1 h in a solution containing 2×SSC, 0.01% PVP, 0.01% Ficoll, and 0.01% BSA. This is followed by a wash in 0.1×SSC at 50 C for 45 min before autoradiography. Other conditions of high stringency which may be used are well known in the art. In other embodiments of the invention, hybridization is performed under moderate of low stringency conditions, such conditions are well-known to the skilled artisan (see e.g., Sambrook et al., 1989, Molecular Cloning, A Laboratory Manual, 2d Ed., Cold Spring Harbor Laboratory Press, Cold Spring Harbor, N.Y.; see also, Ausubel et al., eds., in the Current Protocols in Molecular Biology series of laboratory technique manuals, 1987-1997 Current Protocols, © 1994-1997 John Wiley and Sons, Inc., each of which is incorporated by reference herein in their entirety). An illustrative low stringency condition is, provided by the following system of buffers: hybridization in a buffer comprising 35% formamide, 5×SSC, 50 mM Tris-HCl (pH 7.5), 5 mM EDTA, 0.02% PVP, 0.02% Ficoll, 0.2% BSA, 100 μg/ml denatured salmon sperm DNA, and 10% (wt/vol) dextran sulfate for 18-20 hours at 40DC, washing in a buffer consisting of 2×SSC, 25 mM Tris-HCl (pH 7.4), 5 mM EDTA, and 0.1% SDS for 1.5 hours at 55° C, and washing in a buffer consisting of 2×SSC, 25 mM Tris-HCl (pH 7.4), 5 mM EDTA, and 0.1% SDS for 1.5 hours at 60° C.

In certain embodiments, a mammalian MPV can be classified into one of the variant using probes that are specific for a specific variant of mammalian MPV. Such probes include primers for RT-PCR and antibodies. Illustrative methods for identifying a mammalian MPV as a member of a specific variant are described in section 5.9 below.

In certain embodiments of the invention, the different variants of mammalian MPV can be distinguished from each other by way of the amino acid sequences of the different viral proteins. In other embodiments, the different variants of mammalian MPV can be distinguished from each other by way of the nucleotide sequences of the different ORFs encoded by the viral genome. A variant of mammalian MPV can be, but is not limited to, A1, A2, B1 or B2. The invention, however, also contemplates isolates of mammalian MPV that are members of another variant yet to be identified. The invention also contemplates that a virus may have one or more ORF that are closer related to one variant and one or more ORFs that are closer phylogenetically related to another variant. Such a virus would be classified into the variant to which the majority of its ORFs are closer phylogenetically related. Non-coding sequences may also be used to determine phylogenetic relatedness.

An isolate of mammalian MPV is classified as a variant B1 if it is phylogenetically closer related to the viral isolate NL/1/99 (SEQ ID NO:18) than it is related to any of the following other viral isolates: NL/1/00 (SEQ ID NO:19), NL/17/00 (SEQ ID NO:20) and NL/1/94 (SEQ ID NO:21). One or more of the ORFs of a mammalian MPV can be used to classify the mammalian MPV into a variant. A mammalian MPV can be classified as an MPV variant B1, if the amino acid sequence of its G protein is at least 66%, at least 70%, at least 75%, at least 80%, at least 85%, at least 90%, at least 95%, at least 98%, or at least 99% or at least 99.5% identical to the G protein of a mammalian MPV variant B1 as represented by the prototype NL/1/99 (SEQ ID NO:324); if the amino acid sequence of its N proteint is at least 98.5% or at least 99% or at least 99.5% identical to the N protein of a mammalian MPV variant B1 as represented by the prototype NL/1/99 (SEQ ID NO:368); if the amino acid sequence of its P protein is at least 96%, at least 98%, or at least 99% or at least 99.5% identical to the P protein of a mammalian MPV variant B1 as represented by the prototype NL/1/99 (SEQ ID NO:376); if the amino acid sequence of its M protein is identical to the M protein of a mammalian MPV variant B1 as represented by the prototype NL/1/99 (SEQ ID NO:360); if the amino acid sequence of its F protein is at least 99% identical to the F protein of a mammalian MPV variant B1 as represented by the prototype NL/1/99 (SEQ ID NO:316); if the amino acid sequence of its M2-1 protein is at least 98% or at least 99% or at least 99.5% identical to the M2-1 protein of a mammalian MPV variant B1 as represented by the prototype NL/1/99 (SEQ ID NO:340); if the amino acid sequence of its M2-2 protein is at least 99%or at least 99.5% identical to the M2-2 protein of a mammalian MPV variant B1 as represented by the prototype NL/1/99 (SEQ ID NO:348); if the amino acid sequence of its SH protein is at least 83%, at least 85%, at least 90%, at least 95%, at least 98%, or at least 99% or at least 99.5% identical to the SH protein of a mammalian MPV variant B1 as represented by the prototype NL/1/99 (SEQ ID NO:384); and/or if the amino acid sequence of its L protein is at least 99% or at least 99.5% identical to the L protein a mammalian MPV variant B1 as represented by the prototype NL/1/99 (SEQ ID NO:332).

An isolate of mammalian MPV is classified as a variant A1 if it is phylogenetically closer related to the viral isolate NL/1/00 (SEQ ID NO:19) than it is related to any of the following other viral isolates: NLJ1/99 (SEQ ID NO:18), NL/17/00 (SEQ ID NO:20) and NL/1/94 (SEQ ID NO:21). One or more of the ORFs of a mammalian MPV can be used to classify the mammalian MPV into a variant. A mammalian MPV can be classified as an MPV variant A1, if the amino acid sequence of its G protein is at least 66%, at least 70%, at least 75%, at least 80%, at least 85%, at least 90%, at least 95%, at least 98%, or at least 99% or at least 99.5% identical to the G protein of a mammalian MPV variant A1 as represented by the prototype NL/1/00 (SEQ ID NO:322); if the amino acid sequence of its N protein is at least 99.5% identical to the N protein of a mammalian MPV variant A1 as represented by the prototype NL/1/00 (SEQ ID NO:366); if the amino acid sequence of its P protein is at least 96%, at least 98%, or at least 99% or at least 99.5% identical to the P protein of a mammalian MPV variant A1 as represented by the prototype NL/1/00 (SEQ ID NO:374); if the amino acid sequence of its M protein is at least 99% or at least 99.5% identical to the M protein of a mammalian MPV variant A1 as represented by the prototype NL/1/00 (SEQ ID NO:358); if the amino acid sequence of its F protein is at least 98% or at least 99% or at least 99.5% identical to the F protein of a mammalian MPV variant A1 as represented by the prototype NL/1/00 (SEQ ID NO:314); if the amino acid sequence of its M2-1 protein is at least 99% or at least 99.5% identical to the M2-1 protein of a mammalian MPV variant A1 as represented by the prototype NL/1/00 (SEQ ID NO:338); if the amino acid sequence of its M2-2 protein is at least 96% or at least 99% or at least 99.5% identical to the M2-2 protein of a mammalian MPV variant A1 as represented by the prototype NL/1/00 (SEQ ID NO:346); if the amino acid sequence of its SH protein is at least 84%, at least 90%, at least 95%, at least 98%, or at least 99% or at least 99.5% identical to the SH protein of a mammalian MPV variant A1 as represented by the prototype NL/1/00 (SEQ ID NO:382); and/or if the amino acid sequence of its L protein is at least 99% or at least 99.5% identical to the L protein of a virus of a mammalian MPV variant A1 as represented by the prototype NL/1/00 (SEQ ID NO:330).

An isolate of mammalian MPV is classified as a variant A2 if it is phylogenetically closer related to the viral isolate NL/17/00 (SEQ ID NO:20) than it is related to any of the following other viral isolates: NL/1/99 (SEQ ID NO:18), NL/1/00 (SEQ ID NO:19) and NL/1/94 (SEQ ID NO:21). One or more of the ORFs of a mammalian MPV can be used to classify the mammalian MPV into a variant. A mammalian MPV can be classified as an MPV variant A2, if the amino acid sequence of its G protein is at least 66%, at least 70%, at least 75%, at least 80%, at least 85%, at least 90%, at least 95%, at least 98%, at least 99% or at least 99.5% identical to the G protein of a mammalian MPV variant A2 as represented by the prototype NL/17/00 (SEQ ID NO:323); if the amino acid sequence of its N protein is at least 99.5% identical to the N protein of a mammalian MPV variant A2 as represented by the prototype NL/17/00 (SEQ ID NO:367); if the amino acid sequence of its P protein is at least 96%, at least 98%, at least 99% or at least 99.5% identical to the P protein of a mammalian MPV variant A2 as represented by the prototype NL/17/00 (SEQ ID NO:375); if the amino acid sequence of its M protein is at least 99%, or at least 99.5% identical to the M protein of a mammalian MPV variant A2 as represented by the prototype NL/17/00 (SEQ ID NO:359); if the amino acid sequence of its F protein is at least 98%, at least 99% or at least 99.5% identical to the F protein of a mammalian MPV variant A2 as represented by the prototype NL/17/00 (SEQ ID NO:315); if the amino acid sequence of its M2-1 protein is at least 99%, or at least 99.5% identical to the M2-1 protein of a mammalian MPV variant A2 as represented by the prototype NL/17/00 (SEQ ID NO: 339); if the amino acid sequence of its M2-2 protein is at least 96%, at least 98%, at least 99% or at least 99.5% identical to the M2-2 protein of a mammalian MPV variant A2 as represented by the prototype NL/17/00 (SEQ ED NO:347); if the amino acid sequence of its SH protein is at least 84%, at least 85%, at least 90%, at least 95%, at least 98%, at least 99% or at least 99.5% identical to the SH protein of a mammalian MPV variant A2 as represented by the prototype NL/17/00 (SEQ ID NO:383); if the amino acid sequence of its L protein is at least 99% or at least 99.5% identical to the L protein of a mammalian MPV variant A2 as represented by the prototype NL/17/00 (SEQ ID NO:331).

An isolate of mammalian MPV is classified as a variant B2 if it is phylogenetically closer related to the viral isolate NL/1/94 (SEQ ID NO:21) than it is related to any of the following other viral isolates: NL/1/99 (SEQ ID NO:18), NL/1/00 (SEQ ID NO:19) and NL/17/00 (SEQ ID NO:20). One or more of the ORFs of a mammalian MPV can be used to classify the mammalian MPV into a variant. A mammalian MPV can be classified as an MPV variant B2, if the amino acid sequence of its G protein is at least 66%, at least 70%, at least 75%, at least 80%, at least 85%, at least 90%, at least 95%, at least 98%, or at least 99% or at least 99.5% identical to the G protein of a mammalian MPV variant B2 as represented by the prototype NL/1/94 (SEQ ID NO:325); if the amino acid sequence of its N protein is at least 99% or at least 99.5% identical to the N protein of a mammalian MPV variant B2 as represented by the prototype NL/1/94 (SEQ ID NO:369); if the amino acid sequence of its P protein is at least 96%, at least 98%, or at least 99% or at least 99.5% identical to the P protein of a mammalian MPV variant B2 as represented by the prototype NL/1/94 (SEQ ID NO:377); if the amino acid sequence of its M protein is identical to the M protein of a mammalian MPV variant B2 as represented by the prototype NL/1/94 (SEQ ID NO:361); if the amino acid sequence of its F protein is at least 99% or at least 99.5% identical to the F protein of a mammalian MPV variant B2 as represented by the prototype NL/1/94 (SEQ ID NO:317); if the amino acid sequence of the M2-1 protein is at least 98% or at least 99% or at least 99.5% identical to the M2-1 protein of a mammalian MPV variant B2 as represented by the prototype NL/1/94 (SEQ ID NO:341); if the amino acid sequence that is at least 99% or at least 99.5% identical to the M2-2 protein of a mammalian MPV variant B2 as represented by the prototype NL/1/94 (SEQ ID NO:349); if the amino acid sequence of its SH protein is at least 84%, at least 85%, at least 90%, at least 95%, at least 98%, or at least 99% or at least 99.5% identical to the SH protein of a mammalian MPV variant B2 as represented by the prototype NL/1/94 (SEQ ID NO:385); and/or if the amino acid sequence of its L protein is at least 99% or at least 99.5% identical to the L protein of a mammalian MPV variant B2 as represented by the prototype NL/1/94 (SEQ ID NO:333).

In certain embodiments, the percentage of sequence identity is based on an alignment of the full length proteins. In other embodiments, the percentage of sequence identity is based on an alignment of contiguous amino acid sequences of the proteins, wherein the amino acid sequences can be 25 amino acids, 50 amino acids, 75 amino acids, 100 amino acids, 125 amino acids, 150 amino acids, 175 amino acids, 200 amino acids, 225 amino acids, 250 amino acids, 275 amino acids, 300 amino acids, 325 amino acids, 350 amino acids, 375 amino acids, 400 amino acids, 425 amino acids, 450 amino acids, 475 amino acids, 500 amino acids, 750 amino acids, 1000 amino acids, 1250 amino acids, 1500 amino acids, 1750 amino acids, 2000 amino acids or 2250 amino acids in length.

### 5.2 Functional Characteristics of a Mammalian MPV

In addition to the structural definitions of the mammalian MPV, a mammalian MPV can also be defined by its functional characteristics. In certain embodiments, the mammalian MPV of the invention is capable of infecting a mammalian host. The mammalian host can be a mammalian cell, tissue, organ or a mammal. In a specific embodiment, the mammalian host is a human or a human cell, tissue or organ. Any method known to the skilled artisan can be used to test whether the mammalian host has been infected with the mammalian MPV. In certain embodiments, the virus is tested for its ability to attach to a mammalian cell. In certain other embodiments, the virus is tested for its ability to transfer its genome into the mammalian cell. In an illustrative embodiment, the genome of the virus is detectably labeled, e.g., radioactively labeled. The virus is then incubated with a mammalian cell for at least 1 minute, at least 5 minutes at least 15 minutes, at least 30 minutes, at least 1 hour, at least 2 hours, at least 5 hours, at least 12 hours, or at least 1 day. The cells are subsequently washed to remove any viral particles from the cells and the cells are then tested for the presence of the viral genome by virtue of the detectable label. In another embodiment, the presence of the viral genome in the cells is detected using RT-PCR using mammalian MPV specific primers. (See , PCT WO 02/057302 at pp. 37 to 44, which is incorporated by reference herein).

In certain embodiments, the mammalian virus is capable to infect a mammalian host and to cause proteins of the mammalian MPV to be inserted into the cytoplasmic membrane of the mammalian host. The mammalian host can be a cultured mammalian cell, organ, tissue or mammal. In an illustrative embodiment, a mammalian cell is incubated with the mammalian virus. The cells are subsequently washed under conditions that remove the virus from the surface of the cell. Any technique known to the skilled artisan can be used to detect the newly expressed viral protein inserted in the cytoplasmic membrane of the mammalian cell. For example, after infection of the cell with the virus, the cells are maintained in medium comprising a detectably labeled amino acid. The cells are subsequently harvested, lysed, and the cytoplasmic fraction is separated from the membrane fraction. The proteins of the membrane fraction are then solubilized and then subjected to an immunoprecipitation using antibodies specific to a protein of the mammalian MPV, such as, but not limited to, the F protein or the G protein. The immunoprecipitated proteins are then subjected to SDS PAGE. The presence of viral protein can then be detected by autoradiography. In another embodiment, the presence of viral proteins in the cytoplasmic membrane of the host cell can be detected by immunocytochemistry using one or more antibodies specific to proteins of the mammalian MPV.

In even other embodiments, the mammalian MPV of the invention is capable of infecting a mammalian host and of replicating in the mammalian host. The mammalian host can be a cultured mammalian cell, organ, tissue or mammal. Any technique known to the skilled artisan can be used to determine whether a virus is capable of infecting a mammalian cell and of replicating within the mammalian host. In a specific embodiment, mammalian cells are infected with the virus. The cells are subsequently maintained for at least 30 minutes, at least 1 hour, at least 2 hours, at least 5 hours, at least 12 hours, at least 1 day, or at least 2 days. The level of viral genomic RNA in the cells can be monitored using Northern blot analysis, RT-PCR or in situ hybridization using probes that are specific to the viral genome. An increase in viral genomic RNA demonstrates that the virus can infect a mammalian cell and can replicate within a mammalian cell.

In even other embodiments, the mammalian MPV of the invention is capable of infecting a mammalian host, wherein the infection causes the mammalian host to produce new infectious mammalian MPV. The mammalian host can be a cultured mammalian cell or a mammal. Any technique known to the skilled artisan can be used to determine whether a virus is capable of infecting a mammalian host and cause the mammalian host to produce new infectious viral particles. In an illustrative example, mammalian cells are infected with a mammalian virus. The cells are subsequently washed and incubated for at least 30 minutes, at least 1 hour, at least 2 hours, at least 5 hours, at least 12 hours, at least 1 day, at least 2 days, at least one week, or at least twelve days. The titer of virus can be monitored by any method known to the skilled artisan. For exemplary methods see section 5.8.

In certain, specific embodiments, the mammalian MPV is a human MPV. The tests described in this section can also be performed with a human MPV. In certain embodiments, the human MPV is capable of infecting a mammalian host, such as a mammal or a mammalian cultured cell.

In certain embodiments, the human MPV is capable to infect a mammalian host and to cause proteins of the human MPV to be inserted into the cytoplasmic membrane of the mammalian host.

In even other embodiments, the human MPV of the invention is capable of infecting a mammalian host and of replicating in the mammalian host.

In even other embodiments, the human MPV of the invention is capable of infecting a mammalian host and of replicating in the mammalian host, wherein the infection and replication causes the mammalian host to produce and package new infectious human MPV.

In certain embodiments, the mammalian MPV, even though it is capable of infecting a mammalian host, is also capable of infecting an avian host, such as a bird or an avian cultured cell. In certain embodiments, the mammalian MPV is capable to infect an avian host and to cause proteins of the mammalian MPV to be inserted into the cytoplasmic membrane of the avian host. In even other embodiments, the mammalian MPV of the invention is capable of infecting an avian host and of replicating in the avian host. In even other embodiments, the mammalian MPV of the invention is capable of infecting an avian host and of replicating in the avian host, wherein the infection and replication causes the avian host to produce and package new infectious mammalian MPV.

### 5.3 Recombinant and Chimeric Metapneumovirus

The present invention encompasses recombinant or chimeric viruses encoded by viral vectors derived from the genomes of metapneumovirus, including both mammalian and avian variants. In accordance with the present invention a recombinant virus is one derived from a mammalian MPV or an APV that is encoded by endogenous or native genomic sequences or non-native genomic sequences. In accordance with the invention, a non-native sequence is one that is different from the native or endogenous genomic sequence due to one or more mutations, including, but not limited to, point mutations, rearrangements, insertions, deletions etc., to the genomic sequence that may or may not result in a phenotypic change. The recombinant viruses of the invention encompass those viruses encoded by viral vectors derived from the genomes of metapneumovirus, including both mammalian and avian variants, and may or may not, include nucleic acids that are non-native to the viral genome. In accordance with the present invention, a viral vector which is derived from the genome of a metapneumovirus is one that contains a nucleic acid sequence that encodes at least a part of one ORF of a mammalian metapneumovirus, wherein the polypeptides encoded by the ORF have amino acid sequence identity as set forth in Section 5.1. supra, and Table 1.

In accordance with the present invention, the recombinant viruses of the invention encompass those viruses encoded by viral vectors derived from the genome of a mammalian metapneumovirus (MPV), in particular a human metapneumovirus. In particular embodiments of the invention, the viral vector is derived from the genome of a metapneumovirus A1, A2, B1 or B2 variant. In accordance with the present invention, these viral vectors may or may not include nucleic acids that are non-native to the viral genome

In accordance with the present invention, the recombinant viruses of the invention encompass those viruses encoded by viral vectors derived from the genome of an avian pneumovirus (APV), also known as turkey rhinotracheitis virus (TRTV). In particular embodiments of the invention, the viral vector is derived from the genome of an APV subgroup A, B, C or D. In a preferred embodiment, a viral vector derived from the genome of an APV subgroup C. In accordance with the present invention these viral vectors may or may not include nucleic acids that are non-native to the viral genome.

In another preferred embodiment of the invention, the recombinant viruses of the invention encompass those viruses encoded by a viral vector derived from the genome of an APV that contains a nucleic acid sequence that encodes a F-ORF of APV subgroup C. In certain embodiments, a viral vector derived from the genome of an APV is one that contains a nucleic acid sequence that encodes at least a N-ORF, a P-ORF, a M-ORF, a F-ORF, a M2-1-ORF, a M2-2-ORF or a L-ORF of APV.

In accordance with the invention, a chimeric virus is a recombinant MPV or APV which further comprises a heterologous nucleotide sequence. In accordance with the invention, a chimeric virus may be encoded by a nucleotide sequence in which heterologous nucleotide sequences have been added to the genome or in which endogenous or native nucleotide sequences have been replaced with heterologous nucleotide sequences.

In accordance with the invention, the chimeric viruses are encoded by the viral vectors of the invention which further comprise a heterologous nucleotide sequence. In accordance with the present invention a chimeric virus is encoded by a viral vector that may or may not include nucleic acids that are non-native to the viral genome. In accordance with the invention a chimeric virus is encoded by a viral vector to which heterologous nucleotide sequences have been added, inserted or substituted for native or non-native sequences. In accordance with the present invention, the chimeric virus may be encoded by nucleotide sequences derived from different strains of mammalian MPV. In particular, the chimeric virus is encoded by nucleotide sequences that encode antigenic polypeptides derived from different strains of MPV.

In accordance with the present invention, the chimeric virus may be encoded by a viral vector derived from the genome of an APV, in particular subgroup C, that additionally encodes a heterologous sequence that encodes antigenic polypeptides derived from one or more strains of MPV.

A chimeric virus may be of particular use for the generation of recombinant vaccines protecting against two or more viruses (Tao et al., J. Virol. 72, 2955-2961; Durbin et al., 2000, J. Virol. 74, 6821-6831; Skiadopoulos et al., 1998, J. Virol. 72, 1762-1768; Teng et al., 2000, J. Virol. 74, 9317-9321). For example, it can be envisaged that a MPV or APV virus vector expressing one or more proteins of another negative strand RNA virus, e.g., RSV or a RSV vector expressing one or more proteins of MPV will protect individuals vaccinated with such vector against both virus infections. A similar approach can be envisaged for PIV or other paramyxoviruses. Attenuated and replication-defective viruses may be of use for vaccination purposes with live vaccines as has been suggested for other viruses. (See, PCT WO 02/057302, at pp. 6 and 23, incorporated by reference herein).

In accordance with the present invention the heterologous sequence to be incorporated into the viral vectors encoding the recombinant or chimeric viruses of the invention include sequences obtained or derived from different strains of metapneumovirus, strains of avian pneumovirus, and other negative strand RNA viruses, including, but not limited to, RSV, PIV and influenza virus, and other viruses, including morbillivirus.

In certain embodiments of the invention, the chimeric or recombinant viruses of the invention are encoded by viral vectors derived from viral genomes wherein one or more sequences, intergenic regions, termini sequences, or portions or entire ORF have been substituted with a heterologous or non-native sequence. In certain embodiments of the invention, the chimeric viruses of the invention are encoded by viral vectors derived from viral genomes wherein one or more heterologous sequences have been added to the vector.

In certain embodiments, the virus of the invention contains heterologous nucleic acids. In a preferred embodiment, the heterologous nucleotide sequence is inserted or added at Position 1 of the viral genome. In another preferred embodiment, the heterologous nucleotide sequence is inserted or added at Position 2 of the viral genome. In even another preferred embodiment, the heterologous nucleotide sequence is inserted or added at Position 3 of the viral genome. Insertion or addition of nucleic acid sequences at the lower-numbered positions of the viral genome results in stronger or higher levels of expression of the heterologous nucleotide sequence compared to insertion at higher-numbered positions due to a transcriptional gradient across the genome of the virus. Thus, inserting or adding heterologous nucleotide sequences at lower-numbered positions is the preferred embodiment of the invention if high levels of expression of the heterologous nucleotide sequence is desired.

Without being bound by theory, the position of insertion or addition of the heterologous sequence affects the replication rate of the recombinant or chimeric virus. The higher rates of replication can be achieved if the heterologous sequence is inserted or added at Position 2 or Position 1 of the viral genome. The rate of replication is reduced if the heterologous sequence is inserted or added at Position 3, Position 4, Position 5, or Position 6.

Without being bound by theory, the size of the intergenic region between the viral gene and the heterologous sequence further determines rate of replication of the virus and expression levels of the heterologous sequence.

In certain embodiments, the viral vector of the invention contains two or more different heterologous nucleotide sequences. In a preferred embodiment, one heterologous nucleotide sequence is at Position 1 and a second heterologous nucleotide sequence is at Position 2 of the viral genome. In another preferred embodiment, one heterologous nucleotide sequence is at Position 1 and a second heterologous nucleotide sequence is at Position 3 of the viral genome. In even another preferred embodiment, one heterologous nucleotide sequence is at Position 2 and a second heterologous nucleotide sequence is at Position 3 of the viral genome. In certain other embodiments, a heterologous nucleotide sequence is inserted at other, higher-numbered positions of the viral genome. In accordance with the present invention, the position of the heterologous sequence refers to the order in which the sequences are transcribed from the viral genome, e.g., a heterologous sequence at Position 1 is the first gene sequence to be transcribed from the genome.

The selection of the viral vector may depend on the species of the subject that is to be treated or protected from a viral infection. If the subject is human, then an attenuated mammalian metapneumovirus or an avian pneumovirus can be used to provide the antigenic sequences.

In accordance with the present invention, the viral vectors can be engineered to provide antigenic sequences which confer protection against infection by a metapneumovirus, including sequences derived from mammalian metapneumovirus, human metapneumovirus, MPV variants A1, A2, B1 or B2, sequences derived from avian pneumovirus, including APV subgroups A, B, C or D, although C is preferred. The viral vectors can be engineered to provide antigenic sequences which confer protection against infection or disease by another virus, including negative strand RNA virus, including influenza, RSV or PTV, including PIV3. The viral vectors may be engineered to provide one, two, three or more antigenic sequences. In accordance with the present invention the antigenic sequences may be derived from the same virus, from different strains or variants of the same type of virus, or from different viruses, including morbillivirus.

In certain embodiments of the invention, the heterologous nucleotide sequence to be inserted into the genome of the virus of the invention is derived from a metapneumovirus. In certain specific embodiments of the invention, the heterologous nucleotide sequence is derived from a human metapneumovirus. In another specific embodiment, the heterologous nucleotide sequence is derived from an avian pneumovirus. More specifically, the heterologous nucleotide sequence of the invention encodes a F gene of a human metapneumovirus. More specifically, the heterologous nucleotide sequence of the invention encodes an G gene of a human metapneumovirus. More specifically, the heterologous nucleotide sequence of the invention encodes a F gene of an avian pneumovirus. More specifically, the heterologous nucleotide sequence of the invention encodes a G gene of an avian pneumovirus. In specific embodiments, a heterologous nucleotide sequences can be any one of SEQ ID NO:1 through SEQ ID NO:5, SEQ ID NO:14, and SEQ ID NO:15. In certain specific embodiments, the nucleotide sequence encodes a protein of any one of SEQ ID NO:6 through SEQ ID NO:13, SEQ ID NO:16, and SEQ ID NO:17.

In a specific embodiment of the invention, the heterologous nucleotide sequence encodes a chimeric F protein. In an illustrative embodiment, the ectodomain of the chimeric F-protein is the ectodomain of a human MPV and the transmembrane domain and the luminal domain are derived from the F-protein of an avian metapneumovirus. Without being bound by theory, a chimeric human MPV that encodes the chimeric F-protein consisting of the human ectodomain and the avian lumino/transmembrane domain is attenuated because of the avian part of the F-protein, yet highly immunogenic against hMPV because of the human ectodomain.

In certain embodiments, two different heterologous nucleotide sequences are inserted or added to the viral vectors of the invention, derived from metapneumoviral genomes, including mammalian and avian. For example, the heterologous nucleotide sequence is derived from a human metapneumovirus, an avian pneumovirus, RSV, PIV, or influenza. In a preferred embodiment, the heterologous sequence encodes the F-protein of human metapneumovirus, avian pneumovirus, RSV or PIV respectively. In another embodiment, the heterologous sequence encodes the HA protein of influenza.

In certain embodiments, the viral vector of the invention contains two different heterologous nucleotide sequences wherein a first heterologous nucleotide sequence is derived from a metapneumovirus, such as a human metapneumovirus or an avian pneumovirus, and a second nucleotide sequence is derived from a respiratory syncytial virus (see Table 2). In specific embodiments, the heterologous nucleotide sequence derived from respiratory syncytial virus is a F gene of a respiratory syncytial virus. In other specific embodiments, the heterologous nucleotide sequence derived from respiratory syncytial virus is a G gene of a respiratory syncytial virus. In a specific embodiment, the heterologous nucleotide sequence derived from a metapneumovirus is inserted at a lower-numbered position than the heterologous nucleotide sequence derived from a respiratory syncytial virus. In another specific embodiment, the heterologous nucleotide sequence derived from a metapneumovirus is inserted at a higher-numbered position than the heterologous nucleotide sequence derived from a respiratory syncytial virus.

In certain embodiments, the virus of the invention contains two different heterologous nucleotide sequences wherein a first heterologous nucleotide sequence is derived from a metapneumovirus, such as a human metapneumovirus or an avian pneumovirus, and a second nucleotide sequence is derived from a parainfluenza virus, such as, but not limited to PIV3 (see Table 2). In specific embodiments, the heterologous nucleotide sequence derived from PIV is a F gene of PIV. In other specific embodiments, the heterologous nucleotide sequence derived from PIV is a G gene of a PIV. In a specific embodiment, the heterologous nucleotide sequence derived from a metapneumovirus is inserted at a lower-numbered position than the heterologous nucleotide sequence derived from a PIV. In another specific embodiment, the heterologous nucleotide sequence derived from a metapneumovirus is inserted at a higher-numbered position than the heterologous nucleotide sequence derived from a PIV.

The expression products and/or recombinant or chimeric virions obtained in accordance with the invention may advantageously be utilized in vaccine formulations. The expression products and chimeric virions of the present invention may be engineered to create vaccines against a broad range of pathogens, including viral and bacterial antigens, tumor antigens, allergen antigens, and auto antigens involved in autoimmune disorders. In particular, the chimeric virions of the present invention may be engineered to create vaccines for the protection of a subject from infections with PIV, RSV, and/or metapneumovirus.

In another embodiment, the chimeric virions of the present invention may be engineered to create anti-HIV vaccines, wherein an immunogenic polypeptide from gp160, and/or from internal proteins of HIV is engineered into the glycoprotein HN protein to construct a vaccine that is able to elicit both vertebrate humoral and cell-mediated immune responses. In yet another embodiment, the invention relates to recombinant metapneumoviral vectors and viruses which are engineered to encode mutant antigens. A mutant antigen has at least one amino acid substitution, deletion or addition relative to the wild-type viral protein from which it is derived.

In certain embodiments, the invention relates to trivalent vaccines comprising a recombinant or chimeric virus of the invention. In specific embodiments, the virus used as backbone for a trivalent vaccine is a chimeric avian-human metapneumovirus or a chimeric human-avian metapneumovirus containing a first heterologous nucleotide sequence derived from a RSV and a second heterologous nucleotide sequence derived from PIV. In an exemplary embodiment, such a trivalent vaccine will be specific to (a) the gene products of the F gene and/or the G gene of the human metapneumovirus or avian pneumovirus, respectively, dependent on whether chimeric avian-human or chimeric human-avian metapneumovirus is used; (b) the protein encoded by the heterologous nucleotide sequence derived from a RSV; and (c) the protein encoded by the heterologous nucleotide sequence derived from PIV. In a specific embodiment, the first heterologous nucleotide sequence is the F gene of the respiratory syncytial virus and is inserted in Position 1, and the second heterologous nucleotide sequence is the F gene of the PIV and is inserted in Position 3. Many more combinations are encompassed by the present invention and some are shown by way of example in Table 2. Further, nucleotide sequences encoding chimeric F proteins could be used (see supra). In some less preferred embodiments, the heterologous nucleotide sequence can be inserted at higher-numbered positions of the viral genome.

TABLE 2
Exemplary arrangements of heterologous nucleotide sequences
in the viruses used for trivalent vaccines.

| Combination | Position 1 | Position 2 | Position 3 |
|---|---|---|---|
| 1 | F-gene of PIV | F-gene of RSV | — |
| 2 | F-gene of RSV | F-gene of PIV | — |
| 3 | — | F-gene of PIV | F-gene of RSV |
| 4 | — | F-gene of RSV | F-gene of PIV |
| 5 | F-gene of PIV | — | F-gene of RSV |
| 6 | F-gene of RSV | — | F-gene of PIV |
| 7 | HN-gene of PIV | G-gene of RSV | — |
| 8 | G-gene of RSV | HN-gene of PIV | — |
| 9 | — | HN-gene of PIV | G-gene of RSV |
| 10 | — | G-gene of RSV | HN-gene of PIV |
| 11 | HN-gene of PIV | — | G-gene of RSV |
| 12 | G-gene of RSV | — | HN-gene of PIV |
| 13 | F-gene of PIV | G-gene of RSV | — |
| 14 | G-gene of RSV | F-gene of PIV | — |
| 15 | — | F-gene of PIV | G-gene of RSV |
| 16 | — | G-gene of RSV | F-gene of PIV |
| 17 | F-gene of PIV | — | G-gene of RSV |
| 18 | G-gene of RSV | — | F-gene of PIV |
| 19 | HN-gene of PIV | F-gene of RSV | — |
| 20 | F-gene of RSV | HN-gene of PIV | — |
| 21 | — | HN-gene of PIV | F-gene of RSV |
| 22 | — | F-gene of RSV | HN-gene of PIV |
| 23 | HN-gene of PIV | — | F-gene of RSV |
| 24 | F-gene of RSV | — | HN-gene of PIV |

In certain embodiments, the expression products and recombinant or chimeric virions of the present invention may be engineered to create vaccines against a broad range of pathogens, including viral antigens, tumor antigens and auto antigens involved in autoimmune disorders. One way to achieve this goal involves modifying existing metapneumoviral genes to contain foreign sequences in their respective external domains. Where the heterologous sequences are epitopes or antigens of pathogens, these chimeric viruses may be used to induce a protective immune response against the disease agent from which these determinants are derived.

Thus, the present invention relates to the use of viral vectors and recombinant or chimeric viruses to formulate vaccines against a broad range of viruses and/or antigens. The viral vectors and chimeric viruses of the present invention may be used to modulate a subject's immune system by stimulating a humoral immune response, a cellular immune response or by stimulating tolerance to an antigen. As used herein, a subject means: humans, primates, horses, cows, sheep, pigs, goats, dogs, cats, avian species and rodents.

The invention may be divided into the following stages solely for the purpose of description and not by way of limitation: (a) construction of recombinant cDNA and RNA templates; (b) expression of heterologous gene products using recombinant cDNA and RNA templates; (c) rescue of the heterologous gene in recombinant virus particles; and (d) generation and use of vaccines comprising the recombinant virus particles of the invention.

5.4 Construction of the Recombinant cDNA and RNA

In certain embodiments, the viral vectors are derived from the genomes of human or mammalian metapneumovirus of the invention. In other embodiments, the viral vectors are derived from the genome of avian pneumovirus. In certain embodiments, viral vectors contain sequences derived from mammalian MPV and APV, such that a chimeric human MPV/APV virus is encoded by the viral vector. In an exemplary embodiment, the F-gene and/or the G-gene of human metapneumovirus have been replaced with the F-gene and/or the G-gene of avian pneumovirus to construct chimeric hMPV/APV virus. In other embodiments, viral vectors contain sequences derived from APV and mammalian MPV, such that a chimeric APV/hMPV virus is encoded by the viral vector. In more exemplary embodiments, the F-gene and/or the G-gene of avian pneumovirus have been replaced with the F-gene and/or the G-gene of human metapneumovirus to construct the chimeric APV/hMPV virus.

The present invention also encompasses recombinant viruses comprising a viral vector derived from a mammalian MPV or APV genome containing sequences endogenous or native to the viral genome, and may or may not contain sequences non-native to the viral genome. Non-native sequences include those that are different from native or endogenous sequences which may or may not result in a phenotypic change. The recombinant viruses of the invention may contain sequences which result in a virus having a phenotype more suitable for use in vaccine formulations, e.g., attenuated phenotype or enhanced antigenicity. The mutations and modifications can be in coding regions, in intergenic regions and in the leader and trailer sequences of the virus.

In certain embodiments the viral vectors of the invention comprise nucleotide sequences derived from hMPV, APV, hMPV/APV or APV/hMPV, in which native nucleotide sequences have been substituted with heterologous sequences or in which heterologous sequences have been added to the native metapneumoviral sequences.

In a more specific embodiment, a chimeric virus comprises a viral vector derived from MPV, APV, APV/hMPV, or hMPV/APV in which heterologous sequences derived from PIV have been added. In a more specific embodiment, a recombinant virus comprises a viral vector derived from MPV, APV, APV/hMPV, or hMPV/APV in which sequences have been replaced by heterologous sequences derived from PIV. In other specific embodiments, a chimeric virus comprises a viral vector derived from MPV, APV, APV/hMPV, or hMPV/APV in which heterologous sequences derived from RSV have been added. In a more specific embodiment, a chimeric virus comprises a viral vector derived from MPV, APV, APV/hMPV, or hMPV/APV in which sequences have been replaced by heterologous sequences derived from RSV.

Heterologous gene coding sequences flanked by the complement of the viral polymerase binding site/promoter, e.g., the complement of 3′-hMPV virus terminus of the present invention, or the complements of both the 3′- and 5′-hMPV virus termini may be constructed using techniques known in the art. In more specific embodiments, a recombinant virus of the invention contains the leader and trailer sequence of hMPV or APV. In certain embodiments, the intergenic regions are obtained from hMPV or APV. The resulting RNA templates may be of the negative-polarity and contain appropriate terminal sequences which enable the viral RNA-synthesizing apparatus to recognize the template. Alternatively, positive-polarity RNA templates which contain appropriate terminal sequences which enable the viral RNA-synthesizing apparatus to recognize the template, may also be used. Recombinant DNA molecules containing these hybrid sequences can be cloned and transcribed by a DNA-directed RNA polymerase, such as bacteriophage T7, T3, the SP6 polymerase or eukaryotic polymerase such as polymerase I and the like, to produce in vitro or in vivo the recombinant RNA templates which possess the appropriate viral sequences that allow for viral polymerase recognition and activity. In a more specific embodiment, the RNA polymerase is fowlpox virus T7 RNA polymerase or a MVA T7 RNA polymerase.

An illustrative approach for constructing these hybrid molecules is to insert the heterologous nucleotide sequence into a DNA complement of a hMPV, APV, APV/hMPV or hMPV/APV genome, so that the heterologous sequence is flanked by the viral sequences required for viral polymerase activity; i.e., the viral polymerase binding site/promoter, hereinafter referred to as the viral polymerase binding site, and a polyadenylation site. In a preferred embodiment, the heterologous coding sequence is flanked by the viral sequences that comprise the replication promoters of the 5′ and 3′ termini, the gene start and gene end sequences, and the packaging signals that are found in the 5′ and/or the 3′ termini. In an alternative approach, oligonucleotides encoding the viral polymerase binding site, e.g., the complement of the 3′-terminus or both termini of the virus genomic segment can be ligated to the heterologous coding sequence to construct the hybrid molecule. The placement of a foreign gene or segment of a foreign gene within a target sequence was formerly dictated by the presence of appropriate restriction enzyme sites within the target sequence. However, recent advances in molecular biology have lessened this problem greatly. Restriction enzyme sites can readily be placed anywhere within a target sequence through the use of site-directed mutagenesis (e.g., see, for example, the techniques described by Kunkel, 1985, Proc. Natl. Acad. Sci. U.S.A. 82;488). Variations in polymerase chain reaction (PCR) technology, described infra, also allow for the specific insertion of sequences (i.e., restriction enzyme sites) and allow for the facile construction of hybrid molecules. Alternatively, PCR reactions could be used to prepare recombinant templates without the need of cloning. For example, PCR reactions could be used to prepare double-stranded DNA molecules containing a DNA-directed RNA polymerase promoter (e.g., bacteriophage T3, T7 or SP6) and the hybrid sequence containing the heterologous gene and the PIV polymerase binding site. RNA templates could then be transcribed directly from this recombinant DNA. In yet another embodiment, the recombinant RNA templates may be prepared by ligating RNAs specifying the negative polarity of the heterologous gene and the viral polymerase binding site using an RNA ligase.

In addition, one or more nucleotides can be added in the untranslated region to adhere to the “Rule of Six” which may be important in obtaining virus rescue. The “Rule of Six” applies to many paramyxoviruses and states that the RNA nucleotide genome must be divisible by six to be functional. The addition of nucleotides can be accomplished by techniques known in the art such as using a commercial mutagenesis kits such as the QuikChange mutagenesis kit (Stratagene). After addition of the appropriate number of nucleotides, the correct DNA fragment can then be isolated by digestion with appropriate restriction enzyme and gel purification. Sequence requirements for viral polymerase activity and constructs which may be used in accordance with the invention are described in the subsections below.

Without being bound by theory, several parameters affect the rate of replication of the recombinant virus and the level of expression of the heterologous sequence. In particular, the position of the heterologous sequence in hMPV, APV, hMPV/APV or APV/hMPV and the length of the intergenic region that flanks the heterologous sequence determine rate of replication and expression level of the heterologous sequence.

In certain embodiments, the leader and or trailer sequence of the virus are modified relative to the wild type virus. In certain more specific embodiments, the lengths of the leader and/or trailer are altered. In other embodiments, the sequence(s) of the leader and/or trailer are mutated relative to the wild type virus. For more detail, see section 5.7.

The production of a recombinant virus of the invention relies on the replication of a partial or full-length copy of the negative sense viral RNA (vRNA) genome or a complementary copy thereof (cRNA). This vRNA or cRNA can be isolated from infectious virus, produced upon in-vitro transcription, or produced in cells upon transfection of nucleic acids. Second, the production of recombinant negative strand virus relies on a functional polymerase complex. Typically, the polymerase complex of pneumoviruses consists of N, P, L and possibly M2 proteins, but is not necessarily limited thereto.

Polymerase complexes or components thereof can be isolated from virus particles, isolated from cells expressing one or more of the components, or produced upon transfection of specific expression vectors.

Infectious copies of MPV can be obtained when the above mentioned vRNA, cRNA, or vectors expressing these RNAs are replicated by the above mentioned polymerase complex 16 (Schnell et al., 1994, EMBO J 13: 4195-4203; Collins, et al., 1995, PNAS 92: 11563-11567; Hoffmann, et al., 2000, PNAS 97: 6108-6113; Bridgen, et al., 1996, PNAS 93: 15400-15404; Palese, et al., 1996, PNAS 93: 11354-11358; Peeters, et al., 1999, J. Virol. 73: 5001-5009; Durbin, et al., 1997, Virology 235: 323-332).

The invention provides a host cell comprising a nucleic acid or a vector according to the invention. Plasmid or viral vectors containing the polymerase components of MPV (presumably N, P, L and M2, but not necessarily limited thereto) are generated in prokaryotic cells for the expression of the components in relevant cell types (bacteria, insect cells, eukaryotic cells). Plasmid or viral vectors containing full-length or partial copies of the MPV genome will be generated in prokaryotic cells for the expression of viral nucleic acids in-vitro or in-vivo. The latter vectors may contain other viral sequences for the generation of chimeric viruses or chimeric virus proteins, may lack parts of the viral genome for the generation of replication defective virus, and may contain mutations, deletions or insertions for the generation of attenuated viruses.

Infectious copies of MPV (being wild type, attenuated, replication-defective or chimeric) can be produced upon co-expression of the polymerase components according to the state-of-the-art technologies described above.

In addition, eukaryotic cells, transiently or stably expressing one or more full-length or partial MPV proteins can be used. Such cells can be made by transfection (proteins or nucleic acid vectors), infection (viral vectors) or transduction (viral vectors) and may be useful for complementation of mentioned wild type, attenuated, replication-defective or chimeric viruses.

### 5.4.1 Heterologous Gene Sequences to be Inserted

In accordance with the present invention the viral vectors of the invention may be further engineered to express a heterologous sequence. In an embodiment of the invention, the heterologous sequence is derived from a source other than the viral vector. By way of example, and not by limitation, the heterologous sequence encodes an antigenic protein, polypeptide or peptide of a virus belonging to a different species, subgroup or variant of metapneumovirus than the species, subgroup or variant from which the viral vector is derived. By way of example, and not by limitation, the heterologous sequence encodes an antigenic protein, polypeptide or peptide of a virus other than a metapneumovirus. By way of example, and not by limitation, the heterologous sequence is not viral in origin. In accordance with this embodiment, the heterologous sequence may encode a moiety, peptide, polypeptide or protein possessing a desired biological property or activity. Such a heterologous sequence may encode a tag or marker. Such a heterologous sequence may encode a biological response modifier, examples of which include, lymphokines, interleukines, granulocyte macrophage colony stimulating factor and granulocyte colony stimulating factor.

In certain embodiments, the heterologous nucleotide sequence to be inserted is derived from a metapneumovirus. More specifically, the heterologous nucleotide sequence to be inserted is derived from a human metapneumovirus and/or an avian pneumovirus.

In certain embodiments, the heterologous sequence encodes PIV nucleocapsid phosphoprotein, PIV L protein, PIV matrix protein, PIV HN glycoprotein, PIV RNA-dependent RNA polymerase, PIV Y1 protein, PIV D protein, PIV C protein, PIV F protein or PIV P protein. In certain embodiments, the heterologous nucleotide sequence encodes a protein that is at least 90%, at least 95%, at least 98%, or at least 99% homologous to PIV nucleocapsid phosphoprotein, PIV L protein, PIV matrix protein, PIV HN glycoprotein, PIV RNA-dependent RNA polymerase, PIV Y1 protein, PIV D protein, PIV C protein, PIV F protein or PIV P protein. The heterologous sequence can be obtained from PIV type 1, PIV type 2, or PIV type 3. In more specific embodiments, the heterologouse sequence is obtained from human PIV type 1, PIV type 2, or PIV type 3. In other embodiments, the heterologous sequence encodes RSV nucleoprotein, RSV phosphoprotein, RSV matrix protein, RSV small hydrophobic protein, RSV RNA-dependent RNA polymerase, RSV F protein, RSV G protein, or RSV M2-1 or M2-2 protein. In certain embodiments, the heterologous sequence encodes a protein that is at least 90%, at least 95%, at least 98%, or at least 99% homologous to RSV nucleoprotein, RSV phosphoprotein, RSV matrix protein, RSV small hydrophobic protein, RSV RNA-dependent RNA polymerase, RSV F protein, or RSV G protein. The heterologous sequence can be obtained from RSV subtype A and RSV subtype B. In more specific embodiments, the heterologouse sequence is obtained from human RSV subtype A and RSV subtype B. In other embodiments, the heterologous sequence encodes APV nucleoprotein, APV phosphoprotein, APV matrix protein, APV small hydrophobic protein, APV RNA-dependent RNA polymerase, APV F protein, APV G protein or APV M2-1 or M2-2 protein. In certain embodiments, the heterologous sequence encodes a protein that is at least 90%, at least 95%, at least 98%, or at least 99% homologous to APV nucleoprotein, APV phosphoprotein, APV matrix protein, APV small hydrophobic protein, APV RNA-dependent RNA polymerase, APV F protein, or APV G protein. The avian pneumovirus can be APV subgroup A, APV subgroup B, or APV subgroup C. In other embodiments, the heterologous sequence encodes hMPV nucleoprotein, hMPV phosphoprotein, hMPV matrix protein, hMPV small hydrophobic protein, hMPV RNA-dependent RNA polymerase, hMPV F protein, hMPV G protein or hMPV M2-1 or M2-2. In certain embodiments, the heterologous sequence encodes a protein that is at least 90%, at least 95%, at least 98%, or at least 99% homologous to hMPV nucleoprotein, hMPV phosphoprotein, hMPV matrix protein, hMPV small hydrophobic protein, hMPV RNA-dependent RNA polymerase, hMPV F protein, or hMPV G protein. The human metapneumovirus can be hMPV variant A1, hMPV variant A2, hMPV variant B1, or hMPV variant B2.

In certain embodiments, any combination of different heterologous sequence from PIV, RSV, human metapneumovirus, or avian pneumovirus can be inserted into the virus of the invention.

In certain preferred embodiments of the invention, the heterologous nucleotide sequence to be inserted is derived from a F gene from RSV, PIV, APV or hMPV.

In certain embodiments, the heterologous nucleotide sequence encodes a chimeric protein. In more specific embodiments, the heterologous nucleotide sequence encodes a chimeric F protein of RSV, PIV, APV or hMPV. A chimeric F protein can comprise parts of F proteins from different viruses, such as a human metapneumovirus, avian pneumovirus, respiratory syncytial virus, and parainfluenza virus. In certain other embodiments, the heterologous sequence encodes a chimeric G protein. A chimeric G protein comprises parts of G proteins from different viruses, such as a human metapneumovirus, avian pneumovirus, respiratory syncytial virus, and parainfluenza virus. In a specific embodiment, the F protein comprises an ectodomain of a F protein of a metapneumovirus, a transmembrane domain of a F protein of a parainfluenza virus, and luminal domain of a F protein of a parainfluenza virus.

In certain specific embodiments, the heterologous nucleotide sequence of the invention is any one of SEQ ID NO:1 through SEQ ID NO:5, SEQ ID NO:14, and SEQ ID NO:15. In certain specific embodiments, the nucleotide sequence encodes a protein of any one of SEQ ID NO:6 through SEQ ID NO:13, SEQ ID NO:16, and SEQ ID NO:17.

For heterologous nucleotide sequences derived from respiratory syncytial virus see, e.g., PCT/US98/20230, which is hereby incorporated by reference in its entirety.

In a preferred embodiment, heterologous gene sequences that can be expressed into the recombinant viruses of the invention include but are not limited to antigenic epitopes and glycoproteins of viruses which result in respiratory disease, such as influenza glycoproteins, in particular hemagglutinin H5, H7, respiratory syncytial virus epitopes, New Castle Disease virus epitopes, Sendai virus and infectious Laryngotracheitis virus (ILV). In a preferred embodiment, the heterologous nucleotide sequences are derived from a RSV or PIV. In yet another embodiment of the invention, heterologous gene sequences that can be engineered into the chimeric viruses of the invention include, but are not limited to, viral epitopes and glycoproteins of viruses, such as hepatitis B virus surface antigen, hepatitis A or C virus surface glycoproteins of Epstein Barr virus, glycoproteins of human papilloma virus, simian virus 5 or mumps virus, West Nile virus, Dengue virus, glycoproteins of herpes viruses, VPI of poliovirus, and sequences derived from a lentivirus, preferably, but not limited to human immunodeficiency virus (HIV) type 1 or type 2. In yet another embodiment, heterologous gene sequences that can be engineered into chimeric viruses of the invention include, but are not limited to, Marek's Disease virus (MDV) epitopes, epitopes of infectious Bursal Disease virus (IBDV), epitopes of Chicken Anemia virus, infectious laryngotracheitis virus (ILV), Avian Influenza virus (AIV), rabies, feline leukemia virus, canine distemper virus, vesicular stomatitis virus, and swinepox virus (see Fields et al., (ed.), 1991, Fundamental Virology, Second Edition, Raven Press, New York, incorporated by reference herein in its entirety).

Other heterologous sequences of the present invention include antigens that are characteristic of autoimmune disease. These antigens will typically be derived from the cell surface, cytoplasm, nucleus, mitochondria and the like of mammalian tissues, including antigens characteristic of diabetes mellitus, multiple sclerosis, systemic lupus erythematosus, rheumatoid arthritis, pernicious anemia, Addison's disease, scleroderma, autoimmune atrophic gastritis, juvenile diabetes, and discoid lupus erythematosus.

Antigens that are allergens generally include proteins or glycoproteins, including antigens derived from pollens, dust, molds, spores, dander, insects and foods. In addition, antigens that are characteristic of tumor antigens typically will be derived from the cell surface, cytoplasm, nucleus, organelles and the like of cells of tumor tissue. Examples include antigens characteristic of tumor proteins, including proteins encoded by mutated oncogenes; viral proteins associated with tumors; and glycoproteins. Tumors include, but are not limited to, those derived from the types of cancer: lip, nasopharynx, pharynx and oral cavity, esophagus, stomach, colon, rectum, liver, gall bladder, pancreas, larynx, lung and bronchus, melanoma of skin, breast, cervix, uterine, ovary, bladder, kidney, uterus, brain and other parts of the nervous system, thyroid, prostate, testes, Hodgkin's disease, non-Hodgkin's lymphoma, multiple myeloma and leukemia.

In one specific embodiment of the invention, the heterologous sequences are derived from the genome of human immunodeficiency virus (HIV), preferably human immunodeficiency virus-1 or human immunodeficiency virus-2. In another embodiment of the invention, the heterologous coding sequences may be inserted within a gene coding sequence of the viral backbone such that a chimeric gene product is expressed which contains the heterologous peptide sequence within the metapneumoviral protein. In such an embodiment of the invention, the heterologous sequences may also be derived from the genome of a human immunodeficiency virus, preferably of human immunodeficiency virus-1 or human immunodeficiency virus-2.

In instances whereby the heterologous sequences are HIV-derived, such sequences may include, but are not limited to sequences derived from the env gene (i.e., sequences encoding all or part of gp160, gp120, and/or gp41), the pol gene (i.e., sequences encoding all or part of reverse transcriptase, endonuclease, protease, and/or integrase), the gag gene (i.e., sequences encoding all or part of p7, p6, p55, p17/18, p24/25) tat, rev, nef, vif, vpu, vpr, and/or vpx.

In yet another embodiment, heterologous gene sequences that can be engineered into the chimeric viruses include those that encode proteins with immunopotentiating activities. Examples of immunopotentiating proteins include, but are not limited to, cytokines, interferon type 1, gamma interferon, colony stimulating factors, and interleukin -1, -2, -4, -5, -6, -12.

In addition, other heterologous gene sequences that may be engineered into the chimeric viruses include antigens derived from bacteria such as bacterial surface glycoproteins, antigens derived from fungi, and antigens derived from a variety of other pathogens and parasites. Examples of heterologous gene sequences derived from bacterial pathogens include, but are not limited to, antigens derived from species of the following genera: Salmonella, Shigella, Chlamydia, Helicobacter, Yersinia, Bordatella, Pseudomonas, Neisseria, Vibrio, Haemophilus, Mycoplasma, Streptomyces, Treponema, Coxiella, Ehrlichia, Brucella, Streptobacillus, Fusospirocheta, Spirillum, Ureaplasma, Spirochaeta, Mycoplasma, Actinomycetes, Borrelia, Bacteroides, Trichomoras, Branhamella, Pasteurella, Clostridium, Corynebacterium, Listeria, Bacillus, Erysipelothrix, Rhodococcus, Escherichia, Klebsiella, Pseudomanas, Enterobacter, Serratia, Staphylococcus, Streptococcus, Legionella, Mycobacterium, Proteus, Campylobacter, Enterococcus, Acinetobacter, Morganella, Moraxella, Citrobacter, Rickettsia, Rochlimeae, as well as bacterial species such as: P. aeruginosa; E. coli, P. cepacia, S. epidermis, E. faecalis, S. pneumonias, S. aureus, N. meningitidis, S. pyogenes, Pasteurella multocida, Treponema pallidum, and P. mirabilis.

Examples of heterologous gene sequences derived from pathogenic fungi, include, but are not limited to, antigens derived from fungi such as Cryptococcus neoformans; Blastomyces dermatitidis; Aiellomyces dermatitidis; Histoplasma capsulatum; Coccidioides immitis; Candida species, including C. albicans, C. tropicalis, C. parapsilosis, C. guilliermondii and C. krusei, Aspergillus species, including A. fumigatus, A. flavus and A. niger, Rhizopus species; Rhizomucor species; Cunninghammella species; Apophysomyces species, including A. saksenaea, A. mucor and A. absidia; Sporothrix schenckii, Paracoccidioides brasiliensis; Pseudallescheria boydii, Torulopsis glabrata; Trichophyton species, Microsporum species and Dermatophyres species, as well as any other yeast or fungus now known or later identified to be pathogenic.

Finally, examples of heterologous gene sequences derived from parasites include, but are not limited to, antigens derived from members of the Apicomplexa phylum such as, for example, Babesia, Toxoplasma, Plasmodium, Eimeria, Isospora, Atoxoplasma, Cystoisospora, Hammondia, Besniotia, Sarcocystis, Frenkelia, Haemoproteus, Leucocytozoon, Theileria, Perkinsus and Gregarina spp.; Pneumocystis carinii; members of the Microspora phylum such as, for example, Nosema, Enterocytozoon, Encephalitozoon, Septata, Mrazekia, Amblyospora, Ameson, Glugea, Pleistophora and Microsporidium spp.; and members of the Ascetospora phylum such as, for example, Haplosporidium spp., as well as species including Plasmodium falciparum, P. vivax, P. ovale, P. malaria; Toxoplasma gondii; Leishmania mexicana, L. tropica, L. major, L. aethiopica, L. donovani, Trypanosoma cruzi, T. brucei, Schistosoma mansoni, S. haematobium, S. japonium; Trichinella spiralis; Wuchereria bancrofti; Brugia malayli; Entamoeba histolytica; Enterobius vermiculoarus; Taenia solium, T saginata, Trichomonas vaginatis, T hominis, T. tenax; Giardia lamblia; Cryptosporidium parvum; Pneumocytis carinii, Babesia bovis, B. divergens, B. microti, Isospora belli, L hominis; Dientamoeba fragilis; Onchocerca volvulus; Ascaris lumbricoides; Necator americanis; Ancylostoma duodenale; Strongyloides stercoralis; Capillaria philippinensis; Angiostrongylus cantonensis; Hymenolepis nana; Diphyllobothrium latum; Echinococcus granulosus, E. multilocularis; Paragonimus westermani, P. caliensis; Chlonorchis sinensis; Opisthorchis felineas, G. Viverini, Fasciola hepatica, Sarcoptes scabiei, Pediculus humanus; Phthirlus pubis; and Dermatobia hominis, as well as any other parasite now known or later identified to be pathogenic.

### 5.4.2 Insertin of the Heterologous Gene Sequence

Insertion of a foreign gene sequence into a viral vector of the invention can be accomplished by either a complete replacement of a viral coding region with a heterologous sequence or by a partial replacement or by adding the heterologous nucleotide sequence to the viral genome. Complete replacement would probably best be accomplished through the use of PCR-directed mutagenesis. Briefly, PCR-primer A would contain, from the 5′ to 3′end: a unique restriction enzyme site, such as a class IIS restriction enzyme site (i.e., a “shifter” enzyme; that recognizes a specific sequence but cleaves the DNA either upstream or downstream of that sequence); a stretch of nucleotides complementary to a region of the gene that is to be replaced; and a stretch of nucleotides complementary to the carboxy-terminus coding portion of the heterologous sequence. PCR-primer B would contain from the 5′ to 3′ end: a unique restriction enzyme site; a stretch of nucleotides complementary to the gene that is to be replaced; and a stretch of nucleotides corresponding to the 5′ coding portion of the heterologous or non-native gene. After a PCR reaction using these primers with a cloned copy of the heterologous or non-native gene, the product may be excised and cloned using the unique restriction sites. Digestion with the class HS enzyme and transcription with the purified phage polymerase would generate a RNA molecule containing the exact untranslated ends of the viral gene that carries now a heterologous or non-native gene insertion. In an alternate embodiment, PCR-primed reactions could be used to prepare double-stranded DNA containing the bacteriophage promoter sequence, and the hybrid gene sequence so that RNA templates can be transcribed directly without cloning.

A heterologous nucleotide sequence can be added or inserted at various positions of the virus of the invention. In one embodiment, the heterologous nucleotide sequence is added or inserted at position 1. In another embodiment, the heterologous nucleotide sequence is added or inserted at position 2. In another embodiment, the heterologous nucleotide sequence is added or inserted at position 3. In another embodiment, the heterologous nucleotide sequence is added or inserted at position 4. In another embodiment, the heterologous nucleotide sequence is added or inserted at position 5. In yet another embodiment, the heterologous nucleotide sequence is added or inserted at position 6. As used herein, the term “position” refers to the position of the heterologous nucleotide sequence on the viral genome to be transcribed, e.g., position 1 means that it is the first gene to be transcribed, and position 2 means that it is the second gene to be transcribed. Inserting heterologous nucleotide sequences at the lower-numbered positions of the virus generally results in stronger expression of the heterologous nucleotide sequence compared to insertion at higher-numbered positions due to a transcriptional gradient that occurs across the genome of the virus. However, the transcriptional gradient also yields specific ratios of viral mRNAs. Insertion of foreign genes will perturb these ratios and result in the synthesis of different amounts of viral proteins that may influence virus replication. Thus, both the transcriptional gradient and the replication kinetics must be considered when choosing an insertion site. Inserting heterologous nucleotide sequences at lower-numbered positions is the preferred embodiment of the invention if strong expression of the heterologous nucleotide sequence is desired. In a preferred embodiment, the heterologous sequence is added or inserted at position 1, 2 or 3.

When inserting a heterologous nucleotide sequence into the virus of the invention, the intergenic region between the end of the coding sequence of the heterologous gene and the start of the coding sequence of the downstream gene can be altered to achieve a desired effect. As used herein, the term “intergenic region” refers to nucleotide sequence between the stop signal of one gene and the start codon (e.g., AUG) of the coding sequence of the next downstream open reading frame. An intergenic region may comprise a non-coding region of a gene, i.e., between the transcription start site and the start of the coding sequence (AUG) of the gene. This non-coding region occurs naturally in some viral genes.

In various embodiments, the intergenic region between the heterologous nucleotide sequence and the downstream gene can be engineered, independently from each other, to be at least 10 nt in length, at least 20 nt in length, at least 30 nt in length, at least 50 nt in length, at least 75 nt in length, at least 100 nt in length, at least 125 nt in length, at least 150 nt in length, at least 175 nt in length or at least 200 nt in length. In certain embodiments, the intergenic region between the heterologous nucleotide sequence and the downstream gene can be engineered, independently from each other, to be at most 10 nt in length, at most 20 nt in length, at most 30 nt in length, at most 50 nt in length, at most 75 nt in length, at most 100 nt in length, at most 125 nt in length, at most 150 nt in length, at most 175 nt in length or at most 200 nt in length. In various embodiments, the non-coding region of a desired gene in a virus genome can also be engineered, independently from each other, to be at least 10 nt in length, at least 20 nt in length, at least 30 nt in length, at least 50 nt in length, at least 75 nt in length, at least 100 nt in length, at least 125 nt in length, at least 150 nt in length, at least 175 nt in length or at least 200 nt in length. In certain embodiments, the non-coding region of a desired gene in a virus genome can also be engineered, independently from each other, to be at most 10 nt in length, at most 20 nt in length, at most 30 nt in length, at most 50 nt in length, at most 75 nt in length, at most 100 nt in length, at most 125 nt in length, at most 150 nt in length, at most 175 nt in length or at most 200 nt in length.

When inserting a heterologous nucleotide sequence, the positional effect and the intergenic region manipulation can be used in combination to achieve a desirable effect. For example, the heterologous nucleotide sequence can be added or inserted at a position selected from the group consisting of position 1, 2, 3, 4, 5, and 6, and the intergenic region between the heterologous nucleotide sequence and the next downstream gene can be altered (see Table 3). Some of the combinations encompassed by the present invention are shown by way of example in Table 3.

TABLE 3
Examples of mode of insertion of heterologous nucleotide sequences

| | Position | | | | | Position |
|---|---|---|---|---|---|---|
| | 1 | Position 2 | Position 3 | Position 4 | Position 5 | 6 |
| IGR^{a} | 10-20 | 10-20 | 10-20 | 10-20 | 10-20 | 10-20 |
| IGR | 21-40 | 21-40 | 21-40 | 21-40 | 21-40 | 21-40 |
| IGR | 41-60 | 41-60 | 41-60 | 41-60 | 41-60 | 41-60 |
| IGR | 61-80 | 61-80 | 61-80 | 61-80 | 61-80 | 61-80 |
| IGR | 81-100 | 81-100 | 81-100 | 81-100 | 81-100 | 81-100 |
| IGR | 101-120 | 101-120 | 101-120 | 101-120 | 101-120 | 101-120 |
| IGR | 121-140 | 121-140 | 121-140 | 121-140 | 121-140 | 121-140 |
| IGR | 141-160 | 141-160 | 141-160 | 141-160 | 141-160 | 141-160 |
| IGR | 161-180 | 161-180 | 161-180 | 161-180 | 161-180 | 161-180 |
| IGR | 181-200 | 181-200 | 181-200 | 181-200 | 181-200 | 181-200 |
| IGR | 201-220 | 201-220 | 201-220 | 201-220 | 201-220 | 201-220 |
| IGR | 221-240 | 221-240 | 221-240 | 221-240 | 221-240 | 221-240 |
| IGR | 241-260 | 241-260 | 241-260 | 241-260 | 241-260 | 241-260 |
| IGR | 261-280 | 261-280 | 261-280 | 261-280 | 261-280 | 261-280 |
| IGR | 281-300 | 281-300 | 281-300 | 281-300 | 281-300 | 281-300 |

^{a}Intergenic Region, measured in nucleotide.

Depending on the purpose (e.g., to have strong immunogenicity) of the inserted heterologous nucleotide sequence, the position of the insertion and the length of the intergenic region of the inserted heterologous nucleotide sequence can be determined by various indexes including, but not limited to, replication kinetics and protein or mRNA expression levels, measured by following non-limiting examples of assays: plaque assay, fluorescent-focus assay, infectious center assay, transformation assay, endpoint dilution assay, efficiency of plating, electron microscopy, hemagglutination, measurement of viral enzyme activity, viral neutralization, hemagglutination inhibition, complement fixation, immunostaining, immunoprecipitation and immunoblotting, enzyme-linked immunosorbent assay, nucleic acid detection (e.g., Southern blot analysis, Northern blot analysis, Western blot analysis), growth curve, employment of a reporter gene (e.g., using a reporter gene, such as Green Fluorescence Protein (GFP) or enhanced Green Fluorescence Protein (eGFP), integrated to the viral genome the same fashion as the interested heterologous gene to observe the protein expression), or a combination thereof. Procedures of performing these assays are well known in the art (see, e.g., Flint et al., PRINCIPLES OF VIROLOGY, MOLECULAR BIOLOGY, PATHOGENESIS, AND CONTROL, 2000, ASM Press pp 25-56, the entire text is incorporated herein by reference), and non-limiting examples are given in the Example sections, infra.

For example, expression levels can be determined by infecting cells in culture with a virus of the invention and subsequently measuring the level of protein expression by, e.g., Western blot analysis or ELISA using antibodies specific to the gene product of the heterologous sequence, or measuring the level of RNA expression by, e.g., Northern blot analysis using probes specific to the heterologous sequence. Similarly, expression levels of the heterologous sequence can be determined by infecting an animal model and measuring the level of protein expressed from the heterologous sequence of the recombinant virus of the invention in the animal model. The protein level can be measured by obtaining a tissue sample from the infected animal and then subjecting the tissue sample to Western blot analysis or ELISA, using antibodies specific to the gene product of the heterologous sequence. Further, if an animal model is used, the titer of antibodies produced by the animal against the gene product of the heterologous sequence can be determined by any technique known to the skilled artisan, including but not limited to, ELISA.

As the heterologous sequences can be homologous to a nucleotide sequence in the genome of the virus, care should be taken that the probes and the antibodies are indeed specific to the heterologous sequence or its gene product.

In certain specific embodiments, expression levels of F-protein of hMPV from chimeric avian-human metapneumovirus can be determined by any technique known to the skilled artisan. Expression levels of the F-protein can be determined by infecting cells in a culture with the chimeric virus of the invention and measuring the level of protein expression by, e.g., Western blot analysis or ELISA using antibodies specific to the F-protein and/or the G-protein of hMPV, or measuring the level of RNA expression by, e.g., Northern blot analysis using probes specific to the F-gene and/or the G-gene of human metapneumovirus. Similarly, expression levels of the heterologous sequence can be determined using an animal model by infecting an animal and measuring the level of F-protein and/or G-protein in the animal model. The protein level can be measured by obtaining a tissue sample from the infected animal and then subjecting the tissue sample to Western blot analysis or ELISA using antibodies specific to F-protein and/or G-protein of the heterologous sequence. Further, if an animal model is used, the titer of antibodies produced by the animal against F-protein and/or G-protein can be determined by any technique known to the skilled artisan, including but not limited to, ELISA.

The rate of replication of a recombinant virus of the invention can be determined by any technique known to the skilled artisan.

In certain embodiments, to facilitate the identification of the optimal position of the heterologous sequence in the viral genome and the optimal length of the intergenic region, the heterologous sequence encodes a reporter gene. Once the optimal parameters are determined, the reporter gene is replaced by a heterologous nucleotide sequence encoding an antigen of choice. Any reporter gene known to the skilled artisan can be used with the methods of the invention. For more detail, see section 5.8.

The rate of replication of the recombinant virus can be determined by any standard technique known to the skilled artisan. The rate of replication is represented by the growth rate of the virus and can be determined by plotting the viral titer over the time post infection. The viral titer can be measured by any technique known to the skilled artisan. In certain embodiments, a suspension containing the virus is incubated with cells that are susceptible to infection by the virus. Cell types that can be used with the methods of the invention include, but are not limited to, Vero cells, LLC-MK-2 cells, Hep-2 cells, LF 1043 (HEL) cells, MRC-5 cells, WI-38 cells, tMK cells, 293 T cells, QT 6 cells, QT 35 cells, or chicken embryo fibroblasts (CEF). Subsequent to the incubation of the virus with the cells, the number of infected cells is determined. In certain specific embodiments, the virus comprises a reporter gene. Thus, the number of cells expressing the reporter gene is representative of the number of infected cells. In a specific embodiment, the virus comprises a heterologous nucleotide sequence encoding for eGFP, and the number of cells expressing eGFP, i.e., the number of cells infected with the virus, is determined using FACS.

In certain embodiments, the replication rate of the recombinant virus of the invention is at most 20% of the replication rate of the wild type virus from which the recombinant virus is derived under the same conditions. The same conditions refer to the same initial titer of virus, the same strain of cells, the same incubation temperature, growth medium, number of cells and other test conditions that may affect the replication rate. For example, the replication rate of APV/hMPV with PIV's F gene in position 1 is at most 20% of the replication rate of APV.

In certain embodiments, the replication rate of the recombinant virus of the invention is at most 5%, at most 10%, at most 20%, at most 30%, at most 40%, at most 50%, at most 75%, at most 80%, at most 90% of the replication rate of the wild type virus from which the recombinant virus is derived under the same conditions. In certain embodiments, the replication rate of the recombinant virus of the invention is at least 5%, at least 10%, at least 20%, at least 30%, at least 40%, at least 50%, at least 75%, at least 80%, at least 90% of the replication rate of the wild type virus from which the recombinant virus is derived under the same conditions. In certain embodiments, the replication rate of the recombinant virus of the invention is between 5% and 20%, between 10% and 40%, between 25% and 50%, between 40% and 75%, between 50% and 80%, or between 75% and 90% of the replication rate of the wild type virus from which the recombinant virus is derived under the same conditions.

In certain embodiments, the expression level of the heterologous sequence in the recombinant virus of the invention is at most 20% of the expression level of the F-protein of the wild type virus from which the recombinant virus is derived under the same conditions. The same conditions refer to the same initial titer of virus, the same strain of cells, the same incubation temperature, growth medium, number of cells and other test conditions that may affect the replication rate. For example, the expression level of the heterologous sequence of the F-protein of PIV3 in position 1 of hMPV is at most 20% of the expression level of the F-protein of hMPV.

In certain embodiments, the expression level of the heterologous sequence in the recombinant virus of the invention is at most 5%, at most 10%, at most 20%, at most 30%, at most 40%, at most 50%, at most 75%, at most 80%, at most 90% of the expression level of the F-protein of the wild type virus from which the recombinant virus is derived under the same conditions. In certain embodiments, the expression level of the heterologous sequence in the recombinant virus of the invention is at least 5%, at least 10%, at least 20%, at least 30%, at least 40%, at least 50%, at least 75%, at least 80%, at least 90% of the expression level of the F-protein of the wild type virus from which the recombinant virus is derived under the same conditions. In certain embodiments, the expression level of the heterologous sequence in the recombinant virus of the invention is between 5% and 20%, between 10% and 40%, between 25% and 50%, between 40% and 75%, between 50% and 80%, or between 75% and 90% of the expression level of the F-protein of the wild type virus from which the recombinant virus is derived under the same conditions.

5.4.3 Insertion of the Heterologous Gene Sequence into the G Gene

The G protein is a transmembrane protein of metapneumoviruses. In a specific embodiment, the heterologous sequence is inserted into the region of the G-ORF that encodes for the ectodomain, such that it is expressed on the surface of the viral envelope. In one approach, the heterologous sequence may be inserted within the antigenic site without deleting any viral sequences. In another approach, the heterologous sequences replaces sequences of the G-ORF. Expression products of such constructs may be useful in vaccines against the foreign antigen, and may indeed circumvent problems associated with propagation of the recombinant virus in the vaccinated host. An intact G molecule with a substitution only in antigenic sites may allow for G function and thus allow for the construction of a viable virus. Therefore, this virus can be grown without the need for additional helper functions. The virus may also be attenuated in other ways to avoid any danger of accidental escape.

Other hybrid constructions may be made to express proteins on the cell surface or enable them to be released from the cell.

### 5.4.4 Construction of Bicistronic RNA

Bicistronic mRNA could be constructed to permit internal initiation of translation of viral sequences and allow for the expression of foreign protein coding sequences from the regular terminal initiation site. Alternatively, a bicistronic mRNA sequence may be constructed wherein the viral sequence is translated from the regular terminal open reading frame, while the foreign sequence is initiated from an internal site. Certain internal ribosome entry site (IRES) sequences may be utilized. The IRES sequences which are chosen should be short enough to not interfere with MPV packaging limitations. Thus, it is preferable that the IRES chosen for such a bicistronic approach be no more than 500 nucleotides in length. In a specific embodiment, the IRES is derived from a picornavirus and does not include any additional picornaviral sequences. Specific IRES elements include, but are not limited to the mammalian BiP IRES and the hepatitis C virus IRES.

Alternatively, a foreign protein may be expressed from a new internal transcriptional unit in which the transcriptional unit has an initiation site and polyadenylation site. In another embodiment, the foreign gene is inserted into a MPV gene such that the resulting expressed protein is a fusion protein.

5.5 Expression of Heterologous Gene Products Using Recombinant cDNA and RNA Templates

The viral vectors and recombinant templates prepared as described above can be used in a variety of ways to express the heterologous gene products in appropriate host cells or to create chimeric viruses that express the heterologous gene products. In one embodiment, the recombinant cDNA can be used to transfect appropriate host cells and the resulting RNA may direct the expression of the heterologous gene product at high levels. Host cell systems which provide for high levels of expression include continuous cell lines that supply viral functions such as cell lines superinfected with APV or MPV, respectively, cell lines engineered to complement APV or MPV functions, etc.

In an alternate embodiment of the invention, the recombinant templates may be used to transfect cell lines that express a viral polymerase protein in order to achieve expression of the heterologous gene product. To this end, transformed cell lines that express a polymerase protein such as the L protein may be utilized as appropriate host cells. Host cells may be similarly engineered to provide other viral functions or additional functions such as G or N.

In another embodiment, a helper virus may provide the RNA polymerase protein utilized by the cells in order to achieve expression of the heterologous gene product. In yet another embodiment, cells may be transfected with vectors encoding viral proteins such as the N, P, L, and M2-1 proteins.

### 5.6 Rescue of Recombinant Virus Particles

In order to prepare the chimeric and recombinant viruses of the invention, a cDNA encoding the genome of a recombinant or chimeric virus of the invention in the plus or minus sense may be used to transfect cells which provide viral proteins and functions required for replication and rescue. Alternatively, cells may be transfected with helper virus before, during, or after transfection by the DNA or RNA molecule coding for the recombinant virus of the invention. The synthetic recombinant plasmid DNAs and RNAs of the invention can be replicated and rescued into infectious virus particles by any number of techniques known in the art, as described, e.g., in U.S. Pat. No. 5,166,057 issued Nov. 24, 1992; in U.S. Pat. No. 5,854,037 issued Dec. 29, 1998; in European Patent Publication EP 0702085A1, published Feb. 20, 1996; in U.S. patent application Ser. No. 09/152,845; in International Patent Publications PCT WO97/12032 published Apr. 3, 1997; WO96/34625 published Nov. 7, 1996; in European Patent Publication EP-A780475; WO 99/02657 published Jan. 21, 1999; WO 98/53078 published Nov. 26, 1998; WO 98/02530 published Jan. 22, 1998; WO 99/15672 published Apr. 1, 1999; WO 98/13501 published Apr. 2, 1998; WO 97/06270 published Feb. 20, 1997; and EPO 780 47SA1 published Jun. 25, 1997, each of which is incorporated by reference herein in its entirety.

In one embodiment, of the present invention, synthetic recombinant viral RNAs may be prepared that contain the non-coding regions (leader and trailer) of the negative strand virus RNA which are essential for the recognition by viral polymerases and for packaging signals necessary to generate a mature virion. There are a number of different approaches which may be used to apply the reverse genetics approach to rescue negative strand RNA viruses. First, the recombinant RNAs are synthesized from a recombinant DNA template and reconstituted in vitro with purified viral polymerase complex to form recombinant ribonucleoproteins (RNPs) which can be used to transfect cells. In another approach, a more efficient transfection is achieved if the viral polymerase proteins are present during transcription of the synthetic RNAs either in vitro or in vivo. With this approach the synthetic RNAs may be transcribed from cDNA plasmids which are either co-transcribed in vitro with cDNA plasmids encoding the polymerase proteins, or transcribed in vivo in the presence of polymerase proteins, i.e., in cells which transiently or constitutively express the polymerase proteins.

In additional approaches described herein, infectious chimeric or recombinant virus may be replicated in host cell systems that express a metapneumoviral polymerase protein (e.g., in virus/host cell expression systems; transformed cell lines engineered to express a polymerase protein, etc.), so that infectious chimeric or recombinant virus are replicated and rescued. In this instance, helper virus need not be utilized since this function is provided by the viral polymerase proteins expressed.

In accordance with the present invention, any technique known to those of skill in the art may be used to achieve replication and rescue of recombinant and chimeric viruses. One approach involves supplying viral proteins and functions required for replication in vitro prior to transfecting host cells. In such an embodiment, viral proteins may be supplied in the form of wildtype virus, helper virus, purified viral proteins or recombinantly expressed viral proteins. The viral proteins may be supplied prior to, during or post transcription of the synthetic cDNAs or RNAs encoding the chimeric virus. The entire mixture may be used to transfect host cells. In another approach, viral proteins and functions required for replication may be supplied prior to or during transcription of the synthetic cDNAs or RNAs encoding the chimeric virus. In such an embodiment, viral proteins and functions required for replication are supplied in the form of wildtype virus, helper virus, viral extracts, synthetic cDNAs or RNAs which express the viral proteins are introduced into the host cell via infection or transfection. This infection/transfection takes place prior to or simultaneous to the introduction of the synthetic cDNAs or RNAs encoding the chimeric virus genome.

In a particularly desirable approach, cells engineered to express all viral genes or chimeric or recombinant virus of the invention, i.e., APV, MPV, MPV/APV or APV/MPV, may result in the production of infectious virus which contain the desired genotype; thus eliminating the need for a selection system. Theoretically, one can replace any one of the ORFs or part of any one of the ORFs encoding structural proteins of MPV with a foreign sequence. However, a necessary part of this equation is the ability to propagate the defective virus (defective because a normal viral gene product is missing or altered). A number of possible approaches exist to circumvent this problem. In one approach a virus having a mutant protein can be grown in cell lines which are constructed to constitutively express the wild type version of the same protein. By this way, the cell line complements the mutation in the virus. Similar techniques may be used to construct transformed cell lines that constitutively express any of the MPV genes. These cell lines which are made to express the viral protein may be used to complement the defect in the chimeric or recombinant virus and thereby propagate it. Alternatively, certain natural host range systems may be available to propagate chimeric or recombinant virus.

In yet another embodiment, viral proteins and functions required for replication may be supplied as genetic material in the form of synthetic cDNAs or RNAs so that they are co-transcribed with the synthetic cDNAs or RNAs encoding the chimeric virus. In a particularly desirable approach, plasmids which express the chimeric virus and the viral polymerase and/or other viral functions are co-transfected into host cells. For example, plasmids encoding the genomic or antigenomic APV, MPV, MPV/APV or APV/MPV RNA, with or without one or more heterologous sequences, may be co-transfected into host cells with plasmids encoding the metapneumoviral polymerase proteins N, P, L, or M2-1. Alternatively, rescue of the recombinant viruses of the invention may be accomplished by the use of Modified Vaccinia Virus Ankara (MVA) encoding T7 RNA polymerase, or a combination of MVA and plasmids encoding the polymerase proteins (N, P, and L). For example, MVA-T7 or Fowl Pox-T7 can be infected into Vero cells, LLC-MK-2 cells, HEp-2 cells, LF 1043 (HEL) cells, tMK cells, LLC-MK2, HUT 292, FRHL-2 (rhesus), FCL-1 (green monkey), WI-38 (human), MRC-5 (human) cells, 293 T cells, QT 6 cells, QT 35 cells and CEF cells. After infection with MVA-T7 or Fowl Pox-T7, a full length antigenomic or genomic cDNA encoding the recombinant virus of the invention may be transfected into the cells together with the N, P, L, and M2-1 encoding expression plasmids. Alternatively, the polymerase may be provided by plasmid transfection. The cells and cell supernatant can subsequently be harvested and subjected to a single freeze-thaw cycle. The resulting cell lysate may then be used to infect a fresh Vero cell monolayer in the presence of 1-beta-D-arabinofuranosylcytosine (ara C), a replication inhibitor of vaccinia virus, to generate a virus stock. The supernatant and cells from these plates can then be harvested, freeze-thawed once and the presence of recombinant virus particles of the invention can be assayed by immunostaining of virus plaques using antiserum specific to the particular virus.

Another approach to propagating the chimeric or recombinant virus may involve co-cultivation with wild-type virus. This could be done by simply taking recombinant virus and co-infecting cells with this and another wild-type virus. The wild-type virus should complement for the defective virus gene product and allow growth of both the wild-type and recombinant virus. Alternatively, a helper virus may be used to support propagation of the recombinant virus.

In another approach, synthetic templates may be replicated in cells co-infected with recombinant viruses that express the metapneumovirus polymerase protein. In fact, this method may be used to rescue recombinant infectious virus in accordance with the invention. To this end, the metapneumovirus polymerase protein may be expressed in any expression vector/host cell system, including but not limited to viral expression vectors (e.g., vaccinia virus, adenovirus, baculovirus, etc.) or cell lines that express a polymerase protein (e.g., see Krystal et al., 1986, Proc. Natl. Acad. Sci. USA 83: 2709-2713). Moreover, infection of host cells expressing all metapneumovirus proteins may result in the production of infectious chimeric virus particles. It should be noted that it may be possible to construct a recombinant virus without altering virus viability. These altered viruses would then be growth competent and would not need helper functions to replicate.

In order to recombinantly generate viruses in accordance with the methods of the invention, the genetic material encoding the viral genome must be transcribed (transcription step). This step can be accomplished either in vitro (outside the host cell) or in vivo (in a host cell). The viral genome can be transcribed from the genetic material to generate either a positive sense copy of the viral genome (antigenome copy) or a negative sense copy of the viral genome (genomic copy). The next step requires replication of the viral genome and packaging of the replicated genome into viral particles (replication and packaging step). This step occurs intracellularly in a host cell which has been engineered to provide sufficient levels of viral polymerase and structural proteins necessary for viral replication and packaging.

When the transcription step occurs in vitro, it is followed by intracellular replication and packaging of the viral genome. When the transcription step occurs in vivo, transcription of the viral genome can occur prior to, concurrently or subsequently to expression of the viral genetic material encoding the viral genome can be obtained or generated from a variety of sources and using a variety of methods known to one skilled in the art. The genetic material may be isolated from the virus itself. For example, a complex of the viral RNA genome and the polymerase proteins, ribonucleoprotein complexes (RNP), may be isolated from whole virus. The viral RNA genome is then stripped of the associated proteins, e.g., viral RNA polymerase and nuclear proteins.

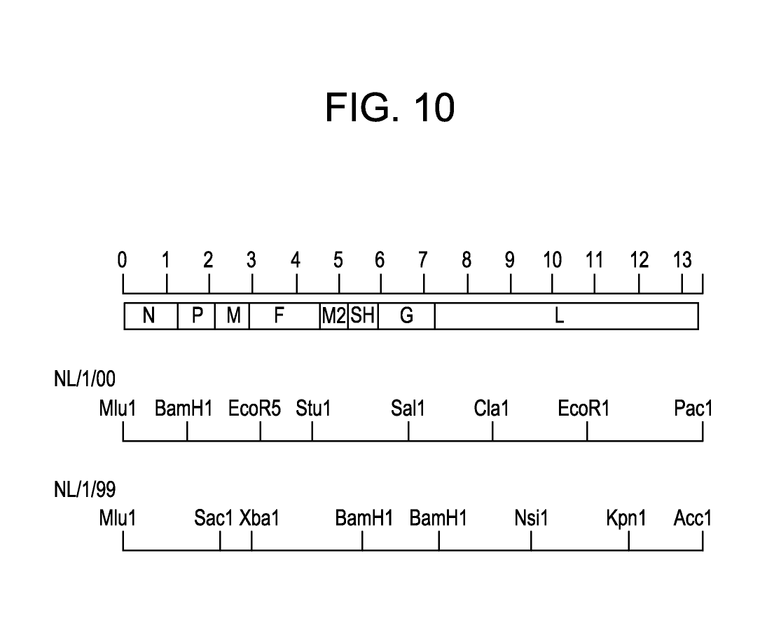

The genetic material encoding the viral genome can be generated using standard recombinant techniques. The genetic material may encode the full length viral genome or a portion thereof. Alternatively, the genetic material may code for a heterologous sequence flanked by the leader and/or trailer sequences of the viral genome. A full-length viral genome can be assembled from several smaller PCR fragments using techniques known in the art. Restriction maps of different isolates of hMPV are shown in FIG. 10. The restriction sites can be used to assemble the full-length construct. In certain embodiments, PCR primers are designed such that the fragment resulting from the PCR reaction has a restriction site close to its 5′ end and a restriction site close to it 3′ end. The PCR product can then be digested with the respective restriction enzymes and subsequently ligated to the neighboring PCR fragments.

In order to achieve replication and packaging of the viral genome, it is important that the leader and trailer sequences retain the signals necessary for viral polymerase recognition. The leader and trailer sequences for the viral RNA genome can be optimized or varied to improve and enhance viral replication and rescue. Alternatively, the leader and trailer sequences can be modified to decrease the efficiency of viral replication and packaging, resulting in a rescued virus with an attenuated phenotype. Examples of different leader and trailer sequences, include, but are not limited to, leader and trailer sequences of a paramyxovirus. In a specific embodiment of the invention, the leader and trailer sequence is that of a wild type or mutated hMPV. In another embodiment of the invention, the leader and trailer sequence is that of a PIV, APV, or an RSV. In yet another embodiment of the invention, the leader and trailer sequence is that of a combination of different virus origins. By way of example and not meant to limit the possible combination, the leader and trailer sequence can be a combination of any of the leader and trailer sequences of hMPV, PIV, APV, RSV, or any other paramyxovirus. Examples of modifications to the leader and trailer sequences include varying the spacing relative to the viral promoter, varying the sequence, e.g., varying the number of G residues (typically 0 to 3), and defining the 5′ or 3′ end using ribozyme sequences, including, Hepatitis Delta Virus (HDV) ribozyme sequence, Hammerhead ribozyme sequences, or fragments thereof, which retain the ribozyme catalytic activity, and using restriction enzymes for run-off RNA produced in vitro.

In an alternative embodiment, the efficiency of viral replication and rescue may be enhanced if the viral genome is of hexamer length. In order to ensure that the viral genome is of the appropriate length, the 5′ or 3′ end may be defined using ribozyme sequences, including, Hepatitis Delta Virus (HDV) ribozyme sequence, Hammerhead ribozyme sequences, or fragments thereof, which retain the ribozyme catalytic activity, and using restriction enzymes for run-off RNA produced in vitro.

In order for the genetic material encoding the viral genome to be transcribed, the genetic material is engineered to be placed under the control of appropriate transcriptional regulatory sequences, e.g., promoter sequences recognized by a polymerase. In preferred embodiments, the promoter sequences are recognized by a T7, Sp6 or T3 polymerase. In yet another embodiment, the promoter sequences are recognized by cellular DNA dependent RNA polymerases, such as RNA polymerase I (Pol I) or RNA polymerase II (Pol II). The genetic material encoding the viral genome may be placed under the control of the transcriptional regulatory sequences, so that either a positive or negative strand copy of the viral genome is transcribed. The genetic material encoding the viral genome is recombinantly engineered to be operatively linked to the transcriptional regulatory sequences in the context of an expression vector, such as a plasmid based vector, e.g. a plasmid with a pol II promoter such as the immediate early promoter of CMV, a plasmid with a T7 promoter, or a viral based vector, e.g., pox viral vectors, including vaccinia vectors, MVA-T7, and Fowl pox vectors.

The genetic material encoding the viral genome may be modified to enhance expression by the polymerase of choice, e.g., varying the number of G residues (typically 0 to 3) upstream of the leader or trailer sequences to optimize expression from a T7 promoter.

Replication and packaging of the viral genome occurs intracellularly in a host cell permissive for viral replication and packaging. There are a number of methods by which the host cell can be engineered to provide sufficient levels of the viral polymerase and structural proteins necessary for replication and packaging, including, host cells infected with an appropriate helper virus, host cells engineered to stably or constitutively express the viral polymerase and structural proteins, or host cells engineered to transiently or inducibly express the viral polymerase and structural proteins.

Protein function required for MPV viral replication and packaging includes, but not limited to, the polymerase proteins P, N, L, and M2-1.

In one embodiment, the proteins expressed are native or wild type MPV proteins. In another embodiment, the proteins expressed may be modified to enhance their level of expression and/or polymerase activity, using standard recombinant techniques. Alternatively, fragments, derivatives, analogs or truncated versions of the polymerase proteins that retain polymerase activity may be expressed. In yet another embodiment, analogous polymerase proteins from other pneumoviruses, such as APV, or from any other paramyxovirus may be expressed. Moreover, an attenuated virus can be produced by expressing proteins of one strain of MPV along with the genome of another strain. For example, a polymerase protein of one strain of MPV can be expressed with the genome of another strain to produce an attenuated phenotype.

The viral polymerase proteins can be provided by helper viruses. Helper viruses that may be used in accordance with the invention, include those that express the polymerase viral proteins natively, such as MPV or APV. Alternatively, helper viruses may be used that have been recombinantly engineered to provide the polymerase viral proteins

Alternatively the viral polymerase proteins can be provided by expression vectors. Sequences encoding the viral polymerase proteins are engineered to be placed under the control of appropriate transcriptional regulatory sequences, e.g., promoter sequences recognized by a polymerase. In preferred embodiments, the promoter sequences are recognized by a T7, Sp6 or T3 polymerase. In yet another embodiment, the promoter sequences are recognized by a Pol I or Pol II polymerase. Alternatively, the promoter sequences are recognized by a viral polymerase, such as CMV. The sequences encoding the viral polymerase proteins are recombinantly engineered to be operatively linked to the transcriptional regulatory sequences in the context of an expression vector, such as a plasmid based vector, e.g. a CMV driven plasmid, a T7 driven plasmid, or a viral based vector, e.g., pox viral vectors, including vaccinia vectors, MVA-T7, and Fowl pox vectors.

In order to achieve efficient viral replication and packaging, high levels of expression of the polymerase proteins is preferred. Such levels are obtained using 100-200 ng L/pCITE, 200-400 ng N/pCITE, 200-400 ng P/pCITE, and 100-200 ng M2-1/pCITE plasmids encoding paramyxovirus proteins together with 2-4 ug of plasmid encoding the full-length viral cDNA transfected into cells infected with MVA-T7. In another embodiment, 0.1-2.0 μg of pSH25 (CAT expressing), 0.1-3.0 μg of pRF542 (expressing T7 polymerase), 0.1-0.8 μg pCITE vector with N cDNA insert, and 0.1-1.0 μg of each of three pCITE vectors containing P, L and M2-1 cDNA insert are used. Alternatively, one or more polymerase and structural proteins can be introduced into the cells in conjunction with the genetic material by transfecting cells with purified ribonucleoproteins. Host cells that are permissive for MPV viral replication and packaging are preferred. Examples of preferred host cells include, but are not limited to, 293T, Vero, tMK, and BHK. Other examples of host cells include, but are not limited to, LLC-MK-2 cells, Hep-2 cells, LF 1043 (HEL) cells, LLC-MK2, HUT 292, FRHL-2 (rhesus), FCL-1 (green monkey), WI-38 (human), MRC-5 (human) cells, QT 6 cells, QT 35 cells and CEF cells.

In alternative embodiments of the invention, the host cells can be treated using a number of methods in order to enhance the level of transfection and/or infection efficiencies, protein expression, in order to optimize viral replication and packaging. Such treatment methods, include, but are not limited to, sonication, freeze/thaw, and heat shock. Furthermore, standard techniques known to the skilled artisan can be used to optimize the transfection and/or infection protocol, including, but are not limited to, DEAE-dextran-mediated transfection, calcium phosphate precipitation, lipofectin treatment, liposome-mediated transfection and electroporation. The skilled artisan would also be familiar with standard techniques available for the optimization of transfection/infection protocols. By way of example, and not meant to limit the available techniques, methods that can be used include, manipulating the timing of infection relative to transfection when a virus is used to provide a necessary protein, manipulating the timing of transfections of different plasmids, and affecting the relative amounts of viruses and transfected plasmids.

In another embodiment, the invention relates to the rescue or production of live virus from cDNA using polymerase from a virus other than the one being rescued. In certain embodiments, hMPV is rescued from a cDNA using any of a number of polymerases, including, but not limited to, interspecies and intraspecies polymerases. In a certain embodiment, hMPV is rescued in a host cell expressing the minimal replication unit necessary for hMPV replication. For example, hMPV can be rescued from a cDNA using a number of polymerases, including, but not limited to, the polymerase of RSV, APV, MPV, or PIV. In a specific embodiment of the invention, hMPV is rescued using the polymerase of an RNA virus. In a more specific embodiment of the invention, hMPV is rescued using the polymerase of a negative stranded RNA virus. In an even more specific embodiment of the invention, hMPV is rescued using RSV polymerase. In another embodiment of the invention, hMPV is rescued using APV polymerase. In yet another embodiment of the invention, hMPV is rescued using an MPV polymerase. In another embodiment of the invention, hMPV is rescued using PIV polymerase.

In a more certain embodiment of the invention, hMPV is rescued from a cDNA using a complex of hMPV polymerase proteins. For example, the hMPV minireplicon can be rescued using a polymerase complex consisting of the L, P, N, and M2-1 proteins. In another embodiment of the invention, the polymerase complex consists of the L, P, and N proteins. In yet another embodiment of the invention, hMPV can be rescued from a cDNA using a polymerase complex consisting of polymerase proteins from other viruses, such as, but not limited to, RSV, PIV, and APV. In particular, hMPV can be rescued from a cDNA using a polymerase complex consisting of the L, P, N, and M2-1 proteins of RSV, PIV, APV, MPV, or any combination thereof. In yet another embodiment of the invention, the polymerase complex used to rescue hMPV from a cDNA consists of the L, P, and N proteins of RSV, PIV, APV, MPV, or any combination thereof. In even another embodiment of the invention, different polymerase proteins from various viruses can be used to form the polymerase complex. In such an embodiment, the polymerase used to rescue hMPV can be formed by different components of the RSV, PIV, APV, or MPV polymerases. By way of example, and not meant to limit the possible combination in forming a complex, the N protein can be encoded by the N gene of RSV, APV, PIV or MPV while the L protein is encoded by the L gene of RSV, APV, PIV or MPV and the P protein can be encoded by the P gene of RSV, APV, PIV or MPV. One skilled in the art would be able to determine the possible combinations that may be used to form the polymerase complex necessary to rescue the hMPV from a cDNA.

In certain embodiments, conditions for the propagation of virus are optimized in order to produce a robust and high-yielding cell culture (which would be beneficial, e.g., for manufacture the virus vaccine candidates of the invention). Critical parameters can be identified, and the production process can be first optimized in small-scale experiments to determine the scalability, robustness, and reproducibility and subsequently adapted to large scale production of virus. In certain embodiments, the virus that is propagated using the methods of the invention is hMPV. In certain embodiments, the virus that is propagated using the methods of the invention is a recombinant or a chimeric hMPV. In certain embodiments, the virus that is propagated using the methods of the invention is a virus of one of the following viral families Adenoviridae, Arenaviridae, Astroviridae, Baculoviridae, Bunyaviridae, Caliciviridae, Caulimovirus, Coronaviridae, Cystoviridae, Filoviridae, Flaviviridae, Hepadnaviridae, Herpesviridae, Hypoviridae, Idaeovirus, Inoviridae, Iridoviridae, Leviviridae, Lipothrixviridae, Luteovirus, Machlomovirus, Marafivirus, Microviridae, Myoviridae, Necrovirus, Nodaviridae, Orthomyxoviridae, Papovaviridae, Paramyxoviridae, Partitiviridae, Parvoviridae, Phycodnaviridae, Picornaviridae, Plasmaviridae, Podoviridae, Polydnaviridae, Potyviridae, Poxviridae, Reoviridae, Retroviridae, Rhabdoviridae, Sequiviridae, Siphoviridae, Sobemovirus, Tectiviridae, Tenuivirus, Tetraviridae, Tobamovirus Tobravirus, Togaviridae, Tombusviridae, Totiviridae, Trichovirus, Mononegavirales. In certain embodiments, the virus that is propagated with the methods of the invention is an RNA virus. In certain embodiments, the virus is not a virus of the family Herpesviridae. In certain embodiments, the virus is not HSV.

In certain embodiments, a cell culture infected with a virus or a viral construct of interest is incubated at a lower post-infection incubation temperature as compared to the standard incubation temperature for the cells in culture. In a specific embodiment, a cell culture infected with a viral construct of interest is incubated at 33° C. or about 33° C. (e.g., 33±1° C.). In certain embodiments, the post-infection incubation temperature is about 25° C., 26° C., 27° C., 28° C., 29 ° C., 30° C., 31° C., 32° C., 33° C., 34° C., 35° C., 36° C. or 37° C.

In certain embodiments, virus is propagated by incubating a cells before infection with the virus at a temperature optimized for the growth of the cells and subsequent to infection of the cells with the virus, i.e., post-infection, the temperature is shifted to a lower temperature. In certain embodiments the shift is at least 1° C., 2° C., 3° C., 4° C., 5° C., 6° C., 7° C., 8° C., 9° C., 10° C., 11° C., or at least 12° C. In certain embodiments the shift is at most 1 ° C., 2° C., 3° C., 4° C., 5° C., 6° C., 7° C., 8° C., 9° C., 10° C., 11° C., or at most 12° C. In a specific embodiment, the shift is 4° C.

In certain embodiments, the cells are cultured in a medium containing serum before infection with a virus or a viral construct of interest and the cells are cultured in a medium without serum after infection with the virus or viral construct. For a more detailed description of growing infected cells without serum, see the section entitled “Plasmid-Only Recovery of Virus in Serum Free Media.” In a specific embodiment, the serum is fetal bovine serum and is present a concentration of 5% of culture volume, 2% of culture volume, or 0.5% of culture volume.

In certain embodiments, virus is propagated by incubating cells that are infected with the virus in the absence of serum. In certain embodiments, virus is propagated by incubating cells that are infected with the virus in a culture medium containing less than 5% of serum, less than 2.5% of serum, less than 1% of serum, less than 0.1% of serum, less than 0.01% of serum, or less than 0.001% of serum.

In certain embodiments, the cells are incubated before infection with the virus in medium containing serum. In certain embodiments, subsequent to infection of the cells with the virus, the cells are incubated in the absence of serum. In other embodiments, the cells are first incubated in medium containing serum; the cells are then transferred into medium without serum; and subsequently, the cells are infected with the virus and further incubated in the absence of virus.

In certain embodiments, the cells are transferred from medium containing serum into medium in the absence of serum, by removing the serum-containing medium from the cells and adding the medium without serum. In other embodiments, the cells are centrifuged and the medium containing serum is removed and medium without serum is added. In certain embodiments, the cells are washed with medium without serum to ensure that cells once infected with the virus are incubated in the absence of serum. In certain, more specific embodiments, the cells are washed with medium without serum at least one time, two times, three times, four times, five times, or at least ten times.

In yet other embodiments, cells are cultured in a medium containing serum and at a temperature that is optimal for the growth of the cells before infection with a virus or a viral construct, and the cell culture is incubated at a lower temperature (relative to the standard incubation temperature for the corresponding virus or viral vector) after infection with the viral construct of interest. In a specific embodiment, cells are cultured in a medium containing serum before infection with a viral construct of interest at 37° C., and the cell culture is incubated at 33° C. or about 33° C. (e.g., 33±1° C.) after infection with the viral construct of interest.

In even other embodiments, cells are cultured in a medium containing serum and at a temperature that is optimal for the growth of the cells before infection with a virus or a viral construct, and the cell culture is incubated without serum at a lower temperature (relative to the standard incubation temperature for the corresponding virus or viral vector) after infection with the viral construct of interest. In a specific embodiment, cells are cultured in a medium containing serum before infection with a viral construct of interest at 37° C., and the cell culture is incubated without serum at 33° C. or about 33° C. (e.g., 33±1° C.) after infection with the viral construct of interest.

The viral constructs and methods of the present invention can be used for commercial production of viruses, e.g., for vaccine production. For commercial production of a vaccine, it is preferred that the vaccine contains only inactivated viruses or viral proteins that are completely free of infectious virus or contaminating viral nucleic acid, or alternatively, contains live attenuated vaccines that do not revert to virulence. Contamination of vaccines with adventitious agents introduced during production should also be avoided. Methods known in the art for large scale production of viruses or viral proteins can be used for commercial production of a vaccine of the invention. In one embodiment, for commercial production of a vaccine of the invention, cells are cultured in a bioreactor or fermenter. Bioreactors are available in volumes from under 1 liter to in excess of 100 liters, e.g., Cyto3 Bioreactor (Osmonics, Minnetonka, Minn.); NBS bioreactors (New Brunswick Scientific, Edison, N.J.); and laboratory and commercial scale bioreactors from B. Braun Biotech International (B. Braun Biotech, Melsungen, Germany). In another embodiment, small-scale process optimization studies are performed before the commercial production of the virus, and the optimized conditions are selected and used for the commercial production of the virus.

Plasmid-Rescue in Serum-Free Medium

In certain embodiments of the invention, virus can be recovered without helper virus. More specifically, virus can be recovered by introducing into a cell a plasmid encoding the viral genome and plasmids encoding viral proteins required for replication and rescue. In certain embodiments, the cell is grown and maintained in serum-free medium. In certain embodiments, the plasmids are introduced into the cell by electroporation. In a specific embodiment, a plasmid encoding the antigenomic cDNA of the virus under the control of the T7 promoter, a plasmid encoding the T7 RNA polymerase, and plasmids encoding the N protein, P protein, and L protein, respectively, under control of the T7 promoter are introduced into SF Vero cells by electroporation. Vero cells were obtained from ATCC and adapted to grow in serum-free media according to the following steps (developed by Mike Berry's laboratory).

1. Thaw ATCC CCL-81 Vial in DMEM +5% v/v FBS in T-25 flask P121;

2. Expand 5 passages in DMEM +5% v/v FBS P126;

3. Directly transfer FBS grown cells to OptiPRO (Invitrogen Corporation) in T-225 flasks;

4. Expand 7 passages in OptiPRO;

5. Freeze down Pre-Master Cell Bank Stock at Passage 133-7;

6. Expand 4 passages in OptiPRO;

7. Freeze down Master Cell Bank Stock at Passage 137;

8. Expand 4 passages in OptiPRO;

9. Freeze down Working Cell Bank Stock at Passage 141; and

10. Thaw and expand for electroporation and virus amplification.

Methods for the rescue of viral particles are described in section 5.6 entitled “Rescue Of Recombinant Virus Particles”.

In certain embodiments, the cells used for viral rescue are cells that can be grown and/or maintained without the addition of components derived from animals or humans. In certain embodiments, the cells used for viral rescue are cells that are adapted to growth without serum. In a specific embodiment, SF Vero cells are used for the rescue of virus. In certain embodiments, the cells are grown and/or maintained in OptiPRO SFM (Invitrogen Corporation) supplemented with 4 mM L-glutamine. In certain embodiments, the cells are grown in medium that is supplemented with serum but for rescue of viral particles the cells are transferred into serum-free medium. In a specific embodiment, the cells are washed in serum-free medium to ensure that the viral rescue takes place in a serum-free environment.

The plasmids are introduced into the cells by any method known to the skilled artisan that can be used with the cells, e.g., by calcium phosphate transfection, DEAE-Dextran transfection, electroporation or liposome mediated transfection (see Chapter 9 of Short Protocols in Molecular Biology, Ausubel et al. (editors), John Wiley & Sons, Inc., 1999). In specific embodiments, electroporation is used to introduce the plasmid DNA into the cells. SF Vero cells are resistant to lipofection. To select cells that have been transfected with the required plasmids, the plasmids can also carry certain markers. Such markers include, but are not limited to, resistancy to certain antibiotics (e.g., kanamycin, blasticidin, ampicillin, Hygromycin B, Puromycin and Zeocin™), makers that confer certain autotrophic properties on a cell that lacks this property without the marker, or a marker can also be a gene that is required for the growth of a cell but that is mutated in the cells into which the plasmid is introduced.

The transcription of the viral genome and/or the viral genes are under transcriptional control of a promoter. Thus, the sequences encoding the viral genome or the viral proteins are operatively linked to the promoter sequence. Any promoter/RNA polymerase system known to the skilled artisan can be used with the methods of the present invention. In certain embodiments, the promoter can be a promoter that allows transcription by an RNA polymerase endogenous to the cell, e.g., a promoter sequences that are recognized by a cellular DNA dependent RNA polymerases, such as RNA polymerase I (Pol I) or RNA polymerase II (Pol II). In certain embodiments, the promoter can be an inducible promoter. In certain embodiments, the promoter can be a promoter that allows transcription by an RNA polymerase that is not endogenous to the cell. In certain, more specific embodiments, the promoter is a T3 promoter, T7 promoter, SP6 promoter, or CMV promoter. Depending on the type of promoter used, a plasmid encoding the RNA polymerase that recognizes the promoter is also introduced into the cell to provide the appropriate RNA polymerase. In specific embodiments, the RNA polymerase is T3 RNA polymerase, T7 RNA polymerase, SP6 RNA polymerase, or CMV RNA polymerase. In a specific embodiment, the viral genes and the viral genome are transcribed under the control of a T7 promoter and a plasmid encoding the T7 RNA polymerase is introduced to provide the T7 RNA polymerase. The transcription of the polymerase can be under the control of any promoter system that would function in the cell type used. In a specific embodiment, the CMV promoter is used.

The viral genome can be in the plus or minus orientation. Thus, the viral genome can be transcribed from the genetic material to generate either a positive sense copy of the viral genome (antigenome copy) or a negative sense copy of the viral genome (genomic copy). In certain embodiments, the viral genome is a recombinant, chimeric and/or attenuated virus of the invention. In certain embodiments, the efficiency of viral replication and rescue may be enhanced if the viral genome is of hexamer length. In order to ensure that the viral genome is of the appropriate length, the 5′ or 3′ end may be defined using ribozyme sequences, including, Hepatitis Delta Virus (HDV) ribozyme sequence, Hammerhead ribozyme sequences, or fragments thereof, which retain the ribozyme catalytic activity.

In certain embodiments, the viral proteins required for replication and rescue include the N, P, and L gene. In certain, more specific, embodiments, the viral proteins required for replication and rescue include the N, P, M2-1 and L gene.

### 5.7 Attenuatin of Recombinant Viruses

The recombinant viruses of the invention can be further genetically engineered to exhibit an attenuated phenotype. In particular, the recombinant viruses of the invention exhibit an attenuated phenotype in a subject to which the virus is administered as a vaccine. Attenuation can be achieved by any method known to a skilled artisan. Without being bound by theory, the attenuated phenotype of the recombinant virus can be caused, e.g., by using a virus that naturally does not replicate well in an intended host (e.g., using an APV in human), by reduced replication of the viral genome, by reduced ability of the virus to infect a host cell, or by reduced ability of the viral proteins to assemble to an infectious viral particle relative to the wild type strain of the virus. The viability of certain sequences of the virus, such as the leader and the trailer sequence can be tested using a minigenome assay (see section 5.8).

The attenuated phenotypes of a recombinant virus of the invention can be tested by any method known to the artisan (see, e.g., section 5.8). A candidate virus can, for example, be tested for its ability to infect a host or for the rate of replication in a cell culture system. In certain embodiments, a mimi-genome system is used to test the attenuated virus when the gene that is altered is N, P, L, M2, F, G, M2-1, M2-2 or a combination thereof. In certain embodiments, growth curves at different temperatures are used to test the attenuated phenotype of the virus. For example, an attenuated virus is able to grow at 35° C., but not at 39° C. or 40° C. In certain embodiments, different cell lines can be used to evaluate the attenuated phenotype of the virus. For example, an attenuated virus may only be able to grow in monkey cell lines but not the human cell lines, or the achievable virus titers in different cell lines are different for the attenuated virus. In certain embodiments, viral replication in the respiratory tract of a small animal model, including but not limited to, hamsters, cotton rats, mice and guinea pigs, is used to evaluate the attenuated phenotypes of the virus. In other embodiments, the immune response induced by the virus, including but not limited to, the antibody titers (e.g., assayed by plaque reduction neutralization assay or ELISA) is used to evaluate the attenuated phenotypes of the virus. In a specific embodiment, the plaque reduction neutralization assay or ELISA is carried out at a low dose. In certain embodiments, the ability of the recombinant virus to elicit pathological symptoms in an animal model can be tested. A reduced ability of the virus to elicit pathological symptoms in an animal model system is indicative of its attenuated phenotype. In a specific embodiment, the candidate viruses are tested in a monkey model for nasal infection, indicated by mucous production.

The viruses of the invention can be attenuated such that one or more of the functional characteristics of the virus are impaired. In certain embodiments, attenuation is measured in comparison to the wild type strain of the virus from which the attenuated virus is derived. In other embodiments, attenuation is determined by comparing the growth of an attenuated virus in different host systems. Thus, for a non-limiting example, an APV is said to be attenuated when grown in a human host if the growth of the APV in the human host is reduced compared to the growth of the APV in an avian host.

In certain embodiments, the attenuated virus of the invention is capable of infecting a host, is capable of replicating in a host such that infectious viral particles are produced. In comparison to the wild type strain, however, the attenuated strain grows to lower titers or grows more slowly. Any technique known to the skilled artisan can be used to determine the growth curve of the attenuated virus and compare it to the growth curve of the wild type virus. For exemplary methods see Example section, infra. In a specific embodiment, the attenuated virus grows to a titer of less than 10⁵ pfu/ml, of less than 10⁴ pfu/ml, of less than 10³ pfu/ml, or of less than 10² pfu/ml in Vero cells under conditions as described in, e.g., Example 22.

In certain embodiments, the attenuated virus of the invention (e.g., a chimeric mammalian MPV) cannot replicate in human cells as well as the wild type virus (e.g., wild type mammalian MPV) does. However, the attenuated virus can replicate well in a cell line that lack interferon functions, such as Vero cells.

In other embodiments, the attenuated virus of the invention is capable of infecting a host, of replicating in the host, and of causing proteins of the virus of the invention to be inserted into the cytoplasmic membrane, but the attenuated virus does not cause the host to produce new infectious viral particles. In certain embodiments, the attenuated virus infects the host, replicates in the host, and causes viral proteins to be inserted in the cytoplasmic membrane of the host with the same efficiency as the wild type mammalian virus. In other embodiments, the ability of the attenuated virus to cause viral proteins to be inserted into the cytoplasmic membrane into the host cell is reduced compared to the wild type virus. In certain embodiments, the ability of the attenuated mammalian virus to replicate in the host is reduced compared to the wild type virus. Any technique known to the skilled artisan can be used to determine whether a virus is capable of infecting a mammalian cell, of replicating within the host, and of causing viral proteins to be inserted into the cytoplasmic membrane of the host. For illustrative methods see section 5.8.

In certain embodiments, the attenuated virus of the invention is capable of infecting a host. In contrast to the wild type mammalian MPV, however, the attenuated mammalian MPV cannot be replicated in the host. In a specific embodiment, the attenuated mammalian virus can infect a host and can cause the host to insert viral proteins in its cytoplasmic membranes, but the attenuated virus is incapable of being replicated in the host. Any method known to the skilled artisan can be used to test whether the attenuated mammalian MPV has infected the host and has caused the host to insert viral proteins in its cytoplasmic membranes.

In certain embodiments, the ability of the attenuated mammalian virus to infect a host is reduced compared to the ability of the wild type virus to infect the same host. Any technique known to the skilled artisan can be used to determine whether a virus is capable of infecting a host. For illustrative methods see section 5.8.

In certain embodiments, mutations (e.g., missense mutations) are introduced into the genome of the virus to generated a virus with an attenuated phenotype. Mutations (e.g., missense mutations) can be introduced into the N-gene, the P-gene, the M-gene, the F-gene, the M2-gene, the SH-gene, the G-gene or the L-gene of the recombinant virus. Mutations can be additions, substitutions, deletions, or combinations thereof. In specific embodiments, a single amino acid deletion mutation for the N, P, L, F, G, M2-1, M2-2 or M2 proteins is introduced, which can be screened for functionality in the mini-genome assay system and be evaluated for predicted functionality in the virus. In more specific embodiments, the missense mutation is a cold-sensitive mutation. In other embodiments, the missense mutation is a heat-sensitive mutation. In one embodiment, major phosphorylation sites of P protein of the virus is removed. In another embodiment, a mutation or mutations are introduced into the L gene of the virus to generate a temperature sensitive strain. In yet another embodiment, the cleavage site of the F gene is mutated in such a way that cleavage does not occur or occurs at very low efficiency. In certain, more specific embodiments, the motif with the amino acid sequence RQSR at amino acid postions 99 to 102 of the F protein of hMPV is mutated. A mutation can be, but is not limited to, a deletion of one or more amino acids, an addition of one or more amino acids, a substitution (conserved or non-conserved) of one or more amino acids or a combination thereof. In some strains of hMPV, the cleavage site is RQPR (see Example “P101S”). In certain embodiments, the cleavage site with the amino acid sequence is RQPR is mutated. In more specific embodiments, the cleavage site of the F protein of hMPV is mutated such that the infectivity of hMPV is reduced. In certain embodiments, the infectivity of hMPV is reduced by a factor of at least 5, 10, 50, 100, 500, 10³, 5×10³, 10⁴, 5×10⁴, 10⁵, 5×10⁵, or at least 10⁶. In certain embodiments, the infectivity of hMPV is reduced by a factor of at most 5, 10, 50, 100, 500, 10³, 5×10³, 10⁴, 5×10⁴, 10⁵, 5×10⁵, or at most 10⁶.

In other embodiments, deletions are introduced into the genome of the recombinant virus. In more specific embodiments, a deletion can be introduced into the N-gene, the P-gene, the M-gene, the F-gene, the M2-gene, the SH-gene, the G-gene or the L-gene of the recombinant virus. In specific embodiments, the deletion is in the M2-gene of the recombinant virus of the present invention. In other specific embodiments, the deletion is in the SH-gene of the recombinant virus of the present invention. In yet another specific embodiment, both the M2-gene and the SH-gene are deleted.

In certain embodiments, the intergenic region of the recombinant virus is altered. In one embodiment, the length of the intergenic region is altered. In another embodiment, the intergenic regions are shuffled from 5′ to 3′ end of the viral genome.

In other embodiments, the genome position of a gene or genes of the recombinant virus is changed. In one embodiment, the F or G gene is moved to the 3′ end of the genome. In another embodiment, the N gene is moved to the 5′ end of the genome.

In certain embodiments, attenuation of the virus is achieved by replacing a gene of the wild type virus with the analogous gene of a virus of a different species (e.g., of RSV, APV, PIV3 or mouse pneumovirus), of a different subgroup, or of a different variant. In illustrative embodiments, the N-gene, the P-gene, the M-gene, the F-gene, the M2-gene, the SH-gene, the G-gene or the L-gene of a mammalian MPV is replaced with the N-gene, the P-gene, the M-gene, the F-gene, the M2-gene, the SH-gene, the G-gene or the L-gene, respectively, of an APV. In other illustrative embodiments, the N-gene, the P-gene, the M-gene, the F-gene, the M2-gene, the SH-gene, the G-gene or the L-gene of APV is replaced with the N-gene, the P-gene, the M-gene, the F-gene, the M2-gene, the SH-gene, the G-gene or the L-gene, respectively, of a mammalian MPV. In a preferred embodiment, attenuation of the virus is achieved by replacing one or more polymerase associated genes (e.g., N, P, L or M2) with genes of a virus of a different species.

In certain embodiments, attenuation of the virus is achieved by replacing one or more specific domains of a protein of the wild type virus with domains derived from the corresponding protein of a virus of a different species. In an illustrative embodiment, the ectodomain of a F protein of APV is replaced with an ectodomain of a F protein of a mammalian MPV. In a preferred embodiment, one or more specific domains of L, N, or P protein are replaced with domains derived from corresponding proteins of a virus of a different species. In certain other embodiments, attenuation of the virus is achieved by deleting one or more specific domains of a protein of the wild type virus. In a specific embodiment, the transmembrane domain of the F-protein is deleted.

In certain embodiments of the invention, the leader and/or trailer sequence of the recombinant virus of the invention can be modified to achieve an attenuated phenotype. In certain, more specific embodiments, the leader and/or trailer sequence is reduced in length relative to the wild type virus by at least 1 nucleotide, at least 2 nucleotides, at least 3 nucleotides, at least 4 nucleotides, at least 5 nucleotides or at least 6 nucleotides. In certain other, more specific embodiments, the sequence of the leader and/or trailer of the recombinant virus is mutated. In a specific embodiment, the leader and the trailer sequence are 100% complementary to each other. In other embodiments, 1 nucleotide, 2 nucleotides, 3 nucleotides, 4 nucleotides, 5 nucleotides, 6 nucleotides, 7 nucleotides, 8 nucleotides, 9 nucleotides, or 10 nucleotides are not complementary to each other where the remaining nucleotides of the leader and the trailer sequences are complementary to each other. In certain embodiments, the non-complementary nucleotides are identical to each other. In certain other embodiments, the non-complementary nucleotides are different from each other. In other embodiments, if the non-complementary nucleotide in the trailer is purine, the corresponding nucleotide in the leader sequence is also a purine. In other embodiments, if the non-complementary nucleotide in the trailer is pyrimidine, the corresponding nucleotide in the leader sequence is also a purine. In certain embodiments of the invention, the leader and/or trailer sequence of the recombinant virus of the invention can be replaced with the leader and/or trailer sequence of a another virus, e.g., with the leader and/or trailer sequence of RSV, APV, PIV3, mouse pneumovirus, or with the leader and/or trailer sequence of a human metapneumovirus of a subgroup or variant different from the human metapneumovirus from which the protein-encoding parts of the recombinant virus are derived.

When a live attenuated vaccine is used, its safety must also be considered. The vaccine must not cause disease. Any techniques known in the art that can make a vaccine safe may be used in the present invention. In addition to attenuation techniques, other. techniques may be used. One non-limiting example is to use a soluble heterologous gene that cannot be incorporated into the virion membrane. For example, a single copy of the soluble RSV F gene, a version of the RSV gene lacking the transmembrane and cytosolic domains, can be used. Since it cannot be incorporated into the virion membrane, the virus tropism is not expected to change.

Various assays can be used to test the safety of a vaccine. See section 5.8, infra. Particularly, sucrose gradients and neutralization assays can be used to test the safety. A sucrose gradient assay can be used to determine whether a heterologous protein is inserted in a virion. If the heterologous protein is inserted in the virion, the virion should be tested for its ability to cause symptoms even if the parental strain does not cause symptoms. Without being bound by theory, if the heterologous protein is incorporated in the virion, the virus may have acquired new, possibly pathological, properties.

In certain embodiments, one or more genes are deleted from the hMPV genome to generate an attenuated virus. In more specific embodiments, the M2-2 ORF, the M2-1 ORF, the M2 gene, the SH gene and/or the G2 gene is deleted.

In other embodiments, small single amino acid deletions are introduced in genes involved in virus replication to generate an attenuated virus. In more specific embodiments, a small single amino acid deletion is introduced in the N, L, or the P gene. In certain specific embodiments, one or more of the following amino acids are mutated in the L gene of a recombinant hMPV: Phe at amino acid position 456, Glu at amino acid position 749, Tyr at amino acid position 1246, Met at amino acid position 1094 and Lys at amino acid position 746 to generate an attenuated virus. A mutation can be, e.g., a deletion or a substitution of an amino acid. An amino acid substitution can be a conserved amino acid substitution or a non-conserved amino acid substitution. Illustrative examples for conserved amino acid exchanges are amino acid substitutions that maintain structural and/or functional properties of the amino acids' side-chains, e.g., an aromatic amino acid is substituted for another aromatic amino acid, an acidic amino acid is substituted for another acidic amino acid, a basic amino acid is substituted for another basic amino acid, and an aliphatic amino acid is substituted for another aliphatic amino acid. In contrast, examples of non-conserved amino acid exchanges are amino acid substitutions that do not maintain structural and/or functional properties of the amino acids' side-chains, e.g., an aromatic amino acid is substituted for a basic, acidic, or aliphatic amino acid, an acidic amino acid is substituted for an aromatic, basic, or aliphatic amino acid, a basic amino acid is substituted for an acidic, aromatic or aliphatic amino acid, and an aliphatic amino acid is substituted for an aromatic, acidic or basic amino acid. In even more specific embodiments Phe at amino acid position 456 is replaced by a Leu.

In certain embodiments, one nucleic acid is substituted to encode one amino acid exchange. In other embodiments, two or three nucleic acids are substituted to encode one amino acid exchange. It is preferred that two or three nucleic acids are substituted to reduce the risk of reversion to the wild type protein sequence.

In other embodiments, small single amino acid deletions are introduced in genes involved in virus assembly to generate an attenuated virus. In more specific embodiments, a small single amino acid deletion is introduced in the M gene or the M2 gene. In a preferred embodiment, the M gene is mutated.

In even other embodiments, the gene order in the genome of the virus is changed from the gene order of the wild type virus to generate an attenuated virus. In a more specific embodiment, the F, SH, and/or the G gene is moved to the 3′ end of the viral genome. In another embodiment, the N gene is moved to the 5′ end of the viral genome.

In other embodiments, one or more gene start sites (for locations of gene start sites see, e.g., Table 8) are mutated or substituted with the analogous gene start sites of another virus (e.g., RSV, PIV3, APV or mouse pneumovirus) or of a human metapneumovirus of a subgroup or a variant different from the human metapneumovirus from which the protein-encoding parts of the recombinant virus are derived. In more specific embodiments, the gene start site of the N-gene, the P-gene, the M-gene, the F-gene, the M2-gene, the SH-gene, the G-gene and/or the L-gene is mutated or replaced with the start site of the N-gene, the P-gene, the M-gene, the F-gene, the M2-gene, the SH-gene, the G-gene and/or the L-gene, respectively, of another virus (e.g., RSV, PIV3, APV or mouse pneumovirus) or of a human metapneumovirus of a subgroup or a variant different from the human metapneumovirus from which the protein-encoding parts of the recombinant virus are derived.

### 5.7.1 Attenuation by Substitution of Viral Genes

In certain embodiments of the invention, attenuation is achieved by replacing one or more of the genes of a virus with the analogous gene of a different virus, different strain, or different viral isolate. In certain embodiments, one or more of the genes of a metapneumovirus, such as a mammalian metapneumovirus, e.g., hMPV, or APV, is replaced with the analogous gene(s) of another paramyxovirus. In a more specific embodiment, the N-gene, the P-gene, the

M-gene, the F-gene, the M2-gene, the M2-1 ORF, the M2-2 ORF, the SH-gene, the G-gene or the L-gene or any combination of two or more of these genes of a mammalian metapneumovirus, e.g., hMPV, is replaced with the analogous gene of another viral species, strain or isolate, wherein the other viral species can be, but is not limited to, another mammalian metapneumovirus, APV, or RSV.

In more specific embodiments, one or more of the genes of human metapneumovirus are replaced with the analogous gene(s) of another isolate of human metapneumovirus. E.g., the N-gene, the P-gene, the M-gene, the F-gene, the M2-gene, the M2-1 ORF, the M2-2 ORF, the SH-gene, the G-gene or the L-gene or any combination of two or more of these genes of isolate NL/1/99 (99-1), NL/1/00 (00-1), NL/17/00, or NL/1/94 is replaced with the analogous gene or combination of genes, i.e., the N-gene, the P-gene, the M-gene, the F-gene, the M2-gene, the M2-1 ORF, the M2-2 ORF, the SH-gene, the G-gene or the L-gene, of a different isolate, e.g., NL/1/99 (99-1), NL/1/00 (00-1), NL/17/00, or NL/1/94.

In certain embodiments, one or more regions of the genome of a virus is/are replaced with the analogous region(s) from the genome of a different viral species, strain or isolate. In certain embodiments, the region is a region in a coding region of the viral genome. In other embodiments, the region is a region in a non-coding region of the viral genome. In certain embodiments, two regions of two viruses are analogous to each other if the two regions support the same or a similar function in the two viruses. In certain other embodiments, two regions of two viruses are analogous if the two regions provide the same of a similar structural element in the two viruses. In more specific embodiments, two regions are analogous if they encode analogous protein domains in the two viruses, wherein analogous protein domains are domains that have the same or a similar function and/or structure.

In certain embodiments, one or more of regions of a genome of a metapneumovirus, such as a mammalian metapneumovirus, e.g., hMPV, or APV, is/are replaced with the analogous region(s) of the genome of another paramyxovirus. In certain embodiments, one or more of regions of the genome of a paramyxovirus is/are replaced with the analogous region(s) of the genome of a mammalian metapneumovirus, e.g., hMPV, or APV. In more specific embodiments, a region of the N-gene, the P-gene, the M-gene, the F-gene, the M2-gene, the M2-1 ORF, the M2-2 ORF, the SH-gene, the G-gene or the L-gene or any combination of two or more regions of these genes of a mammalian metapneumovirus, e.g., hMPV, is replaced with the analogous region of another viral species, strain or isolate. Another viral species can be, but is not limited to, another mammalian metapneumovirus, APV, or RSV.

In more specific embodiments, one or more regions of human metapneumovirus are replaced with the analogous region(s) of another isolate of human metapneumovirus. E.g., one or more region(s) of the N-gene, the P-gene, the M-gene, the F-gene, the M2-gene, the M2-1 ORF, the M2-2 ORF, the SH-gene, the G-gene or the L-gene or any combination of two or more regions of isolate NL/1/99 (99-1), NL/1/00 (00-1), NL/17/00, or NL/1/94 is replaced with the analogous region(s) of a different isolate of hMPV, e.g., NL/1/99 (99-1), NL/1/00 (00-1), NL/17/00, or NL/1/94.

In certain embodiments, the region is at least 5 nucleotides (nt) in length, at least 10 nt, at least 25 nt, at least 50 nt, at least 75 nt, at least 100 nt, at least 250 nt, at least 500 nt, at least 750 nt, at least 1 kb, at least 1.5 kb, at least 2 kb, at least 2.5 kb, at least 3 kb, at least 4 kb, or at least 5 kb in length. In certain embodiments, the region is at most 5 nucleotides (nt) in length, at most 10 nt, at most 25 nt, at most 50 nt, at most 75 nt, at most 100 nt, at most 250 nt, at most 500 nt, at most 750 nt, at most 1 kb, at most 1.5 kb, at most 2 kb, at most 2.5 kb, at most 3 kb, at most 4 kb, or at most 5 kb in length.

5.8 Assays for Use with the Invention

A number of assays may be employed in accordance with the present invention in order to determine the rate of growth of a chimeric or recombinant virus in a cell culture system, an animal model system or in a subject. A number of assays may also be employed in accordance with the present invention in order to determine the requirements of the chimeric and recombinant viruses to achieve infection, replication and packaging of virions.

The assays described herein may be used to assay viral titre over time to determine the growth characteristics of the virus. In a specific embodiment, the viral titre is determined by obtaining a sample from the infected cells or the infected subject, preparing a serial dilution of the sample and infecting a monolayer of cells that are susceptible to infection with the virus at a dilution of the virus that allows for the emergence of single plaques. The plaques can then be counted and the viral titre express as plaque forming units per milliliter of sample. In a specific embodiment of the invention, the growth rate of a virus of the invention in a subject is estimated by the titer of antibodies against the virus in the subject. Without being bound by theory, the antibody titer in the subject reflects not only the viral titer in the subject but also the antigenicity. If the antigenicity of the virus is constant, the increase of the antibody titer in the subject can be used to determine the growth curve of the virus in the subject. In a preferred embodiment, the growth rate of the virus in animals or humans is best tested by sampling biological fluids of a host at multiple time points post-infection and measuring viral titer.

The expression of heterologous gene sequence in a cell culture system or in a subject can be determined by any technique known to the skilled artisan. In certain embodiments, the expression of the heterologous gene is measured by quantifying the level of the transcript. The level of the transcript can be measured by Northern blot analysis or by RT-PCR using probes or primers, respectively, that are specific for the transcript. The transcript can be distinguished from the genome of the virus because the virus is in the antisense orientation whereas the transcript is in the sense orientation. In certain embodiments, the expression of the heterologous gene is measured by quantifying the level of the protein product of the heterologous gene. The level of the protein can be measured by Western blot analysis using antibodies that are specific to the protein.

In a specific embodiment, the heterologous gene is tagged with a peptide tag. The peptide tag can be detected using antibodies against the peptide tag. The level of peptide tag detected is representative for the level of protein expressed from the heterologous gene. Alternatively, the protein expressed from the heterologous gene can be isolated by virtue of the peptide tag. The amount of the purified protein correlates with the expression level of the heterologous gene. Such peptide tags and methods for the isolation of proteins fused to such a peptide tag are well known in the art. A variety of peptide tags known in the art may be used in the modification of the heterologous gene, such as, but not limited to, the immunoglobulin constant regions, polyhistidine sequence (Petty, 1996, Metal-chelate affinity chromatography, in Current Protocols in Molecular Biology, volume 1-3 (1994-1998). Ed. by Ausubel, F. M., Brent, R., Kunston, R. E., Moore, D. D., Seidman, J. G., Smith, J. A. and Struhl, K. Published by John Wiley and sons, Inc., USA, Greene Publish. Assoc. & Wiley Interscience), glutathione S-transferase (GST; Smith, 1993, Methods Mol. Cell Bio. 4:220-229), the E. coli maltose binding protein (Guan et al., 1987, Gene 67:21-30), various cellulose binding domains (U.S. Pat. Nos. 5,496,934; 5,202,247; 5,137,819; Tomme et al., 1994, Protein Eng. 7:117-123), and the FLAG epitope (Short Protocols in Molecular Biology, 1999, Ed. Ausubel et al., John Wiley & Sons, Inc., Unit 10.11) etc. Other peptide tags are recognized by specific binding partners and thus facilitate isolation by affinity binding to the binding partner, which is preferably immobilized and/or on a solid support. As will be appreciated by those skilled in the art, many methods can be used to obtain the coding region of the above-mentioned peptide tags, including but not limited to, DNA cloning, DNA amplification, and synthetic methods. Some of the peptide tags and reagents for their detection and isolation are available commercially.

Samples from a subject can be obtained by any method known to the skilled artisan. In certain embodiments, the sample consists of nasal aspirate, throat swab, sputum or broncho-alveolar lavage.

### 5.8.1 Minireplicon Constructs

The production of live virus from cDNA provides a means for characterizing hMPV and also for producing attenuated vaccine strains and immunogenic compounds. In order to accomplish this goal, cDNA or minireplicon constructs that encode vRNAs containing a reporter gene can be used to rescue virus and also to identify the nucleotide sequences and proteins involved in amplification, expression, and incorporation of RNAs into virions. Any reporter gene known to the skilled artisan can be used with the invention (see section 5.8.2). For example, reporter genes that can be used include, but are not limited to, genes that encode GFP, HRP, LUC, and AP. (Also see section 5.8.2 for a more extensive list of examples of reporters) In one specific embodiment, the reporter gene that is used encodes CAT. In another specific embodiment of the invention, the reporter gene is flanked by leader and trailer sequences. The leader and trailer sequences that can be used to flank the reporter genes are those of any negative-sense virus, including, but not limited to, MPV, RSV, and APV. For example, the reporter gene can be flanked by the negative-sense hMPV or APV leader linked to the hepatitis delta ribozyme (Hep-d Ribo) and T7 polymerase termination (T-T7) signals, and the hMPV or APV trailer sequence preceded by the T7 RNA polymerase promoter.

In certain embodiments, the plasmid encoding the minireplicon is transfected into a host cell. In a more specific embodiment of the invention, hMPV is rescued in a host cell expressing T7 RNA polymerase, the N gene, the P gene, the L gene, and the M2.1 gene. In certain embodiments, the host cell is transfected with plasmids encoding T7 RNA polymerase, the N gene, the P gene, the L gene, and the M2.1 gene. In other embodiments, the plasmid encoding the minireplicon is transfected into a host cell and the host cell is infected with a helper virus.

The hMPV minireplicon can be rescued using a number of polymerases, including, but not limited to, interspecies and intraspecies polymerases. In a certain embodiment, the hMPV minireplicon is rescued in a host cell expressing the minimal replication unit necessary for hMPV replication. For example, hMPV can be rescued from a cDNA using a number of polymerases, including, but not limited to, the polymerase of RSV, APV, MPV, or PIV. In a specific embodiment of the invention, hMPV is rescued using the polymerase of an RNA virus. In a more specific embodiment of the invention, hMPV is rescued using the polymerase of a negative stranded RNA virus. In an even more specific embodiment of the invention, hMPV is rescued using RSV polymerase. In another embodiment of the invention, hMPV is rescued using APV polymerase. In yet another embodiment of the invention, hMPV is rescued using an MPV polymerase. In another embodiment of the invention, hMPV is rescued using PIV polymerase.

In another embodiment of the invention, hMPV is rescued from a cDNA using a complex of hMPV polymerase proteins. For example, the hMPV minireplicon can be rescued using a polymerase complex consisting of the L, P, N, and M2-1 proteins. In another embodiment of the invention, the polymerase complex consists of the L, P, and N proteins. In yet another embodiment of the invention, the hMPV minireplicon can be rescued using a polymerase complex consisting of polymerase proteins from other viruses, such as, but not limited to, RSV, PIV, and APV. In particular, the hMPV minireplicon can be rescued using a polymerase complex consisting of the L, P, N, and M2-1 proteins of RSV, PIV, or APV. In yet another embodiment of the invention, the polymerase complex used to rescue the hMPV minireplicon consists of the L, P, and N proteins of RSV, NV, or APV. In even another embodiment of the invention, different polymerase proteins from various viruses can be used to form the polymerase complex. In such an embodiment, the polymerase used to rescue the hMPV minireplicon can be formed by different components of the RSV, PIV, or APV polymerases. By way of example, and not meant to limit the possible combination, in forming a complex, the N protein can be encoded by the N gene of RSV, APV, or PIV, while the L protein is encoded by the L gene of RSV, APV, or PIV, and P protein can be encoded by the P gene of RSV, APV, or PIV. One skilled in the art would be able to determine the possible combinations that may be used to form the polymerase complex necessary to rescue the hMPV minireplicon. In the minireplicon system, the expression of a reporter gene is measured in order to confirm the successful rescue of the virus and also to characterize the virus. The expression level of the reporter gene and/or its activity can be assayed by any method known to the skilled artisan, such as, but not limited to, the methods described in section 5.8.2.

In certain, more specific, embodiments, the minireplicon comprises the following elements, in the order listed: T7 RNA Polymerase or RNA polymerase I, leader sequence, gene start, GFP, trailer sequence, Hepatitis delta ribozyme sequence or RNA polymerase I termination sequence. If T7 is used as RNA polymerase, Hepatitis delta ribozyme sequence should be used as termination sequence. If RNA polymerase I is used, RNA polymerase I termination sequence may be used as a termination signal. Dependent on the rescue system, the sequence of the minireplicon can be in the sense or antisense orientation. In certain embodiments, the leader sequence can be modified relative to the wild type leader sequence of hMPV. The leader sequence can optionally be preceded by an AC. The T7 promoter sequence can be with or without a G-doublet or triplet, where the G-doublet or triplet provides for increased transcription.

In a specific embodiment, a cell is infected with hMPV at TO. 24 hours later, at T24, the cell is transfected with a minireplicon construct. 48 hours after T0 and 72 hours after T0, the cells are tested for the expression of the reporter gene. If a fluorescent reporter gene product is used (e.g., GFP), the expression of the reporter gene can be tested using FACS.

In another embodiment, a cell is transfected with six plasmids at T=0 hours. Cells are then harvested at T=40 hours and T=60 hours and analyzed for CAT or GFP expression.

In another specific embodiment, a cell is infected with MVA-T7 at T0. 1 hour later, at T1, the cell is transfected with a minireplicon construct. 24 hours after T0, the cell is infected with hMPV. 72 hours after T0, the cells are tested for the expression of the reporter gene. If a fluorescent reporter gene product is used (e.g., GFP), the expression of the reporter gene can be tested using FACS.

### 5.8.2 Reporter Genes

In certain embodiments, assays for measurement of reporter gene expression in tissue culture or in animal models can be used with the methods of the invention. The nucleotide sequence of the reporter gene is cloned into the virus, such as APV, hMPV, hMPV/APV or APV/hMPV, wherein (i) the position of the reporter gene is changed and (ii) the length of the intergenic regions flanking the reporter gene are varied. Different combinations are tested to determine the optimal rate of expression of the reporter gene and the optimal replication rate of the virus comprising the reporter gene.

In certain embodiments, minireplicon constructs are generated to include a reporter gene. The construction of minireplicon constructs is described herein.

The abundance of the reporter gene product can be determined by any technique known to the skilled artisan. Such techniques include, but are not limited to, Northern blot analysis or Western blot analysis using probes or antibodies, respectively, that are specific to the reporter gene.

In certain embodiments, the reporter gene emits a fluorescent signal that can be detected in a FACS. FACS can be used to detect cells in which the reporter gene is expressed.

Techniques for practicing the specific aspect of this invention will employ, unless otherwise indicated, conventional techniques of molecular biology, microbiology, and recombinant DNA manipulation and production, which are routinely practiced by one of skill in the art. See, e.g., Sambrook et al., Molecular cloning, a laboratory manual, second ed., vol. 1-3. (Cold Spring Harbor Laboratory, 1989), A Laboratory Manual, Second Edition; DNA Cloning, Volumes I and II (Glover, Ed. 1985); and Transcription and Translation (Hames & Higgins, Eds. 1984).

The biochemical activity of the reporter gene product represents the expression level of the reporter gene. The total level of reporter gene activity depends also on the replication rate of the recombinant virus of the invention. Thus, to determine the true expression level of the reporter gene from the recombinant virus, the total expression level should be divided by the titer of the recombinant virus in the cell culture or the animal model.

Reporter genes that can be used with the methods of invention include, but are not limited to, the genes listed in the Table 4 below:

TABLE 4
Reporter genes and the biochemical properties of the
respective reporter gene products

| Reporter Gene | Protein Activity & Measurement |
|---|---|
| CAT (chloramphenicol | Transfers radioactive acetyl groups to |
| acetyltransferase) | chloramphenicol or detection by thin layer |
| | chromatography and autoradiography |
| GAL (b-galactosidase) | Hydrolyzes colorless galactosides to yield |
| | colored products. |
| GUS (b-glucuronidase) | Hydrolyzes colorless glucuronides to yield |
| | colored products. |
| LUC (luciferase) | Oxidizes luciferin, emitting photons |
| GFP (green fluorescent | fluorescent protein without substrate |
| protein) | |
| SEAP (secreted alkaline | luminescence reaction with suitable substrates |
| phosphatase) | or with substrates that generate chromophores |
| HRP (horseradish | in the presence of hydrogen oxide, oxidation |
| peroxidase) | of 3,3′,5,5′-tetramethylbenzidine to form a |
| | colored complex |
| AP (alkaline | luminescence reaction with suitable substrates |
| phosphatase) | or with substrates that generate chromophores |

The abundance of the reporter gene can be measured by, inter alia, Western blot analysis or Northern blot analysis or any other technique used for the quantification of transcription of a nucleotide sequence, the abundance of its mRNA its protein (seeShort Protocols in Molecular Biology, Ausubel et al., (editors), John Wiley & Sons, Inc., 4^{th} edition, 1999). In certain embodiments, the activity of the reporter gene product is measured as a readout of reporter gene expression from the recombinant virus. For the quantification of the activity of the reporter gene product, biochemical characteristics of the reporter gene product can be employed (see Table 4). The methods for measuring the biochemical activity of the reporter gene products are well-known to the skilled artisan. A more detailed description of illustrative reporter genes that can be used with the methods of the invention is set forth below.

### 5.8.3 Measurement of Incidence of Infection Rate

The incidence of infection can be determined by any method well-known in the art, for example, but not limited to, clinical samples (e.g., nasal swabs) can be tested for the presence of a virus of the invention by immunofluorescence assay (IFA) using an anti-APV-antigen antibody, an anti-hMPV-antigen antibody, an anti-APV-antigen antibody, and/or an antibody that is specific to the gene product of the heterologous nucleotide sequence, respectively.

In certain embodiments, samples containing intact cells can be directly processed, whereas isolates without intact cells should first be cultured on a permissive cell line (e.g. 1-HEp-2 cells). In an illustrative embodiments, cultured cell suspensions should be cleared by centrifugation at, e.g., 300×g for 5 minutes at room temperature, followed by a PBS, pH 7.4 (Ca++ and Mg++ free) wash under the same conditions. Cell pellets are resuspended in a small volume of PBS for analysis. Primary clinical isolates containing intact cells are mixed with PBS and centrifuged at 300×g for 5 minutes at room temperature. Mucus is removed from the interface with a sterile pipette tip and cell pellets are washed once more with PBS under the same conditions. Pellets are then resuspended in a small volume of PBS for analysis. Five to ten microliters of each cell suspension are spotted per 5 mm well on acetone washed 12-well HTC supercured glass slides and allowed to air dry. Slides are fixed in cold (−20° C.) acetone for 10 minutes. Reactions are blocked by adding PBS-1% BSA to each well followed by a 10 minute incubation at room temperature. Slides are washed three times in PBS-0.1% Tween-20 and air dried. Ten microliters of each primary antibody reagent diluted to 250 ng/ml in blocking buffer is spotted per well and reactions are incubated in a humidified 37° C. environment for 30 minutes. Slides are then washed extensively in three changes of PBS-0.1% Tween-20 and air dried. Ten microliters of appropriate secondary conjugated antibody reagent diluted to 250 ng/ml in blocking buffer are spotted per respective well and reactions are incubated in a humidified 37° C. environment for an additional 30 minutes. Slides are then washed in three changes of PBS-0.1% Tween-20. Five microliters of PBS-50% glycerol-10 mM Tris pH 8.0-1 mM EDTA are spotted per reaction well, and slides are mounted with cover slips. Each reaction well is subsequently analyzed by fluorescence microscopy at 200× power using a B-2A filter (EX 450-490 nm). Positive reactions are scored against an autofluorescent background obtained from unstained cells or cells stained with secondary reagent alone. Positive reactions are characterized by bright fluorescence punctuated with small inclusions in the cytoplasm of infected cells.

### 5.8.4 Measurement of Serum Titer

Antibody serum titer can be determined by any method well-known in the art, for example, but not limited to, the amount of antibody or antibody fragment in serum samples can be quantitated by a sandwich ELISA. Briefly, the ELISA consists of coating microtiter plates overnight at 4° C. with an antibody that recognizes the antibody or antibody fragment in the serum. The plates are then blocked for approximately 30 minutes at room temperature with PBS-Tween-0.5% BSA. Standard curves are constructed using purified antibody or antibody fragment diluted in PBS-TWEEN-BSA, and samples are diluted in PBS-BSA. The samples and standards are added to duplicate wells of the assay plate and are incubated for approximately 1 hour at room temperature. Next, the non-bound antibody is washed away with PBS-TWEEN and the bound antibody is treated with a labeled secondary antibody (e.g., horseradish peroxidase conjugated goat-anti-human IgG) for approximately 1 hour at room temperature. Binding of the labeled antibody is detected by adding a chromogenic substrate specific for the label and measuring the rate of substrate turnover, e.g., by a spectrophotometer. The concentration of antibody or antibody fragment levels in the serum is determined by comparison of the rate of substrate turnover for the samples to the rate of substrate turnover for the standard curve at a certain dilution.

### 5.8.5 Serological Tests

In certain embodiments of the invention, the presence of antibodies that bind to a component of a mammalian MPV is detected. In particular the presence of antibodies directed to a protein of a mammalian MPV can be detected in a subject to diagnose the presence of a mammalian MPV in the subject. Any method known to the skilled artisan can be used to detect the presence of antibodies directed to a component of a mammalian MPV.

In another embodiment, serological tests can be conducted by contacting a sample, from a host suspected of being infected with MPV, with an antibody to an MPV or a component thereof, and detecting the formation of a complex. In such an embodiment, the serological test can detect the presence of a host antibody response to MPV exposure. The antibody that can be used in the assay of the invention to detect host antibodies or MPV components can be produced using any method known in the art. Such antibodies can be engineered to detect a variety of epitopes, including, but not limited to, nucleic acids, amino acids, sugars, polynucleotides, proteins, carbohydrates, or combinations thereof. In another embodiment of the invention, serological tests can be conducted by contacting a sample from a host suspected of being infected with MPV, with an a component of MPV, and detecting the formation of a complex. Examples of such methods are well known in the art, including but are not limited to, direct immunofluoresence, ELISA, western blot, immunochromatography.

In an illustrative embodiment, components of mammalian MPV are linked to a solid support. In a specific embodiment, the component of the mammalian MPV can be, but is not limited to, the F protein or the G protein. Subsequently, the material that is to be tested for the presence of antibodies directed to mammalian MPV is incubated with the solid support under conditions conducive to the binding of the antibodies to the mammalian MPV components. Subsequently, the solid support is washed under conditions that remove any unspecifically bound antibodies. Following the washing step, the presence of bound antibodies can be detected using any technique known to the skilled artisan. In a specific embodiment, the mammalian MPV protein-antibody complex is incubated with detectably labeled antibody that recognizes antibodies that were generated by the species of the subject, e.g., if the subject is a cotton rat, the detectably labeled antibody is directed to rat antibodies, under conditions conducive to the binding of the detectably labeled antibody to the antibody that is bound to the component of mammalian MPV. In a specific embodiment, the detectably labeled antibody is conjugated to an enzymatic activity. In another embodiment, the detectably labeled antibody is radioactively labeled. The complex of mammalian MPV protein-antibody-detectably labeled antibody is then washed, and subsequently the presence of the detectably labeled antibody is quantified by any technique known to the skilled artisan, wherein the technique used is dependent on the type of label of the detectably labeled antibody.

### 5.8.6 BIAcore Assay

Determination of the kinetic parameters of antibody binding can be determined for example by the injection of 250 μL of monoclonal antibody (“mAb”) at varying concentration in HBS buffer containing 0.05% Tween-20 over a sensor chip surface, onto which has been immobilized the antigen. The antigen can be any component of a mammalian MPV. In a specific embodiment, the antigen can be, but is not limited to, the F protein or the G protein of a mammalian MPV. The flow rate is maintained constant at 75 uL/min. Dissociation data is collected for 15 min, or longer as necessary. Following each injection/dissociation cycle, the bound mAb is removed from the antigen surface using brief, 1 min pulses of dilute acid, typically 10-100 mM HCl, though other regenerants are employed as the circumstances warrant.

More specifically, for measurement of the rates of association, kₒₙ, and dissociation, k_{off}, the antigen is directly immobilized onto the sensor chip surface through the use of standard amine coupling chemistries, namely the EDC/NHS method (EDC=N-diethylaminopropyl)-carbodiimide). Briefly, a 5-100 nM solution of the antigen in 10 mM NaOAc, pH4 or pH5 is prepared and passed over the EDC/NHS-activated surface until approximately 30-50 RU's (Biacore Resonance Unit) worth of antigen are immobilized. Following this, the unreacted active esters are “capped” off with an injection of 1M Et-NH2. A blank surface, containing no antigen, is prepared under identical immobilization conditions for reference purposes. Once a suitable surface has been prepared, an appropriate dilution series of each one of the antibody reagents is prepared in HBS/Tween-20, and passed over both the antigen and reference cell surfaces, which are connected in series. The range of antibody concentrations that are prepared varies depending on what the equilibrium binding constant, K_{D}, is estimated to be. As described above, the bound antibody is removed after each injection/dissociation cycle using an appropriate regenerant.

Once an entire data set is collected, the resulting binding curves are globally fitted using algorithms supplied by the instrument manufacturer, BIAcore, Inc. (Piscataway, N.J.). All data are fitted to a 1:1 Langmuir binding model. These algorithm calculate both the Kₒₙ and the k_{off}, from which the apparent equilibrium binding constant, K_{D}, is deduced as the ratio of the two rate constants (i.e. k_{off}/kₒₙ). More detailed treatments of how the individual rate constants are derived can be found in the BIAevaluation Software Handbook (BIAcore, Inc., Piscataway, N.J.).

### 5.8.7 Microneutralization Assay

The ability of antibodies or antigen-binding fragments thereof to neutralize virus infectivity is determined by a microneutralization assay. This microneutralization assay is a modification of the procedures described by Anderson et al., (1985, J. Clin. Microbiol. 22:1050-1052, the disclosure of which is hereby incorporated by reference in its entirety). The procedure is also described in Johnson et al., 1999, J. Infectious Diseases 180:35-40, the disclosure of which is hereby incorporated by reference in its entirety.

Antibody dilutions are made in triplicate using a 96-well plate. 10⁶ TCID₅₀ of a mammalian MPV are incubated with serial dilutions of the antibody or antigen-binding fragments thereof to be tested for 2 hours at 37_C in the wells of a 96-well plate. Cells susceptible to infection with a mammalian MPV, such as, but not limited to Vero cells (2.5×10⁴) are then added to each well and cultured for 5 days at 37_C in 5% CO₂. After 5 days, the medium is aspirated and cells are washed and fixed to the plates with 80% methanol and 20% PBS. Virus replication is then determined by viral antigen, such as F protein expression. Fixed cells are incubated with a biotin-conjugated anti-viral antigen, such as anti-F protein monoclonal antibody (e.g., pan F protein, C-site-specific MAb 133-1H) washed and horseradish peroxidase conjugated avidin is added to the wells. The wells are washed again and turnover of substrate TMB (thionitrobenzoic acid) is measured at 450 nm. The neutralizing titer is expressed as the antibody concentration that causes at least 50% reduction in absorbency at 450 nm (the OD₄₅₀) from virus-only control cells.

The microneutralization assay described here is only one example. Alternatively, standard neutralization assays can be used to determine how significantly the virus is affected by an antibody.

### 5.8.8 Viral Fusion Inhibition Assay

This assay is in principle identical to the microneutralization assay, except that the cells are infected with the respective virus for four hours prior to addition of antibody and the read-out is in terms of presence of absence of fusion of cells (Taylor et al., 1992, J. Gen. Virol. 73:2217-2223).

### 5.8.9 Isothermal Titration Calorimetry

Thermodynamic binding affinities and enthalpies are determined from isothermal titration calorimetry (ITC) measurements on the interaction of antibodies with their respective antigen.

Antibodies are diluted in dialysate and the concentrations were determined by UV spectroscopic absorption measurements with a Perkin-Elmer Lambda 4B Spectrophotometer using an extinction coefficient of 217,000 M⁻¹ cm⁻¹ at the peak maximum at 280 nm. The diluted mammalian MPV-antigen concentrations are calculated from the ratio of the mass of the original sample to that of the diluted sample since its extinction coefficient is too low to determine an accurate concentration without employing and losing a large amount of sample.

### ITC Measurements

The binding thermodynamics of the antibodies are determined from ITC measurements using a Microcal, Inc. VP Titration Calorimeter. The VP titration calorimeter consists of a matched pair of sample and reference vessels (1.409 ml) enclosed in an adiabatic enclosure and a rotating stirrer-syringe for titrating ligand solutions into the sample vessel. The ITC measurements are performed at 25° C. and 35° C. The sample vessel contained the antibody in the phosphate buffer while the reference vessel contains just the buffer solution. The phosphate buffer solution is saline 67 mM PO₄ at pH 7.4 from HyClone, Inc. Five or ten μl aliquots of the 0.05 to 0.1 mM RSV-antigen, PIV-antigen, and/or hMPV-antigen solution are titrated 3 to 4 minutes apart into the antibody sample solution until the binding is saturated as evident by the lack of a heat exchange signal.

A non-linear, least square minimization software program from Microcal, Inc., Origin 5.0, is used to fit the incremental heat of the i-th titration (ΔQ(i)) of the total heat, Qₜ, to the total titrant concentration, Xₜ, according to the following equations (I),

Qₜ=nCₜΔH_{b}∘V{1+Xₜ/nCₜ+1/nK_{b}Cₜ−[(1+Xₜ/nCₜ1/nK_{b}Cₜ)²−4Xₜ/nCₜ]^{1/2}}/2 (1a)

ΔQ(i)=Q(i)+dVi/2V{Q(i)+Q(i−1)}−Q(i−1) (1b)

where Cₜ is the initial antibody concentration in the sample vessel, V is the volume of the sample vessel, and n is the stoichiometry of the binding reaction, to yield values of K_{b}, ΔH_{b}∘, and n. The optimum range of sample concentrations for the determination of K_{b} depends on the value of K_{b} and is defined by the following relationship.

CₜK_{b}n≦500 (2)

so that at 1 μM the maximum K_{b} that can be determined is less than 2.5×10⁸ M⁻¹. If the first titrant addition does not fit the binding isotherm, it was neglected in the final analysis since it may reflect release of an air bubble at the syringe opening-solution interface.

### 5.8.10 Immunoassays

Immunoprecipitation protocols generally comprise lysing a population of cells in a lysis buffer such as RIPA buffer (I % NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, 159 aprotinin, sodium vanadate), adding the antibody of interest to the cell lysate, incubating for a period of time (e.g., to 4 hours) at 4 degrees C., adding protein A and/or protein G sepharose beads to the cell lysate, incubating for about an hour or more at 4 degrees C., washing the beads in lysis buffer and re-suspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al., eds., 1994, Current Protocols in Molecular Biology, Vol. 1, John Wiley & Sons, Inc., New York at pages 10, 16, 1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide get to a membrane such as nitrocellulose, PVDF or nylon, blocking the membrane, in blocking solution (e.g., PBS with 3% BSA or non-fat milk), washing the membrane in washing buffer (e.g., PBSTween20), incubating the membrane with primary antibody (the antibody of interest) diluted in blocking buffer, washing the membrane in washing buffer, incubating the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ¹²P or ¹²¹I ) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding westem blot protocols see, e.g., Ausubel et al., eds, 1994, GinTent Protocols in Molecular Biology, Vol. 1, John Wiley & Sons, Inc., New York at 10.8.1.

ELISAs comprise preparing antigen, coating the well of a 96-well microtiter plate with the antigen, washing away antigen that did not bind the wells, adding the antibody of interest conjugated to a detectable compound such as an enzymatic substrate (e.g., horseradish peroxidase or alkaline phosphatase) to the wells and incubating for a period of time, washing away unbound antibodies or non-specifically bound antibodies, and detecting the presence of the antibodies specifically bound to the antigen coating the well. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, the detectable molecule could be the antigen conjugated to a detectable compound such as an enzymatic substrate (e.g., horseradish peroxidase or alkaline phosphatase). The parameters that can be modified to increase signal detection and other variations of ELISAs are well known to one of skill in the art. For further discussion regarding ELISAs see, e.g., Ausubel et al., eds, 1994, Current Protocols in Molecular Biology, Vol. I, John Wiley & Sons, Inc., New York at 11.2.1.

The binding affinity of an antibody (including a scFv or other molecule comprising, or alternatively consisting of, antibody fragments or variants thereof) to an antigen and the off-rate of an antibody-antigen interaction can be determined by competitive binding assays. One example of a competitive binding assay is a radioimmunoassay comprising the incubation of labeled antigen (e.g., ³H or ¹²¹I ) with the antibody of interest in the presence of increasing amounts of unlabeled antigen, and the detection of the antibody bound to the labeled antigen.

### 5.8.11 Sucrose Gradient Assay

The question of whether the heterologous proteins are incorporated into the virion can be further investigated by use of any biochemical assay known to the skilled artisan. In a specific embodiment, a sucrose gradient assay is used to determine whether a heterologous protein is incorporated into the virion.

Infected cell lysates can be fractionated in 20-60% sucrose gradients, various fractions are collected and analyzed for the presence and distribution of heterologous proteins and the vector proteins by, e.g., Western blot analysis. The fractions and the virus proteins can also be assayed for peak virus titers by plaque assay. If the heterologous protein co-migrates with the virion the heterologous protein is associated with the virion.

### 5.9 Methods to Identify New Isolates of MPV

The present invention relates to mammalian MPV, in particular hMPV. While the present invention provides the characterization of two serological subgroups of MPV, A and B, and the characterization of four variants of MPV A1, A2, B1 and B2, the invention is not limited to these subgroups and variants. The invention encompasses any yet to be identified isolates of MPV, including those which are characterized as belonging to the subgroups and variants described herein, or belonging to a yet to be characterized subgroup or variant.

Immunoassays can be used in order to characterize the protein components that are present in a given sample. Immunoassays are an effective way to compare viral isolates using peptides components of the viruses for identification. For example, the invention provides herein a method to identify further isolates of MPV as provided herein, the method comprising inoculating an essentially MPV-uninfected or specific-pathogen-free guinea pig or ferret (in the detailed description the animal is inoculated intranasally but other was of inoculation such as intramuscular or intradermal inoculation, and using an other experimental animal, is also feasible) with the prototype isolate 1-2614 or related isolates. Sera are collected from the animal at day zero, two weeks and three weeks post inoculation. The animal specifically seroconverted as measured in virus neutralization (VN) assay (For an example of a VN assay, see Example 16) and indirect WA (For an example of IFA, see Example 11 or 14) against the respective isolate I-2614 and the sera from the seroconverted animal are used in the immunological detection of said further isolates. As an example, the invention provides the characterization of a new member in the family of Paramyxoviridae, a human metapneumovirus or metapneumovirus-like virus (since its final taxonomy awaits discussion by a viral taxonomy committee the MPV is herein for example described as taxonomically corresponding to APV) (MPV) which may cause severe RTI in humans. The clinical signs of the disease caused by MPV are essentially similar to those caused by hRSV, such as cough, myalgia, vomiting, fever broncheolitis or pneumonia, possible conjunctivitis, or combinations thereof. As is seen with hRSV infected children, specifically very young children may require hospitalization. As an example an MPV which was deposited Jan. 19, 2001 as 1-2614 with CNCM, Institute Pasteur, Paris or a virus isolate phylogenetically corresponding therewith is herewith provided. Therewith, the invention provides a virus comprising a nucleic acid or functional fragment phylogenetically corresponding to a nucleic acid sequence of SEQ. ID NO:19, or structurally corresponding therewith. In particular the invention provides a virus characterized in that after testing it in phylogenetic tree analysis wherein maximum likelihood trees are generated using 100 bootstraps and 3 jumbles it is found to be more closely phylogenetically corresponding to a virus isolate deposited as I-2614 with CNCM, Paris than it is related to a virus isolate of avian pnuemovirus (APV) also known as turkey rhinotracheitis virus (TRTV), the aetiological agent of avian rhinotracheitis. It is particularly useful to use an AVP-C virus isolate as outgroup in said phylogenetic tree analysis, it being the closest relative, albeit being an essentially non-mammalian virus.

### 5.9.1 Bioinformatics Alignment of Sequences

Two or more amino acid sequences can be compared by BLAST (Altschul, S. F. et al., 1990, J. Mol. Biol. 215:403-410) to determine their sequence homology and sequence identities to each other. Two or more nucleotide sequences can be compared by BLAST (Altschul, S. F. et al., 1990, J. Mol. Biol. 215:403-410) to determine their sequence homology and sequence identities to each other. BLAST comparisons can be performed using the Clustal W method (MacVector(tm)). In certain specific embodiments, the alignment of two or more sequences by a computer program can be followed by manual re-adjustment.

The determination of percent identity between two sequences can be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul et al., 1990, J. Mol. Biol. 215:403-410. BLAST nucleotide comparisons can be performed with the NBLAST program. BLAST amino acid sequence comparisons can be performed with the XBLAST program. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Altschul et al., 1997, supra). When utilizing BLAST, Gapped BLAST, and PSI-Blast programs, the default parameters of the respective programs (e.g., XBLAST and NBLAST) can be used (seehttp://www.ncbi.nlm.nih.gov). Another preferred, non-limiting example of a mathematical algorithm utilized for the comparison of sequences is the algorithm of Myers and Miller, 1988, CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table can be used. The gap length penalty can be set by the skilled artisan. The percent identity between two sequences can be determined using techniques similar to those described above, with or without allowing gaps. In calculating percent identity, typically only exact matches are counted.

### 5.9.2 Hybridization Conditions

A nucleic acid which is hybridizable to a nucleic acid of a mammalian MPV, or to its reverse complement, or to its complement can be used in the methods of the invention to determine their sequence homology and identities to each other. In certain embodiments, the nucleic acids are hybridized under conditions of high stringency. By way of example and not limitation, procedures using such conditions of high stringency are as follows. Prehybridization of filters containing DNA is carried out for 8 h to overnight at 65 C in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65 C in prehybridization mixture containing 100 μg/ml denatured salmon sperm DNA and 5-20×106 cpm of 32P-labeled probe. Washing of filters is done at 37 C for 1 h in a solution containing 2×SSC, 0.01% PVP, 0.01% Ficoll, and 0.01% BSA. This is followed by a wash in 0.1×SSC at 50 C for 45 min before autoradiography. Other conditions of high stringency which may be used are well known in the art. In other embodiments of the invention, hybridization is performed under moderate of low stringency conditions, such conditions are well-known to the skilled artisan (see e.g., Sambrook et al., 1989, Molecular Cloning, A Laboratory Manual, 2d Ed., Cold Spring Harbor Laboratory Press, Cold Spring Harbor, New York; see also, Ausubel et al., eds., in the Current Protocols in Molecular Biology series of laboratory technique manuals, 1987-1997 Current Protocols, ©1994-1997 John Wiley and Sons, Inc.).

### 5.9.3 Phylogenetic Analysis

This invention relates to the inference of phylogenetic relationships between isolates of mammalian MPV. Many methods or approaches are available to analyze phylogenetic relationship; these include distance, maximum likelihood, and maximum parsimony methods (Swofford, D L., et. al., Phylogenetic Inference. In Molecular Systematics. Eds. Hillis, D M, Mortiz, C, and Mable, B K. 1996. Sinauer Associates: Massachusetts, USA. pp. 407-514; Felsenstein, J., 1981, J. Mol. Evol. 17:368-376). In addition, bootstrapping techniques are an effective means of preparing and examining confidence intervals of resultant phylogenetic trees (Felsenstein, J., 1985, Evolution. 29:783-791). Any method or approach using nucleotide or peptide sequence information to compare mammalian MPV isolates can be used to establish phylogenetic relationships, including, but not limited to, distance, maximum likelihood, and maximum parsimony methods or approaches. Any method known in the art can be used to analyze the quality of phylogenetic data, including but not limited to bootstrapping. Alignment of nucleotide or peptide sequence data for use in phylogenetic approaches, include but are not limited to, manual alignment, computer pairwise alignment, and computer multiple alignment. One skilled in the art would be familiar with the preferable alignment method or phylogenetic approach to be used based upon the information required and the time allowed.

In one embodiment, a DNA maximum likehood method is used to infer relationships between hMPV isolates. In another embodiment, bootstrapping techniques are used to determine the certainty of phylogenetic data created using one of said phylogenetic approaches. In another embodiment, jumbling techniques are applied to the phylogenetic approach before the input of data in order to minimize the effect of sequence order entry on the phylogenetic analyses. In one specific embodiment, a DNA maximum likelihood method is used with bootstrapping. In another specific embodiment, a DNA maximum likelihood method is used with bootstrapping and jumbling. In another more specific embodiment, a DNA maximum likelihood method is used with 50 bootstraps. In another specific embodiment, a DNA maximum likelihood method is used with 50 bootstraps and 3 jumbles. In another specific embodiment, a DNA maximum likelihood method is used with 100 bootstraps and 3 jumbles.

In one embodiment, nucleic acid or peptide sequence information from an isolate of hMPV is compared or aligned with sequences of other hMPV isolates. The amino acid sequence can be the amino acid sequence of the L protein, the M protein, the N protein, the P protein, or the F protein. In another embodiment, nucleic acid or peptide sequence information from an hMPV isolate or a number of hMPV isolates is compared or aligned with sequences of other viruses. In another embodiment, phylogenetic approaches are applied to sequence alignment data so that phylogenetic relationships can be inferred and/or phylogenetic trees constructed. Any method or approach that uses nucleotide or peptide sequence information to compare hMPV isolates can be used to infer said phylogenetic relationships, including, but not limited to, distance, maximum likelihood, and maximum parsimony methods or approaches.

Other methods for the phylogenetic analysis are disclosed in International Patent Application PCT/NL02/00040, published as WO 02/057302, which is incorporated in its entirety herein. In particular, PCT/NL02/00040 discloses nucleic acid sequences that are suitable for phylogenetic analysis at page 12, line 27 to page 19, line 29, which is incorporated herein by reference.

For the phylogenetic analyses it is most useful to obtain the nucleic acid sequence of a non-MPV as outgroup with which the virus is to be compared, a very useful outgroup isolate can be obtained from avian pneumovirus serotype C (APV-C).

Many methods and programs are known in the art and can be used in the inference of phylogenetic relationships, including, but not limited to BioEdit, ClustalW, TreeView, and NJPlot. Methods that would be used to align sequences and to generate phylogenetic trees or relationships would require the input of sequence information to be compared. Many methods or formats are known in the art and can be used to input sequence information, including, but not limited to, FASTA, NBRF, EMBL/SWISS, GDE protein, GDE nucleotide, CLUSTAL, and GCG/MSF. Methods that would be used to align sequences and to generate phylogenetic trees or relationships would require the output of results. Many methods or formats can be used in the output of information or results, including, but not limited to, CLUSTAL, NBRF/PIR, MSF, PHYLIP, and GDE. In one embodiment, ClustalW is used in conjunction with DNA maximum likelihood methods with 100 bootstraps and 3 jumbles in order to generate phylogenetic relationships.

### 5.10 Generation of Antibodies

The invention also relates to the generation of antibodies against a protein encoded by a mammalian MPV. In particular, the invention relates to the generation of antibodies against all MPV antigens, including the F protein, N protein, M2-1 protein, M2-2 protein, G protein, or P protein of a mammalian MPV. According to the invention, any protein encoded by a mammalian MPV, derivatives, analogs or fragments thereof, may be used as an immunogen to generate antibodies which immunospecifically bind such an immunogen. Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule. Examples of immunologically active portions of immunoglobulin molecules include F(ab) and F(ab′)2 fragments which can be generated by treating the antibody with an enzyme such as pepsin or papain. In a specific embodiment, antibodies to a protein encoded by human MPV are produced. In another embodiment, antibodies to a domain a protein encoded by human MPV are produced.

Various procedures known in the art may be used for the production of polyclonal antibodies against a protein encoded by a mammalian MPV, derivatives, analogs or fragments thereof. For the production of antibody, various host animals can be immunized by injection with the native protein, or a synthetic version, or derivative (e.g., fragment) thereof, including but not limited to rabbits, mice, rats, etc. Various adjuvants may be used to increase the immunological response, depending on the host species, and including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum.

For preparation of monoclonal antibodies directed toward a protein encoded by a mammalian MPV, derivatives, analogs or fragments thereof, any technique which provides for the production of antibody molecules by continuous cell lines in culture may be used. For example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). In an additional embodiment of the invention, monoclonal antibodies can be produced in germ-free animals utilizing recent technology (PCT/US90/02545). According to the invention, human antibodies may be used and can be obtained by using human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, pp. 77-96). In fact, according to the invention, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for a protein encoded by a mammalian MPV, derivatives, analogs or fragments thereof together with genes from a human antibody molecule of appropriate biological activity can be used; such antibodies are within the scope of this invention.

According to the invention, techniques described for the production of single chain antibodies (U.S. Pat. No. 4,946,778) can be adapted to produce specific single chain antibodies. An additional embodiment of the invention utilizes the techniques described for the construction of Fab expression libraries (Huse et al., 1989, Science 246:1275-1281) to allow rapid and easy identification of monoclonal Fab fragments with the desired specificity for a protein encoded by a mammalian MPV, derivatives, analogs or fragments thereof.

Antibody fragments which contain the idiotype of the molecule can be generated by known techniques. For example, such fragments include but are not limited to: the F(ab′)2 fragment which can be produced by pepsin digestion of the antibody molecule; the Fab′ fragments which can be generated by reducing the disulfide bridges of the F(ab′)2 fragment, the Fab fragments which can be generated by treating the antibody molecule with papain and a reducing agent, and Fv fragments.

In the production of antibodies, screening for the desired antibody can be accomplished by techniques known in the art, e.g. ELISA (enzyme-linked immunosorbent assay). For example, to select antibodies which recognize a specific domain of a protein encoded by a mammalian MPV, one may assay generated hybridomas for a product which binds to a fragment of a protein encoded by a mammalian MPV containing such domain.

The antibodies provided by the present invention can be used for detecting MPV and for therapeutic methods for the treatment of infections with MPV.

The specificity and binding affinities of the antibodies generated by the methods of the invention can be tested by any technique known to the skilled artisan. In certain embodiments, the specificity and binding affinities of the antibodies generated by the methods of the invention can be tested as described in sections 5.8.5, 5.8.6, 5.8.7, 5.8.8 or 5.8.9.

### 5.11 Screening Assays to Identify Antiviral Agents

The invention provides methods for the identification of a compound that inhibits the ability of a mammalian MPV to infect a host or a host cell. In certain embodiments, the invention provides methods for the identification of a compound that reduces the ability of a mammalian MPV to replicate in a host or a host cell. Any technique well-known to the skilled artisan can be used to screen for a compound that would abolish or reduce the ability of a mammalian MPV to infect a host and/or to replicate in a host or a host cell. In a specific embodiment, the mammalian MPV is a human MPV.

In certain embodiments, the invention provides methods for the identification of a compound that inhibits the ability of a mammalian MPV to replicate in a mammal or a mammalian cell. More specifically, the invention provides methods for the identification of a compound that inhibits the ability of a mammalian MPV to infect a mammal or a mammalian cell. In certain embodiments, the invention provides methods for the identification of a compound that inhibits the ability of a mammalian MPV to replicate in a mammalian cell. In a specific embodiment, the mammalian cell is a human cell. For a detailed description of assays that can be used to determine virus titer see section 5.7.

In certain embodiments, a cell is contacted with a test compound and infected with a mammalian MPV. In certain embodiments, a control culture is infected with a mammalian virus in the absence of a test compound. The cell can be contacted with a test compound before, concurrently with, or subsequent to the infection with the mammalian MPV. In a specific embodiment, the cell is a mammalian cell. In an even more specific embodiment, the cell is a human cell. In certain embodiments, the cell is incubated with the test compound for at least 1 minute, at least 5 minutes at least 15 minutes, at least 30 minutes, at least 1 hour, at least 2 hours, at least 5 hours, at least 12 hours, or at least 1 day. The titer of the virus can be measured at any time during the assay. In certain embodiments, a time course of viral growth in the culture is determined. If the viral growth is inhibited or reduced in the presence of the test compound, the test compound is identified as being effective in inhibiting or reducing the growth or infection of a mammalian MPV. In a specific embodiment, the compound that inhibits or reduces the growth of a mammalian MPV is tested for its ability to inhibit or reduce the growth rate of other viruses to test its specificity for mammalian MPV.

In certain embodiments, a test compound is administered to a model animal and the model animal is infected with a mammalian MPV. In certain embodiments, a control model animal is infected with a mammalian virus in without the administration of a test compound. The test compound can be administered before, concurrently with, or subsequent to the infection with the mammalian MPV. In a specific embodiment, the model animal is a mammal. In an even more specific embodiment, the model animal can be, but is not limited to, a cotton rat, a mouse, or a monkey. The titer of the virus in the model animal can be measured at any time during the assay. In certain embodiments, a time course of viral growth in the culture is determined. If the viral growth is inhibited or reduced in the presence of the test compound, the test compound is identified as being effective in inhibiting or reducing the growth or infection of a mammalian MPV. In a specific embodiment, the compound that inhibits or reduces the growth of a mammalian MPV in the model animal is tested for its ability to inhibit or reduce the growth rate of other viruses to test its specificity for mammalian MPV.

### 5.12 Formulations of Vaccines, Antibodies and Antivirals

In a preferred embodiment, the invention provides a proteinaceous molecule or metapneumovirus-specific viral protein or functional fragment thereof encoded by a nucleic acid according to the invention. Useful proteinaceous molecules are for example derived from any of the genes or genomic fragments derivable from a virus according to the invention. Such molecules, or antigenic fragments thereof, as provided herein, are for example useful in diagnostic methods or kits and in pharmaceutical compositions such as sub-unit vaccines. Particularly useful are the F, SH and/or G protein or antigenic fragments thereof for inclusion as antigen or subunit immunogen, but inactivated whole virus can also be used. Particularly useful are also those proteinaceous substances that are encoded by recombinant nucleic acid fragments that are identified for phylogenetic analyses, of course preferred are those that are within the preferred bounds and metes of ORFs useful in phylogenetic analyses, in particular for eliciting MPV specific antibody or T cell responses, whether in vivo (e.g. for protective purposes or for providing diagnostic antibodies) or in vitro (e.g. by phage display technology or another technique useful for generating synthetic antibodies).

Also provided herein are antibodies, be it natural polyclonal or monoclonal, or synthetic (e.g. (phage) library-derived binding molecules) antibodies that specifically react with an antigen comprising a proteinaceous molecule or MPV-specific functional fragment thereof according to the invention. Such antibodies are useful in a method for identifying a viral isolate as an MPV comprising reacting said viral isolate or a component thereof with an antibody as provided herein. This can for example be achieved by using purified or non-purified MPV or parts thereof (proteins, peptides) using ELISA, RIA, FACS or different formats of antigen detection assays (Current Protocols in Immunology). Alternatively, infected cells or cell cultures may be used to identify viral antigens using classical immunofluorescence or immunohistochemical techniques.

A pharmaceutical composition comprising a virus, a nucleic acid, a proteinaceous molecule or fragment thereof, an antigen and/or an antibody according to the invention can for example be used in a method for the treatment or prevention of a MPV infection and/or a respiratory illness comprising providing an individual with a pharmaceutical composition according to the invention. This is most useful when said individual comprises a human, specifically when said human is below 5 years of age, since such infants and young children are most likely to be infected by a human MPV as provided herein. Generally, in the acute phase patients will suffer from upper respiratory symptoms predisposing for other respiratory and other diseases. Also lower respiratory illnesses may occur, predisposing for more and other serious conditions. The compositions of the invention can be used for the treatment of immuno-compromised individuals including cancer patients, transplant recipients and the elderly.

The invention also provides methods to obtain an antiviral agent useful in the treatment of respiratory tract illness comprising establishing a cell culture or experimental animal comprising a virus according to the invention, treating said culture or animal with an candidate antiviral agent, and determining the effect of said agent on said virus or its infection of said culture or animal. An example of such an antiviral agent comprises a MPV-neutralising antibody, or functional component thereof, as provided herein, but antiviral agents of other nature are obtained as well. The invention also provides use of an antiviral agent according to the invention for the preparation of a pharmaceutical composition, in particular for the preparation of a pharmaceutical composition for the treatment of respiratory tract illness, specifically when caused by an MPV infection or related disease, and provides a pharmaceutical composition comprising an antiviral agent according to the invention, useful in a method for the treatment or prevention of an MPV infection or respiratory illness, said method comprising providing an individual with such a pharmaceutical composition.

In certain embodiments of the invention, the vaccine of the invention comprises mammalian metapneumovirus as defined herein. In certain, more specific embodiments, the mammalian metapneumovirus is a human metapneumovirus. In a preferred embodiment, the mammalian metapneumovirus to be used in a vaccine formulation has an attenuated phenotype. For methods to achieve an attenuated phenotype, see section 5.6.

The invention provides vaccine formulations for the prevention and treatment of infections with PIV, RSV, APV, and/or hMPV. In certain embodiments, the vaccine of the invention comprises recombinant and chimeric viruses of the invention. In certain embodiments, the virus is attenuated.

In a specific embodiment, the vaccine comprises APV and the vaccine is used for the prevention and treatment for hMPV infections in humans. Without being bound by theory, because of the high degree of homology of the F protein of APV with the F protein of hMPV, infection with APV will result in the production of antibodies in the host that will cross-react with hMPV and protect the host from infection with hMPV and related diseases.

In another specific embodiment, the vaccine comprises hMPV and the vaccine is used for the prevention and treatment for APV infection in birds, such as, but not limited to, in turkeys. Without being bound by theory, because of the high degree of homology of the F protein of APV with the F protein of hMPV, infection with hMPV will result in the production of antibodies in the host that will cross-react with APV and protect the host from infection with APV and related diseases.

In a specific embodiment, the invention encompasses the use of recombinant and chimeric APV/hMPV viruses which have been modified in vaccine formulations to confer protection against APV and/or hMPV. In certain embodiments, APV/hMPV is used in a vaccine to be administered to birds, to protect the birds from infection with APV. Without being bound by theory, the replacement of the APV gene or nucleotide sequence with a hMPV gene or nucleotide sequence results in an attenuated phenotype that allows the use of the chimeric virus as a vaccine. In other embodiments the APV/hMPV chimeric virus is administered to humans. Without being bound by theory the APV viral vector provides the attenuated phenotype in humans and the expression of the hMPV sequence elicits a hMPV specific immune response.

In a specific embodiment, the invention encompasses the use of recombinant and chimeric hMPV/APV viruses which have been modified in vaccine formulations to confer protection against APV and/or hMPV. In certain embodiments, hMPV/APV is used in a vaccine to be administered to humans, to protect the human from infection with hMPV. Without being bound by theory, the replacement of the hMPV gene or nucleotide sequence with a APV gene or nucleotide sequence results in an attenuated phenotype that allows the use of the chimeric virus as a vaccine. In other embodiments the hMPV/APV chimeric virus is administered to birds. Without being bound by theory the hMPV backbone provides the attenuated phenotype in birds and the expression of the APV sequence elicits an APV specific immune response.

In certain preferred embodiments, the vaccine formulation of the invention is used to protect against infections by a metapneumovirus and related diseases. More specifically, the vaccine formulation of the invention is used to protect against infections by a human metapneumovirus and/or an avian pneumovirus and related diseases. In certain embodiments, the vaccine formulation of the invention is used to protect against infections by (a) a human metapneumovirus and a respiratory syncytial virus; and/or (b) an avian pneumovirus and a respiratory syncytial virus.

In certain embodiments, the vaccine formulation of the invention is used to protect against infections by (a) a human metapneumovirus and a human parainfluenza virus; and/or (b) an avian pneumovirus and a human parainfluenza virus, and related diseases.

In certain embodiments, the vaccine formulation of the invention is used to protect against infections by (a) a human metapneumovirus, a respiratory syncytial virus, and a human parainfluenza virus; and/or (b) an avian pneumovirus, a respiratory syncytial virus, and a human parainfluenza virus, and related diseases.

In certain embodiments, the vaccine formulation of the invention is used to protect against infections by a human metapneumovirus, a respiratory syncytial virus, and a human parainfluenza virus and related diseases. In certain other embodiments, the vaccine formulation of the invention is used to protect against infections by an avian pneumovirus, a respiratory syncytial virus, and a human parainfluenza virus and related diseases.

Due to the high degree of homology among the F proteins of different viral species, the vaccine formulations of the invention can be used for protection from viruses different from the one from which the heterologous nucleotide sequence encoding the F protein was derived. In a specific exemplary embodiment, a vaccine formulation contains a virus comprising a heterologous nucleotide sequence derived from an avian pneumovirus type A, and the vaccine formulation is used to protect from infection by avian pneumovirus type A and avian pneumovirus type B. The invention encompasses vaccine formulations to be administered to humans and animals which are useful to protect against APV, including APV-C and APV-D, hMPV, PIV, influenza, RSV, Sendai virus, mumps, laryngotracheitis virus, simianvirus 5, human papillomavirus, measles, mumps, as well as other viruses and pathogens and related diseases. The invention further encompasses vaccine formulations to be administered to humans and animals which are useful to protect against human metapneumovirus infections and avian pneumovirus infections and related diseases.

In one embodiment, the invention encompasses vaccine formulations which are useful against domestic animal disease causing agents including rabies virus, feline leukemia virus (FLV) and canine distemper virus. In yet another embodiment, the invention encompasses vaccine formulations which are useful to protect livestock against vesicular stomatitis virus, rabies virus, rinderpest virus, swinepox virus, and further, to protect wild animals against rabies virus.

Attenuated viruses generated by the reverse genetics approach can be used in the vaccine and pharmaceutical formulations described herein. Reverse genetics techniques can also be used to engineer additional mutations to other viral genes important for vaccine production—the epitopes of useful vaccine strain variants can be engineered into the attenuated virus. Alternatively, completely foreign epitopes, including antigens derived from other viral or non-viral pathogens can be engineered into the attenuated strain. For example, antigens of non-related viruses such as HIV (gp160, gp120, gp41) parasite antigens (e.g., malaria), bacterial or fungal antigens or tumor antigens can be engineered into the attenuated strain. Alternatively, epitopes which alter the tropism of the virus in vivo can be engineered into the chimeric attenuated viruses of the invention.

Virtually any heterologous gene sequence may be constructed into the chimeric viruses of the invention for use in vaccines. Preferably moieties and peptides that act as biological response modifiers. Preferably, epitopes that induce a protective immune response to any of a variety of pathogens, or antigens that bind neutralizing antibodies may be expressed by or as part of the chimeric viruses. For example, heterologous gene sequences that can be constructed into the chimeric viruses of the invention include, but are not limited to influenza and parainfluenza hemagglutinin neuraminidase and fusion glycoproteins such as the HN and F genes of human PIV3. In yet another embodiment, heterologous gene sequences that can be engineered into the chimeric viruses include those that encode proteins with immuno-modulating activities. Examples of immuno-modulating proteins include, but are not limited to, cytokines, interferon type 1, gamma interferon, colony stimulating factors, interleukin-1, -2, -4, -5, -6, -12, and antagonists of these agents.

In addition, heterologous gene sequences that can be constructed into the chimeric viruses of the invention for use in vaccines include but are not limited to sequences derived from a human immunodeficiency virus (HIV), preferably type 1 or type 2. In a preferred embodiment, an immunogenic HIV-derived peptide which may be the source of an antigen may be constructed into a chimeric PIV that may then be used to elicit a vertebrate immune response. Such HIV-derived peptides may include, but are not limited to sequences derived from the env gene (i.e., sequences encoding all or part of gp160, gp120, and/or gp41), the pol gene (i.e., sequences encoding all or part of reverse transcriptase, endonuclease, protease, and/or integrase), the gag gene (i.e., sequences encoding all or part of p7, p6, p55, p17/18, p24/25), tat, rev, nef, vif, vpu, vpr, and/or vpx.

Other heterologous sequences may be derived from hepatitis B virus surface antigen (HBsAg); hepatitis A or C virus surface antigens, the glycoproteins of Epstein Barr virus; the glycoproteins of human papillomavirus; the glycoproteins of respiratory syncytial virus, parainfluenza virus, Sendai virus, simianvirus 5 or mumps virus; the glycoproteins of influenza virus; the glycoproteins of herpesviruses; VP1 of poliovirus; antigenic determinants of non-viral pathogens such as bacteria and parasites, to name but a few. In another embodiment, all or portions of immunoglobulin genes may be expressed. For example, variable regions of anti-idiotypic immunoglobulins that mimic such epitopes may be constructed into the chimeric viruses of the invention.

Other heterologous sequences may be derived from tumor antigens, and the resulting chimeric viruses be used to generate an immune response against the tumor cells leading to tumor regression in vivo. These vaccines may be used in combination with other therapeutic regimens, including but not limited to chemotherapy, radiation therapy, surgery, bone marrow transplantation, etc. for the treatment of tumors. In accordance with the present invention, recombinant viruses may be engineered to express tumor-associated antigens (TAAs), including but not limited to, human tumor antigens recognized by T cells (Robbins and Kawakami, 1996, Curr. Opin. Immunol. 8:628-636, incorporated herein by reference in its entirety), melanocyte lineage proteins, including gp100, MART-1/MelanA, TRP-1 (gp75), tyrosinase; Tumor-specific widely shared antigens, MAGE-1, MAGE-3, BAGE, GAGE-1, GAGE-1, N-acetylglucosaminyltransferase-V, p15; Tumor-specific mutated antigens, β-catenin, MUM-1, CDK4; Nonmelanoma antigens for breast, ovarian, cervical and pancreatic carcinoma, HER-2/neu, human papillomavirus-E6, -E7, MUC-1.

In even other embodiments, a heterologous nucleotide sequence is derived from a metapneumovirus, such as human metapneumovirus and/or avian pneumovirus. In even other embodiments, the virus of the invention contains two different heterologous nucleotide sequences wherein one is derived from a metapneumovirus, such as human metapneumovirus and/or avian pneumovirus, and the other one is derived from a respiratory syncytial virus. The heterologous nucleotide sequence encodes a F protein or a G protein of the respective virus. In a specific embodiment, a heterologous nucleotide sequences encodes a chimeric F protein, wherein the chimeric F protein contains the ectodomain of a F protein of a metapneumovirus and the transmembrane domain as well as the luminal domain of a F protein of a parainfluenza virus.

Either a live recombinant viral vaccine or an inactivated recombinant viral vaccine can be formulated. A live vaccine may be preferred because multiplication in the host leads to a prolonged stimulus of similar kind and magnitude to that occurring in natural infections, and therefore, confers substantial, long-lasting immunity. Production of such live recombinant virus vaccine formulations may be accomplished using conventional methods involving propagation of the virus in cell culture or in the allantois of the chick embryo followed by purification.

In a specific embodiment, the recombinant virus is non-pathogenic to the subject to which it is administered. In this regard, the use of genetically engineered viruses for vaccine purposes may desire the presence of attenuation characteristics in these strains. The introduction of appropriate mutations (e.g., deletions) into the templates used for transfection may provide the novel viruses with attenuation characteristics. For example, specific missense mutations which are associated with temperature sensitivity or cold adaption can be made into deletion mutations. These mutations should be more stable than the point mutations associated with cold or temperature sensitive mutants and reversion frequencies should be extremely low.

Alternatively, chimeric viruses with “suicide” characteristics may be constructed. Such viruses would go through only one or a few rounds of replication within the host. When used as a vaccine, the recombinant virus would go through limited replication cycle(s) and induce a sufficient level of immune response but it would not go further in the human host and cause disease. Recombinant viruses lacking one or more of the genes of wild type APV and hMPV, respectively, or possessing mutated genes as compared to the wild type strains would not be able to undergo successive rounds of replication. Defective viruses can be produced in cell lines which permanently express such a gene(s). Viruses lacking an essential gene(s) will be replicated in these cell lines but when administered to the human host will not be able to complete a round of replication. Such preparations may transcribe and translate—in this abortive cycle—a sufficient number of genes to induce an immune response. Alternatively, larger quantities of the strains could be administered, so that these preparations serve as inactivated (killed) virus vaccines. For inactivated vaccines, it is preferred that the heterologous gene product be expressed as a viral component, so that the gene product is associated with the virion. The advantage of such preparations is that they contain native proteins and do not undergo inactivation by treatment with formalin or other agents used in the manufacturing of killed virus vaccines. Alternatively, recombinant virus of the invention made from cDNA may be highly attenuated so that it replicates for only a few rounds.

In certain embodiments, the vaccine of the invention comprises an attenuated mammalian MPV. Without being bound by theory, the attenuated virus can be effective as a vaccine even if the attenuated virus is incapable of causing a cell to generate new infectious viral particles because the viral proteins are inserted in the cytoplasmic membrane of the host thus stimulating an immune response.

In another embodiment of this aspect of the invention, inactivated vaccine formulations may be prepared using conventional techniques to “kill” the chimeric viruses. Inactivated vaccines are “dead” in the sense that their infectivity has been destroyed. Ideally, the infectivity of the virus is destroyed without affecting its immunogenicity. In order to prepare inactivated vaccines, the chimeric virus may be grown in cell culture or in the allantois of the chick embryo, purified by zonal ultracentrifugation, inactivated by formaldehyde or β-propiolactone, and pooled. The resulting vaccine is usually inoculated intramuscularly.

Inactivated viruses may be formulated with a suitable adjuvant in order to enhance the immunological response. Such adjuvants may include but are not limited to mineral gels, e.g., aluminum hydroxide; surface active substances such as lysolecithin, pluronic polyols, polyanions; peptides; oil emulsions; and potentially useful human adjuvants such as BCG, Corynebacterium parvum, ISCOMS and virosomes.

Many methods may be used to introduce the vaccine formulations described above, these include but are not limited to oral, intradermal, intramuscular, intraperitoneal, intravenous, subcutaneous, percutaneous, and intranasal and inhalation routes. It may be preferable to introduce the chimeric virus vaccine formulation via the natural route of infection of the pathogen for which the vaccine is designed.

In certain embodiments, the invention relates to immunogenic compositions. The immunogenic compositions comprise a mammalian MPV. In a specific embodiment, the immunogenic composition comprises a human MPV. In certain embodiments, the immunogenic composition comprises an attenuated mammalian MPV or an attenuated human MPV. In certain embodiments, the immunogenic composition further comprises a pharmaceutically acceptable carrier.

### 5.13 Dosage Regimens, Administration and Formulations

The present invention provides vaccines and immunogenic preparations comprising

MPV and APV, including attenuated forms of the virus, recombinant forms of MPV and APV, and chimeric MPV and APV expressing one or more heterologous or non-native antigenic sequences. The vaccines or immunogenic preparations of the invention encompass single or multivalent vaccines, including bivalent and trivalent vaccines. The vaccines or immunogenic formulations of the invention are useful in providing protections against various viral infections. Particularly, the vaccines or immunogenic formulations of the invention provide protection against respiratory tract infections in a host.

A recombinant virus and/or a vaccine or immunogenic formulation of the invention can be administered alone or in combination with other vaccines. Preferably, a vaccine or immunogenic formulation of the invention is administered in combination with other vaccines or immunogenic formulations that provide protection against respiratory tract diseases, such as but not limited to, respiratory syncytial virus vaccines, influenza vaccines, measles vaccines, mumps vaccines, rubella vaccines, pneumococcal vaccines, rickettsia vaccines, staphylococcus vaccines, whooping cough vaccines or vaccines against respiratory tract cancers. In a preferred embodiment, the virus and/or vaccine of the invention is administered concurrently with pediatric vaccines recommended at the corresponding ages. For example, at two, four or six months of age, the virus and/or vaccine of the invention can be administered concurrently with DtaP (IM), Hib (IM), Polio (IPV or OPV) and Hepatitis B (IM). At twelve or fifteen months of age, the virus and/or vaccine of the invention can be administered concurrently with Hib (IM), Polio (IPV or OPV), MMRII® (SubQ); Varivax® (SubQ), and hepatitis B (IM). The vaccines that can be used with the methods of invention are reviewed in various publications, e.g., The Jordan Report 2000, Division of Microbiology and Infectious Diseases, National Institute of Allergy and Infectious Diseases, National Institutes of Health, United States, the content of which is incorporated herein by reference in its entirety.

A vaccine or immunogenic formulation of the invention may be administered to a subject per se or in the form of a pharmaceutical or therapeutic composition. Pharmaceutical compositions comprising an adjuvant and an immunogenic antigen of the invention (e.g., a virus, a chimeric virus, a mutated virus) may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries which facilitate processing of the immunogenic antigen of the invention into preparations which can be used pharmaceutically. Proper formulation is, amongst others, dependent upon the route of administration chosen.

When a vaccine or immunogenic composition of the invention comprises adjuvants or is administered together with one or more adjuvants, the adjuvants that can be used include, but are not limited to, mineral salt adjuvants or mineral salt gel adjuvants, particulate adjuvants, microparticulate adjuvants, mucosal adjuvants, and immunostimulatory adjuvants. Examples of adjuvants include, but are not limited to, aluminum hydroxide, aluminum phosphate gel, Freund's Complete Adjuvant, Freund's Incomplete Adjuvant, squalene or squalane oil-in-water adjuvant formulations, biodegradable and biocompatible polyesters, polymerized liposomes, triterpenoid glycosides or saponins (e.g., QuilA and QS-21, also sold under the trademark STIMULON, ISCOPREP), N-acetyl-muramyl-L-threonyl-D-isoglutamine (Threonyl-MDP, sold under the trademark TERMURTIDE), LPS, monophosphoryl Lipid A (3D-MLAsold under the trademark MPL).

The subject to which the vaccine or an immunogenic composition of the invention is administered is preferably a mammal, most preferably a human, but can also be a non-human animal, including but not limited to, primates, cows, horses, sheep, pigs, fowl (e.g., chickens, turkeys), goats, cats, dogs, hamsters, mice and rodents.

Many methods may be used to introduce the vaccine or the immunogenic composition of the invention, including but not limited to, oral, intradermal, intramuscular, intraperitoneal, intravenous, subcutaneous, percutaneous, intranasal and inhalation routes, and via scarification (scratching through the top layers of skin, e.g., using a bifurcated needle).

For topical administration, the vaccine or immunogenic preparations of the invention may be formulated as solutions, gels, ointments, creams, suspensions, etc. as are well-known in the art.

For administration intranasally or by inhalation, the preparation for use according to the present invention can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

For injection, the vaccine or immunogenic preparations may be formulated in aqueous solutions, preferably in physiologically compatible buffers such as Hanks's solution, Ringer's solution, or physiological saline buffer. The solution may contain formulatory agents such as suspending, stabilizing and/or dispersing agents. Alternatively, the proteins may be in powder form for constitution with a suitable vehicle, e.g., sterile pyrogen-free water, before use.

Determination of an effective amount of the vaccine or immunogenic formulation for administration is well within the capabilities of those skilled in the art, especially in light of the detailed disclosure provided herein.

An effective dose can be estimated initially from in vitro assays. For example, a dose can be formulated in animal models to achieve an induction of an immunity response using techniques that are well known in the art. One having ordinary skill in the art could readily optimize administration to all animal species based on results described herein. Dosage amount and interval may be adjusted individually. For example, when used as an immunogenic composition, a suitable dose is an amount of the composition that when administered as described above, is capable of eliciting an antibody response. When used as a vaccine, the vaccine or immunogenic formulations of the invention may be administered in about 1 to 3 doses for a 1-36 week period. Preferably, 1 or 2 doses are administered, at intervals of about 2 weeks to about 4 months, and booster vaccinations may be given periodically thereafter. Alternate protocols may be appropriate for individual animals. A suitable dose is an amount of the vaccine formulation that, when administered as described above, is capable of raising an immunity response in an immunized animal sufficient to protect the animal from an infection for at least 4 to 12 months. In general, the amount of the antigen present in a dose ranges from about 1 pg to about 100 mg per kg of host, typically from about 10 pg to about 1 mg, and preferably from about 100 pg to about 1 μg. Suitable dose range will vary with the route of injection and the size of the patient, but will typically range from about 0.1 mL to about 5 mL.

In a specific embodiment, the viruses and/or vaccines of the invention are administered at a starting single dose of at least 10³ TCID₅₀, at least 10⁴ TCID₅₀, at least 10⁵ TCID₅₀, at least 10⁶ TCID₅₀. In another specific embodiment, the virus and/or vaccines of the invention are administered at multiple doses. In a preferred embodiment, a primary dosing regimen at 2, 4, and 6 months of age and a booster dose at the beginning of the second year of life are used. More preferably, each dose of at least 10⁵ TCID₅₀, or at least 10⁶ TCID₅₀ is given in a multiple dosing regimen.

### 5.13.1 Challenge Studies

This assay is used to determine the ability of the recombinant viruses of the invention and of the vaccines of the invention to prevent lower respiratory tract viral infection in an animal model system, such as, but not limited to, cotton rats or hamsters. The recombinant virus and/or the vaccine can be administered by intravenous (IV) route, by intramuscular (IM) route or by intranasal route (IN). The recombinant virus and/or the vaccine can be administered by any technique well-known to the skilled artisan. This assay is also used to correlate the serum concentration of antibodies with a reduction in lung titer of the virus to which the antibodies bind.

On day 0, groups of animals, such as, but not limited to, cotton rats (Sigmodon hispidis, average weight 100 g) cynomolgous macacques (average weight 2.0 kg) are administered the recombinant or chimeric virus or the vaccine of interest or BSA by intramuscular injection, by intravenous injection, or by intranasal route. Prior to, concurrently with, or subsequent to administration of the recombinant virus or the vaccine of the invention, the animals are infected with wild type virus wherein the wild type virus is the virus against which the vaccine was generated. In certain embodiments, the animals are infected with the wild type virus at least 1 day, at least 2 days, at least 3 days, at least 4 days, at least 5 days, at least 6 days, 1 week or 1 or more months subsequent to the administration of the recombinant virus and/or the vaccine of the invention.

After the infection, cotton rats are sacrificed, and their lung tissue is harvested and pulmonary virus titers are determined by plaque titration. Bovine serum albumin (BSA) 10 mg/kg is used as a negative control. Antibody concentrations in the serum at the time of challenge are determined using a sandwich ELISA. Similarly, in macacques, virus titers in nasal and lung lavages can be measured.

### 5.13.2 Target Populations

In certain embodiments of the invention, the target population for the therapeutic and diagnostic methods of the invention is defined by age. In certain embodiments, the target population for the therapeutic and/or diagnostic methods of the invention is characterized by a disease or disorder in addition to a respiratory tract infection.

In a specific embodiment, the target population encompasses young children, below 2 years of age. In a more specific embodiment, the children below the age of 2 years do not suffer from illnesses other than respiratory tract infection.

In other embodiments, the target population encompasses patients above 5 years of age. In a more specific embodiment, the patients above the age of 5 years suffer from an additional disease or disorder including cystic fibrosis, leukaemia, and non-Hodgkin lymphoma, or recently received bone marrow or kidney transplantation.

In a specific embodiment of the invention, the target population encompasses subjects in which the hMPV infection is associated with immunosuppression of the hosts. In a specific embodiment, the subject is an immunocompromised individual.

In certain embodiments, the target population for the methods of the invention encompasses the elderly.

In a specific embodiment, the subject to be treated or diagnosed with the methods of the invention was infected with hMPV in the winter months.

### 5.13.3 Clinical Trials

Vaccines of the invention or fragments thereof tested in in vitro assays and animal models may be further evaluated for safety, tolerance and pharmacokinetics in groups of normal healthy adult volunteers. The volunteers are administered intramuscularly, intravenously or by a pulmonary delivery system a single dose of a recombinant virus of the invention and/or a vaccine of the invention. Each volunteer is monitored at least 24 hours prior to receiving the single dose of the recombinant virus of the invention and/or a vaccine of the invention and each volunteer will be monitored for at least 48 hours after receiving the dose at a clinical site. Then volunteers are monitored as outpatients on days 3, 7, 14, 21, 28, 35, 42, 49, and 56 postdose.

Blood samples are collected via an indwelling catheter or direct venipuncture using 10 ml red-top Vacutainer tubes at the following intervals: (1) prior to administering the dose of the recombinant virus of the invention and/or a vaccine of the invention; (2) during the administration of the dose of the recombinant virus of the invention and/or a vaccine of the invention; (3) 5 minutes, 10 minutes, 15 minutes, 20 minutes, 30 minutes, 1 hour, 2 hours, 4 hours, 8 hours, 12 hours, 24 hours, and 48 hours after administering the dose of the recombinant virus of the invention and/or a vaccine of the invention; and (4) 3 days, 7 days 14 days, 21 days, 28 days, 35 days, 42 days, 49 days, and 56 days after administering the dose of the recombinant virus of the invention and/or a vaccine of the invention. Samples are allowed to clot at room temperature and serum will be collected after centrifugation.

The amount of antibodies generated against the recombinant virus of the invention and/or a vaccine of the invention in the samples from the patients can be quantitated by ELISA. T-cell immunity (cytotoxic and helper responses) in PBMC and lung and nasal lavages can also be monitored.

The concentration of antibody levels in the serum of volunteers are corrected by subtracting the predose serum level (background level) from the serum levels at each collection interval after administration of the dose of recombinant virus of the invention and/or a vaccine of the invention. For each volunteer the pharmacokinetic parameters are computed according to the model-independent approach (Gibaldi et al., eds., 1982, Pharmacokinetics, 2nd edition, Marcel Dekker, New York) from the corrected serum antibody or antibody fragment concentrations.

### 5.14 Methods for Detecting and Diagnosing Mammalian MPV

The invention provides means and methods for the diagnosis and/or detection of MPV, said means and methods to be employed in the detection of MPV, its components, and the products of its transcription, translation, expression, propagation, and metabolic processes. More specifically, this invention provides means and methods for the diagnosis of an MPV infection in animals and in humans, said means and methods including but not limited to the detection of components of MPV, products of the life cycle of MPV, and products of a host's response to MPV exposure or infection.

The methods that can be used to detect MPV or its components, and the products of its transcription, translation, expression, propagation and metabolic processes are well known in the art and include, but are not limited to, molecular based methods, antibody based methods, and cell-based methods. Examples of molecular based methods include, but are not limited to polymerase chain reaction (PCR), reverse transcriptase PCR (RT-PCR), real time RT-PCR, nucleic acid sequence based amplification (NASBA), oligonucleotide probing, southern blot hybridization, northern blot hybridization, any method that involves the contacting of a sample with a nucleic acid that is complementary to an MPV or similar or identical to an MPV, and any combination of these methods with each other or with those in the art. Identical or similar nucleic acids that can be used are described herein, and are also well known in the art to be able to allow one to distinguish between MPV and the genomic material or related products of other viruses and organisms. Examples of antibody based methods include, but are not limited to, the contacting of an antibody with a sample suspected of containing MPV, direct immunofluorescence (DIF), enzyme linked immunoabsorbent assay (ELISA), western blot, immunochromatography. Examples of cell-based methods include, but are not limited to, reporter assays that are able to emit a signal when exposed to MPV, its components, or products thereof. In another embodiment, the reporter assay is an in vitro assay, whereby the reporter is expressed upon exposure to MPV, its components, or products thereof. Examples of the aforementioned methods are well-known in the art and also described herein. In a more specific embodiment, NASBA is used to amplify specific RNA or DNA from a pool of total nucleic acids.

In one embodiment, the invention provides means and methods for the diagnosis and detection of MPV, said means and methods including but not limited to the detection of genomic material and other nucleic acids that are associated with or complimentary to MPV, the detection of transcriptional and translational products of MPV, said products being both processed and unprocessed, and the detection of components of a host response to MPV exposure or infection.

In one embodiment, the invention relates to the detection of MPV through the preparation and use of oligonucleotides that are complimentary to nucleic acid sequences and transcriptional products of nucleic acid sequences that are present within the genome of MPV. Furthermore, the invention relates to the detection of nucleic acids, or sequences thereof, that are present in the genome of MPV and its transcription products, using said oligonucleotides as primers for copying or amplification of specific regions of the MPV genome and its transcripts. The regions of the MPV genome and its transcripts that can be copied or amplified include but are not limited to complete and incomplete stretches of one or more of the following: the N-gene, the P-gene, the M-gene, the F-gene, the M2-gene, the SH-gene, the G-gene, and the L-gene. In a specific embodiment, oligonucleotides are used as primers in conjunction with methods to copy or amplify the N-gene of MPV, or transcripts thereof, for identification purposes. Said methods include but are not limited to, PCR assays, RT-PCR assays, real time RT-PCR assays, primer extension or run on assays, NASBA and other methods that employ the genetic material of MPV or transcripts and compliments thereof as templates for the extension of nucleic acid sequences from said oligonucleotides. In another embodiment, a combination of methods is used to detect the presence of MPV in a sample. One skilled in the art would be familiar with the requirements and applicability of each assay. For example, PCR and RT-PCR would be useful for the amplification or detection of a nucleic acid. In a more specific embodiment, real time RT-PCR is used for the routine and reliable quantitation of PCR products.

In another embodiment, the invention relates to detection of MPV through the preparation and use of oligonucleotides that are complimentary to nucleic acid sequences and transcriptional products of nucleic acid sequences that are present within the genome of MPV. Furthermore, the invention relates to the detection of nucleic acids, or sequences thereof, that are present in or complimentary to the genome of MPV and its transcription products, using said oligonucleotide sequences as probes for hybridization to and detection of specific regions within or complimentary to the MPV genome and its transcripts. The regions of the MPV genome and its transcripts that can be detected using hybridization probes include but are not limited to complete and incomplete stretches of one or more of the following: the N-gene, the P-gene, the M-gene, the F-gene, the M2-gene, the SH-gene, the G-gene, and the L-gene. In a specific embodiment, oligonucleotides are used as probes in conjunction with methods to detect, anneal, or hybridize to the N-gene of MPV, or transcripts thereof, for identification purposes. Said methods include but are not limited to, Northern blots, Southern blots and other methods that employ the genetic material of MPV or transcripts and compliments thereof as targets for the hybridization, annealing, or detection of sequences or stretches of sequences within or complimentary to the MPV genome.

A nucleic acid which is hybridizable to a nucleic acid of a mammalian MPV, or to its reverse complement, or to its complement can be used in the methods of the invention to detect the presence of a mammalian MPV. In certain embodiments, the nucleic acids are hybridized under conditions of high stringency. By way of example and not limitation, procedures using such conditions of high stringency are as follows. Prehybridization of filters containing DNA is carried out for 8 h to overnight at 65 C in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65 Cin prehybridization mixture containing 100 μg/ml denatured salmon sperm DNA and 5-20×106 cpm of 32P-labeled probe. Washing of filters is done at 37 C for 1 h in a solution containing 2×SSC, 0.01% PVP, 0.01% Ficoll, and 0.01% BSA. This is followed by a wash in 0.1×SSC at 50 C for 45 min before autoradiography. Other conditions of high stringency which may be used are well known in the art. In other embodiments of the invention, hybridization is performed under moderate of low stringency conditions, such conditions are well-known to the skilled artisan (see e.g., Sambrook et al., 1989, Molecular Cloning, A Laboratory Manual, 2d Ed., Cold Spring Harbor Laboratory Press, Cold Spring Harbor, N.Y.; see also, Ausubel et al., eds., in the Current Protocols in Molecular Biology series of laboratory technique manuals, 1987-1997 Current Protocols, ©1994-1997 John Wiley and Sons, Inc.).

Any size oligonucleotides can be used in the methods of the invention. As described herein, such oligonucleotides are useful in a variety of methods, e.g., as primer or probes in various detection or analysis procedures. In preferred embodiments, oligonucleotide probes and primers are at least 5, at least 8, at least 10, at least 12, at least 15, at least 20, at least 25, at least 30, at least 35, at least 40, at least 45, at least 50, at least 55, at least 60, at least 70, at least 80, at least 100, at least 200, at least 300 at least 400, at least 500, at least 1000, at least 2000, at least 3000, at least 4000 or at least 5000 bases. In another more certain embodiments, oligonucleotide probes and primers comprise at least 5, at least 8, at least 10, at least 12, at least 15, at least 20, at least 25, at least 30, at least 35, at least 40, at least 45, at least 50, at least 55, at least 60, at least 70, at least 80, at least 100, at least 200, at least 300 at least 400, at least 500, at least 1000, at least 2000, at least 3000, at least 4000 or at least 5000 bases, that are at least 50%, at least 60%, at least 70%, at least 80%, at least 90%, at least 99%, at least 99.5% homologous to a target sequence, such as an MPV genomic sequence or complement thereof. In a another specific embodiment, the oligonucleotide that is used as a primer or a probe is of any length, and specifically hybridizes under stringent conditions through at least 8 of its most 3′ terminal bases to a target sequence. In another specific embodiment, the oligonucleotide that is used as a primer or a probe is of any length, and specifically hybridizes under stringent conditions through at least 10 of its most 3′ terminal bases to a target sequence. In another specific embodiment, the oligonucleotide that is used as a primer or a probe is of any length, and specifically hybridizes under stringent conditions through at least 12 of its most 3′ terminal bases to a target sequence. In another specific embodiment, the oligonucleotide that is used as a primer or a probe is of any length, and specifically hybridizes under stringent conditions through at least 15 of its most 3′ terminal bases to a target sequence. In another specific embodiment, the oligonucleotide that is used as a primer or a probe is of any length, and specifically hybridizes under stringent conditions through at least 20 of its most 3′ terminal bases to a target sequence. In another specific embodiment, the oligonucleotide that is used as a primer or a probe is of any length, and specifically hybridizes under stringent conditions through at least 25 of its most 3′ terminal bases to a target sequence. In another embodiment, a degenerate set of oligos is used so that a specific position or nucleotide is substituted. The degeneracy can occur at any position or at any number of positions, most preferably, at least at one position, but also at least at two positions, at least at three positions, at least ten positions, in the region that hybridizes under stringent conditions to the target sequence.

One skilled in the art would be familiar with the structural requirements imposed upon oligonucleotides by the assays known in the art. It is also possible to design oligonucleotide primers and probes using more systematic approaches. For example, one skilled in the art would be able to determine the appropriate length and sequence of an oligonucleotide primer or probe based upon preferred assay or annealing temperatures and the structure of the oligo, i.e., sequence. In addition, one skilled in the art would be able to determine the specificity of the assay employing an oligonucleotide primer or probe, by adjusting the temperature of the assay so that the specificity of the oligo for the target sequence is enhanced or diminished, depending upon the temperature. In a preferred embodiment, the annealing temperature of the primer or probe is determined, using methods known in the art, and the assay is performed at said annealing temperature. One skilled in the art would be familiar with methods to calculate the annealing temperature associated with an oligonucleotide for its specific target sequence. For example, annealing temperatures can be roughly calculated by, assigning 4° C. to the annealing temperature for each G or C nucleotide in the oligonucleotide that hybridizes to the target sequence. In another example, annealing temperatures can be roughly calculated by, assigning 2° C. to the annealing temperature for each A or T nucleotide in the oligonucleotide that hybridizes to the target sequence. The annealing temperature of the oligonucleotide is necessarily dependent upon the length and sequence of the oligonucleotide, as well as upon the complimentarity of the oligo for the target sequence, so that only binding events between the oligo primer or probe are factored into the annealing temperature. The examples described herein for the calculation of annealing temperature are meant to be examples and are not meant to limit the invention from other methods of determination for the annealing temperature. One skilled in the art would be familiar with other methods that can be used, and in addition, other more sophisticated methods of calculating annealing or melting temperatures for an oligonucleotide have been described herein. In a more specific embodiment, oligonucleotide probes and primers are annealed at a temperature of at least 30° C., at least 35° C., at least 40° C., at least 45° C., at least 50° C., at least 55° C., at least 60° C., at least 65° C., at least 70° C., at least 80° C., at least 90° C. or at least 99° C.

The invention provides cell-based and cell-free assays for the identification or detection of MPV in a sample. A variety of methods can be used to conduct the cell-based and cell-free assays of the invention, including but not limited to, those using reporters. Examples of reporters are described herein and can be used for the identification or detection of MPV using high-throughput screening and for any other purpose that would be familiar to one skilled in the art. There are a number of methods that can be used in the reporter assays of the invention. For example, the cell-based assays may be conducted by contacting a sample with a cell containing a nucleic acid sequence comprising a reporter gene, wherein the reporter gene is linked to the promoter of an MPV gene or linked to a promoter that is recognized by an MPV gene product, and measuring the expression of the reporter gene, upon exposure to MPV or a component of MPV. In a further embodiment of the cell-based assay, a host cell that is able to be infected by MPV, is transfected with a nucleic acid construct that encodes one or more reporter genes, such that expression from the reporter gene occurs in the presence of an MPV or an MPV component. In such an embodiment, expression of the reporter gene is operably linked to a nucleic acid sequence that is recognized by MPV or a component thereof, thereby causing expression of the reporter gene. The presence of MPV in the sample induces expression of the reporter gene that can be detected using any method known in the art, and also described herein (section 5.8.2). Examples of host cells that can be transfected and used in the cell-based detection assay, include, but are not limited to, Vero, tMK, COS7 cells. In another embodiment, the host cell is any cell that can be infected with MPV. The expression of the reporter gene is thereby indicative of the presence of an MPV or a component thereof. In a cell-free assay, a sample is contacted with a nucleic acid comprising a reporter gene that is operably linked to a nucleic acid sequence so that the presence of an MPV or a component thereof induces expression of the reporter gene in vitro. For example, the cell-free assay may be conducted by contacting a sample suspected of containing an MPV or a component thereof, with a nucleic acid that comprises a reporter gene, wherein the reporter gene is linked to the promoter of an MPV gene or linked to a promoter that is recognized by an MPV gene product, and measuring the expression of the reporter gene, upon exposure to MPV or a component of MPV. The expression of the reporter gene is thereby indicative of the presence of an MPV or a component thereof. While a large number of reporter compounds are known in the art, a variety of examples are provided herein (see, e.g., section 5.8.2).

In another embodiment, the invention relates to the detection of MPV infection using a minireplicon system. For example, a host cell can be transfected with an hMPV minireplicon construct that encodes one or more reporter genes, such that expression from the reporter gene occurs in the presence of hMPV or hMPV polymerase. Examples of reporter genes are described herein, in section 5.8.2. In such an embodiment, hMPV acts as a helper virus to promote the expression of the reporter gene or genes encoded by the minireplicon system. Without being bound by limitation, hMPV provides polymerase that drives rescue of the minireplicon system and therefore drives expression of the reporter gene or genes. In a certain embodiment, a host cell, that has been transfected with an hMPV minireplicon, encoding a reporter gene, is contacted with a sample suspected to contain hMPV. The presence of hMPV in the sample induces expression of the reporter gene that can be detected using any method known in the art, and also described herein (section 5.8.2). Examples of the host cell, include, but are not limited to, Vero, tMK, COS7 cells. In another embodiment, the host cell is any cell that can be infected with hMPV.

In another embodiment, the invention relates to the detection of an MPV infection in an animal or human host through the preparation and use of antibodies, e.g., monoclonal antibodies (MAbs), that are specific to and can recognize peptides or nucleic acids that are characteristic of MPV or its gene products. The epitopes or antigenic determinants recognized by said MAbs include but are not limited to proteinaceous and nucleic acid products that are synthesized during the life cycle and metabolic processes involved in MPV propagation. The proteinaceous or nucleic acid products that can be used as antigenic determinants for the generation of suitable antibodies include but are not limited to complete and incomplete transcription and expression products of one or more of the following components of MPV: the N-gene, the P-gene, the M-gene, the F-gene, the M2-gene, the SH-gene, the G-gene, and the L-gene. In one specific embodiment, MAbs raised against proteinaceous products of the G-gene or portions thereof are used in conjunction with other methods to detect or confirm the presence of the MPV expressed G peptide in a biological sample, e.g. body fluid. Said methods include but are not limited to ELISA, Radio-Immuno or Competition Assays, Immuno-precipitation and other methods that employ the transcribed or expressed gene products of MPV as targets for detection by MAbs raised against said targets or portions and relatives thereof. In another embodiment of the invention, the antibodies that can be used to detect hMPV, recognize the F, G, N, L, M, M2-1, P, and SH proteins of all four subtypes.

In another embodiment, the invention relates to the detection of factors that are associated with and characteristic of a host's immunologic response to MPV exposure or infection. Upon exposure or infection by MPV, a host's immune system illicits a response to said exposure or infection that involves the generation by the host of antibodies directed at eliminating or attenuating the effects and/or propagation of virus. This invention provides means and methods for the diagnosis of MPV related disease through the detection of said antibodies that may be produced as a result of MPV exposure to or infection of the host. The epitopes recognized by said antibodies include but are not limited to peptides and their exposed surfaces that are accessible to a host immune response and that can serve as antigenic determinants in the generation of an immune response by the host to the virus. Some of the proteinaceous and nuclear material used by a host immune response as epitopes for the generation of antibodies include but are not limited to products of one or more of the following components of MPV: the N-gene, the P-gene, the M-gene, the F-gene, the M2-gene, the SH-gene, the G-gene, and the L-gene. In one embodiment, antibodies to partially or completely accessible portions of the N-gene encoded peptides of MPV are detected in a host sample. In a specific embodiment, proteinaceous products of the G-gene or portions thereof are used in conjunction with other methods to detect the presence of the host derived antibodies in a biological sample, e.g. body fluid. Said methods include but are not limited to ELISA, Radio-Immuno or Competition Assays, and other methods that employ the transcribed or expressed gene products of MPV as targets for detection by host antibodies that recognize said products and that are found in biological samples.

This invention also provides means and methods for diagnostic assays or test kits and for methods to detect agents of an MPV infection from a variety of sources including but not limited to biological samples, e.g., body fluids. In one embodiment, this invention relates to assays, kits, protocols, and procedures that are suitable for identifying an MPV nucleic acid or a compliment thereof. In another embodiment, this invention relates to assays, kits, protocols, and procedures that are suitable for identifying an MPV expressed peptide or a portion thereof. In another embodiment, this invention relates to assays, kits, protocols, and procedures that are suitable for identifying components of a host immunologic response to MPV exposure or infection.

In addition to diagnostic confirmation of MPV infection of a host, the present invention also provides for means and methods to classify isolates of MPV into distinct phylogenetic groups or subgroups. In one embodiment, this feature can be used advantageously to distinguish between the different variant of MPV, variant A1, A2, B1 and B2, in order to design more effective and subgroup specific therapies. Variants of MPV can be differentiated on the basis of nucleotide or amino acid sequences of one or more of the following: the N-gene, the P-gene, the M-gene, the F-gene, the M2-gene, the SH-gene, the G-gene, and the L-gene. In a specific embodiment, MPV can be differentiated into a specific subgroup using the nucleotide or amino acid sequence of the G gene or glycoprotein and neutralization tests using monoclonal antibodies that also recognize the G glycoprotein.

In one embodiment, the diagnosis of an MPV infection in a human is made using any technique well known to one skilled in the art, e.g., immunoassays. Immunoassays which can be used to analyze immunospecific binding and cross-reactivity include, but are not limited to, competitive and non-competitive assay systems using techniques such as western blots, radioimmunoassays, ELISA (enzyme linked immunosorbent assay), sandwich immunoassays, immunoprecipitation assays, precipitin reactions, gel diffusion precipitation reactions, immunodiffusion assays, agglutination assays, complement-fixation assays, and fluorescent immunoassays, to name but a few. Such assays are routine and well known in the art (see, e.g., Ausubel et al., eds, 1994, Current Protocols in Molecular Biology, Vol. 1, John Wiley & Sons, Inc., New York, which is incorporated by reference herein in its entirety) and non-limiting examples of immunoassays are described in section 5.8.

In one embodiment, the invention relates to the detection of an MPV infection using oligonucleotides in conjunction with PCR or primer extension methods to copy or amplify regions of the MPV genome, said regions including but not limited to genes or parts of genes, e.g., the N, M, F, G, L, M, P, and M2 genes. In a specific embodiment, oligonucleotides are used in conjunction with RT-PCR methods. In a further embodiment, the amplification products and/or genetic material can be probed with oligonucleotides that are complimentary to specific sequences that are either conserved between various hMPV strains or are distinct amongst various hMPV strains. The latter set of oligonucletides would allow for identification of the specific strain of hMPV responsible for the infection of the host.

The invention provides methods for distinguishing between different subgroups and variants of hMPV that are capable of infecting a host. In one specific embodiment, the hMPV that is responsible for a host infection is classified into a specific subgroup, e.g., subgroup A or subgroup B. In another specific embodiment, the hMPV that is responsible for a host infection is classified as a specific variant of a subgroup, e.g., variant A1, A2, B1, or B2. In another embodiment, the invention provides means and methods for the classification of an hMPV that is responsible for a host infection into a new subgroup and/or into a new variant of a new or existing subgroup. The methods that are able to distinguish hMPV strains into subgroups and/or variant groups would be known to one skilled in the art. In one embodiment, a polyclonal antibody is used to identify the etiological agent of an infection as a strain of hMPV, and a secondary antibody is used to distinguish said strain as characteristic of a new or known subgroup and/or new or known variant of hMPV. In one embodiment, antibodies that are selective for hMPV are used in conjunction with immunoreactive assays, e.g. ELISA or RIA, to identify the presence of hMPV exposure or infection in biological samples. In a further embodiment, secondary antibodies that are selective for specific epitopes in the peptide sequence of hMPV proteins are used to further classify the etiological agents of said identified hMPV infections into subgroups or variants. In one specific embodiment, an antibody raised against peptide epitopes that are shared between all subgroups of hMPV is used to identify the etioligical agent of an infection as an hMPV. In a further specific embodiment, antibodies raised against peptide epitopes that are unique to the different subgroups and/or variants of hMPV are used to classify the hMPV that is responsible for the host infection into a known or new subgroup and/or variant. In one specific embodiment, the antibody that is capable of distinguishing between different subgroups and/or variants of hMPV recognizes segments of hMPV peptides that are unique to the subgroup or variant, said peptides including but not limited to those encoded by the N, M, F, G, L, M, P, and M2 genes. The peptides or segments of peptides that can be used to generate antibodies capable of distinguishing between different hMPV sugroups or variants can be selected using differences in known peptide sequences of various hMPV proteins in conjunction with hydrophillicity plots to identify suitable peptide segments that would be expected to be solvent exposed or accessible in a diagnostic assay. In one embodiment, the antibody that is capable of distinguishing between the different subgroups of hMPV recognizes differences in the F protein that are unique to different subgroups of hMPV, e.g. the amino acids at positions 286, 296, 312, 348, and 404 of the full length F protein. In another specific embodiment, the antibody that is capable of distinguishing between different subgroups and/or variants of hMPV recognizes segments of the G protein of hMPV that are unique to specific subgroups or variants, e.g., the G peptide sequence corresponding to amino acids 50 through 60 of SEQ ID:119 can be used to distinguish between subgroups A and B as well as between variants A1, A2, B1, and B2. In another embodiment of the invention, the nucleotide sequence of hMPV isolates are used to distinguish between different subgroups and/or different variants of hMPV. In one embodiment, oligonucleotide sequences, primers, and/or probes that are complimentary to sequences in the hMPV genome are used to classify the etiological agents of hMPV infections into distinct subgroups and/or variants in conjunction with methods known to one skilled in the art, e.g. RT-PCR, PCR, primer run on assays, and various blotting techniques. In one specific embodiment, a biological sample is used to copy or amplify a specific segment of the hMPV genome, using RT-PCR. In a further embodiment, the sequence of said segment is obtained and compared with known sequences of hMPV, and said comparison is used to classify the hMPV strain into a distinct subgroup or variant or to classify the hMPV strain into a new subgroup or variant. In another embodiment, the invention relates to diagnostic kits that can be used to distinguish between different subgroups and/or variants of hMPV.

In a preferred embodiment, diagnosis and/or treatment of a specific viral infection is performed with reagents that are most specific for said specific virus causing said infection. In this case this means that it is preferred that said diagnosis and/or treatment of an MPV infection is performed with reagents that are most specific for MPV. This by no means however excludes the possibility that less specific, but sufficiently crossreactive reagents are used instead, for example because they are more easily available and sufficiently address the task at hand. Herein it is for example provided to perform virological and/or serological diagnosis of MPV infections in mammals with reagents derived from APV, in particular with reagents derived from APV-C, in the detailed description herein it is for example shown that sufficiently trustworthy serological diagnosis of MPV infections in mammals can be achieved by using an ELISA specifically designed to detect APV antibodies in birds. A particular useful test for this purpose is an ELISA test designed for the detection of APV antibodies (e.g in serum or egg yolk), one commercially available version of which is known as APV-Ab SVANOVIR® which is manufactured by SVANOVA Biotech AB, Uppsal Science Park Glunten SE-751 83 Uppsala Sweden. The reverse situation is also the case, herein it is for example provided to perform virological and/or serological diagnosis of APV infections in mammals with reagents derived from MPV, in the detailed description herein it is for example shown that sufficiently trustworthy serological diagnosis of APV infections in birds can be achieved by using an ELISA designed to detect MPV antibodies. Considering that antigens and antibodies have a lock-and-key relationship, detection of the various antigens can be achieved by selecting the appropriate antibody having sufficient cross-reactivity. Of course, for relying on such cross-reactivity, it is best to select the reagents (such as antigens or antibodies) under guidance of the amino acid homologies that exist between the various (glyco)proteins of the various viruses, whereby reagents relating to the most homologous proteins will be most useful to be used in tests relying on said cross-reactivity.

For nucleic acid detection, it is even more straightforward, instead of designing primers or probes based on heterologous nucleic acid sequences of the various viruses and thus that detect differences between the essentially mammalian or avian Metapneumoviruses, it suffices to design or select primers or probes based on those stretches of virus-specific nucleic acid sequences that show high homology. In general, for nucleic acid sequences, homology percentages of 90% or higher guarantee sufficient cross-reactivity to be relied upon in diagnostic tests utilizing stringent conditions of hybridisation.

The invention for example provides a method for virologically diagnosing a MPV infection of an animal, in particular of a mammal, more in particular of a human being, comprising determining in a sample of said animal the presence of a viral isolate or component thereof by reacting said sample with a MPV specific nucleic acid a or antibody according to the invention, and a method for serologically diagnosing an MPV infection of a mammal comprising determining in a sample of said mammal the presence of an antibody specifically directed against an MPV or component thereof by reacting said sample with a MPV-specific proteinaceous molecule or fragment thereof or an antigen according to the invention. The invention also provides a diagnostic kit for diagnosing an MPV infection comprising an MPV, an MPV-specific nucleic acid, proteinaceous molecule or fragment thereof, antigen and/or an antibody according to the invention, and preferably a means for detecting said MPV, MPV-specific nucleic acid, proteinaceous molecule or fragment thereof, antigen and/or an antibody, said means for example comprising an excitable group such as a fluorophore or enzymatic detection system used in the art (examples of suitable diagnostic kit format comprise IF, ELISA, neutralization assay, RT-PCR assay). To determine whether an as yet unidentified virus component or synthetic analogue thereof such as nucleic acid, proteinaceous molecule or fragment thereof can be identified as MPV-specific, it suffices to analyse the nucleic acid or amino acid sequence of said component, for example for a stretch of said nucleic acid or amino acid, preferably of at least 10, more preferably at least 25, more preferably at least 40 nucleotides or amino acids (respectively), by sequence homology comparison with known MPV sequences and with known non-MPV sequences APV-C is preferably used) using for example phylogenetic analyses as provided herein. Depending on the degree of relationship with said MPV or non-MPV sequences, the component or synthetic analogue can be identified.

The invention also provides method for virologically diagnosing an MPV infection of a mammal comprising determining in a sample of said mammal the presence of a viral isolate or component thereof by reacting said sample with a cross-reactive nucleic acid derived from APV (preferably serotype C) or a cross-reactive antibody reactive with said APV, and a method for serologically diagnosing an MPV infection of a mammal comprising determining in a sample of said mammal the presence of a cross-reactive antibody that is also directed against an APV or component thereof by reacting said sample with a proteinaceous molecule or fragment thereof or an antigen derived from APV. Furthermore, the invention provides the use of a diagnostic kit initially designed for AVP or AVP-antibody detection for diagnosing an MPV infection, in particular for detecting said MPV infection in humans.

The invention also provides methods for virologically diagnosing an APV infection in a bird comprising determining in a sample of said bird the presence of a viral isolate or component thereof by reacting said sample with a cross-reactive nucleic acid derived from MPV or a cross-reactive antibody reactive with said MPV, and a method for serologically diagnosing an APV infection of a bird comprising determining in a sample of said bird the presence of a cross-reactive antibody that is also directed against an MPV or component thereof by reacting said sample with a proteinaceous molecule or fragment thereof or an antigen derived from MPV. Furthermore, the invention provides the use of a diagnostic kit initially designed for MPV or MPV-antibody detection for diagnosing an APV infection, in particular for detecting said APV infection in poultry such as a chicken, duck or turkey.

For diagnosis as for treatment, use can be made of the high degree of homology among different mammalian MPVs and between MPV and other viruses, such as, e.g., APV, in particular when circumstances at hand make the use of the more homologous approach less straightforward. Vaccinations that can not wait, such as emergency vaccinations against MPV infections can for example be performed with vaccine preparations derived from APV(preferably type C) isolates when a more homologous MPV vaccine is not available, and, vice versa, vaccinations against APV infections can be contemplated with vaccine preparations derived from MPV. Also, reverse genetic techniques make it possible to generate chimeric APV-MPV virus constructs that are useful as a vaccine, being sufficiently dissimilar to field isolates of each of the respective strains to be attenuated to a desirable level. Similar reverse genetic techniques will make it also possible to generate chimeric paramyxovirus-metapneumovirus constructs, such as RSV-MPV or P13-MPV constructs for us in a vaccine preparation. Such constructs are particularly useful as a combination vaccine to combat respiratory tract illnesses.

Since MPV CPE was virtually indistinguishable from that caused by hRSV or hPIV-1 in tMK or other cell cultures, the MPV may have well gone unnoticed until now. tMK (tertiary monkey kidney cells, i.e. MK cells in a third passage in cell culture) are preferably used due to their lower costs in comparison to primary or secondary cultures. The CPE is, as well as with some of the classical Paramyxoviridae, characterized by syncytium formation after which the cells showed rapid internal disruption, followed by detachment of the cells from the monolayer. The cells usually (but not always) displayed CPE after three passages of virus from original material, at day 10 to 14 post inoculation, somewhat later than CPE caused by other viruses such as hRSV or hPIV-1.

As an example, the invention provides a not previously identified paramyxovirus from nasopharyngeal aspirate samples taken from 28 children suffering from severe RTI. The clinical symptoms of these children were largely similar to those caused by hRSV. Twenty-seven of the patients were children below the age of five years and half of these were between 1 and 12 months old. The other patient was 18 years old. All individuals suffered from upper RTI, with symptoms ranging from cough, myalgia, vomiting and fever to broncheolitis and severe pneumonia. The majority of these patients were hospitalised for one to two weeks.

The virus isolates from these patients had the paramyxovirus morphology in negative contrast electron microscopy but did not react with specific antisera against known human and animal paramyxoviruses. They were all closely related to one another as determined by indirect immunofluorescence assays (WA) with sera raised against two of the isolates. Sequence analyses of nine of these isolates revealed that the virus is somewhat related to APV. Based on virological data, sequence homology as well as the genomic organisation we propose that the virus is a member of Metapneumovirus genus. Serological surveys showed that this virus is a relatively common pathogen since the seroprevalence in the Netherlands approaches 100% of humans by the age of five years. Moreover, the seroprevalence was found to be equally high in sera collected from humans in 1958, indicating this virus has been circulating in the human population for more than 40 years. The identification of this proposed new member of the Metapneumovirus genus now also provides for the development of means and methods for diagnostic assays or test kits and vaccines or serum or antibody compositions for viral respiratory tract infections, and for methods to test or screen for antiviral agents useful in the treatment of MPV infections.

Methods and means provided herein are particularly useful in a diagnostic kit for diagnosing a MPV infection, be it by virological or serological diagnosis. Such kits or assays may for example comprise a virus, a nucleic acid, a proteinaceous molecule or fragment thereof, an antigen and/or an antibody according to the invention. Use of a virus, a nucleic acid, a proteinaceous molecule or fragment thereof, an antigen and/or an antibody according to the invention is also provided for the production of a pharmaceutical composition, for example for the treatment or prevention of MPV infections and/or for the treatment or prevention of respiratory tract illnesses, in particular in humans. Attenuation of the virus can be achieved by established methods developed for this purpose, including but not limited to the use of related viruses of other species, serial passages through laboratory animals or/and tissue/cell cultures, site directed mutagenesis of molecular clones and exchange of genes or gene fragments between related viruses.

Four distinct subtypes of hMPV have been described, referred to as subtypes A1, A2, B1 and B2. The invention relates to the detection of hMPV in a host using a single assay that is sensitive for all four subtypes. Any method known in the art can be used to detect the presence of hMPV in a host. In a more specific embodiment of the invention, a sensitive Taqman assay is used to detect the presence of hMPV in a host. One skilled in the art would be familiar with the requirements for the design of olignoucleotides and probes for use in such assays. Such oligonucleotides and probes can be designed to specifically recognize any region of the hMPV genome, transcripts or processed and unprocessed products thereof. In a more specific embodiment of the invention, the oligonucleotides and probes of the invention are complementary to or identical to, or similar to a sequence in all subtypes of hMPV, its transcripts, or processed and unprocessed products thereof, e.g., A1, B1, A2, and B2. In particular, the oligonucleotides and probes are at least 50%, 60%, 70%, 80%, 90%, 95%, 98%, 99%, or 99.5% identical to a negative or positive copy of the sequence in all four subtypes of hMPV, a transcript or processed and unprocessed products thereof. In another embodiment, it is complimentary to the negative or positive copy of the sequence in all four subtypes of hMPV. Any length oligonucleotides and probes can be used in the detection of assay of invention. Typical hybridization and washing conditions that may be used are known in the art. Preferably, the conditions are such as to enable the probe to bind specifically and to prevent the binding or easy removal of nonspecific binding. In yet another more specific embodiment of the invention, the oligonucleotides and probes of the invention are complementary to any of the open reading frames within the hMPV genome, including, but not limited to, the N-gene, P-gene, F-gene, M-gene, M2-gene, SH-gene, G-gene, and L-gene, or processed and unprocessed products thereof. In an even more specific embodiment of the invention, the oligonucleotides and probes of the invention recognize the N-gene, its transcripts, or processed and unprocessed products thereof. In yet another embodiment hMPV from all four subtypes are recognized with equal specificity.

Virus can be isolated from any biological sample obtainable from a host. In a more specific embodiment of the invention, nasopharyngeal samples are collected from a host for use in the detection assays of the invention. Virus can be propagated for detection purposes in a variety of cell lines that are able to support hMPV, including, but not limited to, Vero and tMK cells. The detection of viral RNA can be performed using a number of methods known to the skilled artisan. In one specific embodiment, viral RNA detection is performed using a Taqman PCR based method.

### 5.15 Compositions of the Invention and Components of Mammalian Metapneumovirus

The invention relates to nucleic acid sequences of a mammalian MPV, proteins of a mammalian MPV, and antibodies against proteins of a mammalian MPV. The invention further relates to homologs of nucleic acid sequences of a mammalian MPV and homologs of proteins of a mammalian MPV. The invention further relates to nucleic acid sequences encoding fusion proteins, wherein the fusion protein contains a protein of a mammalian MPV or a fragment thereof and one or more peptides or proteins that are not derived from mammalian MPV. In a specific embodiment, a fusion protein of the invention contains a protein of a mammalian MPV or a fragment thereof and a peptide tag, such as, but not limited to a polyhistidine tag. The invention further relates to fusion proteins, wherein the fusion protein contains a protein of a mammalian MPV or a fragment thereof and one or more peptides or proteins that are not derived from mammalian MPV. The invention also relates to derivatives of nucleic acids encoding a protein of a mammlian MPV. The invention also relates to derivatives of proteins of a mammalian MPV. A derivative can be, but is not limited to, mutant forms of the protein, such as, but not limited to, additions, deletions, truncations, substitutions, and inversions. A derivative can further be a chimeric form of the protein of the mammalian MPV, wherein at least one domain of the protein is derived from a different protein. A derivative can also be a form of a protein of a mammalian MPV that is covalently or non-covalently linked to another molecule, such as, e.g. , a drug.

The viral isolate termed NL/1/00 (also 00-1) is a mammalian MPV of variant A1 and its genomic sequence is shown in SEQ ID NO:19. The viral isolate termed NL/17/00 is a mammalian MPV of variant A2 and its genomic sequence is shown in SEQ ID NO:20. The viral isolate termed NL/1/99 (also 99-1) is a mammalian MPV of variant B1 and its genomic sequence is shown in SEQ ID NO:18. The viral isolate termed NL/1/94 is a mammalian MPV of variant B2 and its genomic sequence is shown in SEQ ID NO:21. A list of sequences disclosed in the present application and the corresponding SEQ ID Nos is set forth in Table 14.

The protein of a mammalian MPV can be a an N protein, a P protein, a M protein, a F protein, a M2-1 protein or a M2-2 protein or a fragment thereof. A fragment of a protein of a mammlian MPV can be can be at least 25 amino acids, at least 50 amino acids, at least 75 amino acids, at least 100 amino acids, at least 125 amino acids, at least 150 amino acids, at least 175 amino acids, at least 200 amino acids, at least 225 amino acids, at least 250 amino acids, at least 275 amino acids, at least 300 amino acids, at least 325 amino acids, at least 350 amino acids, at least 375 amino acids, at least 400 amino acids, at least 425 amino acids, at least 450 amino acids, at least 475 amino acids, at least 500 amino acids, at least 750 amino acids, at least 1000 amino acids, at least 1250 amino acids, at least 1500 amino acids, at least 1750 amino acids, at least 2000 amino acids or at least 2250 amino acids in length. A fragment of a protein of a mammlian MPV can be can be at most 25 amino acids, at most 50 amino acids, at most 75 amino acids, at most 100 amino acids, at most 125 amino acids, at most 150 amino acids, at most 175 amino acids, at most 200 amino acids, at most 225 amino acids, at most 250 amino acids, at most 275 amino acids, at most 300 amino acids, at most 325 amino acids, at most 350 amino acids, at most 375 amino acids, at most 400 amino acids, at most 425 amino acids, at most 450 amino acids, at most 475 amino acids, at most 500 amino acids, at most 750 amino acids, at most 1000 amino acids, at most 1250 amino acids, at most 1500 amino acids, at most 1750 amino acids, at most 2000 amino acids or at most 2250 amino acids in length.

In certain embodiments of the invention, the protein of a mammalian MPV is a N protein, wherein the N protein is phylogenetically closer related to a N protein of a mammalian MPV, such as the N protein encoded by, e.g., the viral genome of SEQ ID NO:18, SEQ ID NO:19, SEQ ID NO:20, or SEQ ID NO:21, (see also Table 14 for a description of the SEQ ID Nos) than it is related to the N protein of APV type C. In certain embodiments of the invention, the protein of a mammalian MPV is a P protein, wherein the P protein is phylogenetically closer related to a P protein of a mammalian MPV, such as the P protein encoded by, e.g., the viral genome of SEQ ID NO:18, SEQ ID NO:19, SEQ ID NO:20, or SEQ ID NO:21, than it is related to the N protein of APV type C. In certain embodiments of the invention, the protein of a mammalian MPV is a M protein, wherein the M protein is closer related to a M protein of a mammalian MPV, such as the M protein encoded by, e.g., the viral genome of SEQ ID NO:18, SEQ ID NO:19, SEQ ID NO:20, or SEQ ID NO:21, than it is related to the M protein of APV type C. In certain embodiments of the invention, the protein of a mammalian MPV is a F protein, wherein the F protein is phylogenetically closer related to a F protein of a mammalian MPV, such as the F protein encoded by, e.g., the viral genome of SEQ ID NO:18, SEQ ID NO:19, SEQ ID NO:20, or SEQ ID NO:21, than it is related to the F protein of APV type C. In certain embodiments of the invention, the protein of a mammalian MPV is a M2-1 protein, wherein the M2-1 protein is phylogenetically closer related to a M2-1 protein of a mammalian MPV, such as the M2-1 protein encoded by, e.g., the viral genome of SEQ ID NO:18, SEQ ID NO:19, SEQ ID NO:20, or SEQ ID NO:21, than it is related to the M2-1 protein of APV type C. In certain embodiments of the invention, the protein of a mammalian MPV is a M2-2 protein, wherein the M2-2 protein is phylogenetically closer related to a M2-2 protein of a mammalian MPV, such as the M2-2 protein encoded by, e.g., the viral genome of SEQ ID NO:18, SEQ ID NO:19, SEQ ID NO:20, or SEQ ID NO:21, than it is related to the M2-2 protein of APV type C. In certain embodiments of the invention, the protein of a mammalian MPV is a G protein, wherein the G protein is phylogenetically closer related to a G protein of a mammalian MPV, such as the G protein encoded by, e.g., the viral genome of SEQ ID NO:18, SEQ ID NO:19, SEQ ID NO:20, or SEQ ID NO:21, than it is related to any protein of APV type C. In certain embodiments of the invention, the protein of a mammalian MPV is a SH protein, wherein the SH protein is phylogenetically closer related to a SH protein of a mammalian MPV, such as the SH protein encoded by, e.g., the viral genome of SEQ ID NO:18, SEQ ID NO:19, SEQ ID NO:20, or SEQ ID NO:21, than it is related to any protein of APV type C. In certain embodiments of the invention, the protein of a mammalian MPV is a L protein, wherein the L protein is phylogenetically closer related to a L protein of a mammalian MPV, such as the SH protein encoded by, e.g., the viral genome of SEQ ID NO:18, SEQ ID NO:19, SEQ ID NO:20, or SEQ ID NO:21, than it is related to any protein of APV type C.

In certain embodiments of the invention, the protein of a mammalian MPV is a N protein, wherein the N protein is at least 60%, at least 65%, at least 70%, at least 75%, at least 80%, at least 85%, at least 90%, at least 95%, at least 98%, at least 99%, or at least 99.5% identical to the amino acid sequence of a N protein encoded by the viral genome of SEQ ID NO:18, SEQ ID NO:19, SEQ ID NO:20, or SEQ ID NO:21 (the amino acid sequences of the respective N proteins are disclosed in SEQ ID NO:366-369; see also Table 14). In certain embodiments of the invention, the protein of a mammalian MPV is a N protein, wherein the P protein is at least 60%, at least 65%, at least 70%, at least 75%, at least 80%, at least 85%, at least 90%, at least 95%, at least 98%, at least 99%, or at least 99.5% identical to the amino acid sequence of a P protein encoded by the viral genome of SEQ ID NO:18, SEQ ID NO:19, SEQ ID NO:20, or SEQ ID NO:21 (the amino acid sequences of the respective P proteins are disclosed in SEQ ID NO:374-377; see also Table 14). In certain embodiments of the invention, the protein of a mammalian MPV is a M protein, wherein the M protein is at least 60%, at least 65%, at least 70%, at least 75%, at least 80%, at least 85%, at least 90%, at least 95%, at least 98%, at least 99%, or at least 99.5% identical to the amino acid sequence of a M protein encoded by the viral genome of SEQ ID NO:18, SEQ ID NO:19, SEQ ID NO:20, or SEQ ID NO:21 (the amino acid sequences of the respective M proteins are disclosed in SEQ ID NO:358-361; see also Table 14). In certain embodiments of the invention, the protein of a mammalian MPV is a F protein, wherein the F protein is at least 60%, at least 65%, at least 70%, at least 75%, at least 80%, at least 85%, at least 90%, at least 95%, at least 98%, at least 99%, or at least 99.5% identical to the amino acid sequence of a F protein encoded by the viral genome of SEQ ID NO:18, SEQ ID NO:19, SEQ ID NO:20, or SEQ ID NO:21 (the amino acid sequences of the respective F proteins are disclosed in SEQ ID NO:314-317; see also Table 14). In certain embodiments of the invention, the protein of a mammalian MPV is a M2-1 protein, wherein the M2-1 protein is at least 60%, at least 65%, at least 70%, at least 75%, at least 80%, at least 85%, at least 90%, at least 95%, at least 98%, at least 99%, or at least 99.5% identical to the amino acid sequence of a M2-1 protein encoded by the viral genome of SEQ ID NO:18, SEQ ID NO:19, SEQ ID NO:20, or SEQ ID NO:21 (the amino acid sequences of the respective M2-1 proteins are disclosed in SEQ ID NO:338-341; see also Table 14). In certain embodiments of the invention, the protein of a mammalian MPV is a M2-2 protein, wherein the M2-2 protein is at least 60%, at least 65%, at least 70%, at least 75%, at least 80%, at least 85%, at least 90%, at least 95%, at least 98%, at least 99%, or at least 99.5% identical to the amino acid sequence of a M2-2 protein encoded by the viral genome of SEQ ID NO:18, SEQ ID NO:19, SEQ ID NO:20, or SEQ ID NO:21 (the amino acid sequences of the respective M2-2 proteins are disclosed in SEQ ID NO:346-349; see also Table 14). In certain embodiments of the invention, the protein of a mammalian MPV is a G protein, wherein the G protein is at least 60%, at least 65%, at least 70%, at least 75%, at least 80%, at least 85%, at least 90%, at least 95%, at least 98%, at least 99%, or at least 99.5% identical to the amino acid sequence of a G protein encoded by the viral genome of SEQ ID NO:18, SEQ ID NO:19, SEQ ID NO:20, or SEQ ID NO:21 (the amino acid sequences of the respective G proteins are disclosed in SEQ ID NO:322-325; see also Table 14). In certain embodiments of the invention, the protein of a mammalian MPV is a SH protein, wherein the SH protein is at least 60%, at least 65%, at least 70%, at least 75%, at least 80%, at least 85%, at least 90%, at least 95%, at least 98%, at least 99%, or at least 99.5% identical to the amino acid sequence of a SH protein encoded by the viral genome of SEQ ID NO:18, SEQ ID NO:19, SEQ ID NO:20, or SEQ ID NO:21 (the amino acid sequences of the respective SH proteins are disclosed in SEQ ID NO:382-385; see also Table 14). In certain embodiments of the invention, the protein of a mammalian MPV is a L protein, wherein the L protein is at least 60%, at least 65%, at least 70%, at least 75%, at least 80%, at least 85%, at least 90%, at least 95%, at least 98%, at least 99%, or at least 99.5% identical to the amino acid sequence of a L protein encoded by the viral genome of SEQ ID NO:18, SEQ ID NO:19, SEQ ID NO:20, or SEQ ID NO:21 (the amino acid sequences of the respective L proteins are disclosed in SEQ ID NO:330-333; see also Table 14).

A fragment of a protein of mammalian MPV is at least 60%, at least 65%, at least 70%, at least 75%, at least 80%, at least 85%, at least 90%, at least 95%, at least 98%, at least 99%, or at least 99.5% identical to the homologous protein encoded by the virus of SEQ ID NO:18, SEQ ID NO:19, SEQ ID NO:20, or SEQ ID NO:21 over the portion of the protein that is homologous to the fragment. In a specific, illustrative embodiment, the invention provides a fragment of the F protein of a mammalian MPV that contains the ectodomain of the F protein and homologs thereof. The homolog of the fragment of the F protein that contains the ectodomain is at least 60%, at least 65%, at least 70%, at least 75%, at least 80%, at least 85%, at least 90%, at least 95%, at least 98%, at least 99%, or at least 99.5% identical to the corresponding fragment containing the ectodomain of the F protein encoded by a virus of SEQ ID NO:18, SEQ ID NO:19, SEQ ID NO:20, or SEQ ID NO:21 (the amino acid sequences of the respective F proteins are disclosed in SEQ ID NO:314-317; see also Table 14).

In certain embodiments, the invention provides a protein of a mammalian MPV of subgroup A and fragments thereof. The invention provides a N protein of a mammalian MPV of subgroup A, wherein the N protein is phylogenetically closer related to the N protein encoded by a virus of SEQ ID NO:19 or SEQ ID NO:20 than it is related to the N protein encoded by a virus encoded by SEQ ID NO:18 or SEQ ID NO:21. The invention provides a G protein of a mammalian MPV of subgroup A, wherein the G protein is phylogenetically closer related to the G protein encoded by a virus of SEQ ID NO:19 or SEQ ID NO:20 than it is related to the G protein encoded by a virus encoded by SEQ ID NO:18 or SEQ ID NO:21. The invention provides a P protein of a mammalian MPV of subgroup A, wherein the P protein is phylogenetically closer related to the P protein encoded by a virus of SEQ ID NO:19 or SEQ ID NO:20 than it is related to the P protein encoded by a virus encoded by SEQ ID NO:18 or SEQ ID NO:21. The invention provides a M protein of a mammalian MPV of subgroup A, wherein the M protein is phylogenetically closer related to the M protein encoded by a virus of SEQ ID NO:19 or SEQ ID NO:20 than it is related to the M protein encoded by a virus encoded by SEQ ID NO:18 or SEQ ID NO:21. The invention provides a N protein of a mammalian MPV of subgroup A, wherein the F protein is phylogenetically closer related to the F protein encoded by a virus of SEQ ID NO:19 or SEQ ID NO:20 than it is related to the F protein encoded by a virus encoded by SEQ ID NO:18 or SEQ ID NO:21. The invention provides a M2-1 protein of a mammalian MPV of subgroup A, wherein the M2-1 protein is phylogenetically closer related to the M2-1 protein encoded by a virus of SEQ ID NO:19 or SEQ ID NO:20 than it is related to the M2-1 protein encoded by a virus encoded by SEQ ID NO:18 or SEQ ID NO:21. The invention provides a M2-2 protein of a mammalian MPV of subgroup A, wherein the M2-2 protein is phylogenetically closer related to the M2-2 protein encoded by a virus of SEQ ID NO:19 or SEQ ID NO:20 than it is related to the M2-2 protein encoded by a virus encoded by SEQ ID NO:18 or SEQ ID NO:21. The invention provides a SH protein of a mammalian MPV of subgroup A, wherein the SH protein is phylogenetically closer related to the SH protein encoded by a virus of SEQ ID NO:19 or SEQ ID NO:20 than it is related to the SH protein encoded by a virus encoded by SEQ ID NO:18 or SEQ ID NO:21. The invention provides a L protein of a mammalian MPV of subgroup A, wherein the L protein is phylogenetically closer related to the L protein encoded by a virus of SEQ ID NO:19 or SEQ ID NO:20 than it is related to the L protein encoded by a virus encoded by SEQ ID NO:18 or SEQ ID NO:21.

In other embodiments, the invention provides a protein of a mammalian MPV of subgroup B or fragments thereof. The invention provides a N protein of a mammalian MPV of subgroup B, wherein the N protein is phylogenetically closer related to the N protein encoded by a virus of SEQ ID NO:18 or SEQ ID NO:21 than it is related to the N protein encoded by a virus encoded by SEQ ID NO:19 or SEQ ID NO:20. The invention provides a G protein of a mammalian MPV of subgroup A, wherein the G protein is phylogenetically closer related to the G protein encoded by a virus of SEQ ID NO:18 or SEQ ID NO:21 than it is related to the G protein encoded by a virus encoded by SEQ ID NO:19 or SEQ ID NO:20. The invention provides a P protein of a mammalian MPV of subgroup A, wherein the P protein is phylogenetically closer related to the P protein encoded by a virus of SEQ ID NO:18 or SEQ ID NO:21 than it is related to the P protein encoded by a virus encoded by SEQ ID NO:19 or SEQ ID NO:20. The invention provides a M protein of a mammalian MPV of subgroup A, wherein the M protein is phylogenetically closer related to the M protein encoded by a virus of SEQ ID NO:18 or SEQ ID NO:21 than it is related to the M protein encoded by a virus encoded by SEQ ID NO:19 or SEQ ID NO:20. The invention provides a N protein of a mammalian MPV of subgroup A, wherein the F protein is phylogenetically closer related to the F protein encoded by a virus of SEQ ID NO:18 or SEQ ID NO:21 than it is related to the F protein encoded by a virus encoded by SEQ ID NO:19 or SEQ ID NO:20. The invention provides a M2-1 protein of a mammalian MPV of subgroup A, wherein the M2-1 protein is phylogenetically closer related to the M2-1 protein encoded by a virus of SEQ ID NO:18 or SEQ ID NO:21 than it is related to the M2-1 protein encoded by a virus encoded by SEQ ID NO:19 or SEQ ID NO:20. The invention provides a M2-2 protein of a mammalian MPV of subgroup A, wherein the M2-2 protein is phylogenetically closer related to the M2-2 protein encoded by a virus of SEQ ID NO:18 or SEQ ID NO:21 than it is related to the M2-2 protein encoded by a virus encoded by SEQ ID NO:19 or SEQ ID NO:20. The invention provides a SH protein of a mammalian MPV of subgroup A, wherein the SH protein is phylogenetically closer related to the SH protein encoded by a virus of SEQ ID NO:18 or SEQ ID NO:21 than it is related to the SH protein encoded by a virus encoded by SEQ ID NO:19 or SEQ ID NO:20. The invention provides a L protein of a mammalian MPV of subgroup A, wherein the L protein is phylogenetically closer related to the L protein encoded by a virus of SEQ ID NO:18 or SEQ ID NO:21 than it is related to the L protein encoded by a virus encoded by SEQ ID NO:19 or SEQ ID NO:20.

The invention further provides proteins of a mammalian MPV of variant A1, A2, B1 or B2. In certain embodiments of the invention, the proteins of the different variants of mammalian MPV can be distinguished from each other by way of their amino acid sequence identities. A variant of mammalian MPV can be, but is not limited to, A1, A2, B1 or B2. The invention, however, also contemplates isolates of mammalian MPV that are members of another variant.

The invention provides a G protein of a mammalian MPV variant B1, wherein the G protein of a mammalian MPV variant B1 is phylogenetically closer related to the G protein of the prototype of variant B1, isolate NL/1/99, than it is related to the G protein of the prototype of variant A1, isolate NL/1/00, the G protein of the prototype of A2, isolate NL/17/00, or the G protein of the prototype of B2, isolate NL/1/94. The invention provides a G protein of a mammalian MPV variant B1, wherein the amino acid sequence of the G protein is at least 66%, at least 70%, at least 75%, at least 80%, at least 85%, at least 90%, at least 95%, at least 98%, or at least 99% or at least 99.5% identical to the G protein of a mammalian MPV variant B1 as represented by the prototype NL/1/99 (SEQ ID NO:324). The invention provides a N protein of a mammalian MPV variant B1, wherein the N protein of a mammalian MPV variant B1 is phylogenetically closer related to the N protein of the prototype of variant B1, isolate NL/1/99, than it is related to the N protein of the prototype of variant A1, isolate NL/1/00, the N protein of the prototype of A2, isolate NL/17/00, or the N protein of the prototype of B2, isolate NL/1/94. The invention provides a N protein of a mammalian MPV variant B1, wherein the amino acid sequence of the N proteint is at least 98.5% or at least 99% or at least 99.5% identical to the N protein of a mammalian MPV variant B1 as represented by the prototype NL/1/99 (SEQ ID NO:368). The invention provides a P protein of a mammalian MPV variant B1, wherein the P protein of a mammalian MPV variant B1 is phylogenetically closer related to the P protein of the prototype of variant B1, isolate NL/1/99, than it is related to the P protein of the prototype of variant A1, isolate NL/1/00, the P protein of the prototype of A2, isolate NL/17/00, or the P protein of the prototype of B2, isolate NL/1/94. The invention provides a P protein of a mammalian MPV variant B1, wherein the amino acid sequence of the P protein is at least 96%, at least 98%, or at least 99% or at least 99.5% identical the P protein of a mammalian MPV variant B1 as represented by the prototype NL/1/99 (SEQ ID NO:376). The invention provides a M protein of a mammalian MPV variant B1, wherein the M protein of a mammalian MPV variant B1 is phylogenetically closer related to the M protein of the prototype of variant B1, isolate NL/1/99, than it is related to the M protein of the prototype of variant A1, isolate NL/1/00, the M protein of the prototype of A2, isolate NL/17/00, or the M protein of the prototype of B2, isolate NL/1/94. The invention provides a M protein of a mammalian MPV variant B1, wherein the amino acid sequence of the M protein is identical the M protein of a mammalian MPV variant B1 as represented by the prototype NL/1/99 (SEQ ID NO:360). The invention provides a F protein of a mammalian MPV variant B1, wherein the F protein of a mammalian MPV variant B1 is phylogenetically closer related to the F protein of the prototype of variant B1, isolate NL/1/99, than it is related to the F protein of the prototype of variant A1, isolate NL/1/00, the F protein of the prototype of A2, isolate NL/17/00, or the F protein of the prototype of B2, isolate NL/1/94. The invention provides a F protein of a mammalian MPV variant B1, wherein the amino acid sequence of the F protein is at least 99% identical to the F protein of a mammalian MPV variant B1 as represented by the prototype NL/1/99 (SEQ ID NO:316). The invention provides a M2-1 protein of a mammalian MPV variant B1, wherein the M2-1 protein of a mammalian MPV variant B1 is phylogenetically closer related to the M2-1 protein of the prototype of variant B1, isolate NL/1/99, than it is related to the M2-1 protein of the prototype of variant A1, isolate NL/1/00, the M2-1 protein of the prototype of A2, isolate NL/17/00, or the M2-1 protein of the prototype of B2, isolate NL/1/94. The invention provides a M2-1 protein of a mammalian MPV variant B1, wherein the amino acid sequence of the M2-1 protein is at least 98% or at least 99% or at least 99.5% identical the M2-1 protein of a mammalian MPV variant B1 as represented by the prototype NL/1/99 (SEQ ID NO:340). The invention provides a M2-2 protein of a mammalian MPV variant B1, wherein the M2-2 protein of a mammalian MPV variant B1 is phylogenetically closer related to the M2-2 protein of the prototype of variant B1, isolate NL/1/99, than it is related to the M2-2 protein of the prototype of variant A1, isolate NL/1/00, the M2-2 protein of the prototype of A2, isolate NL/17/00, or the M2-2 protein of the prototype of B2, isolate NL/1/94. The invention provides a M2-2 protein of a mammalian MPV variant B1, wherein the amino acid sequence of the M2-2 protein is at least 99%or at least 99.5% identical the M2-2 protein of a mammalian MPV variant B1 as represented by the prototype NL/1/99 (SEQ ID NO:348). The invention provides a SH protein of a mammalian MPV variant B1, wherein the SH protein of a mammalian MPV variant B1 is phylogenetically closer related to the SH protein of the prototype of variant B1, isolate NL/1/99, than it is related to the SH protein of the prototype of variant A1, isolate NL/1/00, the SH protein of the prototype of A2, isolate NL/17/00, or the SH protein of the prototype of B2, isolate NL/1/94. The invention provides a SH protein of a mammalian MPV variant B1, wherein the amino acid sequence of the SH protein is at least 83%, at least 85%, at least 90%, at least 95%, at least 98%, or at least 99% or at least 99.5% identical the SH protein of a mammalian MPV variant B1 as represented by the prototype NL/1/99 (SEQ ID NO:384). The invention provides a L protein of a mammalian MPV variant B1, wherein the L protein of a mammalian MPV variant B1 is phylogenetically closer related to the L protein of the prototype of variant B1, isolate NL/1/99, than it is related to the L protein of the prototype of variant A1, isolate NL/1/00, the L protein of the prototype of A2, isolate NLJ17/00, or the L protein of the prototype of B2, isolate NL/1/94. The invention provides a L protein of a mammalian MPV variant B1, wherein the amino acid sequence of the L protein is at least 99% or at least 99.5% identical the L protein a mammalian MPV variant B1 as represented by the prototype NL/1/99 (SEQ ID NO:332).

The invention provides a G protein of a mammalian MPV variant A1, wherein the G protein of a mammalian MPV variant A1 is phylogenetically closer related to the G protein of the prototype of variant A1, isolate NL/1/00, than it is related to the G protein of the prototype of variant B1, isolate NL/1/99, the G protein of the prototype of A2, isolate NL/17/00, or the G protein of the prototype of B2, isolate NL/1/94. The invention provides a G protein of a mammalian MPV variant A1, wherein the amino acid sequence of the G protein is at least 66%, at least 70%, at least 75%, at least 80%, at least 85%, at least 90%, at least 95%, at least 98%, or at least 99% or at least 99.5% identical to the G protein of a mammalian MPV variant A1 as represented by the prototype NL/1/00 (SEQ ID NO:322). The invention provides a N protein of a mammalian MPV variant A1, wherein the N protein of a mammalian MPV variant A1 is phylogenetically closer related to the N protein of the prototype of variant A1, isolate NL/1/00, than it is related to the N protein of the prototype of variant B1, isolate NL/1/99, the N protein of the prototype of A2, isolate NL/17/00, or the N protein of the prototype of B2, isolate NL/1/94. The invention provides a N protein of a mammalian MPV variant A1, wherein the amino acid sequence of the N protein is at least 99.5% identical to the N protein of a mammalian MPV variant A1 as represented by the prototype NL/1/00 (SEQ ID NO:366). The invention provides a P protein of a mammalian MPV variant A1, wherein the P protein of a mammalian MPV variant A1 is phylogenetically closer related to the P protein of the prototype of variant A1, isolate NL/1/00, than it is related to the P protein of the prototype of variant B1, isolate NL/1/99, the P protein of the prototype of A2, isolate NL/17/00, or the P protein of the prototype of B2, isolate NL/1/94. The invention provides a P protein of a mammalian MPV variant A1, wherein the amino acid sequence of the P protein is at least 96%, at least 98%, or at least 99% or at least 99.5% identical to the P protein of a mammalian MPV variant A1 as represented by the prototype NL/1/00 (SEQ ID NO:374). The invention provides a M protein of a mammalian MPV variant A1, wherein the M protein of a mammalian MPV variant A1 is phylogenetically closer related to the M protein of the prototype of variant A1, isolate NL/1/00, than it is related to the M protein of the prototype of variant B1, isolate NL/1/99, the M protein of the prototype of A2, isolate NL/17/00, or the M protein of the prototype of B2, isolate NL/1/94. The invention provides a M protein of a mammalian MPV variant A1, wherein the amino acid sequence of the M protein is at least 99% or at least 99.5% identical to the M protein of a mammalian MPV variant A1 as represented by the prototype NL/1/00 (SEQ ID NO:358). The invention provides a F protein of a mammalian MPV variant A1, wherein the F protein of a mammalian MPV variant A1 is phylogenetically closer related to the F protein of the prototype of variant A1, isolate NL/1/00, than it is related to the F protein of the prototype of variant B1, isolate NL/1/99, the F protein of the prototype of A2, isolate NL/17/00, or the F protein of the prototype of B2, isolate NL/1/94. The invention provides a F protein of a mammalian MPV variant A1, wherein the amino acid sequence of the F protein is at least 98% or at least 99% or at least 99.5% identical to the F protein of a mammalian MPV variant A1 as represented by the prototype NL/1/00 (SEQ ID NO:314). The invention provides a M2-1 protein of a mammalian MPV variant A1, wherein the M2-1 protein of a mammalian MPV variant A1 is phylogenetically closer related to the M2-1 protein of the prototype of variant A1, isolate NL/1/00, than it is related to the M2-1 protein of the prototype of variant B1, isolate NL/1/99, the M2-1 protein of the prototype of A2, isolate NL/17/00, or the M2-1 protein of the prototype of B2, isolate NL/1/94. The invention provides a M2-1 protein of a mammalian MPV variant A1, wherein the amino acid sequence of the M2-1 protein is at least 99% or at least 99.5% identical to the M2-1 protein of a mammalian MPV variant A1 as represented by the prototype NL/1/00 (SEQ ID NO:338). The invention provides a M2-2 protein of a mammalian MPV variant A1, wherein the M2-2 protein of a mammalian MPV variant A1 is phylogenetically closer related to the M2-2 protein of the prototype of variant A1, isolate NL/1/00, than it is related to the M2-2 protein of the prototype of variant B1, isolate NL/1/99, the M2-2 protein of the prototype of A2, isolate NL/17/00, or the M2-2 protein of the prototype of B2, isolate NL/1/94. The invention provides a M2-2 protein of a mammalian MPV variant A1, wherein the amino acid sequence of the M2-2 protein is at least 96% or at least 99% or at least 99.5% identical to the M2-2 protein of a mammalian MPV variant A1 as represented by the prototype NL/1/00 (SEQ ID NO:346). The invention provides a SH protein of a mammalian MPV variant A1, wherein the SH protein of a mammalian MPV variant A1 is phylogenetically closer related to the SH protein of the prototype of variant A1, isolate NL/1/00, than it is related to the SH protein of the prototype of variant B1, isolate NL/1/99, the SH protein of the prototype of A2, isolate NL/17/00, or the SH protein of the prototype of B2, isolate NL/1/94. The invention provides a SH protein of a mammalian MPV variant A1, wherein the amino acid sequence of the SH protein is at least 84%, at least 90%, at least 95%, at least 98%, or at least 99% or at least 99.5% identical to the SH protein of a mammalian MPV variant A1 as represented by the prototype NL/1/00 (SEQ ID NO:382). The invention provides a L protein of a mammalian MPV variant A1, wherein the L protein of a mammalian MPV variant A1 is phylogenetically closer related to the L protein of the prototype of variant A1, isolate NL/1/00, than it is related to the L protein of the prototype of variant B1, isolate NL/1/99, the L protein of the prototype of A2, isolate NL/17/00, or the L protein of the prototype of B2, isolate NL/1/94. The invention provides a L protein of a mammalian MPV variant A1, wherein the amino acid sequence of the L protein is at least 99% or at least 99.5% identical to the L protein of a virus of a mammalian MPV variant A1 as represented by the prototype NL/1/00 (SEQ ID NO:330).

The invention provides a G protein of a mammalian MPV variant A2, wherein the G protein of a mammalian MPV variant A2 is phylogenetically closer related to the G protein of the prototype of variant A2, isolate NL/17/00, than it is related to the G protein of the prototype of variant B1, isolate NL/1/99, the G protein of the prototype of A1, isolate NL/1/00, or the G protein of the prototype of B2, isolate NL/1/94. The invention provides a G protein of a mammalian MPV variant A2, wherein the amino acid sequence of the G protein is at least 66%, at least 70%, at least 75%, at least 80%, at least 85%, at least 90%, at least 95%, at least 98%, at least 99% or at least 99.5% identical to the G protein of a mammalian MPV variant A2 as represented by the prototype NL/17/00 (SEQ ID NO:332). The invention provides a N protein of a mammalian MPV variant A2, wherein the N protein of a mammalian MPV variant A2 is phylogenetically closer related to the N protein of the prototype of variant A2, isolate NL/17/00, than it is related to the N protein of the prototype of variant B1, isolate NL/1/99, the N protein of the prototype of A1, isolate NL/1/00, or the N protein of the prototype of B2, isolate NL/1/94. The invention provides a N protein of a mammalian MPV variant A2, wherein the amino acid sequence of the N protein at least 99.5% identical to the N protein of a mammalian MPV variant A2 as represented by the prototype NL/17/00 (SEQ ID NO:367). The invention provides a P protein of a mammalian MPV variant A2, wherein the P protein of a mammalian MPV variant A2 is phylogenetically closer related to the P protein of the prototype of variant A2, isolate NL/17/00, than it is related to the P protein of the prototype of variant B1, isolate NL/1/99, the P protein of the prototype of A1, isolate NL/1/00, or the P protein of the prototype of B2, isolate NL/1/94. The invention provides a P protein of a mammalian MPV variant A2, wherein the amino acid sequence of the P protein is at least 96%, at least 98%, at least 99% or at least 99.5% identical to the P protein of a mammalian MPV variant A2 as represented by the prototype NL/17/00 (SEQ ID NO:375). The invention provides a M protein of a mammalian MPV variant A2, wherein the M protein of a mammalian MPV variant A2 is phylogenetically closer related to the M protein of the prototype of variant A2, isolate NL/17/00, than it is related to the M protein of the prototype of variant B1, isolate NL/1/99, the M protein of the prototype of A1, isolate NL/1/00, or the M protein of the prototype of B2, isolate NL/1/94. The invention provides a M protein of a mammalian MPV variant A2, wherein the the amino acid sequence of the M protein is at least 99%, or at least 99.5% identical to the M protein of a mammalian MPV variant A2 as represented by the prototype NL/17/00 (SEQ ID NO:359). The invention provides a F protein of a mammalian MPV variant A2, wherein the F protein of a mammalian MPV variant A2 is phylogenetically closer related to the F protein of the prototype of variant A2, isolate NL/17/00, than it is related to the F protein of the prototype of variant B1, isolate NL/1/99, the F protein of the prototype of A1, isolate NL/1/00, or the F protein of the prototype of B2, isolate NL/1/94. The invention provides a F protein of a mammalian MPV variant A2, wherein the amino acid sequence of the F protein is at least 98%, at least 99% or at least 99.5% identical to the F protein of a mammalian MPV variant A2 as represented by the prototype NL/17/00 (SEQ ID NO:315). The invention provides a M2-1 protein of a mammalian MPV variant A2, wherein the M2-1 protein of a mammalian MPV variant A2 is phylogenetically closer related to the M2-1 protein of the prototype of variant A2, isolate NL/17/00, than it is related to the M2-1 protein of the prototype of variant B1, isolate NL/1/99, the M2-1 protein of the prototype of A1, isolate NL/1/00, or the M2-1 protein of the prototype of B2, isolate NL/1/94. The invention provides a M2-1 protein of a mammalian MPV variant A2, wherein the amino acid sequence of the M2-1 protein is at least 99%, or at least 99.5% identical to the M2-1 protein of a mammalian MPV variant A2 as represented by the prototype NL/17/00 (SEQ ID NO: 339). The invention provides a M2-2 protein of a mammalian MPV variant A2, wherein the M2-2 protein of a mammalian MPV variant A2 is phylogenetically closer related to the M2-2 protein of the prototype of variant A2, isolate NL/17/00, than it is related to the M2-2 protein of the prototype of variant B1, isolate NL/1/99, the M2-2 protein of the prototype of A1, isolate NL/1/00, or the M2-2 protein of the prototype of B2, isolate NL/1/94. The invention provides a M2-2 protein of a mammalian MPV variant A2, wherein the amino acid sequence of the M2-2 protein is at least 96%, at least 98%, at least 99% or at least 99.5% identical to the M2-2 protein of a mammalian MPV variant A2 as represented by the prototype NL/17/00 (SEQ ID NO:347). The invention provides a SH protein of a mammalian MPV variant A2, wherein the SH protein of a mammalian MPV variant A2 is phylogenetically closer related to the SH protein of the prototype of variant A2, isolate NL/17/00, than it is related to the SH protein of the prototype of variant B1, isolate NL/1/99, the SH protein of the prototype of A1, isolate NL/1/00, or the SH protein of the prototype of B2, isolate NL/1/94. The invention provides a SH protein of a mammalian MPV variant A2, wherein the amino acid sequence of the SH protein is at least 84%, at least 85%, at least 90%, at least 95%, at least 98%, at least 99% or at least 99.5% identical to the SH protein of a mammalian MPV variant A2 as represented by the prototype NL/17/00 (SEQ ID NO:383). The invention provides a L protein of a mammalian MPV variant A2, wherein the L protein of a mammalian MPV variant A2 is phylogenetically closer related to the L protein of the prototype of variant A2, isolate NL/17/00, than it is related to the L protein of the prototype of variant B1, isolate NL/1/99, the L protein of the prototype of A1, isolate NL/1/00, or the L protein of the prototype of B2, isolate NL/1/94. The invention provides a L protein of a mammalian MPV variant A2, wherein the amino acid sequence of the L protein is at least 99% or at least 99.5% identical to the L protein of a mammalian MPV variant A2 as represented by the prototype NL/17/00 (SEQ ID NO:331).

The invention provides a G protein of a mammalian MPV variant B2, wherein the G protein of a mammalian MPV variant B2 is phylogenetically closer related to the G protein of the prototype of variant B2, isolate NL/1/94, than it is related to the G protein of the prototype of variant B1, isolate NL/1/99, the G protein of the prototype of A1, isolate NL/1/00, or the G protein of the prototype of A2, isolate NL/17/00. The invention provides a G protein of a mammalian MPV variant B2, wherein the amino acid sequence of the G protein is at least 66%, at least 70%, at least 75%, at least 80%, at least 85%, at least 90%, at least 95%, at least 98%, or at least 99% or at least 99.5% identical to the G protein of a mammalian MPV variant B2 as represented by the prototype NL/1/94 (SEQ ID NO:325). The invention provides a N protein of a mammalian MPV variant B2, wherein the N protein of a mammalian MPV variant B2 is phylogenetically closer related to the N protein of the prototype of variant B2, isolate NL/1/94, than it is related to the N protein of the prototype of variant B1, isolate NL/1/99, the N protein of the prototype of A1, isolate NL/1/00, or the N protein of the prototype of A2, isolate NV17/00. The invention provides a N protein of a mammalian MPV variant B2, wherein the amino acid sequence of the N protein is at least 99% or at least 99.5% identical to the N protein of a mammalian MPV variant B2 as represented by the prototype NL/1/94 (SEQ ID NO:369). The invention provides a P protein of a mammalian MPV variant B2, wherein the P protein of a mammalian MPV variant B2 is phylogenetically closer related to the P protein of the prototype of variant B2, isolate NL/1/94, than it is related to the P protein of the prototype of variant B1, isolate NL/1/99, the P protein of the prototype of A1, isolate NL/1/00, or the P protein of the prototype of A2, isolate NL/17/00. The invention provides a P protein of a mammalian MPV variant B2, wherein the amino acid sequence of the P protein is at least 96%, at least 98%, or at least 99% or at least 99.5% identical to the P protein of a mammalian MPV variant B2 as represented by the prototype NL/1/94 (SEQ ID NO:377). The invention provides a M protein of a mammalian MPV variant B2, wherein the M protein of a mammalian MPV variant B2 is phylogenetically closer related to the M protein of the prototype of variant B2, isolate NL/1/94, than it is related to the M protein of the prototype of variant B1, isolate NL/1/99, the M protein of the prototype of A1, isolate NL/1/00, or the M protein of the prototype of A2, isolate NL/17/00. The invention provides a M protein of a mammalian MPV variant B2, wherein the amino acid sequence of its M protein is identical to the M protein of a mammalian MPV variant B2 as represented by the prototype NL/1/94 (SEQ ID NO:361). The invention provides a F protein of a mammalian MPV variant B2, wherein the F protein of a mammalian MPV variant B2 is phylogenetically closer related to the F protein of the prototype of variant B2, isolate NL/1/94, than it is related to the F protein of the prototype of variant B1, isolate NL/1/99, the F protein of the prototype of A1, isolate NL/1/00, or the F protein of the prototype of A2, isolate NL/17/00. The invention provides a F protein of a mammalian MPV variant B2, wherein the amino acid sequence of the F protein is at least 99% or at least 99.5% identical to the F protein of a mammalian MPV variant B2 as represented by the prototype NL/1/94 (SEQ ID NO:317). The invention provides a M2-1 protein of a mammalian MPV variant B2, wherein the M2-1 protein of a mammalian MPV variant B2 is phylogenetically closer related to the M2-1 protein of the prototype of variant B2, isolate NL/1/94, than it is related to the M2-1 protein of the prototype of variant B1, isolate NL/1/99, the M2-1 protein of the prototype of A1, isolate NL/1/00, or the M2-1 protein of the prototype of A2, isolate NL/17/00. The invention provides a M2-1 protein of a mammalian MPV variant B2, wherein the amino acid sequence of the M2-1 protein is at least 98% or at least 99% or at least 99.5% identical to the M2-1 protein of a mammalian MPV variant B2 as represented by the prototype NL/1/94 (SEQ ID NO:341). The invention provides a M2-2 protein of a mammalian MPV variant B2, wherein the M2-2 protein of a mammalian MPV variant B2 is phylogenetically closer related to the M2-2 protein of the prototype of variant B2, isolate NL/1/94, than it is related to the M2-2 protein of the prototype of variant B1, isolate NL/1/99, the M2-2 protein of the prototype of A1, isolate NL/1/00, or the M2-2 protein of the prototype of A2, isolate NL/17/00. The invention provides a M2-2 protein of a mammalian MPV variant B2, wherein the amino acid sequence is at least 99% or at least 99.5% identical to the M2-2 protein of a mammalian MPV variant B2 as represented by the prototype NL/1/94 (SEQ ID NO:349). The invention provides a SH protein of a mammalian MPV variant B2, wherein the SH protein of a mammalian MPV variant B2 is phylogenetically closer related to the SH protein of the prototype of variant B2, isolate NL/1/94, than it is related to the SH protein of the prototype of variant B1, isolate NL/1/99, the SH protein of the prototype of A1, isolate NL/1/00, or the SH protein of the prototype of A2, isolate NL/17/00. The invention provides a SH protein of a mammalian MPV variant B2, wherein the amino acid sequence of the SH protein is at least 84%, at least 85%, at least 90%, at least 95%, at least 98%, or at least 99% or at least 99.5% identical to the SH protein of a mammalian MPV variant B2 as represented by the prototype NL/1/94 (SEQ ID NO:385). The invention provides a L protein of a mammalian MPV variant B2, wherein the L protein of a mammalian MPV variant B2 is phylogenetically closer related to the L protein of the prototype of variant B2, isolate NL/1/94, than it is related to the L protein of the prototype of variant B1, isolate NL/1/99, the L protein of the prototype of A1, isolate NL/1/00, or the L protein of the prototype of A2, isolate NL/17/00. The invention provides a L protein of a mammalian MPV variant B2, wherein the and/or if the amino acid sequence of the L protein is at least 99% or at least 99.5% identical to the L protein of a mammalian MPV variant B2 as represented by the prototype NL/1/94 (SEQ ID NO:333).

In certain embodiments, the percentage of sequence identity is based on an alignment of the full length proteins. In other embodiments, the percentage of sequence identity is based on an alignment of contiguous amino acid sequences of the proteins, wherein the amino acid sequences can be 25 amino acids, 50 amino acids, 75 amino acids, 100 amino acids, 125 amino acids, 150 amino acids, 175 amino acids, 200 amino acids, 225 amino acids, 250 amino acids, 275 amino acids, 300 amino acids, 325 amino acids, 350 amino acids, 375 amino acids, 400 amino acids, 425 amino acids, 450 amino acids, 475 amino acids, 500 amino acids, 750 amino acids, 1000 amino acids, 1250 amino acids, 1500 amino acids, 1750 amino acids, 2000 amino acids or 2250 amino acids in length.

In certain, specific embodiments, the invention provides a G protein of a mammalian MPV wherein the G protein has one of the amino acid sequences set forth in SEQ ID NO:119-153; SEQ ID NO:322-325 or a fragment thereof. In certain, specific embodiments, the invention provides a F protein of a mammalian MPV wherein the F protein has one of the amino acid sequences set forth in SEQ ID NO:234-317. In certain, specific embodiments, the invention provides a L protein of a mammalian MPV wherein the L protein has one of the amino acid sequences set forth in SEQ ID NO:330-333 or a fragment thereof. In certain, specific embodiments, the invention provides a M2-1 protein of a mammalian MPV wherein the M2-1 protein has one of the amino acid sequences set forth in SEQ ID NO:338-341 or a fragment thereof. In certain, specific embodiments, the invention provides a M2-2 protein of a mammalian MPV wherein the M2-2 protein has one of the amino acid sequences set forth in SEQ ID NO:346-349 or a fragment thereof. In certain, specific embodiments, the invention provides a M protein of a mammalian MPV wherein the M protein has one of the amino acid sequences set forth in SEQ ID NO:358-361 or a fragment thereof. In certain, specific embodiments, the invention provides a N protein of a mammalian MPV wherein the N protein has one of the amino acid sequences set forth in SEQ ID NO:366-369 or a fragment thereof. In certain, specific embodiments, the invention provides a P protein of a mammalian MPV wherein the P protein has one of the amino acid sequences set forth in SEQ ID NO:374-377 or a fragment thereof. In certain, specific embodiments, the invention provides a SH protein of a mammalian MPV wherein the SH protein has one of the amino acid sequences set forth in SEQ ID NO:382-385 or a fragment thereof.

In certain embodiments of the invention, a fragment is at least 25 amino acids, 50 amino acids, 75 amino acids, 100 amino acids, 125 amino acids, 150 amino acids, 175 amino acids, 200 amino acids, 225 amino acids, 250 amino acids, 275 amino acids, 300 amino acids, 325 amino acids, 350 amino acids, 375 amino acids, 400 amino acids, 425 amino acids, 450 amino acids, 475 amino acids, 500 amino acids, 750 amino acids, 1000 amino acids, 1250 amino acids, 1500 amino acids, 1750 amino acids, 2000 amino acids or 2250 amino acids in length. In certain embodiments of the invention, a fragment is at most 25 amino acids, 50 amino acids, 75 amino acids, 100 amino acids, 125 amino acids, 150 amino acids, 175 amino acids, 200 amino acids, 225 amino acids, 250 amino acids, 275 amino acids, 300 amino acids, 325 amino acids, 350 amino acids, 375 amino acids, 400 amino acids, 425 amino acids, 450 amino acids, 475 amino acids, 500 amino acids, 750 amino acids, 1000 amino acids, 1250 amino acids, 1500 amino acids, 1750 amino acids, 2000 amino acids or 2250 amino acids in length.

The invention further provides nucleic acid sequences derived from a mammalian MPV. The invention also provides derivatives of nucleic acid sequences derived from a mammalian MPV. In certain specific embodiments the nucleic acids are modified.

In certain embodiments, a nucleic acid of the invention encodes a G protein, a N protein, a P protein, a M protein, a F protein, a M2-1 protein, a M2-2 protein, a SH protein, or a L protein of a mammalian MPV as defined above. In certain embodiments, a nucleic acid of the invention encodes a G protein, a N protein, a P protein, a M protein, a F protein, a M2-1 protein, a M2-2 protein, a SH protein, or a L protein of subgroup A of a mammalian MPV as defined above. In certain embodiments, a nucleic acid of the invention encodes a G protein, a N protein, a P protein, a M protein, a F protein, a M2-1 protein, a M2-2 protein, a SH protein, or a L protein of subgroup B of a mammalian MPV as defined above. In certain embodiments, a nucleic acid of the invention encodes a G protein, a N protein, a P protein, a M protein, a F protein, a M2-1 protein, a M2-2 protein, a SH protein, or a L protein of variant A1 of a mammalian MPV as defined above. In certain embodiments, a nucleic acid of the invention encodes a G protein, a N protein, a P protein, a M protein, a F protein, a M2-1 protein, a M2-2 protein, a SH protein, or a L protein of variant A2 of a mammalian MPV as defined above. In certain embodiments, a nucleic acid of the invention encodes a G protein, a N protein, a P protein, a M protein, a F protein, a M2-1 protein, a M2-2 protein, a SH protein, or a L protein of variant B1 of a mammalian MPV as defined above. In certain embodiments, a nucleic acid of the invention encodes a G protein, a N protein, a P protein, a M protein, a F protein, a M2-1 protein, a M2-2 protein, a SH protein, or a L protein of variant B2 of a mammalian MPV as defined above.

In certain embodiments, the invention provides a nucleotide sequence that is at least 50%, at least 55%, at least 60%, at least 65%, at least 70%, at least 75%, at least 75%, at least 80%, at least 85%, at least 90%, at least 95%, at least 98%, at least 99%, or at least 99.5% identical to the nucleotide sequence of SEQ ID NO:18, SEQ ID NO:19, SEQ ID NO:20, or SEQ ID NO:21. In certain embodiments, the nucleic acid sequence of the invention, is at least 50%, at least 55%, at least 60%, at least 65%, at least 70%, at least 75%, at least 75%, at least 80%, at least 85%, at least 90%, at least 95%, at least 98%, at least 99%, or at least 99.5% identical to a fragment of the nucleotide sequence of SEQ ID NO:18, SEQ ID NO:19, SEQ ID NO:20, or SEQ ID NO:21, wherein the fragment is at least 25 nucleotides, at least 50 nucleotides, at least 75 nucleotides, at least 100 nucleotides, at least 150 nucleotides, at least 200 nucleotides, at least 250 nucleotides, at least 300 nucleotides, at least 400 nucleotides, at least 500 nucleotides, at least 750 nucleotides, at least 1,000 nucleotides, at least 1,250 nucleotides, at least 1,500 nucleotides, at least 1,750 nucleotides, at least 2,000 nucleotides, at least 2,00 nucleotides, at least 3,000 nucleotides, at least 4,000 nucleotides, at least 5,000 nucleotides, at least 7,500 nucleotides, at least 10,000 nucleotides, at least 12,500 nucleotides, or at least 15,000 nucleotides in length. In a specific embodiment, the nucleic acid sequence of the invention is at least 50%, at least 55%, at least 60%, at least 65%, at least 70%, at least 75%, at least 75%, at least 80%, at least 85%, at least 90%, at least 95%, at least 98%, at least 99%, or at least 99.5% or 100% identical to one of the nucleotide sequences of SEQ ID NO:84-118; SEQ ID NO:154-233; SEQ ID NO:318-321; SEQ ID NO:326-329; SEQ ID NO:334-337; SEQ ID NO:342-345; SEQ ID NO:350-353; SEQ ID NO:354-357; SEQ ID NO:362-365; SEQ ID NO:370-373; SEQ ID NO:378-381; or SEQ ID NO:386-389.

In specific embodiments of the invention, a nucleic acid sequence of the invention is capable of hybridizing under low stringency, medium stringency or high stringency conditions to one of the nucleic acid sequences of SEQ ID NO:18, SEQ ID NO:19, SEQ ID NO:20, or SEQ ID NO:21. In specific embodiments of the invention, a nucleic acid sequence of the invention is capable of hybridizing under low stringency, medium stringency or high stringency conditions to one of the nucleic acid sequences of SEQ ID NO:84-118; SEQ ID NO:154-233; SEQ ID NO:318-321; SEQ ID NO:326-329; SEQ ID NO:334-337; SEQ ID NO:342-345; SEQ ID NO:350-353; SEQ ID NO:354-357; SEQ ID NO:362-365; SEQ ID NO:370-373; SEQ ID NO:378-381; or SEQ ID NO:386-389. In certain embodiments, a nucleic acid hybridizes over a length of at least 25 nucleotides, at least 50 nucleotides, at least 75 nucleotides, at least 100 nucleotides, at least 150 nucleotides, at least 200 nucleotides, at least 250 nucleotides, at least 300 nucleotides, at least 400 nucleotides, at least 500 nucleotides, at least 750 nucleotides, at least 1,000 nucleotides, at least 1,250 nucleotides, at least 1,500 nucleotides, at least 1,750 nucleotides, at least 2,000 nucleotides, at least 2,00 nucleotides, at least 3,000 nucleotides, at least 4,000 nucleotides, at least 5,000 nucleotides, at least 7,500 nucleotides, at least 10,000 nucleotides, at least 12,500 nucleotides, or at least 15,000 nucleotides with the nucleotide sequence of SEQ ID NO:18, SEQ ID NO:19, SEQ ID NO:20, or SEQ ID NO:21.

The invention further provides antibodies and antigen-binding fragments that bind specifically to a protein of a mammalian MPV. An antibody of the invention binds specifically to a G protein, a N protein, a P protein, a M protein, a F protein, a M2-1 protein, a M2-2 protein, a SH protein, or a L protein of a mammalian MPV. In specific embodiments, the antibody is a human antibody or a humanized antibody. In certain embodiments, an antibody of the invention binds specifically to a G protein, a N protein, a P protein, a M protein, a F protein, a M2-1 protein, a M2-2 protein, a SH protein, or a L protein of a virus of subgroup A of a mammalian MPV. In certain other embodiments, an antibody of the invention binds specifically to a G protein, a N protein, a P protein, a M protein, a F protein, a M2-1 protein, a M2-2 protein, a SH protein, or a L protein of a virus of subgroup B of a mammalian MPV. In certain, more specific, embodiments, an antibody of the invention binds specifically to a G protein, a N protein, a P protein, a M protein, a F protein, a M2-1 protein, a M2-2 protein, a SH protein, or a L protein of a virus of variant A1 of a mammalian MPV. In other embodiments, the antibody of the invention binds specifically to a G protein, a N protein, a P protein, a M protein, a F protein, a M2-1 protein, a M2-2 protein, a SH protein, or a L protein of a virus of subgroup A2 of a mammalian MPV. In certain embodiments, an antibody of the invention binds specifically to a G protein, a N protein, a P protein, a M protein, a F protein, a M2-1 protein, a M2-2 protein, a SH protein, or a L protein of a virus of subgroup B1 of a mammalian MPV. In certain other embodiments, an antibody of the invention binds specifically to a G protein, a N protein, a P protein, a M protein, a F protein, a M2-1 protein, a M2-2 protein, a SH protein, or a L protein of a virus of subgroup B2 of a mammalian MPV.

### 5.16 Inhibition of Virus Cell Fusion Using Heptad Repeats

Virus-host cell fusion is a necessary step in the infectious life cycle of many enveloped viruses, including MPV. As such, the inhibition of virus cell fusion represents a new approach toward the control of these viruses. This method of inhibition represents an alternative means of preventing the propagation of MPV in a host and the infection by MPV of a host. The inhibition of virus-cell fusion is dependent upon the type of attachment protein required. Wang et al., Biochem Biophys Res Comm 302 (2003) 469-475. Consequently, in one embodiment of the invention, an assay is used to identify the dependency of virus cell fusion on various attachment proteins.

In certain embodiments, the invention provides methods for preventing, treating, or managing an hMPV infection in a subject, the method comprising administering a pharmaceutically effective amount of a heptad repeat (HR) peptide. In certain embodiments, a pharmaceutically effective amount reduces virus host cell fusion by at least 10%, at least 15%, at least 20%, at least 30%, at least 40%, at least 50%, at least 60%, at least 70%, at least 80%, at least 90%, at least 95%, at least 99%, at least 99.5%. In a specific embodiment, the HR is an HR of the virus that causes the infection in the subject. In a certain embodiment, the HR is that of an hMPV of the subtype A1. In a more specific embodiment, the FIR sequence is one of the HR sequences of the F protein of hMPV, designated HRA or HRB, where HRA is the heptad repeat sequence near the N terminus of the peptide and HRB is near the C terminus. In certain embodiments, the HR that is administered to treat, prevent, or manage hMPV infection in the subject is an HR of hMPV subtype of A1, B1, A2, or B2.

In certain embodiments, the HR is at least 50%, 60%, 70%, 80%, 90%, 95%, 98%, 99%, or at least 99.5% identical to a HR of the virus that causes the infection in the subject. In certain embodiments, a derivative of a HR can be used to prevent viral fusion. Such derivatives include, but are not limited to, HR peptides that have been substituted with non native amino acids, truncated so that stretches of amino acids are removed, or lengthened, so that single amino acids or stretches thereof have been added. In yet another embodiment, single HR peptides are used to treat, manage, or prevent hMPV infection. In an even further embodiment, a combination of HR peptides is administered to treat, manage, or prevent hMPV infection.

The tests set forth below can be used to determine the effectiveness of a HR in preventing the fusion of an hMPV with a cell and can thus be used to determine which HRs or analogs or derivatives thereof are best suited for treating, preventing, or managing and hMPV infection in a subject.

In another embodiment of the invention, soluble synthesized HR peptides are assayed to determine whether the peptides are able to prevent viral-cell fusion. Any HR sequence can be used to inhibit hMPV viral-cell fusion, including but not limited to, HR sequences against RSV, PIV, APV, and hMPV. In a preferred embodiment, the HR sequence is that of hMPV. In a more specific embodiment, the HR sequence is one of the HR sequences of the F protein of hMPV, designated HRA or HRB, where HRA is the heptad repeat sequence near the N terminus of the peptide and HRB is near the C terminus. In another embodiment of the invention, the HRA and HRB derived peptides that are used to inhibit hMPV viral-cell fusion, include, but are not limited to HRA and HRB peptides from RSV, APV, and PIV. In even another embodiment of the invention, derivatives of HRA and HRB peptides are used to inhibit hMPV viral-cell fusion. For example, derivatives that are made by mutation of at least one amino acid residue in an HRA or HRB peptide are used to inhibit hMPV viral-cell fusion. In another embodiment of the invention, derivatives are made by truncation or resection of specific regions of an HRA or HRB peptide. In yet even another embodiment, the HRA or HRB peptide that is used is lengthened with respect to the endogenous HR sequence. In an even further embodiment, groups of short peptides that consist of sequences of different regions of an HRA or HRB peptide are used to inhibit hMPV viral-cell fusion. In another embodiment of the invention, hMPV HRA and HRB derived peptides are used against homologous strains of hMPV or against heterologous strains of hMPV. In yet another embodiment of the invention, HRA and HRB peptides, or analogs or derivatives thereof, are used together to inhibit viral-cell fusion. In a more preferred embodiment, either an HRA or HRB peptide or analog or derivative thereof is used alone. In another embodiment, the derivative of an HRA or HRB peptide that is used is at least 90%, 80%, 70%, 60%, or 50% identical to the endogenous HR peptide.

In order to examine the ability of the heptad repeat sequences to inhibit viral fusion, heptad repeat peptides can be expressed and purified so that they may be tested for their viral fusion inhibition ability. Soluble heptad repeat peptides can be expressed and purified and subsequently used in an assay to compete with endogenous heptad repeats in order to test for the blocking of viral fusion. In one embodiment of the invention, synthetic recombinant DNAs may be prepared that encode the heptad repeat sequences of the F protein of hMPV, designated HRA and HRB respectively. In another embodiment of the invention, synthetic recombinant DNAs may be prepared that encode heptad repeat peptides that also contain sequence tags useful in facilitating purification. In a preferred embodiment of the invention, the tag that facilitates purification of the heptad repeat peptide does not interfere with its activity. In yet another embodiment of the invention, the tag is composed of a series of histidine residues, e.g., six consecutive histidines at one of the peptide's termini, and is referred to as a histidine tag. There are a number of different approaches that can be used to express and purify soluble HRA and HRB. First, DNA vectors encoding the HRA and HRB are prepared using methods known to one skilled in the art. The plasmids are subsequently transformed into an appropriate expression host cell, such as, e.g., E. coli strain BL21 (DE3), and the protein is expressed and purified using methods routine in the art. For example, expression of a gene encoding an HR peptide with a histidine tag can be induced from a pET vector using IPTG. Cells can then be lysed and the expressed peptide can be isolated after immobilization on a Ni-chelated Sepharose affinity column following elution with a counter charged species, for e.g., imidazole.

In order to determine the potential effectiveness of the expressed heptad repeat peptides in inhibiting viral fusion, an assay can be used to confirm the assembly of a complex between HR peptides. This method would be advantageous over cell based assays in that it would allow for cell-free screening of peptides in order to determine efficacy in viral fusion inhibition. In one embodiment of the invention, HR peptides are incubated simultaneously for a period of time sufficient to allow complex formation. In a more specific embodiment, the amount of time allowed for complex formation is 1 h at 28° C. Complex formation can be detected using any method known in the art, including but not limited to, chromatogaphy, UV-vis spectroscopy, NMR spectroscopy, X-ray crystallography, centrifugation, or electrophores is. In another specific embodiment of the invention, complex formation is detected using gel filtration methods coupled with electrophoresis in order to determine the molecular weight of the complex. In yet another embodiment of the invention, this complex formation assay is used to identify candidates that are useful in inhibiting viral fusion, e.g., the effectiveness of mutated HR peptides in the inhibition of viral fusion is determined. In yet even another embodiment of the invention, the effectiveness of derivatives of HR peptides in the inhibition of viral fusion is measured using this complex formation assay.

It is known that the heptad repeat segments of the peptides are helical in nature. For this reason, a number of methods can be used to determine whether expressed HR peptides form alpha helices in order to identify appropriate candidates for use in viral fusion inhibition. Such methods, include, but are not limited to, spectroscopy, X-ray crystallography, and microscopy. In one embodiment of the invention, CD (circular dichroism) spectroscopy is used to determine the structural features of the HR peptides.

A cell based assay can be used to determine the effectiveness of HR peptides in the inhibition of viral fusion. Any cell that can be infected with MPV can be used in the assay, including, but not limited to: tMK, Hep2, or Vero cells. In a specific embodiment, the type of cells that are used are Hep2 cells. Upon infection of a host cell with MPV, the cells are incubated with HR protein preparations and scored for fusion after incubation for an appropriate period of time. Cells are subsequently stained for synctium/polykaryon formation in order to determine whether viral-cell fusion was successful.

The present invention may be better understood by reference to the following non-limiting Examples, which are provided as exemplary of the invention. The following examples are presented in order to more fully illustrate the preferred embodiments of the invention. They should in no way be construed, however, as limiting the broad scope of the invention.

### 6. EXAMPLE

### A S101P Substitution in the Putative Cleavage Site of the Human Metapneumovirus Fusion (F) Protein is a Major Determinant for Trypsin-Independent Growth in Vero Cells

### Materials and Methods

Cells and viruses. Vero cells were maintained in minimal essential medium (MEM) (JHR Biosciences) supplemented with 10% fetal bovine serum (FBS) (Hyclone), 2 mM L-glutamine (Gibco BRL), nonessential amino acids (Gibco BRL) and 2% penicillin/streptomycin (Biowhittaker). BSR/T7 cells (kindly provided by Dr. K K Conzelmann) were maintained in Glasgow MEM (Gibco BRL) supplemented with 10% FBS, 5% tryptose phosphate broth (Sigma), nonessential amino acids, and 2% penicillin/streptomycin. tMK cells were maintained as previously described (van den Hoogen et al, 2001). hMPV and chimeric b/h PIV3 viruses were propagated in Vero cells with optiMEM (Gibco/BRL) and 2% penicillin/streptomycin. Some viruses were propagated with 0.2 ug/ml TPCK trypsin (Sigma). Virus stocks were harvested by scraping the cells and supernatant together with SPG (10×SPG is 2.18 M sucrose, 0.038 M KH₂PO₄, 0.072 M K₂HPO₄, 0.054 M L-Glutamate at pH 7.1) to a final concentration of 1×SPG and freezing at −70 C.

The virus isolates wt hMPV/NL/1/93, wt hMPV/NL/1/94, wt hMPV/NL/1/99 and wt hMPV/NL/1/00 were described previously (Hersft et al, 2004; van den Hoogen, 2001). The following recombinant viruses were generated by reverse genetics from full-length cDNA plasmids: rhMPV/NL/1/00/101P, rhMPV/NL/1/00/101S, rhMPV/NL/1/99/101S, rhMPV/93K/101S, rhMPV/93K/101P, b/h PIV3/hMPV F/101P and b/h PIV3/hMPV F/101S. The variant viruses vhMPV/93K/101P and vhMPV/100K/101P were derived from rhMPV/93K/101P.

Titer by immunostaining of hMPV plaques. Virus titers (plaque forming units (PFU)/ml) were determined by plaque assay in Vero cells. Vero cells were grown to near confluency in TC6-well plates. Following a 1 hr adsorption at 35° C. with virus diluted in optiMEM, the cells were overlaid with 2% methyl cellulose diluted 1:1 with optiMEM with 2% penicillin/streptomycin and incubated at 35° C. for 6 days. To prepare for immunostaining, the overlay was removed and the cells were fixed in methanol for 15 minutes. Plaques were immunostained with antisera to hMPV obtained from ferrets immunized with wt hMPV/NL/1/00 (MedImmune Vaccines, Inc.). The antisera were diluted approximately 1:500 in PBS containing 5% powdered milk (w/v) (PBS-milk). The cells were then incubated with horseradish peroxidase-conjugated anti-ferret Ab (Dako) followed by 3-amino-9-ethylcarbazole (AEC) (Dako) to visualize plaques for counting.

Construction of full-length hMPV cDNA plasmids. cDNAs of hMPV/NL/1/00 (containing 101S) and hMPV/NL/1/99 (containing 101S) were constructed as previously described and used to recover the recombinant viruses named rhMPV/NL/1/00/101S and rhMPV/NL/1/99/101S (Herfst et al 2004). The nucleotide substitution T3367C that encodes S 101P in the predicted amino acid sequence of hMPV F glycoprotein was introduced using the primer GCAAATTGAAAATCCCAGACAACCTAGATTCGTTCTAGG and its anti-sense primer in order to construct the plasmid used to recover recombinant virus rhMPV/NL/1/00/101P. The nucleotide substitution G3343A that encodes the predicted amino acid substitution E93K in hMPV F glycoprotein was likewise introduced with the primer GCTGATCAACTGGCAAGAGAGAAGCAAATTGAAAATCCC and its anti-sense primer.

Recovery of recombinant hMPV viruses by reverse genetics. Recombinant virus was recovered by reverse genetics as described previously (Herfst et al 2004). Briefly, 1.2 ug of pCITE hMPV N, 1.2 ug of pCITE hMPV P, 0.9 ug of pCITE hMPV M2, 0.6 ug pCITE hMPV L, and 5 ug of full-length cDNA plasmid in 500 uL optiMEM containing 10 uL lipofectamine 2000 (Invitrogen), was applied to a monolayer of 10⁶ BSR/T7 cells. The medium was replaced with optiMEM 15 h post transfection and incubated at 35° C. for 2 to 3 days. After one freeze thaw cycle, the cells and supernatant were used to infect a 90% confluent monolayer of Vero cells and incubated for 6 days to amplify rescued virus. Virus recovery was verified by positive immunostaining with ferret polyclonal Ab directed to hMPV as described. Recovered viruses were amplified in Vero cells by inoculating at a multiplicity of infection (MOI) of 0.1 PFU/cell, feeding with optiMEM and collecting after 6 days incubation at 35° C. Some transfections and growth were done in the presence of 0.2 ug/ml TPCK trypsin (Sigma) as described.

RT-PCR of recovered viruses. DNA for sequencing was produced by inoculating Vero cell monolayers with hMPV viruses at a MOI of 0.1 PFU/cell. Cells and supernatants were collected 6 days post inoculation and subjected to one freeze-thaw cycle. RNA was extracted using TRizol reagent according to the manufacturer's instructions. RT-PCR was done using one step RT-PCR kit (Invitrogen) and overlapping sets of primers. Chromatograms of RT-PCR fragments were generated from DNA isolated from agarose gels using a gel extraction kit (Qiagen gel extraction kit).

Multicycle growth of hMPV viruses in Vero cells. Subconfluent monolayers of Vero cells in TC6-well plates were inoculated at a MOI of 0.1 PFU/cell with hMPV virus diluted in optiMEM either in the absence or presence of 0.2 ug/ml TPCK trypsin (Sigma). The viral inoculum was aspirated and cells were fed with 2 ml per well of optiMEM ±0.2 ug/ml TPCK trypsin. Cells plus supernatant were collected at 24 h intervals for 6 days and frozen at −70° C. Collected samples were titered in Vero cells ±0.2 ug/ml TPCK trypsin. Plaques were visualized by immunostaining with ferret anti-hMPV polyclonal Ab (MedImmune Vaccines, Inc.) as described above.

Immunostaining for surface expression of hMPV F glycoprotein. Vero cells were seeded onto glass coverslips. Subconfluent monolayers of Vero cells were inoculated at a MOI of 5 PFU/cell. The viral inoculum was aspirated and the cells were fed with optiMEM containing 2% penicillin/streptomycin. Following incubation at 35° C. for 3 days, the cells were fixed in 3% paraformaldehyde for 10 minutes. The monolayers were then washed in PBS and blocked in PBS-milk. The cells were incubated for 1 hr at room temperature with anti-hMPV F monoclonal antibody (Mab) 121-1017-133 (unpublished) diluted 1:250 in PBS-milk followed by 2 washes in PBS. The cells were then incubated for 1 hr at room temperature with fluorescein isothiocyanate (FITC)-conjugated anti-Armenian hamster Ab (Jackson Laboratories) diluted 1:1000 in PBS-milk followed by 2 washes in PBS. The inverted coverslips were mounted onto glass slides using 10 uL Vecta-shield mounting medium (Vector Laboratories) and viewed with a Nikon eclipse TE2000-U microscope.

Western blot of hMPV F protein. hMPV viruses were used to infect subconfluent monolayers of Vero cells in TC6-well tissue culture dishes at a MOI of 0.1 PFU/cell and incubated at 35° C. 4 to 6 days post-infection, cells and supernatant were collected and frozen at −70° C. Samples were thawed, lysed in Laemmli buffer (Bio-Rad) containing 5% beta-mercaptoethanol (Sigma), separated in a 12% polyacrylamide Tris-HCl Ready Gel (Bio-Rad), and transferred to a Hybond-P PVDF membrane (Amersham Biosciences) using a wet transfer cell (Bio-Rad). Membranes were blocked with PBS containing 5% (w/v) dry milk (PBS-milk), incubated with anti-hMPV F Mab 121-1017-133 diluted 1:2000 in PBS-milk, followed by incubation with horseradish peroxidase-conjugated anti-hamster Mab diluted 1:1000 in PBS-milk. Membranes were washed four times with PBS containing 0.5% (v/v) Tween 20 (Sigma), developed with a chemiluminescence substrate (Amersham Biosciences), and exposed to Biomax MR film (Kodak) for visualization of hMPV F protein.

b/h PIV3/hMPV F2 full length cDNA. b/h PIV3/hMPV F2 (expressing hMPV F containing 101S) was previously described (Tang et al 2003). Briefly, the hMPV F gene was inserted between the N and P genes of a chimeric bovine/human parainfluenza virus type 3 (b/h PIV3) cDNA (Haller et al 2000; Haller et al 2001). The nucleotide change corresponding to T3367C in the hMPV/NL/1/00 genome was introduced in the hMPV F gene of b/h PIV3/hMPV F2 using a Quik change mutagenesis kit (Stratagene) resulting in b/h PIV3/hMPV/ F2/101P that expresses hMPV F with proline at amino acid 101.

Quantitation of fused nuclei in Vero cells. Monolayers of confluent Vero cells in TC6-well plates were inoculated, in duplicate, at a MOI of 3 PFU/cell or mock infected. Following 1 hr incubation at 35° C., the inoculum was aspirated and the cells were overlaid with 2% methyl cellulose mixed 1:1 with optiMEM containing 2% penicillin/streptomycin ±0.2 ug/ml TPCK trypsin (Sigma). At 48 h or 72 h, the media was aspirated and the monolayers were fixed with methanol for 15 minutes. The fixed monolayers were washed with PBS, incubated for 1 h with Hoechst stain solution (0.25 ug/ml of bisbenzimide H 33258 (Sigma) in PBS) and examined by a Nikon eclipse TE2000-U microscope equipped with DAPI lens. Fused and unfused nuclei in 10 randomly selected fields of view (totaling more than 2000 nuclei) were counted and the percent of fused nuclei was calculated.

### Results

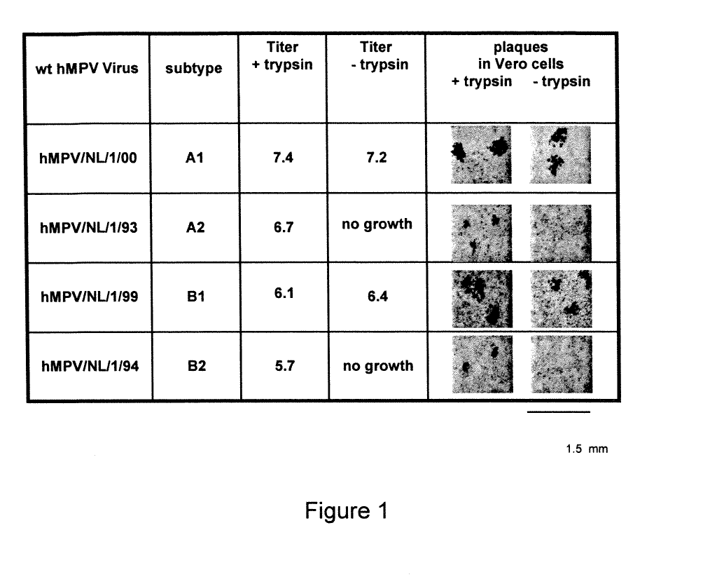

Trypsin requirement for growth in Vero cells varies among the 4 representative subtypes of wt hMPV. Biologically derived strains of hMPV virus representing all 4 subtypes A1, A2, B1 and B2 were grown in Vero cells. wt hMPV/NL/1/00 and wt hMPV/NL/1/99, representative of subtypes A1 and B1, respectively, grew to peak titers of 10⁶ to 10⁷ PFU/ml in the absence as well as the presence of trypsin. The plaque size, as visualized by immunostaining, was roughly 0.3 to 0.5 mm in diameter after 6 days of growth in Vero cells under 1% methylcellulose (FIG. 1).

In marked contrast, wt hMPV/NL/1/93 and wt hMPV/NL/1/94, representative of subtypes A2 and B2, respectively, grew only when trypsin was present in the media. wt hMPV/NL/1/93 grew to peak titers between 10⁶ and 10⁷ PFU/ml while titers of wt hMPV/NL/1/94 were one log lower. In addition, no plaques were produced when trypsin was not present in the media overlay. The diameters of plaques produced in the presence of trypsin by wt hMPV/NL/1/93 and wt hMPV/NL/1/94 were markedly smaller than plaques produced by wt hMPV/NL/1/00 or wt hMPV/NL/1/99 with or without trypsin (FIG. 1).

The published sequences of the F glycoproteins of all 4 hMPV subtypes predict a RQSR motif at the putative cleavage site. Sequencing of the F gene confirmed that wt hMPV/NL/1/93 and wt hMPV/NL/1/94 (subtypes A2 and B2, respectively) have the predicted RQSR sequence as expected. However, the sequences of wt hMPV/NL/1/00 and wt hMPV/NL/1/99 (subtypes A1 and B1, respectively) acquired a T3367C change that results in a predicted S101P amino acid substitution in F protein so that the putative cleavage site is RQPR. The effect of S101P substitution on trypsin-independent growth of hMPV was further characterized.

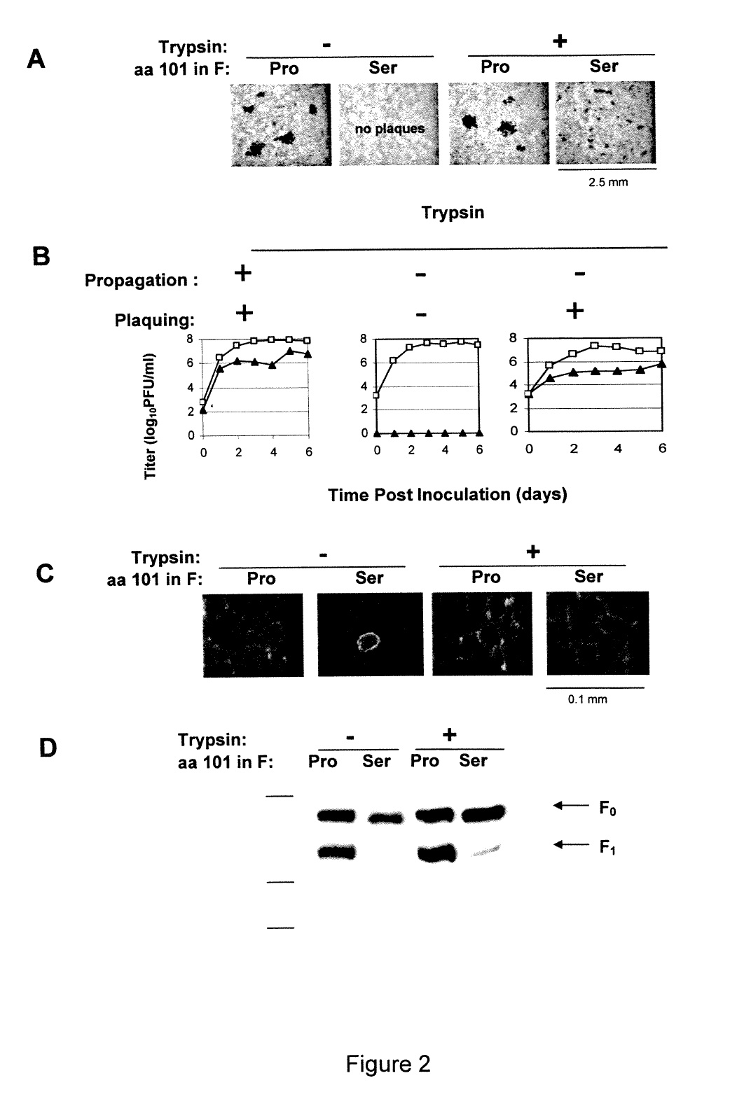

rhMPV/NL/1/00/101P, but not rhMPV/NL/1/00/101S, can be recovered from cDNA without trypsin. To investigate the effect of the S101P amino acid substitution in hMPV F on trypsin-independent growth of hMPV/NL/1/00, we introduced a T at nt 3367 to generate rhMPV/NL/1/00/101S or a C at nt 3367 to generate rhMPV/NL/1/00/101P. rhMPV/NL/1/00/101P was readily recovered in the absence of trypsin and formed plaques comparable to wt hMPV/NL/1/00. In marked contrast, rhMPV/NL/1/00/101S was recovered only in the presence of trypsin and formed plaques significantly smaller than plaques of rhMPV/NL/1/00/101P (FIG. 2A).

Comparison of rhMPV/NL/1/00/101S and rhMPV/NL/1/00/101P replication in Vero cells. To characterize the trypsin-independent growth of recombinant hMPV/NL/1/00 viruses harboring either 101S or 101P in the F protein, multi-cycle growth curves were performed in the presence or absence of trypsin.

Quantification of infectious virus at each time point was carried out by plaque assays either in the presence or absence of trypsin (FIG. 2B). In the presence of trypsin, both rhMPV/NL/1/00/101S and rhMPV/NL/1/00/101P demonstrated efficient multicycle growth. rhMPV/NL/1/00/101P reached a peak titer of 7.8 log₁₀ PFU/ml on day 3 while rhMPV/NL/1/00/101S achieved a peak titer of 7 log₁₀ PFU/ml on day 5 (FIG. 2B).

In the absence of trypsin, only rhMPV/NL/1/00/101P underwent multicycle growth, reaching a peak titer of 7.6 log₁₀ on day 3, similar to growth in the presence of trypsin. No rhMPV/NL/1/00/101S was detected when trypsin was omitted in the plaque assay (FIG. 2B).

However, single cycle growth of rhMPV/NL/1/00/101S appeared to have occurred in the absence of trypsin because viruses collected during growth without trypsin formed infectious foci upon the addition of trypsin in the plaque assay. This suggested that virus particles of rhMPV/NL/1/00/101S were generated during replication without trypsin, however, they were not infectious unless trypsin was in the media. The peak titer of rhMPV/NL/1/00/101S propagated without trypsin was about 2 log₁₀ lower relative to rhMPV/NL/1/00/101P (FIG. 2B).

Effect of S101P on surface expression of hMPV F protein in rhMPV-infected cells. Paramyxovirus fusion proteins are transported to the plasma membrane where they promote membrane fusion. To determine whether the poor growth of rhMPV/NL/1/00/101S is caused by impaired cell surface expression of hMPV F, Vero cells were inoculated at a MOI of 5 PFU/cell and fixed for immunostaining 3 days post inoculation. hMPV F was detected in nearly 100% of the cells inoculated with rhMPV/NL/1/00/101P both with and without trypsin. Similar levels of expression of hMPV F was observed in the Vero cells inoculated with rhMPV/NL/1/00/101S in the presence of trypsin (FIG. 2C).

In contrast, surface expression of F protein was detected on the plasma membranes of only a few individual cells in the monolayer infected with rhMPV/NL/1/00/101S without trypsin (FIG. 2C). This suggested that, without trypsin, hMPV F/101S was indeed expressed on the plasma membrane but resulted in inefficient rhMPV/NL/1/00/101S infection that did not spread to adjacent cells. The inability of hMPV F/101S to promote vigorous spread of rhMPV/NL/1/00/101S infection in the absence of trypsin can be partly attributed to the failure to produce infectious virus particles. However, efficient cleavage of the fusion protein precursor is also required for cell-to-cell fusion and spread of virus infection.

Cleavage of hMPV F protein of rhMPV/NL/1/00/101S compared to rhMPV/NL/1/00/101P. Without being limited by theory, cleavage of the F₀ precursor into the F₁ and F₂ fragments exposes the fusion peptide at the N terminus of the F1 fragment that is required for fusion activity and multi-cycle virus growth. In order to demonstrate the effect of the S101P substitution on the efficiency of F cleavage, Vero cells were inoculated at a MOI of 0.1 PFU/cell either with or without trypsin. Cells and supernatant were harvested 5 days post infection and analyzed by Western blot to visualize relative cleavage of hMPV F.

For F protein containing 101P, approximately half the amount of the full-length hMPV F protein (F₀) was cleaved to form an F species that corresponds to the predicted size of the putative F₁ fragment. The efficiency of processing for F protein containing 101P is comparable with or without trypsin (FIG. 2D).

In contrast, hMPV F containing 101S was cleaved only when the protein was exposed to trypsin. The relative efficiency of cleavage was significantly less compared to hMPV F/101P (FIG. 2D). The relative amount of cleavage of F protein containing 101S with and without trypsin was found to variable between experiments due to differences in the specific activity of trypsin added. However, the relative cleavage of hMPV F/101S was reproducibly less than for hMPV F/101P.

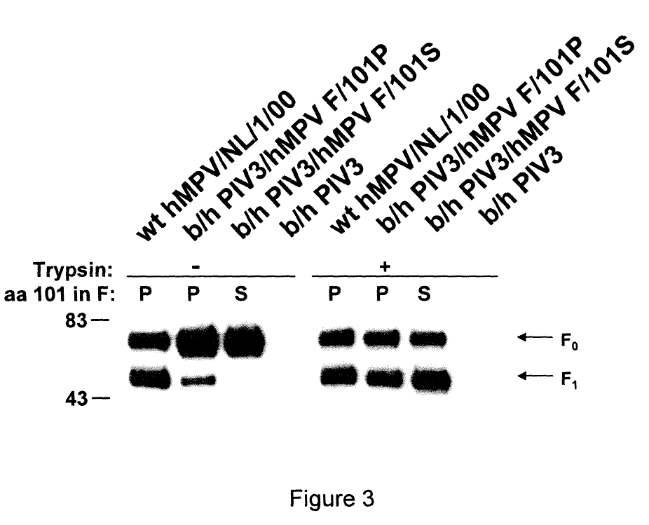

Cleavage of F of hMPV/101S compared to hMPV/101P when expressed from b/h PIV3 viral vector. To determine whether hMPV F cleavage was dependent upon the native viral context provided by other hMPV viral proteins, hMPV F protein harboring either a predicted 101S or 101P was cloned into b/h PIV3, a bovine PIV3 virus in which the F and HN genes have been replaced with the human PIV3 F and HN genes. Previous studies showed that b/h PIV3 accommodated insertion of various paramyxovirus fusion glycoproteins (Skiadopoulos et al 2002; Tang et al, 2003, 2004a and 2004b). Without exogenously added trypsin, vectored hMPV/NL/1/00/101P F protein was partially cleaved in Vero cells while hMPV/NL/1/00/101S F protein was uncleaved as determined by Western blot of infected cell lysates (FIG. 3). However, the degree of cleavage of vectored hMPVF/101P protein was reduced compared to cleavage of F of hMPV F/101P in hMPV-infected cells (compare FIGS. 70D and 71). This difference was no longer apparent when trypsin was added. In the presence of trypsin, the vectored F proteins of both hMPV/NL/1/00/101P and hMPV/NL/1/00/101S were partially cleaved to the same extent as the F protein expressed from the wt hMPV/NL/1/00 (FIG. 3).

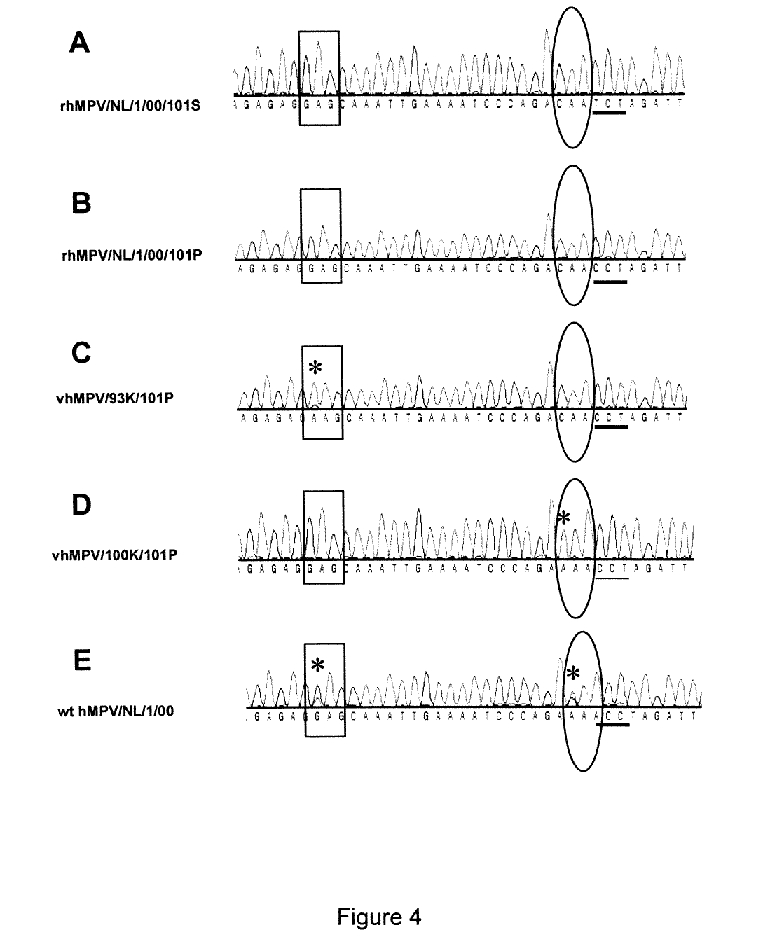

Spontaneous hMPV F variants of hMPV/NL/1/00. rhMPV/NL/1/00/101P rapidly developed other codon changes in or upstream of the RQPR motif at the putative cleavage site of the fusion protein. One stock of rhMPV/NL/1/00/101P spontaneously developed the mutation G3343A encoding a predicted E93K amino acid substitution in F (boxed codon of FIG. 4C). A second stock developed the mutation C3364A encoding a predicted Q100K substitution in F (circled codon in FIG. 4D). These mutations remained genetically stable for 10 additional passages in Vero cells. During these passages, no other mutations were detected in the F protein. One of these variant viruses, vhMPV/93K/101P, was sequenced in its entirety (excluding 30 nucleotides at the extreme 3’ and 5′ ends of the genome) and G3343A was the only mutation detected. No other mutations were found in the other hMPV ORFs or non-coding regions, suggesting that replication of the hMPV genome by the polymerase complex was not inherently error-prone.

Among independently rescued stocks of rhMPV/NL/1/00/101P a polymorphism at G3343A was the most frequently observed. 5 other polymorphisms at nucleotides upstream of the putative cleavage site were also found in 5 different virus stocks of rhMPV/NL/1/00/101P, albeit with less frequency than G3343A. These were G3340A, A3344T, T3350G, G3352A and A3355C that would encode predicted amino acid substitutions E92K, E93V, I95S, E96K and N97H (Table 20a and 20b). Each virus stock of rhMPV/NL/1/00/101P that developed one of these polymorphisms presented with only one, never two or more of these additional mutations and it arose in less than 6 passages in cell culture. Thus, any of these additional mutations individually provides a growth advantage in Vero cells.

Table 20a and 20b: Mutations and polymorphisms in hMPV F gene of rhMPV/NL/1/00/101P, wt hMPV/NL/1/00 and wt hMPV/NL/1/99. Stocks of the indicated hMPV viruses developed polymorphisms in the F gene in less than 6 passages in Vero cells. The mutations and consequent predicted amino acid substitutions in hMPV F protein are indicated above each column

TABLE 20a

| | | E92K | E93K | E93V | Q94K | Q94H |
|---|---|---|---|---|---|---|
| Virus | Trypsin | G3340A | G3343A | A3344T | C3346A | A3348C |
| rhMPV/ | − | | X | X | | |
| NL/1/00/ | + | X | X | | | |
| 101P | | | | | | |
| wt hMPV/ | − | | X | X | | X |
| NL/1/00 | | | | | | |
| wt hMPV | − | | | | X | |
| /NL/1/99 | | | | | | |

TABLE 20b

| | I95S | E96K | N97H | N97K | Q100K | S101P |
|---|---|---|---|---|---|---|
| Virus | T3350G | G3352A | A3355C | T3357A | C3364A | T3367C |
| rhMPV/ | X | X | X | | X | X |
| NL/1/ | | | | | | X |
| 00/101P | | | | | | |
| wt hMPV/ | | | | X | X | X |
| NL/1/00 | | | | | | |
| wt hMPV | | X | | | | X |
| /NL/1/99 | | | | | | |

To demonstrate that growth without trypsin provided the selective pressure for the spontaneous mutations to occur in rhMPV/NL/1/00/101P, 10 independent transfections using the same full-length cDNA clone were done with trypsin and 10 were done without trypsin. Recovery of virus was equally efficient with or without trypsin. However, after the third passage without trypsin, 7 out of 10 virus stocks had developed a subpopulation with a G3343A or C3364A mutation, while only 1 out of 10 virus stocks grown with trypsin had developed a mutation and it was G3343A.

Similarly, for rhMPV/NL/1/00/101S, 10 independent transfections using the same full-length cDNA clone were done with trypsin and 10 without trypsin. No virus was recovered in the absence of trypsin. Sequencing of RT-PCR fragments from 10 independently rescued rhMPV/NL/1/00/101S stocks that were recovered and amplified with trypsin showed no mutations in the F gene even after 10 serial passages.

These data show that the G3343A or C3364A variants of rhMPV/NL/1/00/101P arose rapidly in the absence of trypsin to facilitate more efficient cleavage of the fusion protein in the absence of trypsin. In the presence of trypsin, the function of hMPV F cleavage was assumed by the exogenous protease obviating the selection of cleavage-enhancing mutations.

Nucleotide polymorphisms in the fusion gene of wt hMPV/NL/1/00 and wt hMPV/NL/1/99 were investigated. wt hMPV/NL/1/00 virus stock was derived from 3 passages in tertiary monkey kidney cells and further passaged 3 times in Vero cells (“P6”). The entire genome of this P6 virus stock had previously been subjected to sequence analyses and shown to have a proline at position 101 (underlined codon in FIG. 4E). On close examination of the chromatogram, polymorphisms at nucleotides 3343 and 3364 in the F gene were revealed (boxed and circled codons in FIG. 4E). Clonal analysis was performed using RT-PCR fragments spanning nt 3200 to nt 3500 derived from a P6 stock of wt hMPV/NL/1/00. Of the 20 clones analyzed, 9 had the C3364A mutation (Q100K) and 4 had the G3343A mutation (E93K). These 2 mutations were identical to the predominant mutations found in rhMPV/NL/1/00. Of the remaining clones, 3 had A3344T, 1 had A3348C, and 1 had T3357A encoding E93V, Q94H, and N97K, respectively (Table 20). No clone contained more than one of these mutations. Attempts to isolate plaques of wt hMPV/NL/1/00 were not successful due to the poor cytopathic effects of hMPV infections. These results show that wt hMPV/NL/1/00 expanded to P6 was a mixed population that contained two predominant quasispecies. Thus, both biologically derived and recombinant hMPV readily acquired mutations in the hMPV F gene that facilitated their growth in tissue culture.

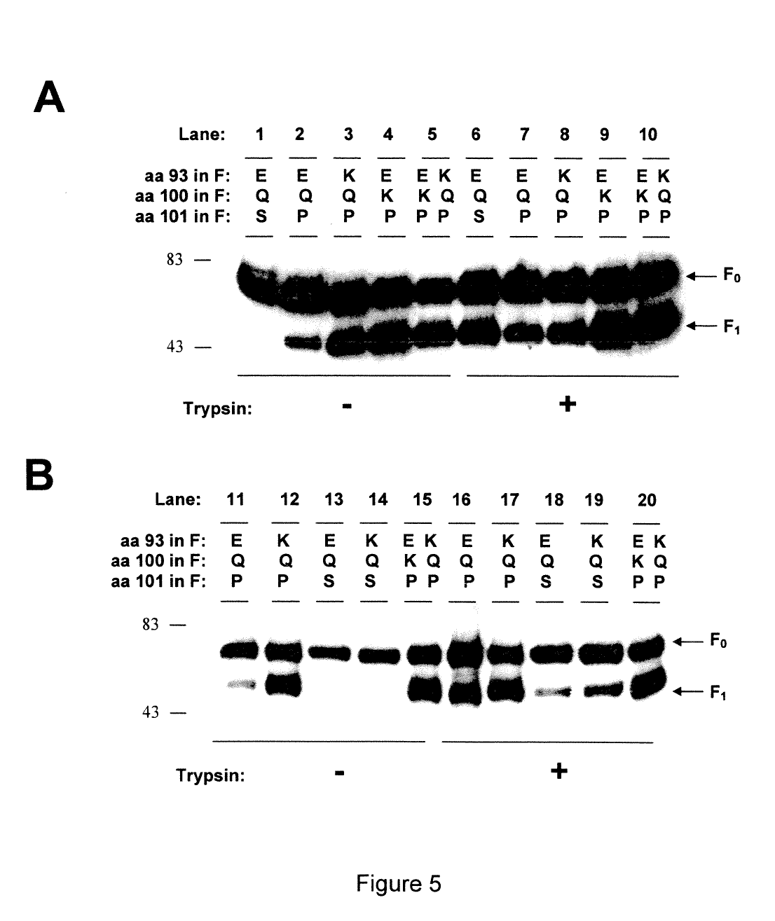

Effects of E93K and Q100K on the cleavage of hMPV F. To determine the effects of E93K and Q100K on the efficiency of hMPV F cleavage, Vero cells were inoculated with the wild-type, recombinant and variant hMPV/NL/1/00 viruses with or without trypsin. Cells and supernatants were harvested 6 days post infection and analyzed by Western blot to visualize relative cleavage of hMPV F protein (FIG. 5A). Without trypsin, the cleavage of F protein with 101P was noticeably more efficient in variant viruses with either an E93K or Q100K amino acid substitution compared to the fusion protein with only the 101P substitution (compare lanes 3, 4 and 5 to lane 2 of FIG. 5A). In the presence of trypsin, the relative cleavage of wild type and mutant hMPV F proteins was comparable (lanes 6 through 10 of FIG. 5A). Trypsin did not further increase the cleavage efficiency of hMPV F containing the cleavage-enhancing E93K or Q100K amino acid substitutions.

E93K alone is not sufficient to confer trypsin-independent cleavage of hMPV F. E93K was the most frequently observed mutation in recombinant hMPV/NL/1/00/101P and the variant F protein containing E93K resulted in enhanced cleavage activity.

The nucleotide change G3343A was introduced into each of the full-length cDNAs hMPV/NL/1/00/101S and hMPV/NL/1/00/101P. The recombinant viruses rhMPV/93K/101P and rhMPV/93K/101S were recovered using reverse genetics and their genotypes were shown to be stable for up to 10 passages in Vero cells. Western blot analysis showed that in the absence of trypsin, the F proteins of viruses with 101P were partially cleaved whereas F proteins with 101S were not cleaved (FIG. 5B). The presence of the E93K greatly enhanced the efficiency of hMPV F/101P cleavage (lanes 11 and 12, FIG. 5B). However, E93K did not increase the cleavage of hMPV F/101S (lanes 13 and 14, FIG. 5B). Therefore, the E93K substitution increased the efficiency of hMPV F cleavage only when proline was present at position 101, demonstrating a synergistic effect between 101P and 93K on hMPV F protein processing.

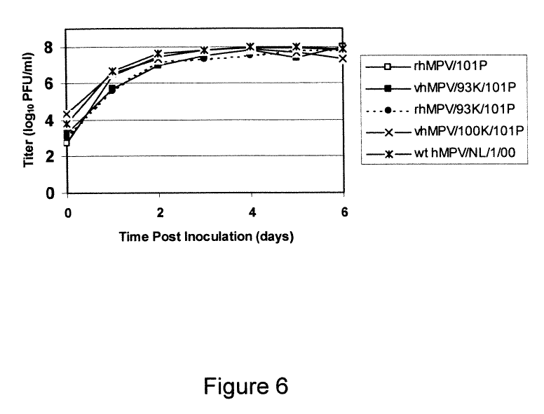

Effect of E93K and Q100K on growth kinetics in Vero cells. To determine the effect of enhanced trypsin-independent cleavage of F protein on multi-cycle growth of hMPV in Vero cells, rhMPV/NL/1/00/101P, vhMPV/93K/101P, rhMPV/93K/101P, vhMPV/100K/101P or wt hMPV/NL/1/00 were used to infect cells at a MOI of 0.1 PFU/cell without trypsin. Virus titers were obtained in the absence of trypsin. The growth curves for each of the trypsin-independent viruses that contain S101P were comparable, indicating that there is no enhancement in the viral peak titers or growth kinetics with increased cleavage efficiency of the hMPV F that resulted from acquisition of E93K or Q100K (FIG. 6).

Enhanced hMPV F cleavage correlates with increased fusion activity in hMPV-infected Vero cells. Analogous to other paramyxoviruses, cleavage of full-length hMPV F protein (F₀) into two fragments, F₁ and F₂, may have exposed a fusion peptide at the N-terminus of the F₁ fragment that can promote fusion between cells (Morrison 2003; White, 1990). Visual inspection of wt hMPV/NL/1/00-infected Vero cell monolayers showed that by day 2 to 3 most of the cells had fused to form many large syncytia, whereas rhMPV/NL/1/00/101S-infected cells showed fewer and smaller syncytia.

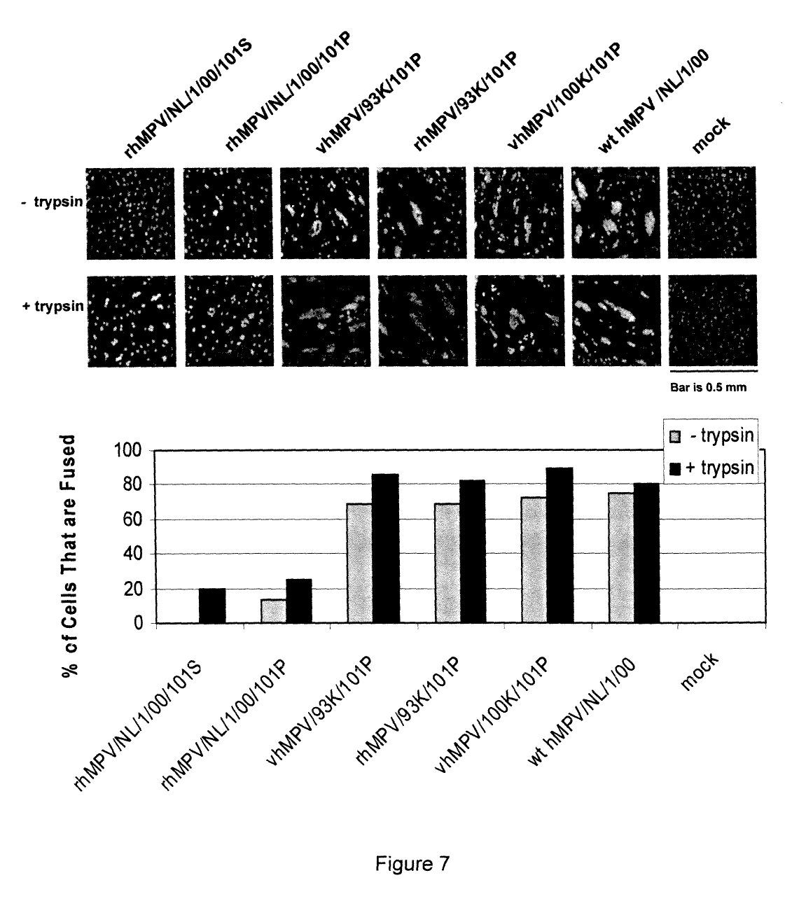

To demonstrate that an increase in cell-to-cell fusion activity correlated with enhanced cleavage of F protein, confluent monolayers of Vero cells were inoculated with wild type, recombinant and variant hMPV/NL/1/00 viruses with or without trypsin. Fusion activity of wild type and variant viruses was quantified by counting fused and unfused nuclei in 10 randomly selected fields of view. By 48 hours, giant syncytia were visible in the Vero cell monolayers infected with vhMPV/93K/101P, rhMPV/93K/101P, vhMPV/100K/101P or wt hMPV/NL/1/00. When allowed to progress, by 80 hours, the multi-nucleated syncytia covered 100% of the monolayers infected with these viruses. To count fused and unfused nuclei, the cells were fixed at 48 hours when the fusion was less than 100% (FIG. 7). For one representative experiment, without trypsin, 65-75% of the Vero cells infected with vhMPV/93K/101P, rhMPV/93K/101P, vhMPV/100K/101P or wt hMPV/NL/1/00 showed fused nuclei, and, with trypsin, 80% and 90% of the cells were fused (FIG. 7). For rhMPV/NL/1/00/101P that did not contain hMPV F cleavage-enhancing mutations, syncytia formation was considerably reduced; the percent of fused nuclei was 13% without trypsin and 25% with trypsin. For rhMPV/NL/1/00/101S, formation of small syncytia was only observed in the presence of trypsin, with 20% of nuclei fused (FIG. 7). The data shown in FIG. 7 is representative of one of three independently performed experiments. Since enhancement of hMPV F cleavage did not increase the replication efficiency of hMPV, there is a direct correlation between efficiency of hMPV F cleavage and the fusion activity that gave rise to syncytia formation.

Characterization of subtype B1 hMPV/NL/1/99 with S101P substitution in the RQSR cleavage motif of F protein. hMPV/NL/1/00 used in the above experiments is of the A1 subtype. Biologically derived wt hMPV/NL/1/99, a representative B1 subtype, also was found to have the S101P substitution in the predicted RQSR cleavage site of its F protein. The growth of hMPV/NL/1/99 compared to hMPV/NL/1/00 was previously described (Herfst et al, 2003).

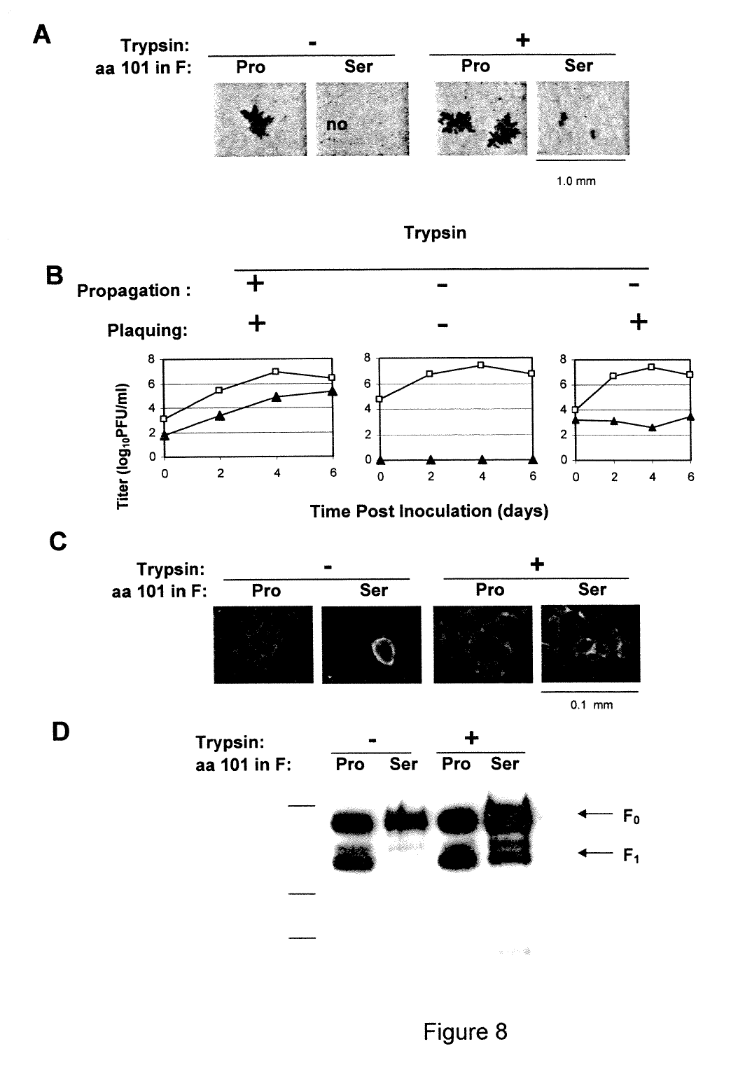

Growth characteristics of rhMPV/NL/1/99/101S were compared to wt hMPV/NL/1/99. Like rhMPV/NL/1/00/101S, rhMPV/NL/1/99/101S also required exogenously added trypsin for plaque formation, multicycle growth, cell-to-cell spread and cleavage of the F protein in Vero cells (FIG. 8A to D). In contrast, wt hMPV/NL/1/99 (that has 101P) grew efficiently without trypsin. Even in the presence of trypsin, the peak titer of rhMPV/NL/1/99/101S was approximately 2 log₁₀ lower than the peak titer displayed by wt hMPV/NL/1/99 (FIGS. 76B). Western blot of subtype B1 hMPV F also showed that the S101P substitution resulted in greater cleavage without addition of exogenous trypsin. hMPV F/101S showed no cleavage in the absence of trypsin, but in the presence of trypsin, the F1 fragment was readily detected. In addition, a small band migrated below the 31 kDa marker (likely a product of trypsin cleavage) was also recognized by the Mab directed to hMPV F (FIG. 8D). Sequencing of RT-PCR fragments derived from the F gene of wt hMPV/NL/1/99 indicated two nucleotide polymorphisms, C3346A and G3352A, encoding predicted Q94K and E96K amino acid substitutions in F, respectively.

Therefore, the S101P in the RQSR motif at the cleavage site of both subtype A1 and B1 fusion proteins alters the protease specificity resulting in efficient hMPV growth in the absence of trypsin.

Discussion

hMPV has been reported to require trypsin for growth (Bastien et al 2003a and 2003b, Biacchesi et al, 2003; Boivin et al, 2002; Hamelin et al, 2004; Peret et al, 2002 and 2004; Skiadolopous et al 2004; van den Hoogen et al 2001 and 2004b). However it was observed that hMPV/NL/1/00 (subtype A1) and hMPV/NL/1/99 (subtype B1) passaged 3 times in tertiary monkey cells and 3 times in Vero cells (strains “P6”) exhibited comparable growth kinetics and peak titers in the presence or absence of trypsin. For a different paramyxovirus, Sendai virus, it has been demonstrated that mutations that altered the processing site of the fusion protein precursor (F₀) significantly affected the trypsin requirement for virus growth (Ishida and M. Homma 1978.; Kido et al, 1992; Tashiro and M. Homma, 1983: Tashiro, M. et al 1988 and 1992).

To demonstrate the genetic basis for trypsin-independent growth of hMPV/NL/1/00 and hMPV/NL/1/99, sequencing was performed on the hMPV fusion gene to identify amino acid changes (van den Hoogen, 2001, 2002). Several nucleotides near and one nucleotide in the RQSR motif at the putative F₁/F₂ cleavage site were found to display nucleotide polymorphisms. One of these nucleotide changes encoded an S to P substitution in the RQSR motif at position 101. By analogy with other paramyxovirus fusion proteins, cleavage at the RQS/PR motif likely exposed the fusion domain located at the N-terminus of the F₁ fragment that is required for fusion with host cell membrane, syncytia formation and efficient virus amplification (Morrison, T. 2003; Scheid and Choppin 1974 and 1977).

To investigate the role of S101P substitution in trypsin-independent growth in Vero cells, recombinant hMPV/NL/1/00 viruses were generated that contained serine or proline at position 101 in the RQSR motif. It was found that hMPV that expressed fusion protein with 101S was incapable of initiating multi-cycle growth without the addition of trypsin in marked contrast to rhMPV/NL/1/00/101P. rhMPV/NL/1/00/101P showed comparable growth kinetics and mean peak titers with or without exogeneous trypsin and this correlated with comparable hMPV F/101P cleavage efficiency in the presence and absence of trypsin. In contrast, rhMPV/NL/1/00/F101S was able to initiate multi-cycle growth only once hMPV F/101S was cleaved by the addition of exogeneous trypsin. Thus, the S101P substitution at the RQSR motif is the major determinant of trypsin independent growth phenotype and plays a major role in promoting the hMPV F₁/F₂ cleavage.

hMPV expressing hMPV F/101P rapidly acquired mutations at other amino acid positions in the putative F₂ fragment but not the F₁ fragment. Most of these mutations are adjacent to the RQPR motif although the Q100K mutation is located in the motif. Of the F₂ mutations that occurred outside the RQPR motif, E93K was identified most frequently and hMPV engineered to express hMPV F/93K/101P showed enhanced F₀ processing and cell fusion activity. The rapidity with which mutations that enhanced hMPV F cleavage arose showed that they confer a growth advantage in Vero cell culture. Even though this growth advantage was not apparent from the comparative multi-cycle growth curves done at a MOI of 0.1, increased efficiency of hMPV F cleavage did result in the production of more infectious virus when comparing the growth of rhMPV/NL/1/00/101P to rhMPV/NL/1/00/101S in the presence of trypsin (FIG. 2). However, the growth of rhMPV/NL/1/00/101P may be sufficiently efficient such that further enhancement in hMPV F cleavage efficiency is unlikely to significantly increase the peak titers (FIG. 6).

This phenotype was also observed for hMPV/NL/1/99, a subtype B1 hMPV. The F proteins of subtypes A1 and B1 share amino acid homology of 94% and most of the non-homologous amino acids are located at the C terminus of the hMPV F protein that includes the putative transmembrane domain (van den Hoogen et al, 2004a and b). While a S to P substitution at position 101 of the fusion protein also resulted in trypsin independent growth of hMPV/NL/1/99, sequencing of the P6 stock revealed that the major F₂ polymorphisms are at amino acids 94 and 96 in contrast to 93 and 100 for subtype A1 hMPV F. Since the F proteins of the two subtypes are highly conserved around the F₁/F₂ cleavage site, it is surprising to find different cleavage-enhancing mutations. Without being bound by theory, more extensive passaging of hMPV/NL/1/99/F 101P may result in amino acid substitutions similar to those found in the subtype A1 F₂ fragment. However, the differences in the F2 mutations may reflect flexibility in the binding of the protease that catalyzed hMPV F cleavage or higher order conformational differences in this region of the hMPV F A1 and B1 glycoproteins.

The S101P substitution also increased the cleavage efficiency of hMPV F following expression from a chimeric bovine/human PIV3 virus vector indicating that cleavage of the hMPV fusion protein occurred in the absence of interaction with other hMPV proteins. However, the amount of hMPV F₁ fragment derived from PIV3-infected cells was relatively less than that observed in hMPV infected cells showing that interactions with other hMPV proteins resulted in more cleavage activity. Other possibilities include inhibitory effects of PIV3 proteins or differences in cellular states induced by hMPV versus PIV3 infections. Nonetheless these observations serve as further confirmation that the S101P substitution in the RQSR motif of hMPV F is an important determinant of cleavage activity in Vero cells.

The surface expression of hMPV F/101S suggested that the uncleaved hMPV F₀ precursor was trafficked to the cell surface. In Vero cells, a substantial amount of the hMPV F₀ precursor was protected from cleavage even in the presence of trypsin, in contrast to the processing of RSV fusion proteins (Gonzalez-Reyes 2001; Collins, 1991). This suggested that the processing of hMPV F precursor is inefficient and/or hMPV F₀ has a functional role in the replication cycle of hMPV in vitro. hMPV F/101S appeared to be cleaved extracellularly after exposure to exogeneously added trypsin. However, it is unclear whether hMPV F/101P is cleaved intra- or extracellularly. Other paramyxovirus virus fusion proteins that contain multiple basic residues at the cleavage site are thought to be cleaved by an intracellular protease such as furin (Bosch, 1981; Kawaoka et al 1984; Klenk 1988 and1994).

For paramyxovirus fusion proteins, cleavage of the F₀ precusor is a prerequisite for infectivity and pathogenicity (Kido et al 1996; Klenk 1994) . For some respiratory viruses such as influenza, Newcastle's Disease virus (NDV), parainfluenza virus type 2 (PIV2) and Sendai virus (SeV), changes in the F protein that altered recognition by a tissue-specific protease (e.g. Clara tryptase secreted by bronchial epithelium) to a non-specific ubiquitious protease such as furin has given rise to an increase in virulence. (Bosch et al, 1981; Collins et al, 1993 and 2001; Glickman et al 1988; Kawoaka et al, 1984; Klenk et al, 1988 and 1994; Nagai et al, 1989, Seal et al 2000; Toyoda et al, 1987; Towatari et al 2002).

### REFERENCES

Barr, P. J. 1991. Mammalian subtilisins: the long-sought dibasic processing endoproteases. Cell. 66: 1-3.
Bastien, N., S. Normand, T. Taylor, D. Ward, T. C. T. Peret, G. Boivin, L. J. Anderson, Y. Li. 2003a. Sequence analysis of the N, P, M and F genes of Canadian human metapneumovirus strains. Virus Research. 93: 51-62.
Bastien, N, D. Ward, P. Van Caeseele, K. Brandt, S. H. Lee, G. McNabb, B. Klisko, E. Chan and Y. Li. 2003b. Human metapneumovirus infection in the Canadian population. J. Clin. Microbiol. 41: 4642-4646.
Biacchesi, S., M. H. Skiadopoulos, G. Boivin, C. T. Hanson, B. R. Murphy, P. L. Collins, and U. J. Buchholz. 2003. Genetic diversity between human metapneumovirus subgroups. Virology 315: 1-9.
Boivin, G., Y. Abed, G. Pelletier, L. Ruel, D. Moisan, S. Cote, T. C. T. Peret, D. D. Erdman, and L. J. Anderson. 2002. Virological features and clinical manifestations associated with human metapeumovirus: a new paramyxovirus responsible for acute respiratory-tract infections in all age groups. J. Infect. Dis. 186: 1330-1334.
Boivin, G., G. De Serres, S. Cote, R. Gilca, Y. Abed, L. Rochette, M. G. Bergeron and P. Dery. 2003. Human metapneumovirus infections in hospitalized children. Emerg. Infect. Dis. 9: 634-640.
Bosch, F. S., W. Garten, H.-D. Klenk, and R. Rott. 1981. Proteolytic cleavage of influenza virus hemagglutinin. Primary structure of the connecting peptide between HA1 and HA2 determines proteolytic cleavability and pathogenicity of avian influenza viruses. Virology 113: 725-735.
Chen, Y., M. Shiota, M. Ohuchi, T. Towatari, J. Tashiro, M. Murakami, M. Yano, B. Yang and H. Kido. 2000. Mast cell tryptase from pig lungs triggers infection by pneumotropic Sendai and influenza A viruses. Eur. J. Biochem. 267: 3189-3197.
Collins, M. S., J. B. Bashiruddin, D. J. Alexander. 1993. Deduced amino acid sequences at the fusion protein cleavage site of Newcastle disease viruses showing variation in antigenicity and pathogenicity. Arch. Virol. 128: 363-370.
Collins, P. L. and G. Mottett. 1991. Post translational processing and oligomerization of the fusion glycoprotein of human respiratory syncytial virus. J. Gen. Virol. 72: 3095-4101.
Collins, P. L., R. M. Chanock, B. R. Murphy. 2001. Respiratory syncytial virus. In: Knipe, D. M., Howley, P. M., D. E Griffin, R. A. Lamb, M. A. Martin, B. Roizman, and S. E. Straus (Eds.), Fields Virology, fourth ed. Lippincott Williams and Wilkins, Philadelphia, Pa., pp. 1443-1486.
Glickman, R. L., R. J. Syddall, R. M. Iorio, J. P. Sheehan, and M. A. Bratt. 1988. Quantitative basic residue requirements in the cleavage-activation site of the fusion glycoprotein as a determinant of virulence for Newcastle disease virus. J. Virol. 62: 354-356.
Gonzalez-Reyes, L., M. B. Ruiz-Arguello, B. Garcia-Barreno, L. Calder, J. A. Lopez, J. P. Albar, J. J. Skehel, D. C. Wiley and J. A. Melero. 2001. Cleavage of the human respiratory syncytial virus fusion protein at two distinct sites is required for activation of membrane fusion. PNAS. 98: 9859-9864.
Haller, A. A., T. Miller, M. Mitiku and K. Coelingh. 2000. Expression of the surface glycoproteins of human parainfluenza virus type 3 by bovine parainfluenza virus type 3, a novel attenuated virus vaccine vector. J. Virol. 74: 11626-11635.
Haller, A. A., M. MacPhail, M. Mitiku and R. S. Tang. 2001. A single amino acid substitution in the viral polymerase creates a temperature-sensitive and attenuated recombinant bovine parainfluenza virus type 3. Virology. 288: 342-350.
Hamelin, M-E., Y. Abed, and G. Boivin. 2004. Human metapneumovirus: a new player among respiratory viruses. Clinical Infectious Diseases 38: 983-990.
Herfst, S., M. de Graaf, J. H. Schickli, R. S. Tang, J. Kaur, R. R. Spaete, A. A. Haller, B. G. van den Hoogen, A. D. M. E. Osterhaus and R. A. M. Fouchier. 2004. Recovery of human metapneumovirus genetic lineages A and B from cloned cDNA. J. Virol. 78:8264-8270.
Howe, M. 2002. Australian find suggests worldwide reach for metapneumovirus. Lancet Infect. Dis. 2:202.
IJpma, F. F., D. Beekhuis, M. F. Cotton, C. H. Pieper, J. L. Kimpen, B. G. van den Hoogen, G. J. van Doornum, and D. M. Osterhaus. 2004. Human metapneumovirus infection in hospital referred South African children. J. Med. Virol. 73: 486
Ishida, N. and M. Homma. 1978. Sendai virus. Adv. Virus Res. 23: 349-383.
Kawaoka, T., C. W. Naeve, and R. G. Webster. 1984. Is virulence of H5N2 influenza viruses in chickens associated with loss of carbohydrate from the hemagglutinin? Virology 139: 303-316.
Kido, H., T. Yokogoshi, K. Sakai, M. Tashiro, Y. A. Kishino, A. Fukutomi, and N. Katunuma. 1992. Isolation and characterization of a novel trypsin-like protease found in rat bronchiolar epithelial Clara cells: a possible activator of the viral fusion glycoprotein. J. Biol. Chem. 267: 13573-13579.
Kido, H., Y. Niwa, Y. Beppu, and T. Towatari. 1996. Cellular proteases involved in the pathogenicity of enveloped animal viruses, human immunodeficiency virus, influenza virus A and Sendai virus. Adv. Anzyme Regul. 36: 325-47.
Klenk, H-D. and W. Garten. 1994. Host cell proteases controlling virus pathogenicity. Trends Microbiol. 2 (2): 39-43.
Klenk, H.-D. and R. Rott. 1988. The molecular biology of influenza virus pathogenicity. Adv. Virus Res. 34: 247-281.
Lamb, R. A. 1993. Paramyxovirus fusion: A hypothesis for changes. Virology. 197: 1-11.
Maggi, F., M. Pifferi, M. Vatteroni, C. Fornai, E. Tempestini, S. Anzilotti, L. Lanini, E Andreoli, V. Ragazzo, M. Pistello, S. Specter, M. Bendinelli. 2003. Human metapneumovirus associated with respiratory tract infections in a 3-year study of nasal swabs from infants in Italy. J. Clinical Microbiology. 41: 2987-2991.
Morrison, T. G., 2003. Structure and function of a paramyxovirus fusion protein. Biochimica et Biophysica Acta 1614: 73-84.
Nagai, Y., M. Hamaguchi, and T. Toyoda. 1989. Molecular biology of Newcastle disease virus. Prog. Vet. Microbiol. 5: 16-64.
Nagai, Y., H.-D. Klenk, and R. Rott. 1976. Proteolytic cleavage of the viral glycoproteins and its significance for the virulence of Newcastle disease virus. Virology 72: 494-508.
Nissen M. D., D. J. Siebert, I. M., Mackay, T. P., Sloots, and S. J. Withers. 2002. Evidence of human metapneumovirus in Australian children. Med. J. Aust. 176: 188.
Okada, H., J. T. Seto, N. L. McQueen, H.-D. Klenk, R. Rott, and M. Tashiro. 1998. Determinants of pantropism of the F1-R mutant of Sendai virus: specific mutations involved are in the F and M genes. Arch. Virol. 143: 2342-2352.
Peiris, J. S., W. H. Tang, K. H. Chan, P. L. Khong, Y. Guan, Y. L. Lau and S. S. Chiu. 20023, Children with respiratory disease associated with metapneumovirus in Hong Kong. Emerg. Infect. Dis. 9: 628-633.
Peret, T. C. T., G. Boivin, Y. Li, M. Couillard, C. Humphrey, A. D. M. E. Osterhaus, D. D. Erdman, and L. J. Anderson. 2002. Characterization of human metapneumoviruses isolated from patients in North America. J. Infect. Dis. 185: 1660-1663.
Peret, T. C. T., Y. Abed, L. J. Anderson, D. D. Erdman, and G. Boivin. 2004. Sequence polymorphism of the predicted human metapneumovirus G glycoprotein. J. Gen Virol. 85: 679-686.
Russell, C. J., T. S. Jardertzky, R. A. Lamb. 2001. Membrane fusion machines of paramyxoviruses: capture of intermediates of fusion. EMBO. J. 20: 4024-4034.
Seal, B. S., H. S. Sellers, R. J. Meinersmann, 2000. Fusion protein predicted amino acid sequence of the first US avian pneumovirus isolate and lack of heterogeneity among other US isolates. Virus. Res. 66: 139-147.
Scheid, A and P. W. Choppin. 1974. Identification of the biological activities of paramyxovirus glycoproteins. Activation of cell fusion, hemolysis and infectivity by proteolytic cleavage of an inactive precursor protein of Sendaivirus. Virology, 57:475-490.
Scheid, A and P. W. Choppin, 1977. Two disulfide linked polypeptide chains constitute the active F protein of paramyxoviruses. Virology 80: 54-66.
Skiadopoulos, M. H., S. Biacchesi, U. J. Buchholz, J. M. Riggs, S. R. Sunman, E. Amaro-Carambot, J. M. McAuliffe, W. R. Elkins, M. St. Claire, P.L. Collins and B. R Murphy. 2004. The two major human metapneumovirus genetic lineages are highly related antigenically, and the fusion (F) protein is a major contributor to this antigenic relatedness. J. V. 78: 6927-6937.
Skiadopoulos, M. H., S. R. Surman, J. M. Riggs, C. Orvell, P. L. Collins, and B. R. Murphy. 2002. Evaluation of the replication and immunogenicity of recombinant human parainfluenza virus type 3 vectors expressing up to three foreign glycoproteins. Virology 297: 136-152.
Stockton, J. I. Stephenson, D. Fleming, M. Zambon, 2002. Human metapneumovirus as a cause of community-acquired respiratory illness. Emerg. Infect. Dis. 8, 897-901.
Tang, R. S., M. MacPhail, J. H. Schickli, J. Kaur, C. L. Robinson, H. A. Lawlor, J. M. Guzzetta, R. R. Spaete and A. A. Haller. 2004a. Parainfluenza virus type 3 expressing the native or soluble fusion (F) protein of respiratory syncytial virus (RSV) confers protection from RSV infection in African green monkeys. J. Virol. 78: 11198-11207.
Tang, R. S., K. Mahmood, M. MacPhail, J. M. Guzzetta, A. A. Haller, H. Liu, J. Kaur, H. A. Lawlor, E. A. Stillman, J. H. Schickli R. A. M. Fouchier, A. D. M. E. Osterhaus, R. R. Spaete. 2004b. A Host-range restricted parainfluenza virus type 3 (PIV3) expressing the human metapneumovirus (hMPV) fusion protein elicits protective immunity in African green monkeys. Vaccine (in press)
Tang, R. S., J. H. Schickli, M. MacPhail, F. Fernandes, L. Bicha, J. Spaete, R. A. M. Fouchier, A. D. M. E. Osterhaus, R. Spaete and A. A. Haller. 2003. Effects of human metapneumovirus and respiratory syncytial virus antign insertion in two 3′ proximal genome positions of bovine/human parainfluenza virus type 3 on virus replication and immunogenicity. J. Virol. 77: 10819-10828
Tashiro, M. and M. Homma. 1983. Pneumotropism of Sendai virus in relation to protease-mediated activation in mouse lungs. Infect. Immun. 39: 879-888.
Tashiro, M., N. L. McQueen, J. T. Seto, H.-D. Klenk, and R. Rott. 1996. Involvement of the mutated M protein in altered budding polarity of a pantropic mutant, F1-R, of Sendai virus. J. of Vir. 70: 5990-5997.
Tashiro, M., E. Pritzer, M. A. Khoshnan, M. Yamakawa, K. Kuroda, H.-D. Klenk, R. Rott, and J. T. Seto. 1988. Characterization of a pantropic variant of Sendai virus derived from a host-range mutant. Virology 165: 577-583.
Tashiro, M. J. T. Seto, S. Choosakul, M. Yamakawa, H.-D. Klenk and R. Rott. 1992a. Budding site of Sendai virus in polarized epithelial cells is one of the determinants for tropism and pathogenicity in mice. Virology 187: 413-422.
Tashiro, M., S. T. Seto, H.-D. Klenk, and R. Rott. 1993. Possible involvement of microtubule disruption in bipolar budding of a Sendai virus mutant, F1-R, in epithelial MDCK cells. J. of Vir. 67: 5902-5910.
Tashiro, M., M. Yamakawa, K. Tobita, H.-D. Klenk, R. Rott, and J. T. Seto. 1990 Organ tropism of Sendai virus in mice: proteolytic activation of the fusion glycoprotein in mouse organs and budding site at the bronchial epithelium. J. of Vir. 64: 3627-3634.
Tashiro, M., Y. Yokogoshi, K. Tobita, J. T. Seto, R. Rott and H. Kondo. 1992b. Tryptase Clara: an activating protease for Sendai virus in rat lungs that is involved in pneumopathogenicity. J. Viro. 66: 7211-7216.
Toquin, D., C. de Boisseson, V. Beven, D. A. Senne, and N Eterradossi. 2003. Subgroup C avian metapneumovirus (MPV) and the recently isolated human MPV exhibit a common organization but have extensive sequence divergence in their putative SH and G genes. J. of General Virology. 84: 2169-2178.
Towatari, T., M. Ide, K. Ohba, Y. Chiba, M. Murakami, M. Shiota, M. Kawachi, H. Yamada and H. Kido. 2002. Identification of ectopic anionic trypsin I in rat lungs potentiating pneumotropic virus infectivity and increased enzyme level after virus infection. Eur. J. Biochem. 269: 2613-2621.
Toyoda, T., T. Sakaguchi, K. Imai, N. M. Inocencio, B. Gotoh, M. Hamaguchi, and T. Nagai. 1987. Structural comparison of the cleavage-activation site of the fusion glycoprotein between virulent and avirulent strains of Newcastle disease virus. Virology 158: 242-247.
van den Hoogen, B. G., T. M. Bestebroer, A. D. M. E. Osterhaus, and R. A. M. Fouchier. 2002. Analysis of the genomic sequence of human metapneumovirus. Virology. 295: 119-132.
van den Hoogen, B. G., G. J. van Doornum, J. C. Fockens, J. J. Cornelissen, W. E. Beyer, R. deGroot, A. D. Osterhaus and R. A. Fouchier. 2003. Prevalence and clinical symptoms of human metapneumovirus infection in hospitalized patients. J. Infect. Dis. 188: 1571-1577.
van den Hoogen, B. G., S. Herfst, L. Sprong, P. A. Cane, E. Forleo-Neto, R. L. de Swart, A. D. M. E. Osterhaus, and R. A. M. Fouchier. 2004a. Antigenic and genetic variability of human metapneumoviruses. 2004b. Emerging Infectious Diseases. 10: 658-666.
van den Hoogen, B. G., J. C. de Jong, J. Groen, T. Kuiken, R. de Groot, R. A. M. Fouchier, and A. D. M. E. Osterhaus. 2001. A newly discovered human pneumovirus isolated from young children with respiratory tract disease. Nat. Med. 7: 719-724.
van den Hoogen, B. G., A. D. M. E. Osterhaus and R. A. M. Fouchier. 2004b. Clinical impact and diagnosis of hMPV infections. Pediatric Infectious Disease Journal, 23: S25-32.
White, J. M. 1990. Viral and cellular membrane fusion proteins. Annual Review Physiology. 52: 675-697.
Willams, J. V., P. A. Harris, S. J. Tollefson, L. L. Halburnt-Rush, J. M. Pingsterhaus, K. M. Edwards, P. F. Wright, and J. E. Crowe Jr. 2004. Human metapneumovirus and lower respiratory tract disease in otherwise healthy infants and children. N. Engl. J. Med. 350: 443-450.
Wolf, D., Z. Zakay-Rones, A. Fadeela, D. Greenberg and R. Dagan. 2003. High seroprevalence of human metapneumovirus among young children in Israel. J. Inf. Dis. 188: 1865-1867.

### REFERENCES CITED

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled. Such modifications are intended to fall within the scope of the appended claims.

All references, patent and non-patent, cited herein are incorporated herein by reference in their entireties and for all purposes to the same extent as if each individual publication or patent or patent application was specifically and individually indicated to be incorporated by reference in its entirety for all purposes.

Additionally, U.S. patent application Ser. No. 10/831,780 entitled “Metapneumovirus Strains And Their Use In Vaccine Formulations And As Vectors For Expression Of Antigenic Sequences And Methods For Propagating Virus” filed on Apr. 23, 2004 published as US 2005/0019891 A1 on Jan. 27, 2005 is incorporated herein by reference in its entirety.

TABLE 14
LEGEND FOR SEQUENCE LISTING

| | |
|---|---|
| SEQ ID NO: 1 | Human metapneumovirus isolate 00-1 matrix protein (M) and fusion protein (F) genes |
| SEQ ID NO: 2 | Avian pneumovirus fusion protein gene, partial cds |
| SEQ ID NO: 3 | Avian pneumovirus isolate 1b fusion protein mRNA, complete cds |
| SEQ ID NO: 4 | Turkey rhinotracheitis virus gene for fusion protein (F1 and F2 subunits), complete cds |
| SEQ ID NO: 5 | Avian pneumovirus matrix protein (M) gene, partial cds and Avian pneumovirus fusion |
| | glycoprotein (F) gene, complete cds |
| SEQ ID NO: 6 | paramyxovirus F protein hRSV B |
| SEQ ID NO: 7 | paramyxovirus F protein hRSV A2 |
| SEQ ID NO: 8 | human metapneumovirus01-71 (partial sequence) |
| SEQ ID NO: 9 | Human metapneumovirus isolate 00-1 matrix protein(M) and fusion protein (F) genes |
| SEQ ID NO: 10 | Avian pneumovirus fusion protein gene, partial cds |
| SEQ ID NO: 11 | Avian pneumovirus isolate 1b fusion protein mRNA, complete cds |
| SEQ ID NO: 12 | Turkey rhinotracheitis virus gene for fusion protein (F1 and F2 subunits), complete cds |
| SEQ ID NO: 13 | Avian pneumovirus fusion glycoprotein (F) gene, complete cds |
| SEQ ID NO: 14 | Turkey rhinotracheitis virus (strain CVL14/1)attachment protien (G) mRNA, complete cds |
| SEQ ID NO: 15 | Turkey rhinotracheitis virus (strain 6574)attachment protein (G), complete cds |
| SEQ ID NO: 16 | Turkey rhinotracheitis virus (strain CVL14/1)attachment protein (G) mRNA, complete cds |
| SEQ ID NO: 17 | Turkey rhinotracheitis virus (strain 6574)attachment protein (G), complete cds |
| SEQ ID NO: 18 | isolate NL/1/99 (99-1) HMPV (Human Metapneumovirus)cDNA sequence |
| SEQ ID NO: 19 | isolate NL/1/00 (00-1) HMPV cDNA sequence |
| SEQ ID NO: 20 | isolate NL/17/00 HMPV cDNA sequence |
| SEQ ID NO: 21 | isolate NL/1/94 HMPV cDNA sequence |
| SEQ ID NO: 22 | RT-PCR primer TR1 |
| SEQ ID NO: 23 | RT-PCR primer N1 |
| SEQ ID NO: 24 | RT-PCR primer N2 |
| SEQ ID NO: 25 | RT-PCR primer M1 |
| SEQ ID NO: 26 | RT-PCR primer M2 |
| SEQ ID NO: 27 | RT-PCR primer F1 |
| SEQ ID NO: 28 | RT-PCR primer N3 |
| SEQ ID NO: 29 | RT-PCR primer N4 |
| SEQ ID NO: 30 | RT-PCR primer M3 |
| SEQ ID NO: 31 | RT-PCR primer M4 |
| SEQ ID NO: 32 | RT-PCR primer F7 |
| SEQ ID NO: 33 | RT-PCR primer F8 |
| SEQ ID NO: 34 | RT-PCR primer L6 |
| SEQ ID NO: 35 | RT-PCR primer L7 |
| SEQ ID NO: 36 | Oligonucleotide probe M |
| SEQ ID NO: 37 | Oligonucleotide probe N |
| SEQ ID NO: 38 | Oligonucleotide probe L |
| SEQ ID NO: 39 | TaqMan primer and probe sequences for isolates NL/1/00, BI/1/01, FI/4/01, NL/8/01, |
| | FI/2/01 |
| SEQ ID NO: 40 | TaqMan primer and probe sequences for isolates NL/30/01 |
| SEQ ID NO: 41 | TaqMan primer and probe sequences for isolates NL/22/01 and NL/23/01 |
| SEQ ID NO: 42 | TaqMan primer and probe sequences for isolate NL/17/01 |
| SEQ ID NO: 43 | TaqMan primer and probe sequences for isolate NL/17/00 |
| SEQ ID NO: 44 | TaqMan primer and probe sequences for isolates NL/9/01, NL/21/01, and NL/5/01 |
| SEQ ID NO: 45 | TaqMan primer and probe sequences for isolates FI/1/01 and FI/10/01 |
| SEQ ID NO: 46 | Primer ZF1 |
| SEQ ID NO: 47 | Primer ZF4 |
| SEQ ID NO: 48 | Primer ZF7 |
| SEQ ID NO: 49 | Primer ZF10 |
| SEQ ID NO: 50 | Primer ZF13 |
| SEQ ID NO: 51 | Primer ZF16 |
| SEQ ID NO: 52 | Primer CS1 |
| SEQ ID NO: 53 | Primer CS4 |
| SEQ ID NO: 54 | Primer CS7 |
| SEQ ID NO: 55 | Primer CS10 |
| SEQ ID NO: 56 | Primer CS13 |
| SEQ ID NO: 57 | Primer CS16 |
| SEQ ID NO: 58 | Forward primer for amplification of HPIV-1 |
| SEQ ID NO: 59 | Reverse primer for amplification of HPIV-1 |
| SEQ ID NO: 60 | Forward primer for amplification of HPIV-2 |
| SEQ ID NO: 61 | Reverse primer for amplification of HPIV-2 |
| SEQ ID NO: 62 | Forward primer for amplification of HPIV-3 |
| SEQ ID NO: 63 | Reverse primer for amplification of HPIV-3 |
| SEQ ID NO: 64 | Forward primer for amplification of HPIV-4 |
| SEQ ID NO: 65 | Reverse primer for amplification of HPIV-4 |
| SEQ ID NO: 66 | Forward primer for amplification of Mumps |
| SEQ ID NO: 67 | Reverse primer for amplification of Mumps |
| SEQ ID NO: 68 | Forward primer for amplification of NDV |
| SEQ ID NO: 69 | Reverse primer for amplification of NDV |
| SEQ ID NO: 70 | Forward primer for amplification of Tupaia |
| SEQ ID NO: 71 | Reverse primer for amplification of Tupaia |
| SEQ ID NO: 72 | Forward primer for amplification of Mapuera |
| SEQ ID NO: 73 | Reverse primer for amplification of Mapuera |
| SEQ ID NO: 74 | Forward primer for amplification of Hendra |
| SEQ ID NO: 75 | Reverse primer for amplification of Hendra |
| SEQ ID NO: 76 | Forward primer for amplification of Nipah |
| SEQ ID NO: 77 | Reverse primer for amplification of Nipah |
| SEQ ID NO: 78 | Forward primer for amplification of HRSV |
| SEQ ID NO: 79 | Reverse primer for amplification of HRSV |
| SEQ ID NO: 80 | Forward primer for amplification of Measles |
| SEQ ID NO: 81 | Reverse primer for amplification of Measles |
| SEQ ID NO: 82 | Forward primer to amplify general paramyxoviridae viruses |
| SEQ ID NO: 83 | Reverse primer to amplify general paramyxoviridae viruses |
| SEQ ID NO: 84 | G-gene coding sequence for isolate NL/1/00 (A1) |
| SEQ ID NO: 85 | G-gene coding sequence for isolate BR/2/01 (A1) |
| SEQ ID NO: 86 | G-gene coding sequence for isolate FL/4/01 (A1) |
| SEQ ID NO: 87 | G-gene coding sequence for isolate FL/3/01 (A1) |
| SEQ ID NO: 88 | G-gene coding sequence for isolate FL/8/01 (A1) |
| SEQ ID NO: 89 | G-gene coding sequence for isolate FL/10/01 (A1) |
| SEQ ID NO: 90 | G-gene coding sequence for isolate NL/10/01 (A1) |
| SEQ ID NO: 91 | G-gene coding sequence for isolate NL/2/02 (A1) |
| SEQ ID NO: 92 | G-gene coding sequence for isolate NL/17/00 (A2) |
| SEQ ID NO: 93 | G-gene coding sequence for isolate NL/1/81 (A2) |
| SEQ ID NO: 94 | G-gene coding sequence for isolate NL/1/93 (A2) |
| SEQ ID NO: 95 | G-gene coding sequence for isolate NL/2/93 (A2) |
| SEQ ID NO: 96 | G-gene coding sequence for isolate NL/3/93 (A2) |
| SEQ ID NO: 97 | G-gene coding sequence for isolate NL/1/95 (A2) |
| SEQ ID NO: 98 | G-gene coding sequence for isolate NL/2/96 (A2) |
| SEQ ID NO: 99 | G-gene coding sequence for isolate NL/3/96 (A2) |
| SEQ ID NO: 100 | G-gene coding sequence for isolate NL/22/01 (A2) |
| SEQ ID NO: 101 | G-gene coding sequence for isolate NL/24/01 (A2) |
| SEQ ID NO: 102 | G-gene coding sequence for isolate NL/23/01 (A2) |
| SEQ ID NO: 103 | G-gene coding sequence for isolate NL/29/01 (A2) |
| SEQ ID NO: 104 | G-gene coding sequence for isolate NL/3/02 (A2) |
| SEQ ID NO: 105 | G-gene coding sequence for isolate NL/1/99 (B1) |
| SEQ ID NO: 106 | G-gene coding sequence for isolate NL/11/00 (B1) |
| SEQ ID NO: 107 | G-gene coding sequence for isolate NL/12/00 (B1) |
| SEQ ID NO: 108 | G-gene coding sequence for isolate NL/5/01 (B1) |
| SEQ ID NO: 109 | G-gene coding sequence for isolate NL/9/01 (B1) |
| SEQ ID NO: 110 | G-gene coding sequence for isolate NL/21/01 (B1) |
| SEQ ID NO: 111 | G-gene coding sequence for isolate NL/1/94 (B2) |
| SEQ ID NO: 112 | G-gene coding sequence for isolate NL/1/82 (B2) |
| SEQ ID NO: 113 | G-gene coding sequence for isolate NL/1/96 (B2) |
| SEQ ID NO: 114 | G-gene coding sequence for isolate NL/6/97 (B2) |
| SEQ ID NO: 115 | G-gene coding sequence for isolate NL/9/00 (B2) |
| SEQ ID NO: 116 | G-gene coding sequence for isolate NL/3/01 (B2) |
| SEQ ID NO: 117 | G-gene coding sequence for isolate NL/4/01 (B2) |
| SEQ ID NO: 118 | G-gene coding sequence for isolate UK/5/01 (B2) |
| SEQ ID NO: 119 | G-protein sequence for isolate NL/1/00 (A1) |
| SEQ ID NO: 120 | G-protein sequence for isolate BR/2/01 (A1) |
| SEQ ID NO: 121 | G-protein sequence for isolate FL/4/01 (A1) |
| SEQ ID NO: 122 | G-protein sequence for isolate FL/3/01 (A1) |
| SEQ ID NO: 123 | G-protein sequence for isolate FL/8/01 (A1) |
| SEQ ID NO: 124 | G-protein sequence for isolate FL/10/01 (A1) |
| SEQ ID NO: 125 | G-protein sequence for isolate NL/10/01 (A1) |
| SEQ ID NO: 126 | G-protein sequence for isolate NL/2/02 (A1) |
| SEQ ID NO: 127 | G-protein sequence for isolate NL/17/00 (A2) |
| SEQ ID NO: 128 | G-protein sequence for isolate NL/1/81 (A2) |
| SEQ ID NO: 129 | G-protein sequence for isolate NL/1/93 (A2) |
| SEQ ID NO: 130 | G-protein sequence for isolate NL/2/93 (A2) |
| SEQ ID NO: 131 | G-protein sequence for isolate NL/3/93 (A2) |
| SEQ ID NO: 132 | G-protein sequence for isolate NL/1/95 (A2) |
| SEQ ID NO: 133 | G-protein sequence for isolate NL/2/96 (A2) |
| SEQ ID NO: 134 | G-protein sequence for isolate NL/3/96 (A2) |
| SEQ ID NO: 135 | G-protein sequence for isolate NL/22/01 (A2) |
| SEQ ID NO: 136 | G-protein sequence for isolate NL/24/01 (A2) |
| SEQ ID NO: 137 | G-protein sequence for isolate NL/23/01 (A2) |
| SEQ ID NO: 138 | G-protein sequence for isolate NL/29/01 (A2) |
| SEQ ID NO: 139 | G-protein sequence for isolate NL/3/02 (A2) |
| SEQ ID NO: 140 | G-protein sequence for isolate NL/1/99 (B1) |
| SEQ ID NO: 141 | G-protein sequence for isolate NL/11/00 (B1) |
| SEQ ID NO: 142 | G-protein sequence for isolate NL/12/00 (B1) |
| SEQ ID NO: 143 | G-protein sequence for isolate NL/5/01 (B1) |
| SEQ ID NO: 144 | G-protein sequence for isolate NL/9/01 (B1) |
| SEQ ID NO: 145 | G-protein sequence for isolate NL/21/01 (B1) |
| SEQ ID NO: 146 | G-protein sequence for isolate NL/1/94 (B2) |
| SEQ ID NO: 147 | G-protein sequence for isolate NL/1/82 (B2) |
| SEQ ID NO: 148 | G-protein sequence for isolate NL/1/96 (B2) |
| SEQ ID NO: 149 | G-protein sequence for isolate NL/6/97 (B2) |
| SEQ ID NO: 150 | G-protein sequence for isolate NL/9/00 (B2) |
| SEQ ID NO: 151 | G-protein sequence for isolate NL/3/01 (B2) |
| SEQ ID NO: 152 | G-protein sequence for isolate NL/4/01 (B2) |
| SEQ ID NO: 153 | G-protein sequence for isolate NL/5/01 (B2) |
| SEQ ID NO: 154 | F-gene coding sequence for isolate NL/1/00 |
| SEQ ID NO: 155 | F-gene coding sequence for isolate UK/1/00 |
| SEQ ID NO: 156 | F-gene coding sequence for isolate NL/2/00 |
| SEQ ID NO: 157 | F-gene coding sequence for isolate NL/13/00 |
| SEQ ID NO: 158 | F-gene coding sequence for isolate NL/14/00 |
| SEQ ID NO: 159 | F-gene coding sequence for isolate FL/3/01 |
| SEQ ID NO: 160 | F-gene coding sequence for isolate FL/4/01 |
| SEQ ID NO: 161 | F-gene coding sequence for isolate FL/8/01 |
| SEQ ID NO: 162 | F-gene coding sequence for isolate UK/1/01 |
| SEQ ID NO: 163 | F-gene coding sequence for isolate UK/7/01 |
| SEQ ID NO: 164 | F-gene coding sequence for isolate FL/10/01 |
| SEQ ID NO: 165 | F-gene coding sequence for isolate NL/6/01 |
| SEQ ID NO: 166 | F-gene coding sequence for isolate NL/8/01 |
| SEQ ID NO: 167 | F-gene coding sequence for isolate NL/10/01 |
| SEQ ID NO: 168 | F-gene coding sequence for isolate NL/14/01 |
| SEQ ID NO: 169 | F-gene coding sequence for isolate NL/20/01 |
| SEQ ID NO: 170 | F-gene coding sequence for isolate NL/25/01 |
| SEQ ID NO: 171 | F-gene coding sequence for isolate NL/26/01 |
| SEQ ID NO: 172 | F-gene coding sequence for isolate NL/28/01 |
| SEQ ID NO: 173 | F-gene coding sequence for isolate NL/30/01 |
| SEQ ID NO: 174 | F-gene coding sequence for isolate BR/2/01 |
| SEQ ID NO: 175 | F-gene coding sequence for isolate BR/3/01 |
| SEQ ID NO: 176 | F-gene coding sequence for isolate NL/2/02 |
| SEQ ID NO: 177 | F-gene coding sequence for isolate NL/4/02 |
| SEQ ID NO: 178 | F-gene coding sequence for isolate NL/5/02 |
| SEQ ID NO: 179 | F-gene coding sequence for isolate NL/6/02 |
| SEQ ID NO: 180 | F-gene coding sequence for isolate NL/7/02 |
| SEQ ID NO: 181 | F-gene coding sequence for isolate NL/9/02 |
| SEQ ID NO: 182 | F-gene coding sequence for isolate FL/1/02 |
| SEQ ID NO: 183 | F-gene coding sequence for isolate NL/1/81 |
| SEQ ID NO: 184 | F-gene coding sequence for isolate NL/1/93 |
| SEQ ID NO: 185 | F-gene coding sequence for isolate NL/2/93 |
| SEQ ID NO: 186 | F-gene coding sequence for isolate NL/4/93 |
| SEQ ID NO: 187 | F-gene coding sequence for isolate NL/1/95 |
| SEQ ID NO: 188 | F-gene coding sequence for isolate NL/2/96 |
| SEQ ID NO: 189 | F-gene coding sequence for isolate NL/3/96 |
| SEQ ID NO: 190 | F-gene coding sequence for isolate NL/1/98 |
| SEQ ID NO: 191 | F-gene coding sequence for isolate NL/17/00 |
| SEQ ID NO: 192 | F-gene coding sequence for isolate NL/22/01 |
| SEQ ID NO: 193 | F-gene coding sequence for isolate NL/29/01 |
| SEQ ID NO: 194 | F-gene coding sequence for isolate NL/23/01 |
| SEQ ID NO: 195 | F-gene coding sequence for isolate NL/17/01 |
| SEQ ID NO: 196 | F-gene coding sequence for isolate NL/24/01 |
| SEQ ID NO: 197 | F-gene coding sequence for isolate NL/3/02 |
| SEQ ID NO: 198 | F-gene coding sequence for isolate NL/3/98 |
| SEQ ID NO: 199 | F-gene coding sequence for isolate NL/1/99 |
| SEQ ID NO: 200 | F-gene coding sequence for isolate NL/2/99 |
| SEQ ID NO: 201 | F-gene coding sequence for isolate NL/3/99 |
| SEQ ID NO: 202 | F-gene coding sequence for isolate NL/11/00 |
| SEQ ID NO: 203 | F-gene coding sequence for isolate NL/12/00 |
| SEQ ID NO: 204 | F-gene coding sequence for isolate NL/1/01 |
| SEQ ID NO: 205 | F-gene coding sequence for isolate NL/5/01 |
| SEQ ID NO: 206 | F-gene coding sequence for isolate NL/9/01 |
| SEQ ID NO: 207 | F-gene coding sequence for isolate NL/19/01 |
| SEQ ID NO: 208 | F-gene coding sequence for isolate NL/21/01 |
| SEQ ID NO: 209 | F-gene coding sequence for isolate UK/11/01 |
| SEQ ID NO: 210 | F-gene coding sequence for isolate FL/1/01 |
| SEQ ID NO: 211 | F-gene coding sequence for isolate FL/2/01 |
| SEQ ID NO: 212 | F-gene coding sequence for isolate FL/5/01 |
| SEQ ID NO: 213 | F-gene coding sequence for isolate FL/7/01 |
| SEQ ID NO: 214 | F-gene coding sequence for isolate FL/9/01 |
| SEQ ID NO: 215 | F-gene coding sequence for isolate UK/10/01 |
| SEQ ID NO: 216 | F-gene coding sequence for isolate NL/1/02 |
| SEQ ID NO: 217 | F-gene coding sequence for isolate NL/1/94 |
| SEQ ID NO: 218 | F-gene coding sequence for isolate NL/1/96 |
| SEQ ID NO: 219 | F-gene coding sequence for isolate NL/6/97 |
| SEQ ID NO: 220 | F-gene coding sequence for isolate NL/7/00 |
| SEQ ID NO: 221 | F-gene coding sequence for isolate NL/9/00 |
| SEQ ID NO: 222 | F-gene coding sequence for isolate NL/19/00 |
| SEQ ID NO: 223 | F-gene coding sequence for isolate NL/28/00 |
| SEQ ID NO: 224 | F-gene coding sequence for isolate NL/3/01 |
| SEQ ID NO: 225 | F-gene coding sequence for isolate NL/4/01 |
| SEQ ID NO: 226 | F-gene coding sequence for isolate NL/11/01 |
| SEQ ID NO: 227 | F-gene coding sequence for isolate NL/15/01 |
| SEQ ID NO: 228 | F-gene coding sequence for isolate NL/18/01 |
| SEQ ID NO: 229 | F-gene coding sequence for isolate FL/6/01 |
| SEQ ID NO: 230 | F-gene coding sequence for isolate UK/5/01 |
| SEQ ID NO: 231 | F-gene coding sequence for isolate UK/8/01 |
| SEQ ID NO: 232 | F-gene coding sequence for isolate NL/12/02 |
| SEQ ID NO: 233 | F-gene coding sequence for isolate HK/1/02 |
| SEQ ID NO: 234 | F-protein sequence for isolate NL/1/00 |
| SEQ ID NO: 235 | F-protein sequence for isolate UK/1/00 |
| SEQ ID NO: 236 | F-protein sequence for isolate NL/2/00 |
| SEQ ID NO: 237 | F-protein sequence for isolate NL/13/00 |
| SEQ ID NO: 238 | F-protein sequence for isolate NL/14/00 |
| SEQ ID NO: 239 | F-protein sequence for isolate FL/3/01 |
| SEQ ID NO: 240 | F-protein sequence for isolate FL/4/01 |
| SEQ ID NO: 241 | F-protein sequence for isolate FL/8/01 |
| SEQ ID NO: 242 | F-protein sequence for isolate UK/1/01 |
| SEQ ID NO: 243 | F-protein sequence for isolate UK/7/01 |
| SEQ ID NO: 244 | F-protein sequence for isolate FL/10/01 |
| SEQ ID NO: 245 | F-protein sequence for isolate NL/6/01 |
| SEQ ID NO: 246 | F-protein sequence for isolate NL/8/01 |
| SEQ ID NO: 247 | F-protein sequence for isolate NL/10/01 |
| SEQ ID NO: 248 | F-protein sequence for isolate NL/14/01 |
| SEQ ID NO: 249 | F-protein sequence for isolate NL/20/01 |
| SEQ ID NO: 250 | F-protein sequence for isolate NL/25/01 |
| SEQ ID NO: 251 | F-protein sequence for isolate NL/26/01 |
| SEQ ID NO: 252 | F-protein sequence for isolate NL/28/01 |
| SEQ ID NO: 253 | F-protein sequence for isolate NL/30/01 |
| SEQ ID NO: 254 | F-protein sequence for isolate BR/2/01 |
| SEQ ID NO: 255 | F-protein sequence for isolate BR/3/01 |
| SEQ ID NO: 256 | F-protein sequence for isolate NL/2/02 |
| SEQ ID NO: 257 | F-protein sequence for isolate NL/4/02 |
| SEQ ID NO: 258 | F-protein sequence for isolate NL/5/02 |
| SEQ ID NO: 259 | F-protein sequence for isolate NL/6/02 |
| SEQ ID NO: 260 | F-protein sequence for isolate NL/7/02 |
| SEQ ID NO: 261 | F-protein sequence for isolate NL/9/02 |
| SEQ ID NO: 262 | F-protein sequence for isolate FL/1/02 |
| SEQ ID NO: 263 | F-protein sequence for isolate NL/1/81 |
| SEQ ID NO: 264 | F-protein sequence for isolate NL/1/93 |
| SEQ ID NO: 265 | F-protein sequence for isolate NL/2/93 |
| SEQ ID NO: 266 | F-protein sequence for isolate NL/4/93 |
| SEQ ID NO: 267 | F-protein sequence for isolate NL/1/95 |
| SEQ ID NO: 268 | F-protein sequence for isolate NL/2/96 |
| SEQ ID NO: 269 | F-protein sequence for isolate NL/3/96 |
| SEQ ID NO: 270 | F-protein sequence for isolate NL/1/98 |
| SEQ ID NO: 271 | F-protein sequence for isolate NL/17/00 |
| SEQ ID NO: 272 | F-protein sequence for isolate NL/22/01 |
| SEQ ID NO: 273 | F-protein sequence for isolate NL/29/01 |
| SEQ ID NO: 274 | F-protein sequence for isolate NL/23/01 |
| SEQ ID NO: 275 | F-protein sequence for isolate NL/17/01 |
| SEQ ID NO: 276 | F-protein sequence for isolate NL/24/01 |
| SEQ ID NO: 277 | F-protein sequence for isolate NL/3/02 |
| SEQ ID NO: 278 | F-protein sequence for isolate NL/3/98 |
| SEQ ID NO: 279 | F-protein sequence for isolate NL/1/99 |
| SEQ ID NO: 280 | F-protein sequence for isolate NL/2/99 |
| SEQ ID NO: 281 | F-protein sequence for isolate NL/3/99 |
| SEQ ID NO: 282 | F-protein sequence for isolate NL/11/00 |
| SEQ ID NO: 283 | F-protein sequence for isolate NL/12/00 |
| SEQ ID NO: 284 | F-protein sequence for isolate NL/1/01 |
| SEQ ID NO: 285 | F-protein sequence for isolate NL/5/01 |
| SEQ ID NO: 286 | F-protein sequence for isolate NL/9/01 |
| SEQ ID NO: 287 | F-protein sequence for isolate NL/19/01 |
| SEQ ID NO: 288 | F-protein sequence for isolate NL/21/01 |
| SEQ ID NO: 289 | F-protein sequence for isolate UK/11/01 |
| SEQ ID NO: 290 | F-protein sequence for isolate FL/1/01 |
| SEQ ID NO: 291 | F-protein sequence for isolate FL/2/01 |
| SEQ ID NO: 292 | F-protein sequence for isolate FL/5/01 |
| SEQ ID NO: 293 | F-protein sequence for isolate FL/7/01 |
| SEQ ID NO: 294 | F-protein sequence for isolate FL/9/01 |
| SEQ ID NO: 295 | F-protein sequence for isolate UK/10/01 |
| SEQ ID NO: 296 | F-protein sequence for isolate NL/1/02 |
| SEQ ID NO: 297 | F-protein sequence for isolate NL/1/94 |
| SEQ ID NO: 298 | F-protein sequence for isolate NL/1/96 |
| SEQ ID NO: 299 | F-protein sequence for isolate NL/6/97 |
| SEQ ID NO: 300 | F-protein sequence for isolate NL/7/00 |
| SEQ ID NO: 301 | F-protein sequence for isolate NL/9/00 |
| SEQ ID NO: 302 | F-protein sequence for isolate NL/19/00 |
| SEQ ID NO: 303 | F-protein sequence for isolate NL/28/00 |
| SEQ ID NO: 304 | F-protein sequence for isolate NL/3/01 |
| SEQ ID NO: 305 | F-protein sequence for isolate NL/4/01 |
| SEQ ID NO: 306 | F-protein sequence for isolate NL/11/01 |
| SEQ ID NO: 307 | F-protein sequence for isolate NL/15/01 |
| SEQ ID NO: 308 | F-protein sequence for isolate NL/18/01 |
| SEQ ID NO: 309 | F-protein sequence for isolate FL/6/01 |
| SEQ ID NO: 310 | F-protein sequence for isolate UK/5/01 |
| SEQ ID NO: 311 | F-protein sequence for isolate UK/8/01 |
| SEQ ID NO: 312 | F-protein sequence for isolate NL/12/02 |
| SEQ ID NO: 313 | F-protein sequence for isolate HK/1/02 |
| SEQ ID NO: 314 | F protein sequence for HMPV isolate NL/1/00 |
| SEQ ID NO: 315 | F protein sequence for HMPV isolate NL/17/00 |
| SEQ ID NO: 316 | F protein sequence for HMPV isolate NL/1/99 |
| SEQ ID NO: 317 | F protein sequence for HMPV isolate NL/1/94 |
| SEQ ID NO: 318 | F-gene sequence for HMPV isolate NL/1/00 |
| SEQ ID NO: 319 | F-gene sequence for HMPV isolate NL/17/00 |
| SEQ ID NO: 320 | F-gene sequence for HMPV isolate NL/1/99 |
| SEQ ID NO: 321 | F-gene sequence for HMPV isolate NL/1/94 |
| SEQ ID NO: 322 | G protein sequence for HMPV isolate NL/1/00 |
| SEQ ID NO: 323 | G protein sequence for HMPV isolate NL/17/00 |
| SEQ ID NO: 324 | G protein sequence for HMPV isolate NL/1/99 |
| SEQ ID NO: 325 | G protein sequence for HMPV isolate NL/1/94 |
| SEQ ID NO: 326 | G-gene sequence for HMPV isolate NL/1/00 |
| SEQ ID NO: 327 | G-gene sequence for HMPV isolate NL/17/00 |
| SEQ ID NO: 328 | G-gene sequence for HMPV isolate NL/1/99 |
| SEQ ID NO: 329 | G-gene sequence for HMPV isolate NL/1/94 |
| SEQ ID NO: 330 | L protein sequence for HMPV isolate NL/1/00 |
| SEQ ID NO: 331 | L protein sequence for HMPV isolate NL/17/00 |
| SEQ ID NO: 332 | L protein sequence for HMPV isolate NL/1/99 |
| SEQ ID NO: 333 | L protein sequence for HMPV isolate NL/1/94 |
| SEQ ID NO: 334 | L-gene sequence for HMPV isolate NL/1/00 |
| SEQ ID NO: 335 | L-gene sequence for HMPV isolate NL/17/00 |
| SEQ ID NO: 336 | L-gene sequence for HMPV isolate NL/1/99 |
| SEQ ID NO: 337 | L-gene sequence for HMPV isolate NL/1/94 |
| SEQ ID NO: 338 | M2-1 protein sequence for HMPV isolate NL/1/00 |
| SEQ ID NO: 339 | M2-1 protein sequence for HMPV isolate NL/17/00 |
| SEQ ID NO: 340 | M2-1 protein sequence for HMPV isolate NL/1/99 |
| SEQ ID NO: 341 | M2-1 protein sequence for HMPV isolate NL/1/94 |
| SEQ ID NO: 342 | M2-1 gene sequence for HMPV isolate NL/1/00 |
| SEQ ID NO: 343 | M2-1 gene sequence for HMPV isolate NL/17/00 |
| SEQ ID NO: 344 | M2-1 gene sequence for HMPV isolate NL/1/99 |
| SEQ ID NO: 345 | M2-1 gene sequence for HMPV isolate NL/1/94 |
| SEQ ID NO: 346 | M2-2 protein sequence for HMPV isolate NL/1/00 |
| SEQ ID NO: 347 | M2-2 protein sequence for HMPV isolate NL/17/00 |
| SEQ ID NO: 348 | M2-2 protein sequence for HMPV isolate NL/1/99 |
| SEQ ID NO: 349 | M2-2 protein sequence for HMPV isolate NL/1/94 |
| SEQ ID NO: 350 | M2-2 gene sequence for HMPV isolate NL/1/00 |
| SEQ ID NO: 351 | M2-2 gene sequence for HMPV isolate NL/17/00 |
| SEQ ID NO: 352 | M2-2 gene sequence for HMPV isolate NL/1/99 |
| SEQ ID NO: 353 | M2-2 gene sequence for HMPV isolate NL/1/94 |
| SEQ ID NO: 354 | M2 gene sequence for HMPV isolate NL/1/00 |
| SEQ ID NO: 355 | M2 gene sequence for HMPV isolate NL/17/00 |
| SEQ ID NO: 356 | M2 gene sequence for HMPV isolate NL/1/99 |
| SEQ ID NO: 357 | M2 gene sequence for HMPV isolate NL/1/94 |
| SEQ ID NO: 358 | M protein sequence for HMPV isolate NL/1/00 |
| SEQ ID NO: 359 | M protein sequence for HMPV isolate NL/17/00 |
| SEQ ID NO: 360 | M protein sequence for HMPV isolate NL/1/99 |
| SEQ ID NO: 361 | M protein sequence for HMPV isolate NL/1/94 |
| SEQ ID NO: 362 | M gene sequence for HMPV isolate NL/1/00 |
| SEQ ID NO: 363 | M gene sequence for HMPV isolate NL/17/00 |
| SEQ ID NO: 364 | M gene sequence for HMPV isolate NL/1/99 |
| SEQ ID NO: 365 | M gene sequence for HMPV isolate NL/1/94 |
| SEQ ID NO: 366 | N protein sequence for HMPV isolate NL/1/00 |
| SEQ ID NO: 367 | N protein sequence for HMPV isolate NL/17/00 |
| SEQ ID NO: 368 | N protein sequence for HMPV isolate NL/1/99 |
| SEQ ID NO: 369 | N protein sequence for HMPV isolate NL/1/94 |
| SEQ ID NO: 370 | N gene sequence for HMPV isolate NL/1/00 |
| SEQ ID NO: 371 | N gene sequence for HMPV isolate NL/17/00 |
| SEQ ID NO: 372 | N gene sequence for HMPV isolate NL/1/99 |
| SEQ ID NO: 373 | N gene sequence for HMPV isolate NL/1/94 |
| SEQ ID NO: 374 | P protein sequence for HMPV isolate NL/1/00 |
| SEQ ID NO: 375 | P protein sequence for HMPV isolate NL/17/00 |
| SEQ ID NO: 376 | P protein sequence for HMPV isolate NL/1/99 |
| SEQ ID NO: 377 | P protein sequence for HMPV isolate NL/1/94 |
| SEQ ID NO: 378 | P gene sequence for HMPV isolate NL/1/00 |
| SEQ ID NO: 379 | P gene sequence for HMPV isolate NL/17/00 |
| SEQ ID NO: 380 | P gene sequence for HMPV isolate NL/1/99 |
| SEQ ID NO: 381 | P gene sequence for HMPV isolate NL/1/94 |
| SEQ ID NO: 382 | SH protein sequence for HMPV isolate NL/1/00 |
| SEQ ID NO: 383 | SH protein sequence for HMPV isolate NL/17/00 |
| SEQ ID NO: 384 | SH protein sequence for HMPV isolate NL/1/99 |
| SEQ ID NO: 385 | SH protein sequence for HMPV isolate NL/1/94 |
| SEQ ID NO: 386 | SH gene sequence for HMPV isolate NL/1/00 |
| SEQ ID NO: 387 | SH gene sequence for HMPV isolate NL/17/00 |
| SEQ ID NO: 388 | SH gene sequence for HMPV isolate NL/1/99 |
| SEQ ID NO: 389 | SH gene sequence for HMPV isolate NL/1/94 |

What is claimed is:

1. A method for propagating mammalian metapneumovirus, wherein the method comprises culturing the mammalian metapneumovirus in medium with a specific trypsin activity of less than 20 milliunits per milliliter of medium.

2. The method of claim 1, wherein the mammalian metapneumovirus is human metapneumovirus.

3. The method of claim 1 or 2, wherein no trypsin is added exogenously to the medium.

4. The method of claim 1 or 2, wherein no serum is added to the medium.

5. The method of claim 1 or 2, wherein an RQSR cleavage motif in the cleavage site of the F protein of mammalian metapneumovirus comprises at least one amino acid substitution.

6. The method of claim 5, wherein the F protein of mammalian metapneumovirus comprises at least one additional amino acid substitution relative to SEQ ID NO:314.

7. The method of claim 5, wherein the amino acid substitution in the RQSR cleavage motif is a serine to proline substitution resulting in a RQPR sequence.

8. The method of claim 6, wherein the additional amino acid substitution in the F protein is at least one of the following E93K, Q100K, E92K, E93V, I95S, E96K, Q94K, Q94H, I95S, N97K or N97H.

9. The method of claim 8, wherein the additional amino acid substitution in the F protein is E93K.

10. The method of claim 6, wherein the additional amino acid substitution stabilizes the amino acid substitution in the RQSR motif.

11. An isolated mammalian metapneumovirus, wherein the mammalian metapneumovirus is capable of growth in the absence of trypsin.

12. The virus of claim 11, wherein the mammalian metapneumovirus is human metapneumovirus.

13. The virus of claim 11 or 12, wherein an RQSR cleavage motif in the cleavage site of the F protein of mammalian metapneumovirus comprises at least one amino acid substitution.

14. The virus of claim 13, wherein the F protein of mammalian metapneumovirus comprises at least one additional amino acid substitution relative to SEQ ID NO:314.

15. The virus of claim 13, wherein the amino acid substitution in the RQSR cleavage motif is a serine to proline substitution resulting in a RQPR sequence.

16. The virus of claim 14, wherein the additional amino acid substitution in the F protein is at least one of the following E93K, Q100K, E92K, E93V, I95S, E96K, Q94K, Q94H, I95S, N97K or N97H.

17. The virus of claim 14, wherein the additional amino acid substitution stabilizes the amino acid substitution in the RQSR motif.

18. The virus of claim 16, wherein the additional amino acid substitution in the F protein is E93K.

19. An isolated nucleic acid, wherein the isolated nucleic acid encodes an F protein of a mammalian metapneumovirus, wherein the F protein comprises the S101P amino acid substitution and at least one of the following amino acid substitutions E93K, Q100K, E92K, E93V, I95S, E96K, Q94K, Q94H, I95S, N97K or N97H.

20. The nucleic acid of claim 19, wherein the mammalian metapneumovirus is human metapneumovirus.

21. A method for identifying an F protein of a mammalian metapneumovirus that supports stable growth of the mammalian metapneumovirus in the absence of trypsin, the method comprising:
(a) growing the mammalian metapneumovirus in the absence of trypsin for at least two passages, wherein the mammalian metapneumovirus comprises a RQPR motif in the cleavage site of the F protein; and
(b) measuring syncytia formation;
wherein increased syncytia formation relative to syncytia formation by a mammalian metapneumovirus prior to step (a) indicates that the F protein of the mammalian metapneumovirus has acquired an additional amino acid substitution that supports stable growth of the mammalian metapneumovirus in the absence of trypsin.

22. A method for identifying an F protein of a mammalian metapneumovirus that supports stable growth of the mammalian metapneumovirus in the absence of trypsin, the method comprising:
(a) growing the mammalian metapneumovirus in the absence of trypsin for at least two passages, wherein the mammalian metapneumovirus comprises a RQPR motif in the cleavage site of the F protein; and
(b) measuring F protein cleavage;
wherein increased F protein cleavage relative to F protein cleavage by mammalian metapneumovirus prior to step (a) indicates that the F protein of the mammalian metapneumovirus has acquired an additional amino acid substitution that supports stable growth of the mammalian metapneumovirus in the absence of trypsin.

23. A method for identifying an F protein mutant of a mammalian metapneumovirus that enhances trypsin-independent cleavage of the F protein, wherein the F protein comprises a RQPR motif in the cleavage site, said method comprising:
(a) growing the mammalian metapneumovirus in the absence of trypsin for at least two passages; and
(b) determining the cleave efficiency of the F protein,
wherein increased cleavage efficiency of the F protein indicates that the F protein has acquired a mutation that enhances trypsin-independent cleavage of the F protein.

24. A method for identifying a protease that catalyzes the cleavage of an F protein of mammalian metapneumovirus, wherein the F protein comprises a RQPR motif in the cleavage site, said method comprising:
(a) contacting the F protein with a test protease; and
(b) determining whether cleavage of the F protein has occurred;
wherein the occurrence of cleavage of the F protein indicates that the protease catalyzes the cleavage of the F protein.

25. The method of claim 21, 22, 23, or 24, wherein the mammalian metapneumovirus is human metapneumovirus.

26. The method of claim 21, 22, 23, or 24, wherein the mammalian metapneumovirus carries the S101P mutation.
